(19) United States
(12) Patent Application Publication
(10) Pub. No.: US 2023/0365568 A1
(43) Pub. Date: Nov. 16, 2023

(54) PLK1 POLO BOX DOMAIN INHIBITORS AND METHOD OF TREATING CANCER

(71) Applicant: The United States of America,as represented by the Secretary,Department of Health and Human Services, Bethesda, MD (US)

(72) Inventors: Kyung S. Lee, Gaithersburg, MD (US); Kenneth A. Jacobson, Silver Spring, MD (US); Celeste N. Alverez, North Ridgeville, OH (US); Jung-Eun Park, North Bethesda, MD (US); Paola Oliva, Bethesda, MD (US); Hobin Lee, Bethesda, MD (US); Klara Pongorne Kirsch, Bethesda, MD (US)

(73) Assignee: The United States of America,as represented by the Secretary,Department of Health and Human Services, Bethesda, MD (US)

(21) Appl. No.: 18/028,463

(22) PCT Filed: Sep. 24, 2021
(86) PCT No.: PCT/US2021/052054

§ 371 Date: Mar. 24, 2023

Related U.S. Application Data
(60) Provisional application No. 63/082,813, filed on Sep. 24, 2020.

(51) Int. Cl. C07D 487/04; C07D 495/14; C07D 471/14; C07F 9/6561

(52) U.S. Cl. CPC C07D 487/04; C07D 495/14; C07D 471/14; C07F 9/6561

(57) ABSTRACT

Provided is a method of treating cancer, particularly cancers associated with an overexpression of polo-like kinase (Plk1), comprising administering a compound of formula (I) or a pharmaceutically acceptable salt thereof in which ring A, X¹, X², X³, X⁴, X⁵, R², R³, R⁴, n, bond a, and bond b are described herein. Exemplary compounds of formula (I) and pharmaceutically acceptable salts thereof, especially those that selectively inhibit the polo box domain of Plk1, also are provided.

### CROSS-REFERENCE TO A RELATED APPLICATION

This patent application claims the benefit of U.S. Provisional Patent Application No. 63/082,813, filed Sep. 24, 2020, which is incorporated by reference.

### STATEMENT REGARDING FEDERALLY SPONSORED RESEARCH OR DEVELOPMENT

This invention was made with Government support under project number Z01BC010681 by the National Institutes of Health, National Cancer Institute. The Government has certain rights in the invention.

### BACKGROUND OF THE INVENTION

Members of the Polo subfamily of Ser/Thr protein kinases (collectively, polo-like kinases) play a key role in regulating various aspects of the cell cycle and cell proliferation (Zitouni et al., Nat. Rev. Mol. Cell Biol. 2014, 15 (7), 433-452). Among them, polo-like kinase 1 (Plk1) is critically required for proper mitotic progression, whereas other members play distinct roles during interphase progression and exhibit little functional overlap with other Plk family members (Zitouni et al., vide supra; and Lee et al., Trends Pharmacol. Sci. 2015, 36 (12), 858-877). In accordance with its importance in promoting mitosis, Plk1 is largely upregulated in a broad range of human cancers and its level of overexpression appears to correlate with aggressiveness and poor prognosis for a wide spectrum of human cancers (Strebhardt et al., Nat. Rev. Drug Discov. 2010, 9 (8), 643-660; and de Carcer et al., Genes (Basel) 2019, 10 (3), 208-221). Notably, various cancer cells, but not their isogenic normal cells, are addicted to high Plk1 levels and consequently require Plk1 overexpression for their viability (Luo et al., Cell 2009, 137 (5), 835-848; Sur et al., Proc. Natl. Acad. Sci. USA. 2009, 106 (10), 3964-3969; and Park et al., Cell Cycle 2015, 14 (22), 3624-3634). Since the reversal of addicted protein functions in cancer cells has proven to be an attractive strategy to selectively kill cancer cells (Weinstein et al., Science 2002, 297 (5578), 63-64; McMurray rt al., Nature 2008, 453 (7198), 1112-1126; and Luo et al., Cell 2009, 136 (5), 823-837), Plk1 is considered a discriminating target for anticancer therapy.

Plk1 contains an N-terminal kinase domain (KD) for ATP-dependent catalysis and is characterized by the presence of the C-terminal non-catalytic, but functionally essential, polo-box domain (PBD) (Elia et al., Cell 2003, 115 (1), 83-95; and Lee et al., Proc. Natl. Acad. Sci. USA 1998, 95 (16), 9301-9306). The PBD plays a key role in mediating Plk1 functions by targeting its N-terminal catalytic activity to distinct subcellular structures, such as centrosomes, kinetochores, and midbody, through specific protein-protein interactions (PPIs) (Lee et al., 1998, vide supra; and Seong et al., J Biol. Chem. 2002, 277 (35), 32282-32293). For more than a decade, extensive efforts were made to develop Plk1 inhibitors targeting the KD, resulting in several Plk1 ATP-competitive inhibitors, such as Volasertib/BI6727 (Rudolph et al., Clin. Cancer Res. 2009, 15 (9), 3094-3102), BI2536 (Steegmaier et al., Curr. Biol. 2007, 17 (4), 316-322), GSK461364 (Russo et al., Thyroid 2013, 23 (10), 1284-1293), NMS-P937 (Beria et al., Bioorg. Med. Chem. Lett. 2011, 21 (10), 2969-2974), and TAK-960 (Nie et al., Bioorg. Med. Chem. Lett. 2013, 23 (12), 3662-3666) that have been examined against various cancers (Lee, 2015, vide supra). However, they all showed limited efficacy with more-than-acceptable dose-limiting toxicity in various preclinical or clinical trials. Since dose-limiting toxicity arises mainly from non-specific activity of these inhibitors (Karaman et al., Nat. Biotechnol. 2008, 26 (1), 127-132), improving Plk1 specificity is likely the major concern that needs to be addressed to achieve better clinical outcomes. In fact, one of the common problems associated with the currently available Plk1 ATP-competitive inhibitors appears to be their low degree of selectivity against other kinases (Lee et al., Trends Pharmacol. Sci. 2015, 36 (12), 858-877; and Park et al., F1000Res. 2017, 6, 1024), including two closely related Plk2 and Plk3 with potential tumor suppressor roles (Syed et al., Blood 2006, 107 (1), 250-256; and Yang et al., Cancer Res. 2008, 68 (11), 4077-4085).

Thus, there remains an unmet need to provide small molecule compounds that exhibit specific anti-Plk1 PBD activity to be used in a method of treating cancer.

### BRIEF SUMMARY OF THE INVENTION

The invention provides a method of treating cancer comprising administering to a subject in need thereof a compound of formula (I) or a pharmaceutically acceptable salt thereof

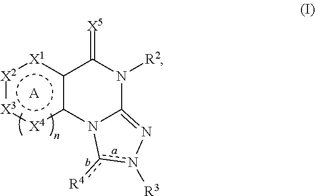

wherein ring A, X¹, X², X³, X⁴, X⁵, R², R³, R⁴, n, bond a, and bond b are described herein.

Also provided is a compound of formula (I) that is of formula (Ib-1) or a pharmaceutically acceptable salt thereof:

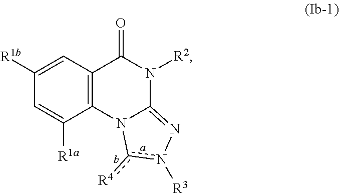

wherein R^{1a}, R^{1b}, R², R³, R⁴, bond a, and bond b are described herein.

Further provided is a compound of formula (I) that is of formula (Ib-2) or a pharmaceutically acceptable salt thereof:

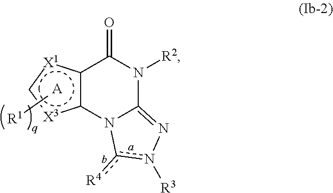

wherein ring A, R¹, R², R³, R⁴, q, bond a, and bond b are described herein.

### BRIEF DESCRIPTION OF THE SEVERAL VIEWS OF THE DRAWINGS

FIG. 1 shows triazoloquinazolinone 7, a substituted derivative thereof 8, and the delineation of zones of a structure activity relationship (SAR) 9 in accordance with an aspect of the invention.

FIG. 2 depicts Scheme 1 showing a general synthesis method of 7-fluoro analogs in accordance with an aspect of the invention.

FIG. 3 depicts Scheme 2 showing a synthesis of zone 4 amides in accordance with an aspect of the invention.

FIG. 4 demonstrates the antiproliferative effect of 143 in HeLa cells. Asynchronously growing HeLa cells were treated with 100 μM of compound 143 for 12 h, subjected to phase-contrast micrography, and stained with DAPI to visualize chromosome morphologies, and then quantified. FIG. 4A is a bar graph showing the percentage of mitotic cells or cells with apoptotic or aberrant chromosome morphologies quantified from three independent experiments (n>515 cells/sample/experiment). Bars, mean±s.d., ***, P<0.001 (unpaired two-tailed t-test). FIG. 4B is a bar graph showing the relative number of asynchronously growing HeLa cells treated with either dimethylsulfoxide (DMSO) or 100 μM of compound 143 for 0, 36, or 72 hours. Cell numbers at the indicated time points were quantified from three independent experiments (n=an average of 2,547 cells/experiment for DMSO and 1,673 cells/experiment for 143). Bars, mean±s.d. For FIGS. 4C and 4D, cells from FIG. 4A were immunostained with the indicated antibodies to reveal delocalized Plk1 signals from centrosomes (marker: Cep63) and kinetochores (marker: CREST) (FIG. 4C) and abnormal spindle morphologies (marker: α-tubulin) (FIG. 4D). Quantification was carried out from three independent experiments: ≥20 (DMSO) or 32 (143) centrosomes/sample/experiment and ≥42 (DMSO) or 64 (143) kinetochores (˜4 kinetochores per cell)/sample/experiment) for FIG. 4C and ≥106 (DMSO) or 102 (143) cells/sample/experiment for FIG. 4D. Bars, mean±s.d., ***, P<0.001 (unpaired two-tailed t-test).

FIG. 5A depicts structures for phosphopeptides: PLHSpT (6a) and the nonphosphorylated form of PLHST (6b). FIGS. 5B-5D depict comparative FP-based assays. Nonphosphorylated peptide 6b, exhibits no detectable activity (FIG. 5B). PLHSpT 6a (prior art) shows the IC₅₀ of 22 μM with preferential inhibition of the PBD of Plk 1 (FIG. 5C). Under the same experimental conditions, compound 79 potently inhibits the PBD of Plk1 but not Plk2 and Plk3 with an IC₅₀ of 0.47 μM (FIG. 5D).

FIG. 6 show the zones of modification of the 1-thioxo-2,4-dihydro-[1,2,4]triazolo[4,3-a]quinazolin-5(1H)-one scaffold, as shown for compound 4, in a structure activity relationship (SAR) in a further aspect of the invention.

FIG. 7 depicts Scheme 3 showing the synthesis of derivatives containing substituted N-alkyl groups.

FIG. 8 depicts Scheme 4 showing the synthesis of phenylacetyl amide derivatives.

FIG. 9 depicts Scheme 5 showing the synthesis of representative S-alkyl and disulfide prodrugs, designed to penetrate cancer cells and lead to liberation of the thio-containing active drug.

FIG. 10 depicts Scheme 6 showing the synthesis of 1-methyl-4-substituted imidazole 5-thioether derivatives, prepared as cell-penetrant prodrugs.

FIGS. 11A-11I is a table of compounds of formula (I).

FIG. 12 shows representative inhibition curves obtained from ELISA assays. For comparative analyses, a previously characterized phosphopeptide, PLHSpT 2 (K_{d} of ˜450 nM) was included as a positive control.

FIGS. 13A-13F are PBD inhibition curves obtained from FP-based assays (● Plk1 PBD; ▴ Plk2 PBD; and ▪ Plk3 PBD). FIG. 13A is for compound 3 (PLHST); FIG. 13B is for compound 2 (PLHSpT); FIG. 13C is for compound 43 (NCK 149); FIG. 13D is for compound 45 (NCK 158); FIG. 13E is for compound 46 (NCK 165); FIG. 13F is for compound 49e (NCK 181). All the data are quantified from three independent experiments. Bars: mean±standard deviation.

FIG. 14 is a line graph of an MTS assay used to determine the effect of the indicated prodrugs (75, 81, 82, and 85) on the proliferation of multiple myeloma-derived L363 cells. The assays were carried out two days after treating the cells with different concentrations of the compounds. Corresponding parental nonprodrugs, 43, 45, 46, and 49e were included as negative controls. All the data are quantified from three independent experiments. Bars: mean±standard deviation.

FIGS. 15A-15C show the oral pharmacokinetic analyses for prodrug 73 (NCK167) and its metabolites (FIG. 15A), parent drug 14 (9-fluoro-N-propyl) (NCK103) (FIG. 15B), and glucuronidated 14 (14-glu) (FIG. 15C) in mice. The marker ● represents a dose of 50 mg/kg, and ▪ represents a dose of 20 mg/kg.

FIGS. 16A-16B shows the inhibitory effects of CYP-specific inhibitors on metabolism of N-ethyl-9-fluoro 60 (NCK139) derivative (FIG. 16A) to 14 (NCK103) (FIG. 16B) in mouse liver microsomes. The following markers were used: ▪ Control; ● ketoconazole; ▴ sulphaphenazole; and ▾ furafylline.

FIGS. 17A-17C show the intraperitoneal (i.p.) pharmacokinetic analyses for prodrug 85 (NCK182) and its metabolites (FIG. 17A), parent drug 49e (NCK181) (FIG. 17B), and glucuronidated 49e (49e-glu) (FIG. 17C), in mice following i.p. injection of 85 at a dose of 15 mg/kg.

### DETAILED DESCRIPTION OF THE INVENTION

In an aspect, the invention provides a method of treating cancer comprising administering to a subject in need thereof a compound of formula (I) or a pharmaceutically acceptable salt thereof

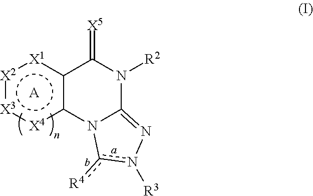

wherein
ring A is phenyl, a 5-membered heteroaryl, or a 6-membered heteroaryl;
X¹, X², X³, and X⁴ are the same or different and each is CR¹, N, S, or O, wherein no more than three of X¹, X², X³, and X⁴ are N, S, or O;
n is 0 or 1; provided that when n is 0, at least one of X¹, X², X³, and X⁴ is N, S, or O;
X⁵ is O or S;
each instance of R¹ is the same or different and each is selected from the group consisting of H, deuterium, C₁₋₆ alkyl (including deuterated alkyl), alkenyl, alkynyl, cycloalkyl, hydroxy, alkoxy, cycloalkoxy, halo, haloalkyl, alkylthio (—S-alkyl), alkylthioalkylenyl (-alkylene-S-alkyl), cyano, amino, alkylamino, dialkylamino, amido, aryl, and heterocycloalkyl or
more than one instance of R¹ are linked to form a cycloalkyl or a phenyl, each of which is optionally substituted;
R² is selected from the group consisting of H, alkyl, cycloalkyl, alkenyl, and aryl;
wherein the alkyl, cycloalkyl, alkenyl, and aryl of R² is optionally substituted with one or more substituents selected from the group consisting of deuterium, alkyl, alkoxy, halo, hydroxy, haloalkyl, alkoxy, alkyl, cycloalkyl, heterocycloalkyl, aryl, heteroaryl, amino, alkylamino, dialkylamino, amido, alkylsulfonamido, phosphonato, cyano, thiocyano, carboxylate, a protecting group, an amino acid residue, and a peptide residue,
R³ is selected from the group consisting of H, C₁₋₁₀ alkyl, cycloalkyl, aminoalkyl, alkylaminoalkyl, dialkylaminoalkyl, and arylcarbonylalkyl or R³ is absent;
bond a is a single bond or double bond;
bond b is a single bond or double bond;
provided when bond a is a single bond, then R³ is present, bond b is a double bond, and R⁴ is S, and
when bond a is a double bond, then R³ is absent, bond b is a single bond and R⁴ is H, alkyl, hydroxy, amino, or S—R⁵,
R⁵ is selected from the group consisting of C₁₋₁₀ alkyl, cycloalkyl, acetyl, and optionally substituted 4-imidazolyl of the structure

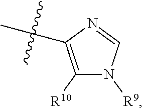

wherein R⁹ is H or alkyl, and R¹⁰ is H, alkyl, halo, haloalkyl, nitro, or sulfonamido (—SO₂NH₂); and
wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl,
provided that
when ring A is phenyl, X¹, X², X³, and X⁴ are each CR¹, X⁵ is O, R² is n-propyl, bond a is a single bond, bond b is a double bond, R³ is hydrogen, and R⁴ is S, then R¹ is not alkylamido (—C(O)NHalkyl);
when ring A is phenyl, X¹, X², X³, and X⁴ are each CH, X⁵ is O, bond a is a double bond, bond b is a single bond, and R³ is absent, then R⁴ is not H or alkyl;
when ring A is phenyl, X¹, X², X³, and X⁴ are each CR¹, X⁵ is O, bond a is a double bond, bond b is a single bond, and R³ is absent, then R⁴ is not alkyl;
when ring A is phenyl, X¹, X², and X⁴ are each CH, X³ is CR¹, n is 1, X⁵ is O, R² is alkyl, bond a is a single bond, bond b is a double bond, R³ is hydrogen, and R⁴ is S, then R¹ at the X³ position is not halo or hydrogen;
when ring A is phenyl, X¹, X³, and X⁴ are each CH, X² is CCH₃, n is 1, X⁵ is O, bond a is a single bond, bond b is a double bond, R³ is hydrogen, and R⁴ is S, then R² is not aryl;
when ring A is phenyl, X¹, X³, and X⁴ are each CH, X² is C(halo), n is 1, X⁵ is O, bond a is a double bond, bond b is a single bond, R² is phenyl, R³ is absent, and R⁴ is SR⁵, then R⁵ is not alkyl;
when ring A is thiophenyl, X¹ is S, X² and X³ are both CR¹, n is O, X⁵ is O, bond a is a double bond, bond b is a single bond, R³ is absent, R⁴ is SR⁵, and R⁵ is alkyl, then R² is not aryl; and
when ring A is thiophenyl, X¹ is S, X² and X³ are both CH, n is 0, X⁵ is O, bond a is a single bond, bond b is a double bond, R³ is hydrogen, and R⁴ is S, then R² is not n-butyl, benzyl, or —CH₂-Ph-(4-ethyl).

In some aspects of formula (I) or a pharmaceutically acceptable salt thereof, bond a is a single bond, R³ is present, bond b is a double bond, and R⁴ is S or a pharmaceutically acceptable salt thereof.

In some aspects of formula (I) or a pharmaceutically acceptable salt thereof, bond a is a double bond, R³ is absent, bond b is a single bond, and R⁴ is H, alkyl, hydroxy, amino, or S—R⁵. In such aspects, R⁴ preferably is S—R⁵. In some aspects of these aspects, R⁵ is C₁₋₆ alkyl, cycloalkyl, arylalkyl, or optionally substituted 4-imidazolyl of the structure

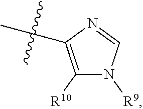

wherein R⁹ is H or alkyl, and R¹⁰ is H, alkyl, halo, haloalkyl, nitro, or sulfonamido (—SO₂NH₂). In a preferred aspect, R⁵ is C₁₋₆ alkyl, especially methyl, ethyl, n-propyl, or n-butyl, or alkyl substituted with cyclopropyl, cyclobutyl, hydroxy, cyano, alkylthio (e.g., —S-alkyl, such as —SEt), amino, amido, phenyl, or 4-Cl-phenyl. In another preferred aspect, R⁵ is substituted 4-imidazolyl of the structure

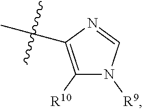

wherein R⁹ is methyl, and R¹⁰ is haloalkyl (e.g., —CF₃), nitro, or sulfonamido (—SO₂NH₂).

In some aspects of formula (I) or a pharmaceutically acceptable salt thereof, n is 1 and each of X¹, X², X³, and X⁴ is CR¹. In certain aspects of these aspects, one instance of R¹ is halo (e.g., F or Cl) or H, and the remaining three instances of R¹ are each H. As a specific example, X¹, X², and X³ are each CH and X⁴ is C—F or C—Cl.

In some aspects of formula (I) or a pharmaceutically acceptable salt thereof, n is 1, one of X¹, X², X³, and X⁴ is N, and the remaining three of X¹, X², X³, and X⁴ are each CR¹. In certain aspects of these aspects, each instance of R¹ is H.

In some aspects of formula (I) or a pharmaceutically acceptable salt thereof, n is 0, and either X¹ or X³ is O or S and the remainder of X¹, X², and X³ is CR¹.

In some aspects of formula (I) or a pharmaceutically acceptable salt thereof, n is 0, and either X¹ and X² or X² and X³ are both N and the remainder of X¹ and X³ is CR¹.

In some aspects of formula (I) or a pharmaceutically acceptable salt thereof, n is 0, and X¹ is N, X² is CR¹, and X³ is O or S.

In some aspects of formula (I) or a pharmaceutically acceptable salt thereof, n is 0, and X¹ and X³ are both N, X² is O.

In any of the aspects of formula (I), ring A is phenyl, a 5-membered heteroaryl, or a 6-membered heteroaryl. In particular, the 5- or 6-membered heteroaryl group can be pyridinyl, pyridazinyl, pyrimidyl, pyrazinyl, triazinyl, imidazolyl, 1,2,3-triazolyl, pyrazolyl, furyl, pyrrolyl, thienyl, isothiazolyl, thiazolyl, isoxazolyl, or oxadiazolyl. Specific examples of ring A of formula (I) or a pharmaceutically acceptable salt thereof include

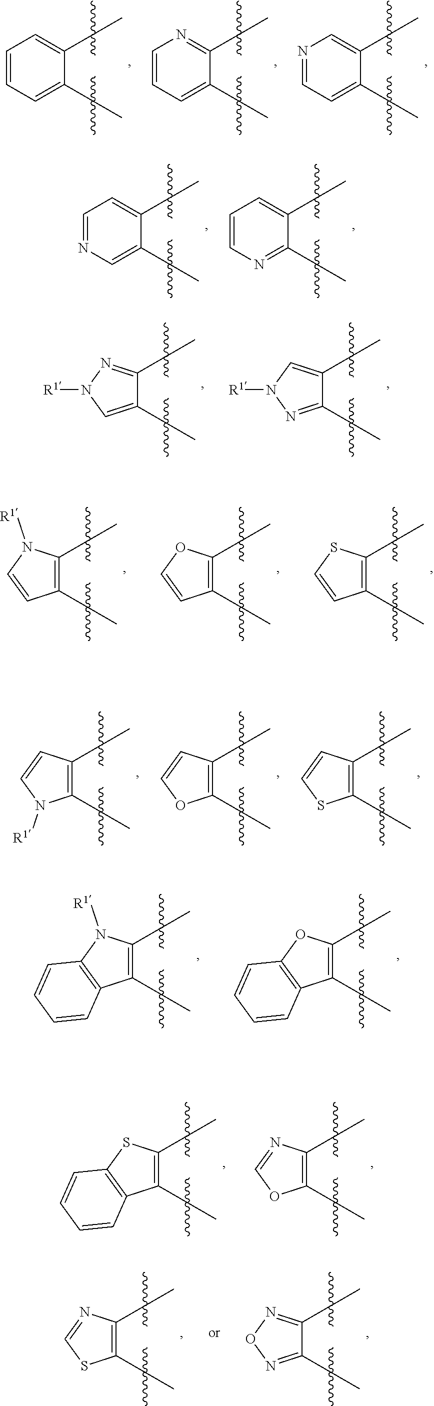

wherein each carbon is optionally substituted with R¹, and R^{1′} is H or alkyl.

In any of the aspects of ring A, one or more (e.g., 1, 2, or 3) non-hydrogen R¹ groups are present on the carbon atoms in the ring. Each instance of R¹ is the same or different and is selected from the group consisting of deuterium, C₁₋₆ alkyl (including deuterated alkyl), alkenyl, alkynyl, cycloalkyl, hydroxy, alkoxy, cycloalkoxy, halo, haloalkyl, alkylthio (—S-alkyl), alkylthioalkylenyl (-alkylene-S-alkyl), cyano, amino, alkylamino, dialkylamino, amido, and heterocycloalkyl. In some preferred aspects, R¹ is F, Cl, haloalkyl (e.g., CF₃), cyano, or alkylthio (—S-alkyl). More preferably, R¹ is F. Alternatively, two instances of R¹ are linked to form a cycloalkyl or a phenyl, each of which is optionally substituted, thereby forming a bicyclic compound. For example, two R¹ moieties can be linked to form phenyl that is optionally substituted with another R¹ group as defined herein (e.g., alkyl or halo (e.g., F, Cl)).

In some aspects, the compound of formula (I) is a triazoloquinazolinone compound or a pharmaceutically acceptable salt of formula (Ia-1):

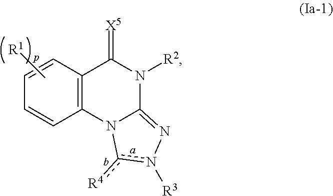

wherein
X⁵ is O or S;
each instance of R¹ is the same or different and each is selected from the group consisting of H, deuterium, C₁₋₆ alkyl (including deuterated alkyl), alkenyl, alkynyl, cycloalkyl, hydroxy, alkoxy, cycloalkoxy, halo, haloalkyl, alkylthio (—S-alkyl), alkylthioalkylenyl (-alkylene-S-alkyl), cyano, amino, alkylamino, dialkylamino, amido, and heterocycloalkyl or
more than one instance of R¹ are linked to form a cycloalkyl or a phenyl, each of which is optionally substituted;
p is an integer of 1-4;
R² is selected from the group consisting of H, alkyl, cycloalkyl, alkenyl, and aryl;
wherein the alkyl, cycloalkyl, alkenyl, and aryl of R² is optionally substituted with one or more substituents selected from the group consisting of deuterium, alkyl, alkoxy, halo, hydroxy, haloalkyl, alkoxy, alkyl, cycloalkyl, heterocycloalkyl, aryl, heteroaryl, amino, alkylamino, dialkylamino, amido, alkylsulfonamido, phosphonato, cyano, thiocyano, carboxylate, a protecting group, an amino acid residue, and a peptide residue,
R³ is selected from the group consisting of H, C₁₋₁₀ alkyl, cycloalkyl, aminoalkyl, alkylaminoalkyl, dialkylaminoalkyl, and arylcarbonylalkyl or R³ is absent;
bond a is a single bond or double bond;
bond b is a single bond or double bond;
provided when bond a is a single bond, then R³ is present, bond b is a double bond, and R⁴ is S, and
when bond a is a double bond, then R³ is absent, bond b is a single bond, and R⁴ is H, alkyl, hydroxy, amino, or S—R⁵;
R⁵ is selected from the group consisting of C₁₋₁₀ alkyl, cycloalkyl, acetyl, and optionally substituted 4-imidazolyl of the structure

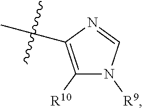

wherein R⁹ is H or alkyl, and R¹⁰ is H, alkyl, halo, haloalkyl, nitro, or sulfonamido (—SO₂NH₂); and
wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl
provided that
when ring A is phenyl, X¹, X², X³, and X⁴ are each CR¹, X⁵ is O, R² is n-propyl, bond a is a single bond, bond b is a double bond, R³ is hydrogen, and R⁴ is S, then R¹ is not alkylamido (—C(O)NHalkyl);
when ring A is phenyl, X¹, X², X³, and X⁴ are each CH, X⁵ is O, bond a is a double bond, bond b is a single bond, and R³ is absent, then R⁴ is not H or alkyl;
when ring A is phenyl, X¹, X², X³, and X⁴ are each CR¹, X⁵ is O, bond a is a double bond, bond b is a single bond, and R³ is absent, then R⁴ is not alkyl;
when ring A is phenyl, X¹, X², and X⁴ are each CH, X³ is CR¹, n is 1, X⁵ is O, R² is alkyl, bond a is a single bond, bond b is a double bond, R³ is hydrogen, and R⁴ is S, then R¹ at the X³ position is not halo or hydrogen;
when ring A is phenyl, X¹, X³, and X⁴ are each CH, X² is CCH₃, n is 1, X⁵ is O, bond a is a single bond, bond b is a double bond, R³ is hydrogen, and R⁴ is S, then R² is not aryl; and
when ring A is phenyl, X¹, X³, and X⁴ are each CH, X² is C(halo), n is 1, X⁵ is O, bond a is a double bond, bond b is a single bond, R² is phenyl, R³ is absent, and R⁴ is SR⁵, then R is not alkyl.

In some aspects of the inventive method, the compound of formula (I) is a triazoloquinazolinone compound or a pharmaceutically acceptable salt of formula (Ia-2):

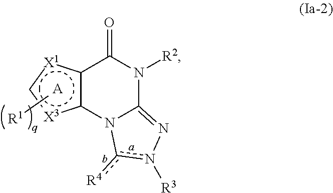

wherein
ring A is a 5-membered heteroaryl;
X¹ and X³ are each CR¹, NR^{1′}, S, or O, provided that at least one of X¹ and X³ is NR^{1′}, S, or O and the other is CR¹;
each instance of R¹ is the same or different and each is selected from the group consisting of H, deuterium, C₁₋₆ alkyl (including deuterated alkyl), alkoxy, halo, haloalkyl, alkylthio (—S-alkyl), alkylthioalkylenyl (-alkylene-S-alkyl), cyano, amino, alkylamino, and dialkylamino, or
more than one instance of R¹ are linked to form a cycloalkyl or a phenyl, each of which is optionally substituted;
q is 1 or 2;
R^{1′} is H or alkyl;
R² is selected from the group consisting of H, alkyl, cycloalkyl, alkenyl, and aryl;
wherein the alkyl, cycloalkyl, alkenyl, and aryl of R² is optionally substituted with one or more substituents selected from the group consisting of deuterium, alkyl, alkoxy, halo, hydroxy, haloalkyl, alkoxy, alkyl, cycloalkyl, heterocycloalkyl, aryl, heteroaryl, amino, alkylamino, dialkylamino, amido, alkylsulfonamido, phosphonato, cyano, thiocyano, carboxylate, a protecting group, an amino acid residue, and a peptide residue,
R³ is H or R³ is absent;
bond a is a single bond or double bond;
bond b is a single bond or double bond;
provided when bond a is a single bond, then R³ is present, bond b is a double bond, and R⁴ is S, and
when bond a is a double bond, then R³ is absent, bond b is a single bond, and R⁴ is S—R⁵;
R⁵ is selected from the group consisting of C₁₋₁₀ alkyl, cycloalkyl, acetyl, and optionally substituted 4-imidazolyl of the structure

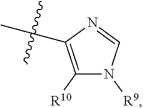

wherein R⁹ is H or alkyl, and R¹⁰ is H, alkyl, halo, haloalkyl, nitro, or sulfonamido (—SO₂NH₂); and
wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl
provided that
when ring A is thiophenyl, X¹ is S, X³ is CR¹, bond a is a double bond, bond b is a single bond, R³ is absent, R⁴ is SR⁵, and R⁵ is alkyl, then R² is not aryl; and
when ring A is thiophenyl, X¹ is S, X³ is CR¹, R¹ is H, bond a is a single bond, bond b is a double bond, R³ is hydrogen, and R⁴ is S, then R² is not n-butyl, benzyl, or —CH₂-Ph-(4-ethyl).

In the compound of formula (Ia-2), ring A, X¹, and X³ form a 5-membered heteroaryl of the structure:

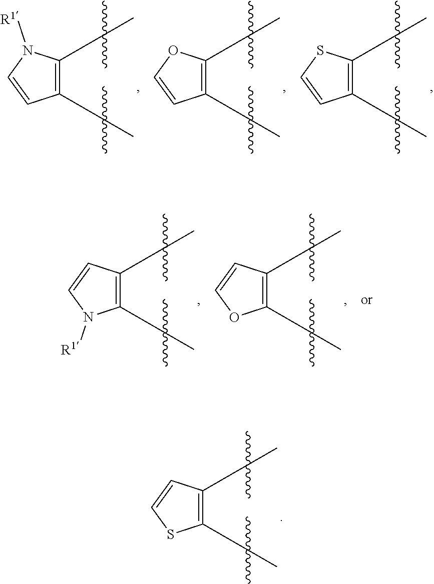

In the compound of formula (Ia-2), two instances of R¹ can be linked to form a cycloalkyl or a phenyl, each of which is optionally substituted, thereby forming a bicyclic compound. For example, two R¹ moieties can be linked to form phenyl that is optionally substituted with another R¹ group as defined herein (e.g., alkyl or halo (e.g., F, Cl)).

Exemplary compounds of formula (Ia-2) include:

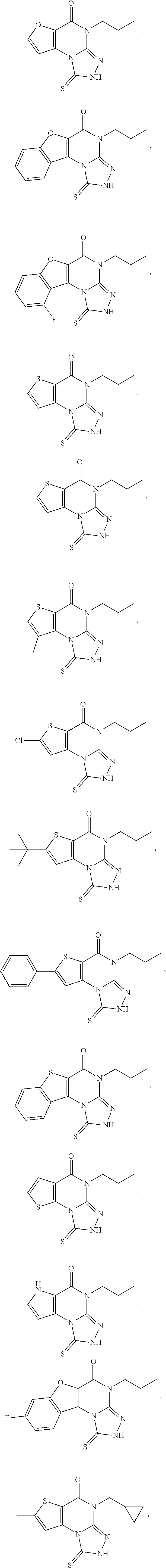

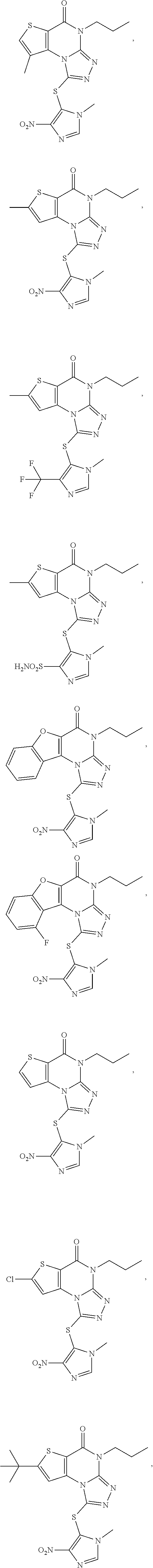

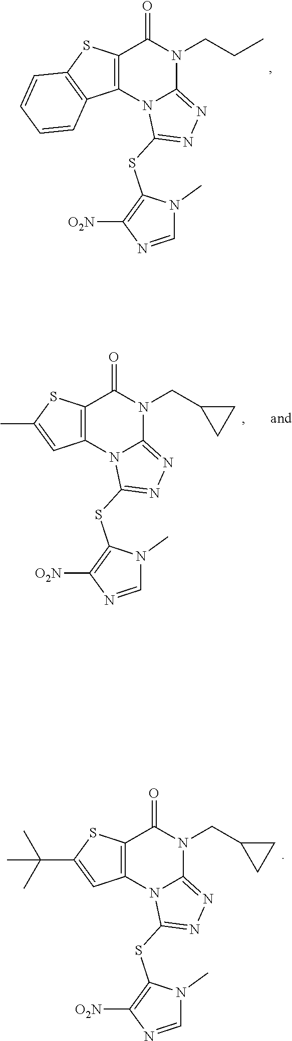

In some aspects, the compound of formula (Ia-2) or a pharmaceutically acceptable salt thereof is a compound of formula (Ia-3):

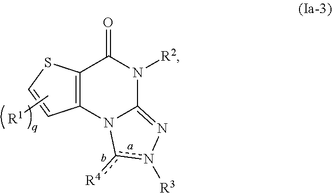

wherein
each instance of R¹ is the same or different and each is selected from the group consisting of H, deuterium, C₁₋₆ alkyl (including deuterated alkyl), alkenyl, alkynyl, cycloalkyl, hydroxy, alkoxy, cycloalkoxy, halo, haloalkyl, alkylthio (—S-alkyl), alkylthioalkylenyl (-alkylene-S-alkyl), cyano, amino, alkylamino, dialkylamino, amido, aryl, and heterocycloalkyl or
two instances of R¹ are linked to form phenyl that is optionally substituted;
q is 1 or 2;
R² is selected from the group consisting of H, alkyl, cycloalkyl, alkenyl, and aryl;
wherein the alkyl, cycloalkyl, alkenyl, and aryl of R² is optionally substituted with one or more substituents selected from the group consisting of deuterium, alkyl, alkoxy, halo, hydroxy, haloalkyl, alkoxy, alkyl, cycloalkyl, heterocycloalkyl, aryl, heteroaryl, amino, alkylamino, dialkylamino, amido, alkylsulfonamido, phosphonato, cyano, thiocyano, carboxylate, a protecting group, an amino acid residue, and a peptide residue,
R³ is selected from the group consisting of H, C₁₋₁₀ alkyl, cycloalkyl, aminoalkyl, alkylaminoalkyl, dialkylaminoalkyl, and arylcarbonylalkyl or R³ is absent;
bond a is a single bond or double bond;
bond b is a single bond or double bond;
provided when bond a is a single bond, then R³ is present, bond b is a double bond, and R⁴ is S, and
when bond a is a double bond, then R³ is absent, bond b is a single bond, and R⁴ is H, alkyl, hydroxy, amino, or S—R⁵;
R⁵ is selected from the group consisting of C₁₋₁₀ alkyl, cycloalkyl, acetyl, and optionally substituted 4-imidazolyl of the structure

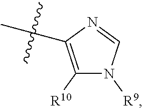

wherein R⁹ is H or alkyl, and R¹⁰ is H, alkyl, halo, haloalkyl, nitro, or sulfonamido (—SO₂NH₂); and
wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl,
provided that
when bond a is a double bond, bond b is a single bond, R³ is absent, R⁴ is SR⁵, and R⁵ is alkyl, then R² is not aryl; and
when R¹ is H, bond a is a single bond, bond b is a double bond, R³ is hydrogen, and R⁴ is S, then R² is not n-butyl, benzyl, or —CH₂-Ph-(4-ethyl).

In some aspects of the compound of formula (Ia-3), R¹ is 5-methyl, p is 1, R² is —(CH₂)₂-cyclopropyl, and either (i) bond a is double, bond b is single, R³ is absent, R⁴ is —SR⁵, and R⁵ is substituted 4-imidazolyl of the structure

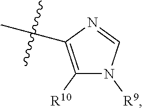

wherein R⁹ is methyl, and R¹⁰ is haloalkyl (e.g., —CF₃), nitro, or sulfonamido (—SO₂NH₂), or (ii) bond a is single, bond b is double, R³ is H, and R⁴ is S. In a preferred aspect, the compound of formula (Ia-3) is selected from

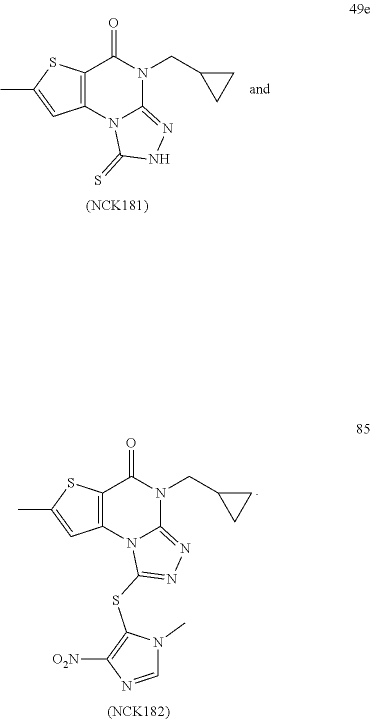 [SMILES: Cc1cc2c(s1)c(=O)n(CC1CC1)c1n[nH]c(=S)n21 ; Cc1cc2c(s1)c(=O)n(CC1CC1)c1nnc(Sc3c([N+](=O)[O-])ncn3C)n21 ; "(NCK181)" ; "(NCK182)"]

In some aspects, the compound of formula (Ia-3) is 85 (NCK182).

In any of the foregoing aspects of formula (I) or a pharmaceutically acceptable salt thereof, X⁵ preferably is O.

In any of the foregoing aspects of formula (I) or a pharmaceutically acceptable salt thereof, R² can be (i) phenyl optionally substituted with alkyl, (ii) allyl, or (iii) alkyl optionally substituted with hydroxy, alkoxy, haloalkyl, cycloalkyl, heterocycloalkyl, aryl, heteroaryl, alkylsulfonamido, amino, or amido of the formula —NHC(O)(CH₂)ₘR⁶, in which m is 0-5, and R⁶ is alkyl, cycloalkyl, heterocycloalkyl, phenyl, and heteroaryl, each of which is optionally substituted with alkyl, alkoxy, hydroxy, halo, haloalkyl, cyano, alkoxycarbonyl (—C(O)O-alkyl), thiocyanato (—NCS), heteroarylalkyl, or a combination thereof, or a pharmaceutically acceptable salt thereof. In certain aspects of these aspects, m is 0. In other aspects of these aspects, m is 1, 2, or 3. In a preferred aspect, R² is —(CH₂)₂—NHC(O)-Ph-R⁸, wherein R⁸ is at the 2-, 3-, or 4-position on the phenyl and is alkyl, alkoxy, halo, hydroxy, cyano, alkoxycarbonyl (—C(O)O-alkyl), thiocyanato (—NCS), or 4-morpholinylmethyl.

In preferred aspects of formula (I) or a pharmaceutically acceptable salt thereof, R² is ethyl, n-propyl, n-butyl, —(CH₂)₃OMe, —(CH₂)₃OH, —(CH₂)₂-cyclopropyl, —(CH₂)₂-cyclobutyl, —(CH₂)₂-(4-pyridinyl), —(CH₂)₂—CF₃, —(CH₂)₂—CHF₂, —CH₂—CF₂(Me), —CH₂—CF₂CF₃, —(CH₂)₂-1-morpholino, —(CH₂)₂—NH₂, —(CH₂)₃—NHSO₂CH₃, —(CH₂)₃—NHC(O)cyclopentyl, —(CH₂)₃—NHC(O)CH₂Ph-(2-Cl), —(CH₂)₃—NHC(O)CH₂Ph-(3-OMe), —(CH₂)₃—NHC(O)CH₂Ph-(2-OMe), —(CH₂)₂—NHC(O)-Ph-(4-OH), —(CH₂)₂—NHC(O)-Ph-(3-OMe), —(CH₂)₂—NHC(O)-Ph-(4-CN), —(CH₂)₂—NHC(O)-Ph-(4-I), —(CH₂)₂—NHC(O)-Ph-(C(O)OMe), —(CH₂)₂—NHC(O)-Ph-(NCS), or —(CH₂)₂—NHC(O)-Ph-(4-morpholinylmethyl).

In any of the foregoing aspects of formula (I) or a pharmaceutically acceptable salt thereof, R³ is present and is H or dialkylaminoalkyl. Preferably, R³ is H.

Further exemplary compounds of formula (I) and pharmaceutically acceptable salts thereof for the inventive method include:

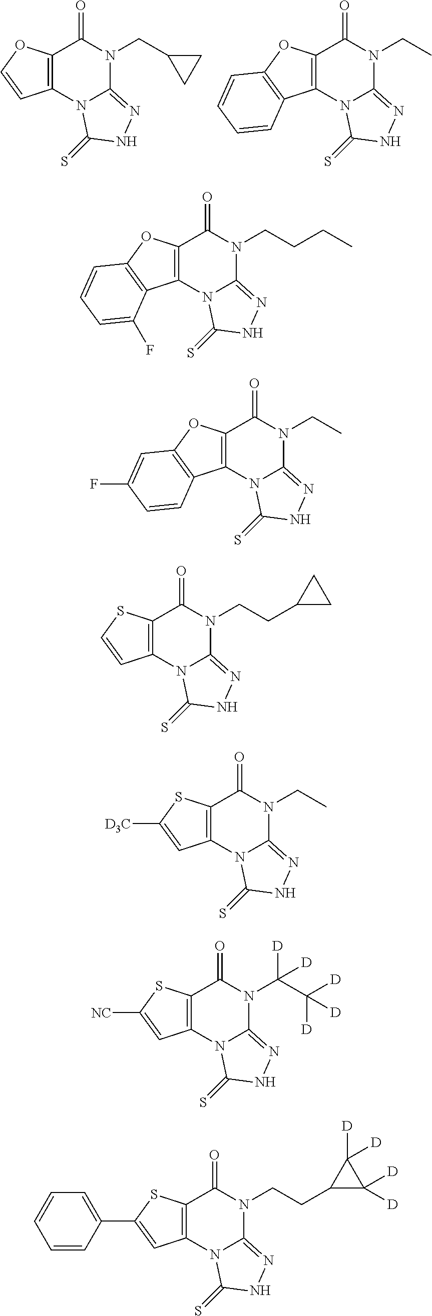

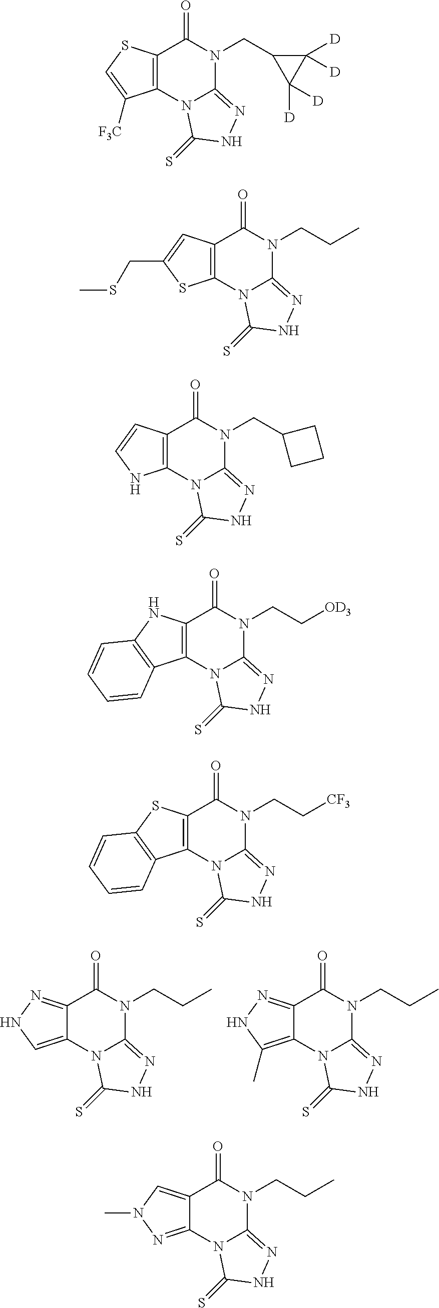

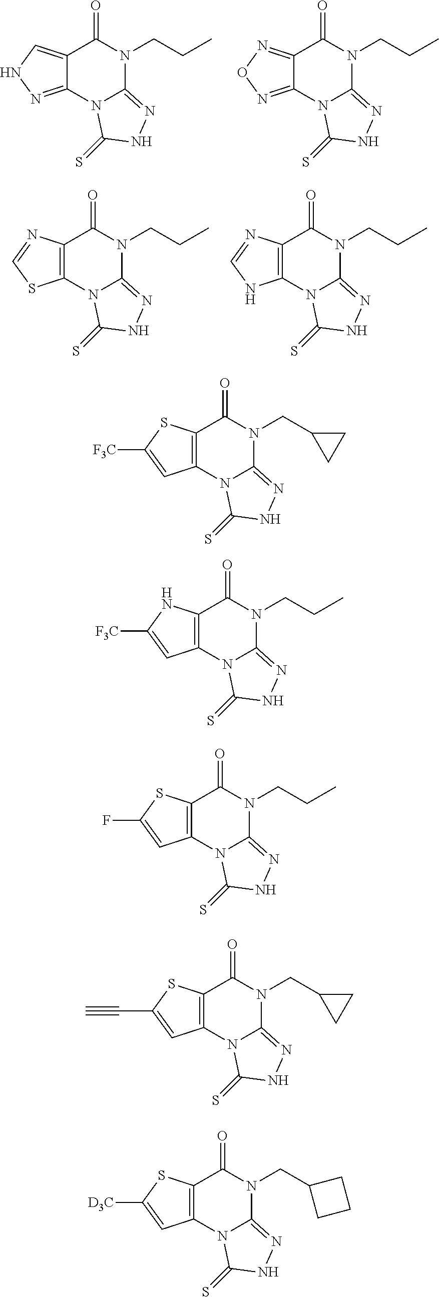

or a prodrug thereof, in which the sulfur moiety (═S) (i.e., R⁴) in the foregoing compounds is replaced with —SR⁵, wherein in the prodrug of each of the foregoing compounds.
bond a is a double bond,
R³ is absent, bond b is a single bond, and
R⁵ is selected from the group consisting of C₁₋₁₀ alkyl, cycloalkyl, acetyl, and optionally substituted 4-imidazolyl of the structure

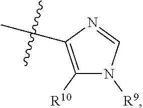

R⁹ is H or alkyl, and R¹⁰ is H, alkyl, halo, haloalkyl, nitro, or sulfonamido (—SO₂NH₂); and
wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl.

In some aspects, the following compounds of formula (I) can be used in a method of treating Plk1-mediated cancer in a subject in need thereof (e.g., a subject with cancer cells that overexpress Plk1 relative to normal tissue of the same type). The method comprises administering to the subject in need a compound of formula (I) selected from the group
ring A is phenyl, X¹, X², X³, and X⁴ are each CR¹, one R¹ is alkylamido (—C(O)NHalkyl) and the rest are H, X⁵ is O, R² is n-propyl, bond a is a single bond, bond b is a double bond, R³ is hydrogen, and R⁴ is S;
ring A is phenyl, X¹, X², X³, and X⁴ are each CH, X⁵ is O, bond a is a double bond, bond b is a single bond, R³ is absent, and R⁴ is H or alkyl;
ring A is phenyl, X¹, X², X³, and X⁴ are each CR¹, X⁵ is O, bond a is a double bond, bond b is a single bond, R³ is absent, and R⁴ is alkyl;
ring A is phenyl, X¹, X², and X⁴ are each CH, X³ is CR¹, R¹ at the X³ position is halo or hydrogen; n is 1, X⁵ is O, R² is alkyl, bond a is a single bond, bond b is a double bond, R³ is hydrogen, and R⁴ is S;
ring A is phenyl, X¹, X³, and X⁴ are each CH, X² is CCH₃, n is 1, X⁵ is O, bond a is a single bond, bond b is a double bond, R² is aryl, R³ is hydrogen, and R⁴ is S;
ring A is phenyl, X¹, X³, and X⁴ are each CH, X² is C(halo), n is 1, X⁵ is O, bond a is a double bond, bond b is a single bond, R² is phenyl, R³ is absent, R⁴ is SR⁵, and R⁵ is alkyl;
ring A is thiophenyl, X¹ is S, X² and X³ are both CR¹, n is 0, X⁵ is O, bond a is a double bond, bond b is a single bond, R² is aryl, R³ is absent, R⁴ is SR⁵, and R⁵ is alkyl; and
ring A is thiophenyl, X¹ is S, X² and X³ are both CH, n is 0, X⁵ is O, bond a is a single bond, bond b is a double bond, R² is n-butyl, benzyl, or —CH₂-Ph-(4-ethyl), R³ is hydrogen, and R⁴ is S.

In some aspects, the invention provides a compound of formula (I) that is of formula (Ib-1) or a pharmaceutically acceptable salt thereof.

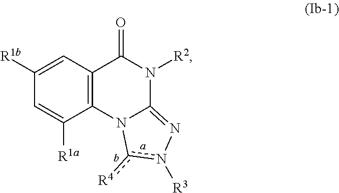

wherein
R^{1a} is H, F, deuterium, or hydroxy;
R^{1b} is H, deuterium, or alkoxy;
R² is selected from the group consisting of alkyl, haloalkyl, cyclopropylalkyl, 2- or 4-pyridinylalkyl, or optionally substituted benzylamidoalkyl, each of which is optionally substituted with deuterium;
R³ is H or absent;
R⁴ is S or S—R⁵,
R⁵ is selected from the group consisting of C₁₋₁₀ alkyl, alkylthio (—S-alkyl), or optionally substituted 4-imidazolyl of the structure

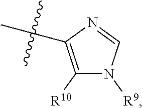

wherein R⁹ is H or alkyl, and R¹⁰ is H, alkyl, halo, haloalkyl, nitro, or sulfonamido (—SO₂NH₂); and
wherein the C₁₋₁₀ alkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl,
bond a is a single bond or double bond;
bond b is a single bond or double bond;
provided when bond a is a single bond, then R³ is H, bond b is a double bond, and R⁴ is S, and
when bond a is a double bond, then R³ is absent, bond b is a single bond, and R⁴ is SR⁵,
further provided that
when ring A is phenyl, X¹, X², X³, and X⁴ are each CR¹, X⁵ is O, R² is n-propyl, bond a is a single bond, bond b is a double bond, R³ is hydrogen, and R⁴ is S, then R¹ is not alkylamido (—C(O)NHalkyl);
when ring A is phenyl, X¹, X², and X⁴ are each CH, X³ is CR¹, n is 1, X⁵ is O, R² is alkyl, bond a is a single bond, bond b is a double bond, R³ is hydrogen, and R⁴ is S, then R¹ at the X³ position is not halo or hydrogen;
when ring A is phenyl, X¹, X³, and X⁴ are each CH, X² is CCH₃, n is 1, X⁵ is O, bond a is a single bond, bond b is a double bond, R³ is hydrogen, and R⁴ is S, then R² is not aryl; and
when ring A is phenyl, X¹, X³, and X⁴ are each CH, X² is C(halo), n is 1, X⁵ is O, bond a is a double bond, bond b is a single bond, R² is phenyl, R³ is absent, and R⁴ is SR⁵, then R⁵ is not alkyl.

The optionally substituted benzylamidoalkyl in R² typically has the structure —(CH₂)ₓ—NHC(O)-Ph-R⁸, wherein x is an integer of 1-4 (i.e., 1, 2, 3, or 4; preferably 2) and R⁸ is at the 2-, 3-, or 4-position on the phenyl and is hydrogen, alkyl, alkoxy, halo, hydroxy, cyano, alkoxycarbonyl (—C(O)O-alkyl), thiocyanato (—NCS), or morpholinylmethyl (e.g., 1- or 4-morpholinylmethyl). Specific examples of the optionally substituted benzylamidoalkyl include, e.g., 3-cyanobenzylamidoalkyl, 3-methoxybenzylamidoalkyl, 4-cyanobenzylamidoalkyl, 4-iodobenzylamidoalkyl, 4-methoxycarbonylbenzylamidoalkyl, 4-(1-morpholinylmethyl)benzylamidoalkyl, and 4-thiocyanatobenzylamidoalkyl.

In preferred aspects, the compound of formula (I) is compound 65, 79, 83, 85, 129, 134, a salt thereof, or a combination thereof. More preferably, the compound of formula (I) is compound 129 or a salt thereof.

In an aspect, the present invention further provides exemplary compounds of formula (Ib-1), including

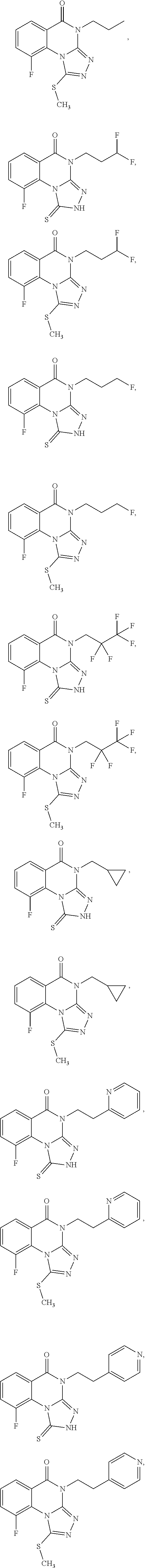

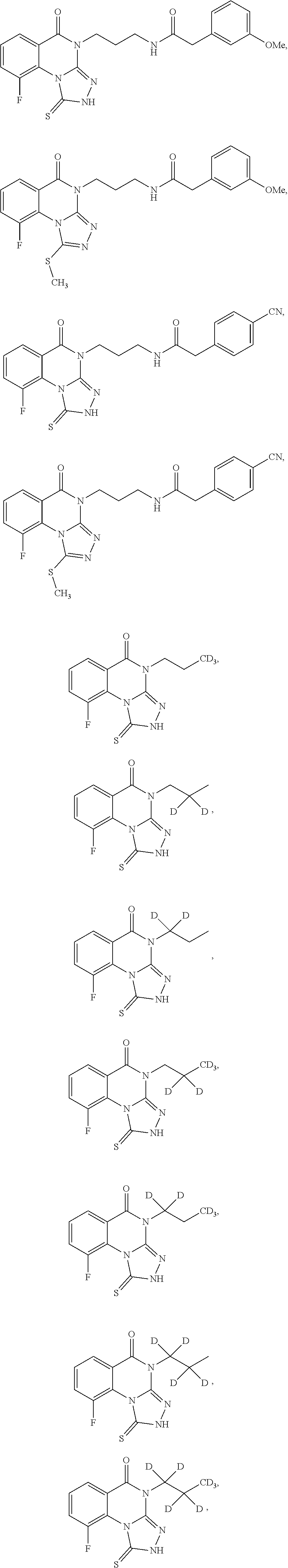

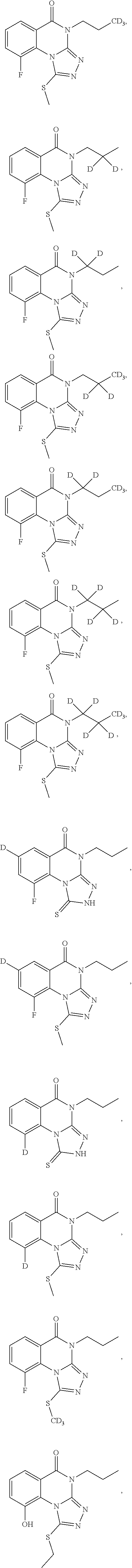

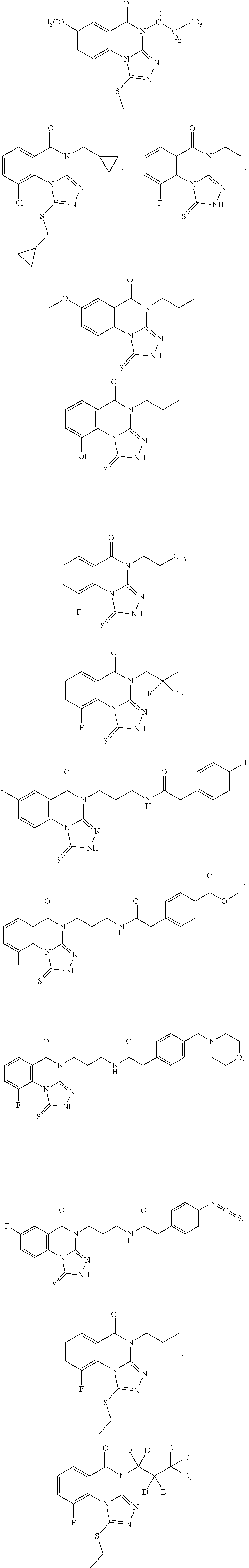

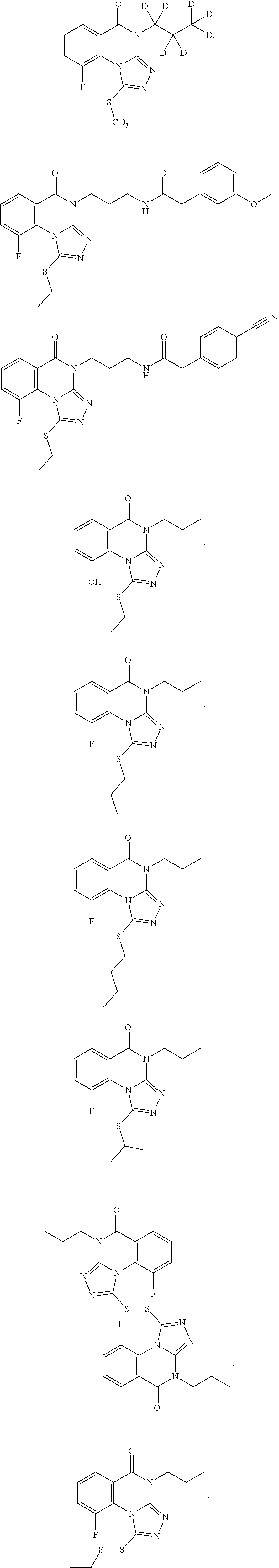

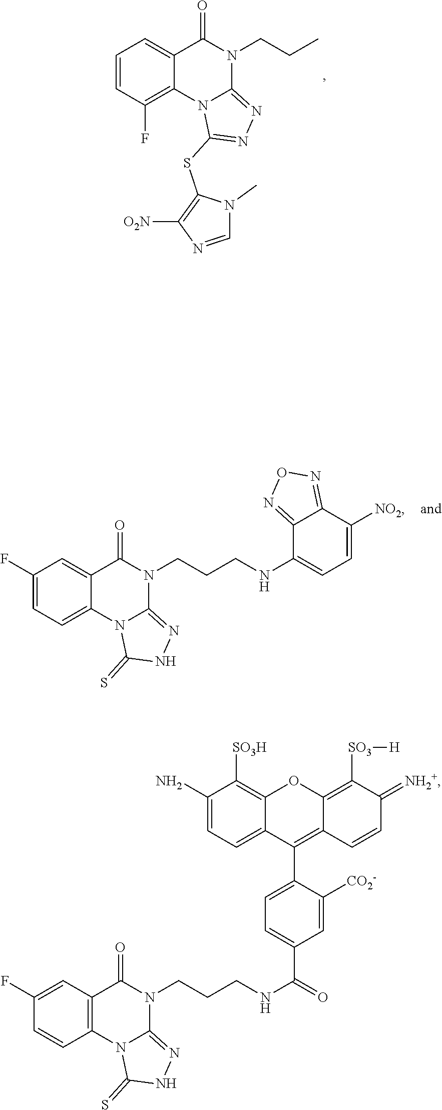

or a pharmaceutically acceptable salt thereof.

The invention provides a compound of formula (I) that is a triazoloquinazolinone compound or a pharmaceutically acceptable salt of formula (Ib-2):

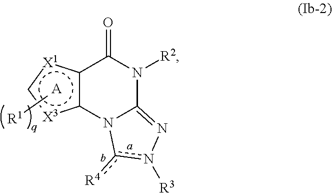

wherein
ring A is a 5-membered heteroaryl;
X¹ and X³ are each CR¹, NR^{1′}, S, or O, provided that at least one of X¹ and X³ is NR^{1′}, S, or O and the other is CR¹;
each instance of R¹ is the same or different and each is selected from the group consisting of H, deuterium, C₁₋₆ alkyl (including deuterated alkyl), alkoxy, halo, haloalkyl, alkylthio (—S-alkyl), alkylthioalkylenyl (-alkylene-S-alkyl), cyano, amino, alkylamino, and dialkylamino, or two instances of R¹ are linked to form a cycloalkyl or a phenyl, each of which is optionally substituted;
q is 1 or 2;
R^{1′} is H or alkyl;
R² is selected from the group consisting of H, alkyl, cycloalkyl, alkenyl, and aryl;
wherein the alkyl, cycloalkyl, alkenyl, and aryl of R² is optionally substituted with one or more substituents selected from the group consisting of deuterium, alkyl, alkoxy, halo, hydroxy, haloalkyl, alkoxy, alkyl, cycloalkyl, heterocycloalkyl, aryl, heteroaryl, amino, alkylamino, dialkylamino, amido, alkylsulfonamido, phosphonato, cyano, thiocyano, carboxylate, a protecting group, an amino acid residue, and a peptide residue,
R³ is H or R³ is absent;
bond a is a single bond or double bond;
bond b is a single bond or double bond;
provided when bond a is a single bond, then R³ is present, bond b is a double bond, and R⁴ is S, and
when bond a is a double bond, then R³ is absent, bond b is a single bond, and R⁴ is S—R⁵;
R⁵ is selected from the group consisting of C₁₋₁₀ alkyl, cycloalkyl, acetyl, and optionally substituted 4-imidazolyl of the structure

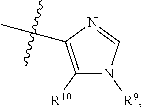

wherein R⁹ is H or alkyl, and R¹⁰ is H, alkyl, halo, haloalkyl, nitro, or sulfonamido (—SO₂NH₂); and
wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl,
provided that
when ring A is thiophenyl, X¹ is S, X³ is CR¹, bond a is a double bond, bond b is a single bond, R³ is absent, R⁴ is SR⁵, and R⁵ is alkyl, then R² is not aryl; and
when ring A is thiophenyl, X¹ is S, X³ is CR¹, R¹ is H, bond a is a single bond, bond b is a double bond, R³ is hydrogen, and R⁴ is S, then R² is not n-butyl, benzyl, or —CH₂-Ph-(4-ethyl).

Two instances of R¹ can be linked to form a cycloalkyl or a phenyl, each of which is optionally substituted, thereby forming a bicyclic compound. For example, two R¹ moieties can be linked to form phenyl that is optionally substituted with another R¹ group as defined herein (e.g., alkyl or halo (e.g., F, Cl)).

Exemplary compounds of formula (Ib-2) include:

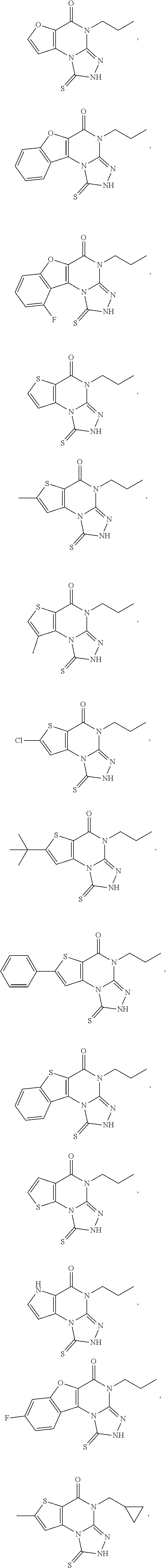

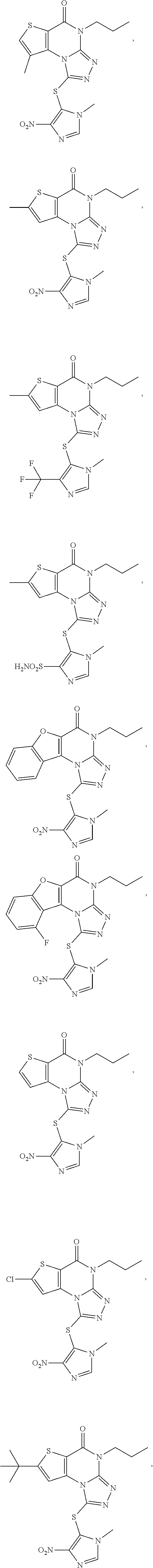

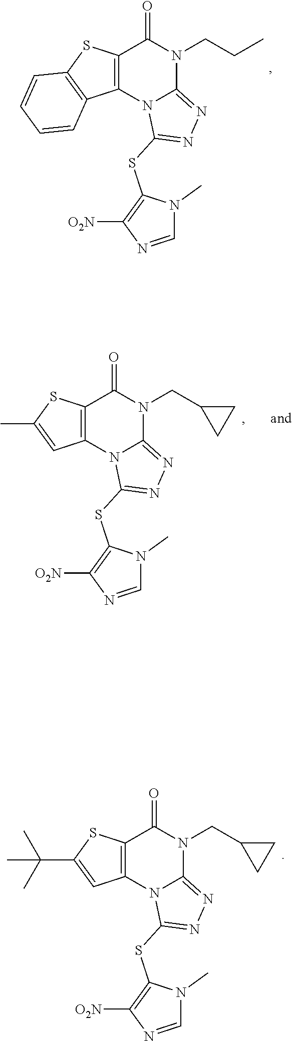

In some aspects, the compound of formula (Ib-2) or a pharmaceutically acceptable salt thereof is a compound of formula (Ib-3):

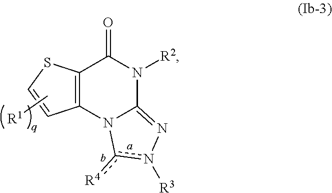

wherein
each instance of R¹ is the same or different and each is selected from the group consisting of H, deuterium, C₁₋₆ alkyl (including deuterated alkyl), alkenyl, alkynyl, cycloalkyl, alkoxy, cycloalkoxy, halo, haloalkyl, alkylthio (—S-alkyl), alkylthioalkylenyl (-alkylene-S-alkyl), cyano, amino, alkylamino, dialkylamino, amido, aryl, and heterocycloalkyl or
more than one instance of R¹ are linked to form a cycloalkyl or a phenyl, each of which is optionally substituted;
q is 1 or 2;
R² is selected from the group consisting of H, alkyl, cycloalkyl, alkenyl, and aryl;
wherein the alkyl, cycloalkyl, alkenyl, and aryl of R² is optionally substituted with one or more substituents selected from the group consisting of deuterium, alkyl, alkoxy, halo, hydroxy, haloalkyl, alkoxy, alkyl, cycloalkyl, heterocycloalkyl, aryl, heteroaryl, amino, alkylamino, dialkylamino, amido, alkylsulfonamido, phosphonato, cyano, thiocyano, carboxylate, a protecting group, an amino acid residue, and a peptide residue;
R³ is H or R³ is absent;
bond a is a single bond or double bond;
bond b is a single bond or double bond;
provided when bond a is a single bond, then R³ is present, bond b is a double bond, and R⁴ is S, and
when bond a is a double bond, then R³ is absent, bond b is a single bond, and R⁴ is SR⁵;
R⁵ is selected from the group consisting of C₁₋₁₀ alkyl, cycloalkyl, acetyl, and optionally substituted 4-imidazolyl of the structure

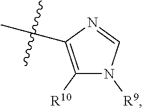

wherein R⁹ is H or alkyl, and R¹⁰ is H, alkyl, halo, haloalkyl, nitro, or sulfonamido (—SO₂NH₂); and
wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl,
provided that
when bond a is a double bond, bond b is a single bond, R³ is absent, R⁴ is SR⁵, and R⁵ is alkyl, then R² is not aryl; and
when R¹ is H, bond a is a single bond, bond b is a double bond, R³ is hydrogen, and R⁴ is S, then R² is not n-butyl, benzyl, or —CH₂-Ph-(4-ethyl).

In some aspects of the compound of formula (Ib-3), R¹ is 5-methyl, p is 1, R² is —(CH₂)₂-cyclopropyl, and either (i) bond a is double, bond b is single, R³ is absent, R⁴ is SR⁵, and R⁵ is substituted 4-imidazolyl of the structure

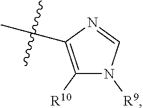

wherein R⁹ is methyl, and R¹⁰ is haloalkyl (e.g., —CF₃), nitro, or sulfonamido (—SO₂NH₂), or (ii) bond a is single, bond b is double, R³ is H, and R⁴ is S. In a preferred aspect, the compound of formula (Ib-3) is selected from

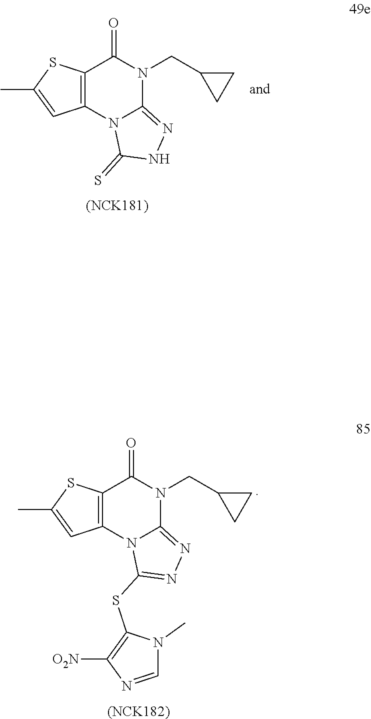 [SMILES: Cc1cc2c(s1)c(=O)n(CC1CC1)c1n[nH]c(=S)n21 ; Cc1cc2c(s1)c(=O)n(CC1CC1)c1nnc(Sc3c([N+](=O)[O-])ncn3C)n21 ; "(NCK181)" ; "(NCK182)"]

In some aspects, the compound of formula (Ia-3) is 85 (NCK182).

Further exemplary compounds of formula (I) and pharmaceutically acceptable salts thereof include:

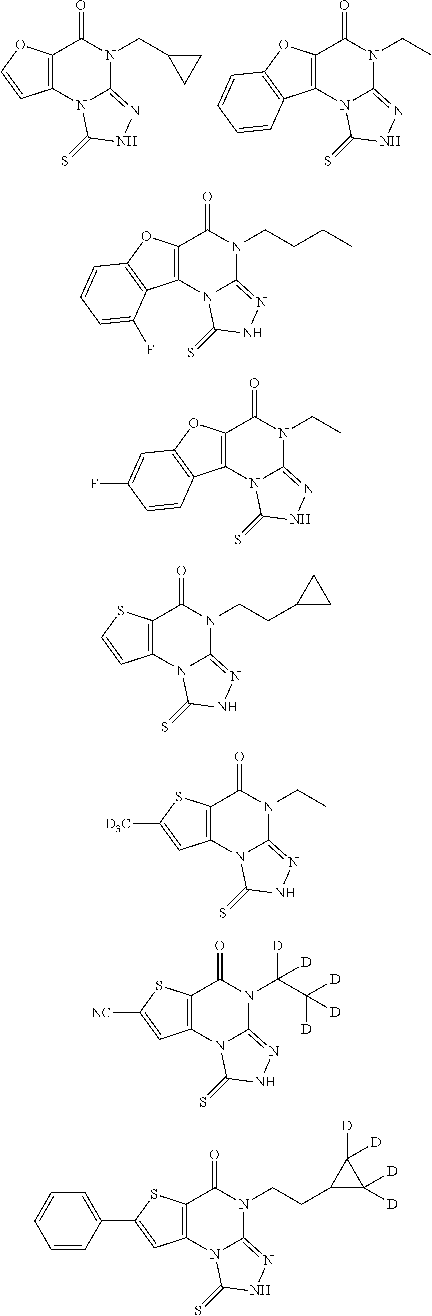

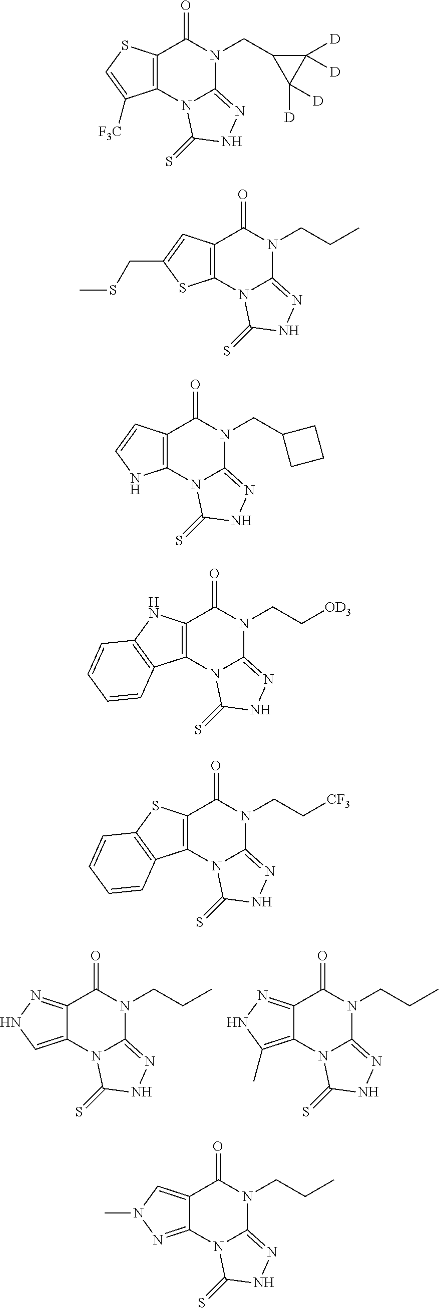

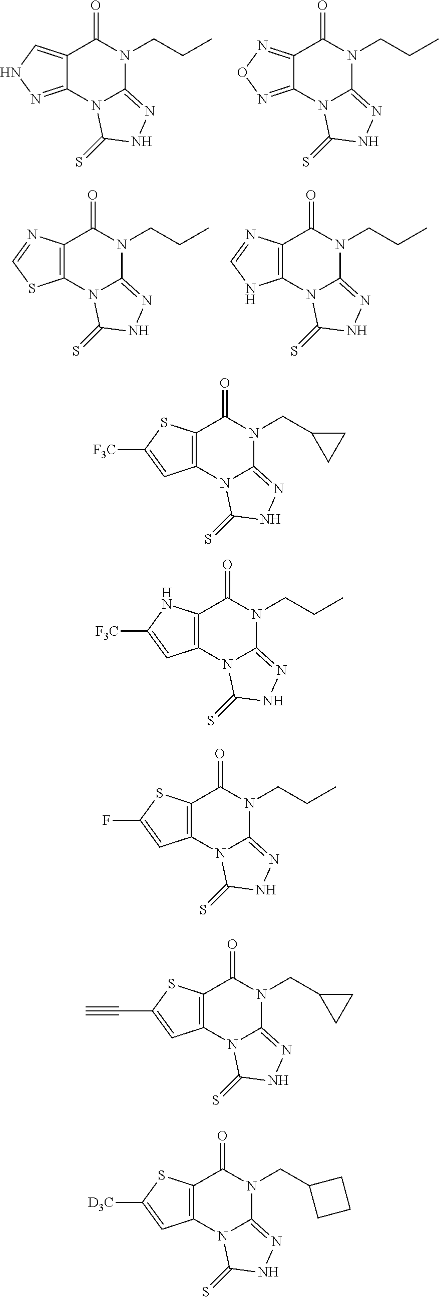

or a prodrug thereof, in which the sulfur moiety (═S) (i.e., R⁴) in the foregoing compounds is replaced with —SR⁵, wherein in the prodrug of each of the foregoing compounds.
bond a is a double bond,
bond b is a single bond,
R³ is absent, and
R⁵ is selected from the group consisting of C₁₋₁₀ alkyl, cycloalkyl, acetyl, and optionally substituted 4-imidazolyl of the structure

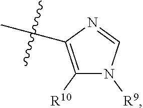

wherein R⁹ is H or alkyl, and R¹⁰ is H, alkyl, halo, haloalkyl, nitro, or sulfonamido (—SO₂NH₂); and
wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl.

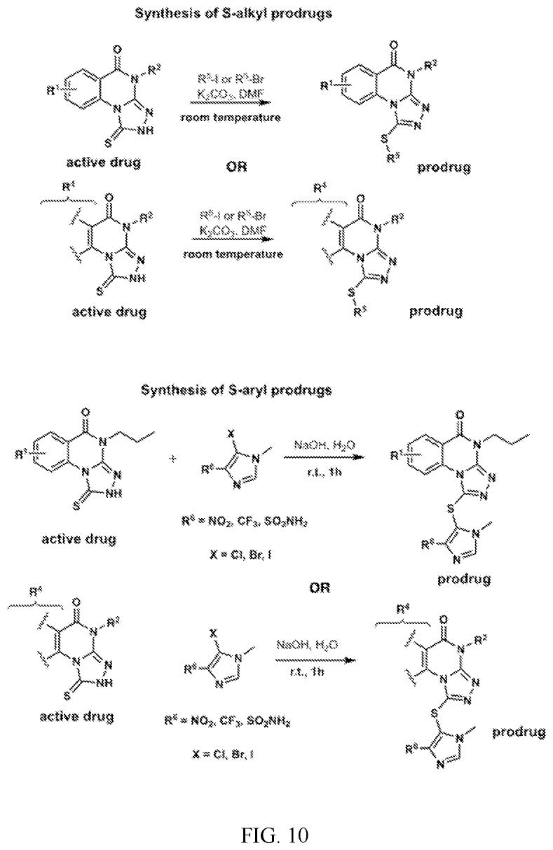

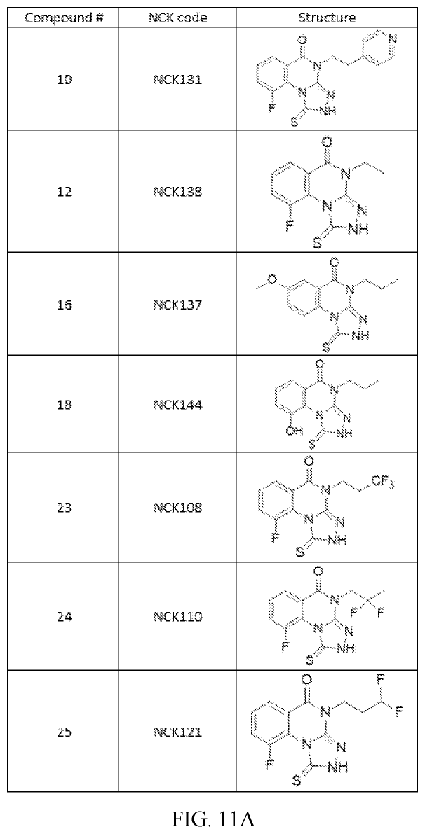

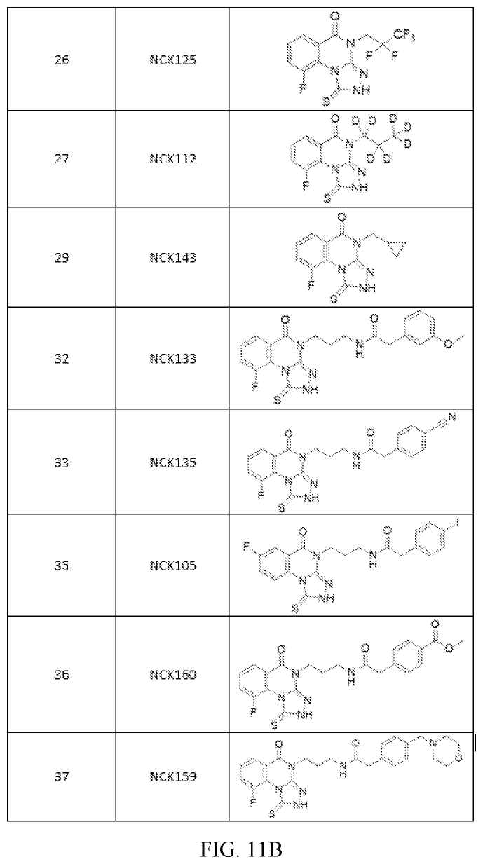

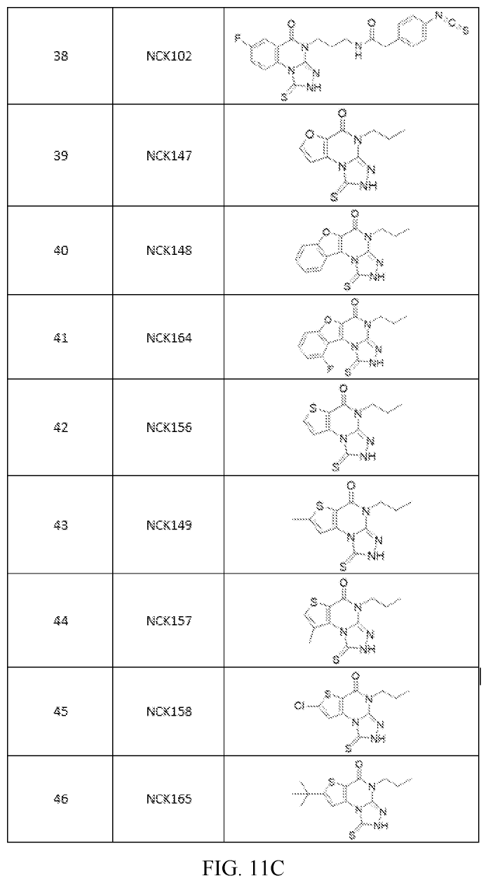

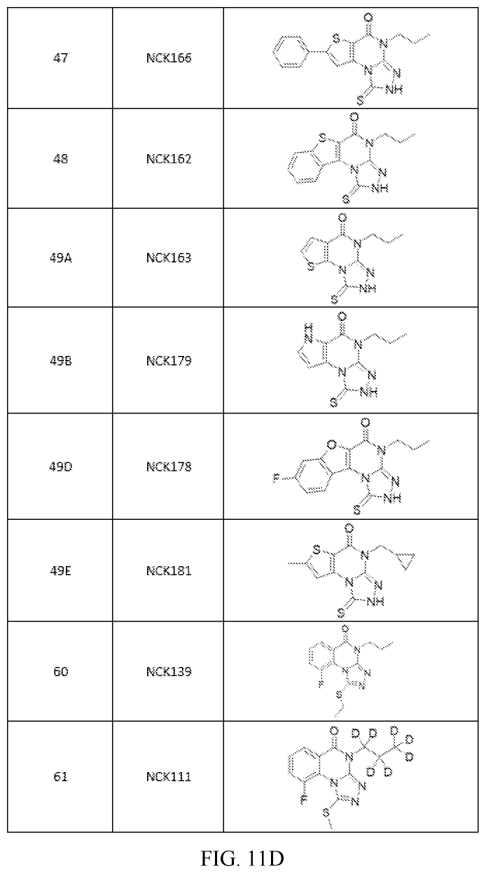

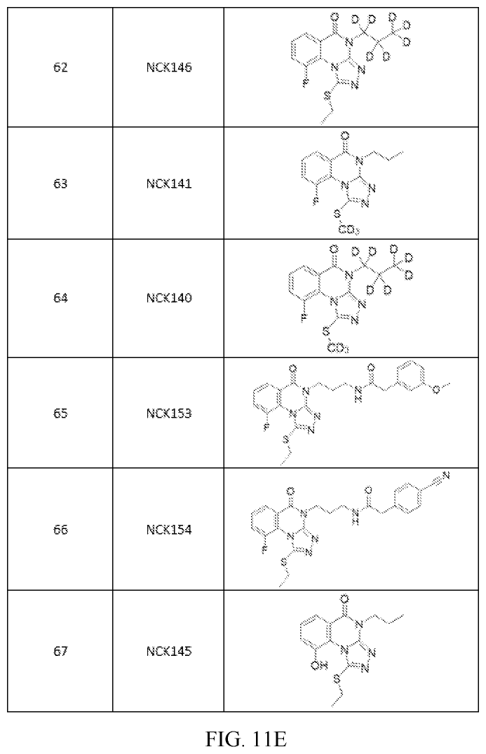

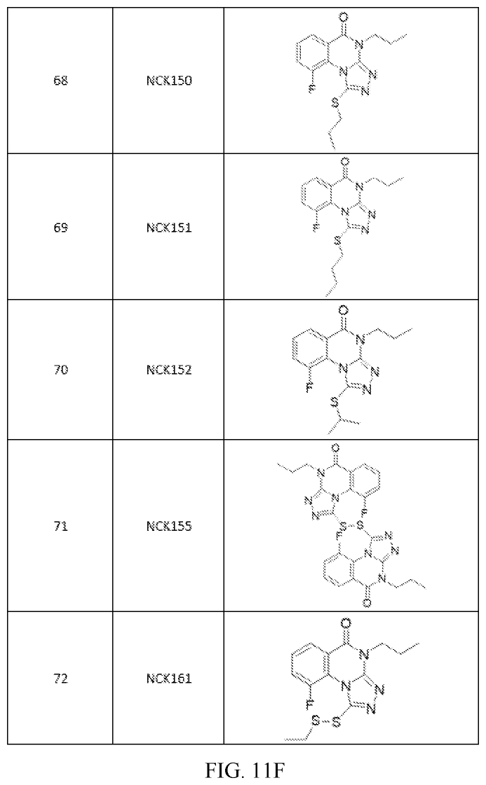

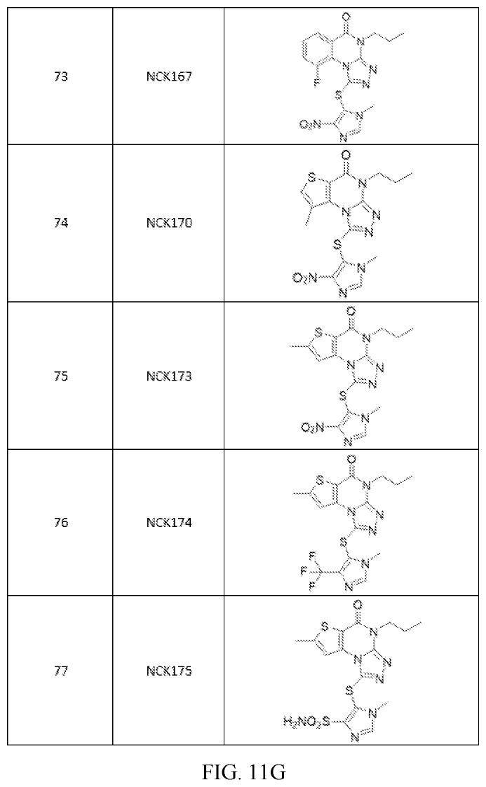

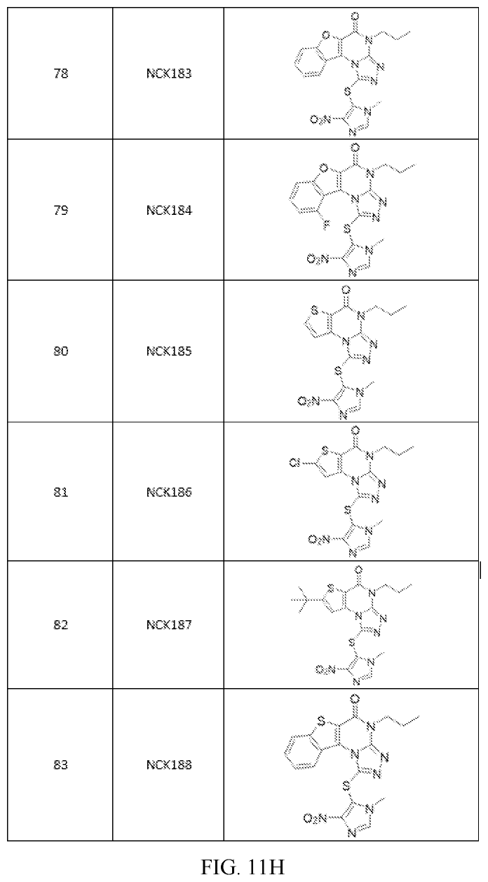

The invention further provides compounds of formula (I), including formulas (Ib-1), (Ib-2), and (Ib-3), as listed in the table of FIGS. 11A-11I.

In any of the aspects above, the term “alkyl” implies a straight-chain or branched alkyl substituent containing from, for example, from about 1 to about 6 carbon atoms, e.g., from about 1 to about 4 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., in C₃-C₆ cycloalkylalkyl, hydroxyalkyl, haloalkyl (e.g., monohaloalkyl, dihaloalkyl, and trihaloalkyl), cyanoalkyl, aminoalkyl, alkylamino, dialkylamino, alkylaminoalkyl, dialkylaminoalkyl, arylcarbonylalkyl (-(alkyl)C(O)aryl), arylalkyl, etc. The alkyl can be substituted or unsubstituted, as described herein. Even in instances in which the alkyl is an alkylene chain (e.g., —(CH₂)ₙ—, in which n is 1 to 10, 1 to 8, 1 to 6, 1 to 4, 1 to 3, 1 to 2, or 2), the alkyl group can be substituted or unsubstituted.

In any of the aspects above, the term “alkenyl,” as used herein, means a linear alkenyl substituent containing from, for example, about 2 to about 6 carbon atoms (branched alkenyls are about 3 to about 6 carbons atoms), e.g., from about 3 to about 5 carbon atoms (branched alkenyls are about 3 to about 6 carbons atoms). In accordance with an aspect, the alkenyl group is a C₂-C₄ alkenyl. Examples of alkenyl group include ethenyl, allyl, 2-propenyl, 1-butenyl, 2-butenyl, 1-pentenyl, 2-pentenyl, 3-pentenyl, 1-hexenyl, and the like. The alkenyl can be substituted or unsubstituted, as described herein.

As used herein, the term “alkynyl” refers to unsaturated hydrocarbon radicals having at least one carbon-carbon triple bond. Cₓ alkynyl and Cₓ-C_{y} alkynyl are typically used where X and Y indicate the number of carbon atoms in the chain (e.g., C₂-C₁₀ alkynyl, C₂-C₈ alkynyl, C₂-C₆ alkynyl, or C₂-C₄ alkynyl). For example, C₂-C₆ alkynyl includes alkynls that have a chain of between 2 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. The alkynyl can be substituted or unsubstituted, as described herein.

In any of the aspects above, the term “cycloalkyl,” as used herein, means a cyclic alkyl moiety containing from, for example, 3 to 6 carbon atoms or from 5 to 6 carbon atoms. Examples of such moieties include cyclopropyl, cyclobutyl, cyclopentyl, cyclohexyl, and the like. The cycloalkyl can be substituted or unsubstituted, as described herein.

In any of the aspects above, the term “aryl” refers to a mono, bi, or tricyclic carbocyclic ring system having one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2π electrons, according to Hückel's Rule, wherein n=1, 2, or 3. This definition also applies wherever “aryl” occurs as part of a group, such as, e.g., in haloaryl (e.g., monohaloaryl, dihaloaryl, and trihaloaryl), arylalkyl, etc. The aryl can be substituted or unsubstituted, as described herein.

In any of the aspects above, the term “heteroaryl” refers to aromatic 5 or 6 membered monocyclic groups, 9 or 10 membered bicyclic groups, and 11 to 14 membered tricyclic groups which have at least one heteroatom (O, S, or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized, and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. Illustrative examples of heteroaryl groups are pyridinyl, pyridazinyl, pyrimidyl, pyrazinyl, benzimidazolyl, triazinyl, imidazolyl, (1,2,3)- and (1,2,4)-triazolyl, pyrazinyl, tetrazolyl, furyl, pyrrolyl, thienyl, isothiazolyl, thiazolyl, isoxazolyl, and oxadiazolyl. The heteroaryl can be substituted or unsubstituted, as described herein.

The term “heterocycloalkyl” means a stable, saturated, or partially unsaturated monocyclic, bicyclic, and spiro ring system containing 3 to 7 ring members of carbon atoms and other atoms selected from nitrogen, sulfur, and/or oxygen. In an aspect, a heterocycloalkyl is a 5, 6, or 7-membered monocyclic ring and contains one, two, or three heteroatoms selected from nitrogen, oxygen, and sulfur. The heterocycloalkyl may be attached to the parent structure through a carbon atom or through any heteroatom of the heterocycloalkyl that results in a stable structure. Examples of such heterocycloalkyl rings are isoxazolyl, thiazolinyl, imidazolidinyl, piperazinyl, homopiperazinyl, pyrrolyl, pyrrolinyl, pyrazolyl, pyranyl, piperidyl, oxazolyl, and morpholinyl. The heterocycloalkyl can be substituted or unsubstituted, as described herein.

In any of the aspects above, the term “hydroxy” refers to the group —OH.

In any of the aspects above, the term “cyano” refers to the group —CN, whereas the term “thiocyano” refers to —SCN.

In any of the aspects above, the terms “alkoxy” and “cycloalkyloxy” embrace linear or branched alkyl and cycloalkyl groups, respectively, that are attached to a divalent oxygen. The alkyl and cycloalkyl groups are the same as described herein.

In any of the aspects above, the term “halo” refers to a halogen selected from fluorine, chlorine, bromine, and iodine.

In any of the aspects above, the term “carboxylato” refers to the group —C(O)OH.

In any of the aspects above, the term “amino” refers to the group —NH2. The term “alkylamino” refers to —NHR, whereas the term “dialkylamino” refers to —NRR′. R and R′ are the same or different and each is a substituted or unsubstituted alkyl group, as described herein.

In any of the aspects above, the term “amido” refers to the group —C(O)NRR′, which R and R′ are the same or different and each is hydrogen or a substituted or unsubstituted alkyl group, as described herein.

In any of the aspects above, the term “alkylsulfonamido” refers to the group —NHSO₂R, which R is a substituted or unsubstituted alkyl group, as described herein.

In any of the aspects above, the term “phosphonato” refers to the group —P(O)(OR)₂, which R is hydrogen or a substituted or unsubstituted alkyl group, as described herein.

In any of the aspects above, the term “amino acid residue” refers to any naturally occurring amino acid with an open valency at either terminus for bonding to an alkylene chain, alkenylene chain, or aryl. Suitable amino acid residues include arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan. The term “peptide residue” refers to two or more amino acids linked together to form a peptide and has an open valency at a terminus for bonding to an alkylene chain, alkenylene chain, or aryl.

In any of the aspects above, the term “protecting group” refers to any suitable nitrogen protecting group, including, e.g., t-butyloxtcarbonyl (Boc), 9-fluorenylmethyloxycarbonyl (Fmoc), carbobenzyloxy (Cbz), acetyl, trifluoroacetyl, benzyl, benzoyl, triphenylmethyl (Tr), benzylideneamine, tosyl (Ts), p-methoxybenzyl carbonyl (Moz), p-methoxybenzyl (PMB), p-methoxyphenyl (PMP), carbamate, 3,4-dimethoxybenzyl (DMPM), and trichloroethyl chloroformate (Troc). In some preferred aspects, the protecting group is Boc.

In other aspects, any substituent that is not hydrogen (e.g., alkyl, alkenyl, alkynyl, cycloalkyl, cycloalkylalkyl, aryl, arylalkyl, heteroaryl, heteroarylalkyl, heterocycloalkyl, or heterocycloalkylalkyl) can be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group is substituted with a substituent, e.g., halo, amino, alkyl, OH, alkoxy, and others, the aromatic ring hydrogen is replaced with the substituent and this can take place in any of the available hydrogens, e.g., 2, 3, 4, 5, and/or 6-position wherein the 1-position is the point of attachment of the aryl group in the compound of the present invention. Suitable substituents include, e.g., halo, alkyl, alkenyl, hydroxy, nitro, cyano, amino, alkylamino, alkoxy, aryloxy, aralkoxy, carboxyl, carboxyalkyl, carboxyalkyloxy, amido, alkylamido, haloalkylamido, aryl, heteroaryl, and heterocycloalkyl, each of which is described herein. In some instances, the substituent is at least one alkyl, halo, and/or haloalkyl (e.g., 1 or 2).

In any of the aspects above, whenever a range of the number of atoms in a structure is indicated (e.g., a C₁₋₁₂, C₁₋₈, C₁₋₆, C₁₋₄, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, cycloalkyl, etc.) referenced herein encompasses and specifically describes 1, 2, 3, 4, 5, 6, 7, and/or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, etc., as appropriate).

The subscript “n” represents the size of aromatic ring A. The subscript n is either 0 or 1. When n is 0, ring A is a 5-membered heteroaryl, and X⁴ is not present in the compound of formula (I). When n is 1, ring A is ring A is phenyl or a 6-membered heteroaryl, and X⁴ is present in the compound of formula (I).

The subscript “m” represents the number of methylene repeat units. The subscript m is either 0 or an integer from 1-5 (i.e., 1, 2, or 3, 4, or 5). When m is 0, then the moiety —NHC(O)(CH₂)ₘR⁶ does not contain any methylene repeat units.

The subscript “p” represents the number of R¹ substituents. The subscript p can be an integer of 1-4 (i.e., 1, 2, or 3, or 4). In some aspects of formula (I), p preferably is 1.

The subscript “q” represents the number of R¹ substituents in certain subsets of formula (I). The subscript q can be an integer of 1 or 2. In some aspects of formula (I), q preferably is 1.

In any of the aspects herein, the phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. For example, an inorganic acid (e.g., hydrochloric acid, sulfuric acid, phosphoric acid, or hydrobromic acid), an organic acid (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base(e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typical. Lists of suitable salts are found in Remington's Pharmaceutical Sciences, 18th ed., Mack Publishing Company, Easton, P A, 1990, p. 1445, and Journal of Pharmaceutical Science, 66, 2-19 (1977). For example, they can be a salt of an alkali metal (e.g., sodium or potassium), alkaline earth metal (e.g., calcium), or ammonium of salt.

In any of the aspects herein, the term “prodrug” is intended to include any compound that releases an active parent drug according to a structure described herein in vivo when such prodrug is administered to a subject. Prodrugs of a compound described herein are prepared by modifying functional groups present in the compound described herein in such a way that the modifications can be cleaved in vivo to release the active parent compound. An example of a specific prodrug is as follows:

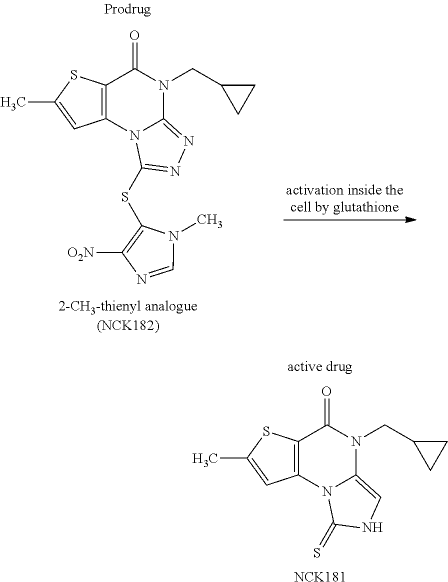 [SMILES: Cc1cc2c(s1)c(=O)n(CC1CC1)c1nnc(Sc3c([N+](=O)[O-])ncn3C)n21 ; Cc1cc2c(s1)c(=O)n(CC1CC1)c1c[nH]c(=S)n21 ; "NCK181"]

A small chemical library of about 400 drug-like molecules in the Molecular Recognition Section, National Institute of Diabetes and Digestive and Kidney Diseases (NIDDK), National Institutes of Health (NIH), was screened for the ability to bind to the PBD of Plk1. The principal screening assay consisted of an ELISA-based Plk1 PBD inhibition assay that utilizes the specific interaction between the full-length human Plk1 and a specific phosphopeptide (Biotin-Ahx-C-ETFDPPLHS-pT-AI-NH2) derived from a kinetochore-localizing Plk1-binding protein, PBIP1 (Yun et al., Nat. Struct. Mol. Biol. 2009, 16 (8), 876-882; and Kang et al., Mol. Cell 2006, 24 (3), 409-422). When necessary, secondary fluorescence polarization (FP) assays were carried out using a 5-carboxyfluorescein-labeled peptide (FITC-Ahx-DPPLHS-pT-AI-NH₂) (Qian et al., Biopolymers 2014, 102 (6), 444-455) to confirm the anti-Plk1 PBD activity from the primary ELISA-based assay and eliminate false positives.

Among the hits identified was the triazoloquinazolinone 7 (FIG. 1), which inhibited PBD binding in the ELISA assay with an IC₅₀ of 4.38 μM. When compared to the previously characterized phosphopeptide, PLHSpT 6a (IC₅₀ of 14.74 μM), showing a K_{d} of ˜450 nM (Yun et al., Nat. Struct. Mol. Biol. 2009, 16 (8), 876-882), the affinity of the lead compound 7 is anticipated to be at least three-fold higher than the peptide 6a (Table 1). Compound 7 was previously reported as a weak hit in a structure-based in silico screen to identify ligands of the adenosine receptors (Carlsson et al., J Med. Chem. 2010, 53 (9), 3748-3755). From this hit, a family of congeners was synthesized, by substitutions introduced on all of the moieties of the lead compound 7 (FIG. 1) to form the genus compound of formula (I). Some of the analogs were obtained from commercial sources and their identity confirmed by ¹H-NMR and mass spectrometry. Other analogs were prepared using synthetic methods described herein.

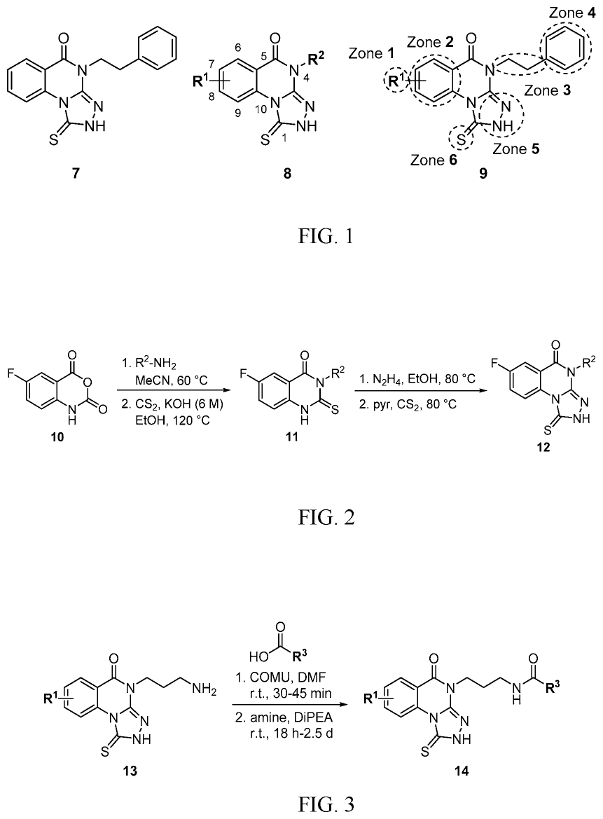

In general, the synthesis of analogs of 7 generally began with either the corresponding isatoic anhydride, as shown in Scheme 1 of FIG. 2, or anthranilic acid. An isatoic anhydride (e.g., 10) was heated in the presence of the desired amine to give the corresponding, intermediate 2-aminobenzamide (structure not shown). This intermediate was heated in the presence of carbon disulfide and potassium hydroxide to give the cyclized 2-thioxo-2,3-dihydroquinazolin-4(1H)-one derivative (e.g., 11), which was isolated by precipitation with treatment of water followed by filtration. The corresponding 3-thione-1,2,4-triazole (e.g., 12) was typically formed in a one-pot procedure by first stirring with hydrazine in refluxing ethanol to form the hydrazine intermediate. This intermediate, after cooling, was treated with pyridine and carbon disulfide. The desired triazoloquinazolinone 12 was formed after heating the reaction again to 80° C. The 2-thioxo-2,3-dihydroquinazolin-4(1H)-one intermediate could also be formed by reacting the desired 2-aminobenzoic acid with an isothiocyanate. However, due to the limited availability of isothiocyanates, a preferred route for preparation of the triazoloquinazolinones is shown in Scheme 1 of FIG. 2. With the core triazoloquinazolinone formed, additional analogs could be generated with a terminal amine (e.g., 13, Scheme 2 of FIG. 3). Amides of this amino congener (e.g., 14) were selected as a primary focus, and (1-cyano-2-ethoxy-2-oxoethylidenaminooxy)dimethylamino-morpholino-carbenium hexafluorophosphate (COMU) was found to be the best coupling agent and therefore used to synthesize all additional analogs.

The methods described herein comprise administering, to a subject in need thereof, a compound of formula (I) or a pharmaceutically acceptable salt thereof in the form of a pharmaceutical composition. In particular, a pharmaceutical composition will comprise at least one compound of formula (I), including a compound of formula (Ia-1), (Ia-2), (Ia-3), (Ib-1), (Ib-2), or (Ib-3), or a pharmaceutically acceptable salt thereof and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compounds and one that has no detrimental side effects or toxicity under the conditions of use.

The pharmaceutical compositions can be administered as oral, sublingual, transdermal, subcutaneous, topical, absorption through epithelial or mucocutaneous linings, intravenous, intranasal, intraarterial, intraperitoneal, intramuscular, intratumoral, peritumoral, intraperitoneal, intrathecal, rectal, vaginal, or aerosol formulations. In some aspects, the pharmaceutical composition is administered orally or intravenously.

In accordance with any of the aspects, the compound of formula (I) or a pharmaceutically acceptable salt thereof can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound of formula (I) or a salt thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the inhibitors in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The inhibitors may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some aspects, the composition contains at least one active component and a suitable vehicle or carrier. It may also contain other components, such as an anti-irritant. The carrier can be a liquid, solid or semi-solid. In aspects, the composition is an aqueous solution, such as a mouthwash. Alternatively, the composition can be a dispersion, emulsion, gel, lotion or cream vehicle for the various components. In one aspect, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In aspects of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

The compound of formula (I) or a pharmaceutically acceptable salt thereof, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

The dose administered to the mammal, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular inhibitor and the desired effect. It will be appreciated by one of skill in the art that various conditions or disease states may require prolonged treatment involving multiple administrations.

The inventive methods comprise administering an effective amount of a compound of formula (I) or a pharmaceutically acceptable salt thereof. An “effective amount” means an amount sufficient to show a meaningful benefit in an individual, e.g., promoting at least one aspect of tumor cell cytotoxicity (e.g., inhibition of growth, inhibiting survival of a cancer cell, reducing proliferation, reducing size and/or mass of a tumor (e.g., solid tumor)), or treatment, healing, prevention, delay of onset, halting, or amelioration of other relevant medical condition(s) associated with a particular cancer. The meaningful benefit observed in the subject can be to any suitable degree (10, 20, 30, 40, 50, 60, 70, 80, 90% or more). In some aspects, one or more symptoms of the cancer are prevented, reduced, halted, or eliminated subsequent to administration of a compound of formula (I) or a pharmaceutically acceptable salt thereof, thereby effectively treating the cancer to at least some degree.

Effective amounts may vary depending upon the biological effect desired in the individual, condition to be treated, and/or the specific characteristics of the compound of formula (I) or a pharmaceutically acceptable salt thereof, and the individual. In this respect, any suitable dose of the compound of formula (I) or a pharmaceutically acceptable salt thereof can be administered to the subject (e.g., human), according to the type of cancer to be treated. Various general considerations taken into account in determining the “effective amount” are known to those of skill in the art and are described, e.g., in Gilman et al., eds., Goodman and Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the compound of formula (I) or a pharmaceutically acceptable salt thereof desirably comprises about 0.01 mg per kilogram (kg) of the body weight of the subject (mg/kg) or more (e.g., about 0.05 mg/kg or more, 0.1 mg/kg or more, 0.5 mg/kg or more, 1 mg/kg or more, 2 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints can be used to define a close-ended range, or a single endpoint can be used to define an open-ended range.

For purposes of the present invention, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

In an aspect, a compound formula (I) inhibits Plk1, particularly the polo-box domain (PBD) of Plk1. Structure-activity relationship studies led to multiple inhibitors having ≥10-fold higher inhibitory activity than the previously characterized Plk1 PBD-specific phosphopeptide, PLHSpT 6a (K_{d} ˜450 nM). In an aspect, a compound of formula (I) is selective for Plk1 relative to other polo-like kinases (e.g., Plk2 and/or Plk3). For example, the compound can be at least 2 times (e.g., at least 3 times, at least 4 times, at least 5 times, at least 6 time, at least 8 times, at least 10 times, at least 15 times, at least 20 times, at least 30 times, at least 40 times, at least 50 times, at least 60 times, or at least 100 times) more selective for Plk1 compared to one or more other polo-like kinases.

The invention is further directed to a method of inhibiting PBD Plk1 activity in a cell comprising contacting a compound of formula (I), including compounds of formula (Ia-1), (Ia-2), (Ib-1), (Ib-2), and (Ib-3), or a pharmaceutically acceptable salt thereof to a cell, whereby activity of PBD Plk1 is inhibited. In some aspects, the inhibitory activity is by an active metabolite (e.g., a parent drug of a prodrug) of the compound of formula (I). The PDB Plk1 activity can be measured by any method, including the assay described herein. Typically, inhibition of PBD Plk1 activity will be demonstrated by an ELISA-based Plk1 PBD inhibition assay that utilizes the specific interaction between the full-length human Plk1 and a specific phosphopeptide (Biotin-Ahx-C-ETFDPPLHS-pT-AI-NH₂) derived from a kinetochore-localizing Plk1-binding protein, PBIP1, as described herein.

Since PBD inhibitors interfere only with the PBD-dependent Plk1 functions, they are anticipated to incur mitotic stress sufficient to induce cell death in cancer cells but not in normal cells. See, e.g., Park et al., Cell Cycle 2015, 14 (22), 3624-3634. While not wishing to be bound by any particular theory, it is believed that complete inhibition of Plk1 would be detrimental even for normal cell proliferation. Thus, inhibition of the PBD of Plk1 is seen as a viable treatment of cancer. Accordingly, certain compounds of formula (I) or a pharmaceutically acceptable salt thereof, can be administered to a subject in need thereof to treat cancer. In aspects in which a compound of formula (I) is used, such as a compound of formula (Ib-1), the compound of formula (I) will not contain deuterium. Anti-cancer activity can be measured by any suitable method, including the assays described herein.

The type of cancer is not particularly limited, but in certain aspects, the cancer comprises cancer cells that overexpress Plk1 relative to normal tissue of the same type. Examples of cancer treatable with the inventive method include cancers, including cancerous cells and tissue, of the head and neck, eye, skin, mouth, throat, esophagus, chest, bone, lung, colon, sigmoid, rectum, stomach, prostate, breast, ovaries, kidney, liver, pancreas, brain, intestine, heart, or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-borne tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute nonlymphocytic leukemia, acute undifferentiated leukemia, chronic myelocytic leukemia, chronic lymphocytic leukemia, hairy cell leukemia, or multiple myeloma. See, e.g., Harrison's Principles of Internal Medicine, Eugene Braunwald et al., eds., pp. 491 762 (15th Ed. 2001). In some aspects, the cancer is breast cancer, lung cancer, renal cancer, liver cancer, uterine cancer, prostate cancer, pancreatic cancer, glioma, thyroid carcinoma, head and neck squamous cell carcinoma, melanoma, colorectal cancer, esophageal carcinoma, or ovarian carcinoma.

In certain aspects of this method, the compound of formula (I) or a pharmaceutically acceptable salt thereof can be co-administered with an anti-cancer agent (e.g., a chemotherapeutic agent) and/or radiation therapy. In an aspect, the method comprises administering an amount of a compound of formula (I) or a pharmaceutically acceptable salt thereof that is effective to sensitize the cancer cells to one or more therapeutic regimens (e.g., chemotherapy or radiation therapy). The terms “co-administered” or “co-administration” refer to simultaneous or sequential administration. A compound of formula (I) or a pharmaceutically acceptable salt thereof can be administered before, concurrently with, or after administration of another anti-cancer agent (e.g., a chemotherapeutic agent).

One or more than one, e.g., two, three, or more anti-cancer agents can be administered. In this regard, the present invention is directed a pharmaceutical composition comprising a pharmaceutically acceptable carrier and a combination of the compound of formula (I) or a pharmaceutically acceptable salt thereof and at least one anti-cancer agent (e.g., chemotherapeutic agent).

Examples of anti-cancer agents include platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine), antitumor antibiotics (e.g., daunorubicin, doxorubicin, idarubicin, epirubicin, mitoxantrone, bleomycin, mitomycin C, plicamycin, dactinomycin), taxanes (e.g., paclitaxel and docetaxel), antimetabolites (e.g., 5-fluorouracil, cytarabine, pemetrexed, thioguanine, floxuridine, capecitabine, and methotrexate), nucleoside analogues (e.g., fludarabine, clofarabine, cladribine, pentostatin, nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vincristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panitumumab, tositumomab, trastuzumab, alemtuzumab, gemtuzumab ozogamicin, bevacizumab), nitrosoureas (e.g., carmustine, fotemustine, and lomustine), enzymes (e.g., L-Asparaginase), biological agents (e.g., interferons and interleukins), hexamethylmelamine, mitotane, angiogenesis inhibitors (e.g., thalidomide, lenalidomide), steroids (e.g., prednisone, dexamethasone, and prednisolone), hormonal agents (e.g., tamoxifen, raloxifene, leuprolide, bicalutamide, granisetron, flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, cellular immunotherapy (e.g., chimeric antigen receptor T cell therapy, tumor-infiltrating lymphocyte therapy), or any combination thereof. In some aspects, the anti-cancer agent is cisplatin, cytarabine, methotrexate, doxorubicin, or a combination thereof. In other aspects, the anti-cancer agent is imatinib, idelalisib, lapatinib, dasatinib, ceritinib, crizotinib, or a combination thereof.

In certain aspects of the method, the compound of formula (I) or a pharmaceutically acceptable salt thereof can be co-administered with one or more antifungal agents, particularly, an antifungal agent that inhibits CYP 3A4, such as ketoconazole, fluconazole, itraconazole, miconazole, posaconazole, voriconizole, or ritonavir. In some preferred aspects, the antifungal agent is ketoconazole.

One or more than one, e.g., two, three, or more antifungal agents can be administered. In this regard, the present invention is directed a pharmaceutical composition comprising a pharmaceutically acceptable carrier and a combination of the compound of formula (I) or a pharmaceutically acceptable salt thereof and at least one antifungal agent.

The invention is further illustrated by the following aspects.

Aspect (1) A method of treating cancer comprising administering to a subject in need thereof a compound of formula (I) or a pharmaceutically acceptable salt thereof

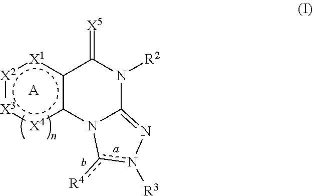

wherein
ring A is phenyl, a 5-membered heteroaryl, or a 6-membered heteroaryl;
X¹, X², X³, and X⁴ are the same or different and each is CR¹, N, S, or O, wherein no than three of X¹, X², X³, and X⁴ are N, S, or O;
n is 0 or 1; provided that when n is 0, at least one of X¹, X², X³, and X⁴ is N, S, or O;
X⁵ is O or S;
each instance of R¹ is the same or different and each is selected from the group consisting of H, deuterium, C₁₋₆ alkyl, cycloalkyl, alkoxy, cycloalkoxy, halo, amino, alkylamino, dialkylamino, amido, and heterocycloalkyl or
more than one instance of R¹ are linked to form a cycloalkyl or a phenyl;
R² is selected from the group consisting of H, alkyl, cycloalkyl, alkenyl, and aryl;
wherein the alkyl, cycloalkyl, alkenyl, and aryl of R² is optionally substituted with one or more substituents selected from the group consisting of deuterium, alkyl, alkoxy, halo, hydroxy, haloalkyl, alkoxy, alkyl, cycloalkyl, heterocycloalkyl, aryl, heteroaryl, amino, alkylamino, dialkylamino, amido, alkylsulfonamido, phosphonato, cyano, thiocyano, carboxylate, a protecting group, an amino acid residue, and a peptide residue;
R³ is selected from the group consisting of H, C₁₋₁₀ alkyl, cycloalkyl, aminoalkyl, alkylaminoalkyl, dialkylaminoalkyl, and arylcarbonylalkyl;
bond a is a single bond or double bond;
bond b is a single bond or double bond;
provided when bond a is a single bond, then R³ is present, bond b is a double bond, and R⁴ is S, and
when bond a is a double bond, then R³ is absent, bond b is a single bond, and R⁴ is H, alkyl, hydroxy, amino, or S—R⁵,
R⁵ is selected from the group consisting of C₁₋₁₀ alkyl, cycloalkyl, or acetyl; and
wherein the C₁₋₁₀ alkyl or cycloalkyl of R⁵ is optionally substituted with one or more substituents selected from the group consisting of deuterium, cycloalkyl, hydroxy, cyano, amido, and phenyl that is optionally further substituted with one or more substituents selected from alkyl, halo, and alkenyl.

Aspect (2) The method of aspect (1), wherein bond a is a single bond, bond b is a double bond, and R⁴ is S or a pharmaceutically acceptable salt thereof.

Aspect (3) The method of aspect (1), wherein bond a is a double bond, bond b is a single bond, and R⁴ is H, alkyl, hydroxy, amino, or S—R⁵ or a pharmaceutically acceptable salt thereof.

Aspect (4) The method of aspect (3), wherein R⁴ is S—R⁵ or a pharmaceutically acceptable salt thereof.

Aspect (5) The method of aspect (4), wherein R⁵ is C₁₋₆ alkyl, cycloalkyl, or arylalkyl or a pharmaceutically acceptable salt thereof.

Aspect (6) The method of aspect (5), wherein R⁵ is methyl or a pharmaceutically acceptable salt thereof.

Aspect (7) The method according to any one of aspects (1)-(6), wherein n is 1 and each of X¹, X², X³, and X⁴ is CR¹ or a pharmaceutically acceptable salt thereof.

Aspect (8) The method of aspect (7), wherein one instance of R¹ is halo or H, and the remaining three instances of R¹ are each H or a pharmaceutically acceptable salt thereof.

Aspect (9) The method of aspect (8), wherein X⁴ is CF or a pharmaceutically acceptable salt thereof.

Aspect (10) The method according to any one of aspects (1)-(6), wherein n is 1, one of X¹, X², X³, and X⁴ is N, and the remaining three of X¹, X², X³, and X⁴ are each CR¹ or a pharmaceutically acceptable salt thereof.

Aspect (11) The method of aspect (10), wherein each instance of R¹ is H or a pharmaceutically acceptable salt thereof.

Aspect (12) The method according to any one of aspects (1)-(6), wherein n is 0, and either X¹ or X³ is O or S and the remainder of X¹, X², and X³ is CR¹ or a pharmaceutically acceptable salt thereof.

Aspect (13) The method according to any one of aspects (1)-(6), wherein n is 0, and either X¹ and X² or X² and X³ are both N and the remainder of X¹ and X³ is CR1 or a pharmaceutically acceptable salt thereof.

Aspect (14) The method according to any one of aspects (1)-(6), wherein n is 0, and X¹ is N, X² is CR¹, and X³ is O or S or a pharmaceutically acceptable salt thereof.

Aspect (15) The method according to any one of aspects (1)-(6), wherein n is 0, and X¹ and X³ are both N, X² is O or a pharmaceutically acceptable salt thereof.

Aspect (16) The method according to any one of aspects (1)-(6), wherein ring A is selected from the group consisting of

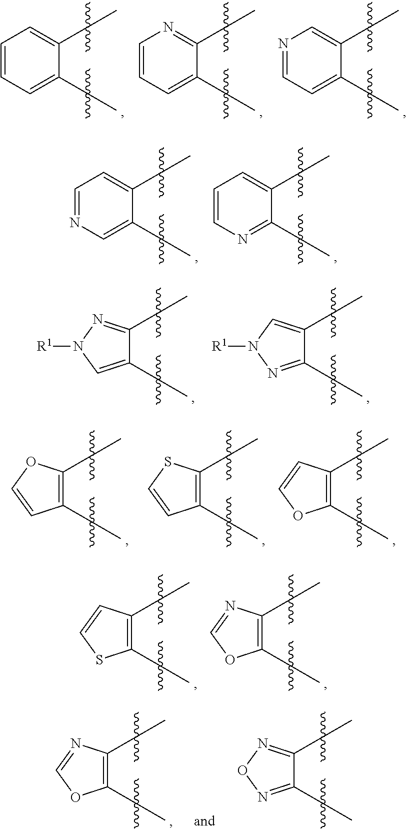

or a pharmaceutically acceptable salt thereof.

Aspect (17) The method according to any one of aspects (1)-(16), wherein X⁵ is O. or a pharmaceutically acceptable salt thereof.

Aspect (18) The method according to any one of aspects (1)-(17,) wherein R² is phenyl optionally substituted with alkyl, allyl, or alkyl optionally substituted with hydroxy, alkoxy, haloalkyl, cycloalkyl, heterocycloalkyl, aryl, heteroaryl, alkylsulfonamido, or amido of the formula —NHC(O)(CH₂)ₘR⁶, wherein m is 0-5, and R⁶ is alkyl, cycloalkyl, heterocycloalkyl, phenyl, and heteroaryl, each of which is optionally substituted with alkyl, alkoxy, hydroxy, halo, haloalkyl, cyano, heteroarylalkyl, or a combination thereof, or a pharmaceutically acceptable salt thereof.

Aspect (19) The method of aspect (18), wherein m is 0 or a pharmaceutically acceptable salt thereof.

Aspect (20) The method of aspect (18), wherein m is 1, 2, or 3 or a pharmaceutically acceptable salt thereof.

Aspect (21) The method of aspect (18), wherein R² is ethyl, n-propyl, —(CH₂)₃OMe, —(CH₂)₃OH, —(CH₂)₂-cyclopropyl, —(CH₂)₂-1-morpholino, —(CH₂)₃—NHSO₂CH₃, —(CH₂)₃—NHC(O)cyclopentyl, —(CH₂)₃—NHC(O)CH₂Ph-(2-Cl), —(CH₂)₃—NHC(O)CH₂Ph-(3-OMe), or —(CH₂)₃—NHC(O)CH₂Ph-(2-OMe) or a pharmaceutically acceptable salt thereof.

Aspect (22) The method according to any one of aspects (1)-(21), wherein R³ is H or dialkylaminoalkyl or a pharmaceutically acceptable salt thereof.

Aspect (23) The method of aspect (22), wherein R³ is H or a pharmaceutically acceptable salt thereof.

Aspect (24) The method according to any one of aspects (1)-(22), wherein the cancer comprises cancer cells that overexpress polo-like kinase 1 (Plk 1) relative to normal cells of the same tissue type.

Aspect (25) The method of aspect (24), wherein the cancer is breast cancer, lung cancer, renal cancer, liver cancer, uterine cancer, prostate cancer, pancreatic cancer, glioma, thyroid carcinoma, head and neck squamous cell carcinoma, melanoma, colorectal cancer, esophageal carcinoma, or ovarian carcinoma.

Aspect (26) The method according to any one of aspects (1)-(25), wherein the compound of formula (I) or a pharmaceutically acceptable salt thereof is co-administered with an anti-cancer agent and/or radiation therapy.

Aspect (27) A compound formula (Ib-1) or a pharmaceutically acceptable salt thereof:

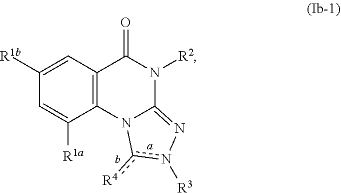

wherein
R^{1a} is H, F, or deuterium;
R^{1b} is H or deuterium;
R² is selected from the group consisting of alkyl, fluoroalkyl, cyclopropylalkyl, 2- or 4-pyridinylalkyl, 3-methoxybenzylamidoalkyl, 3-cyanobenzylamidoalkyl, each of which is optionally substituted with deuterium;
R³ is H or absent;
R⁴ is S or SCH₃;
bond a is a single bond or double bond;
bond b is a single bond or double bond; R⁴ is S or SCH₃;
   provided when bond a is a single bond, then R³ is H, bond b is a double bond, and R⁴ is S, and
   when bond a is a double bond, then R³ is absent, bond b is a single bond, and R⁴ is SCH₃.

Aspect (28) The compound of aspect (27) that is selected from the group consisting of

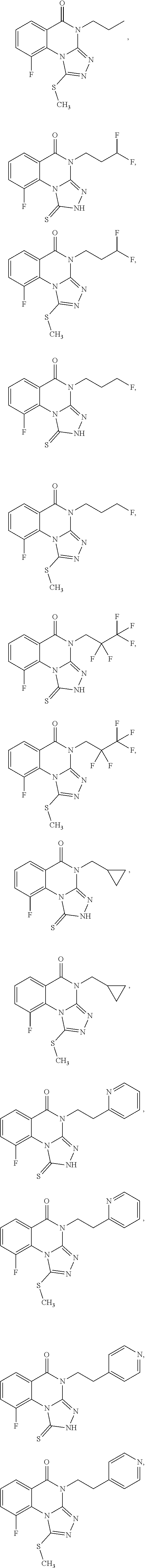

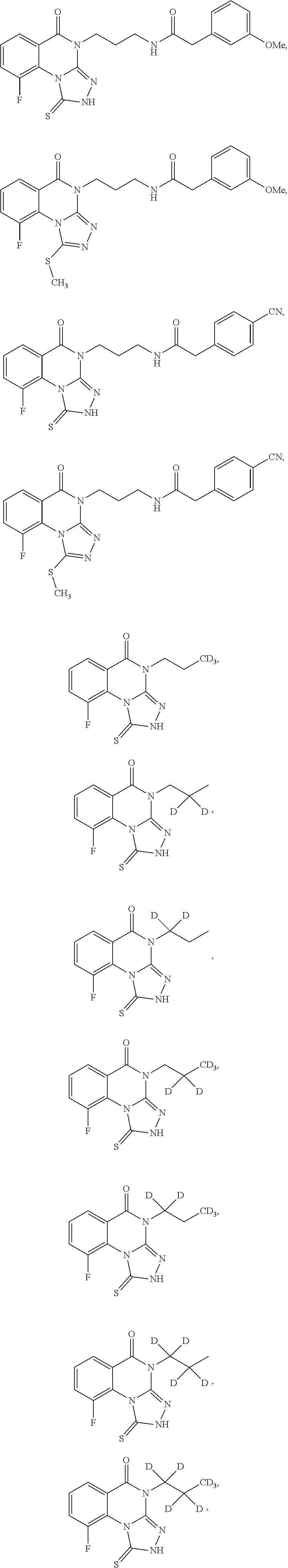

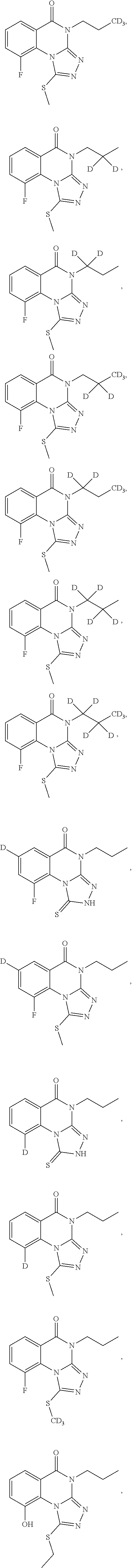

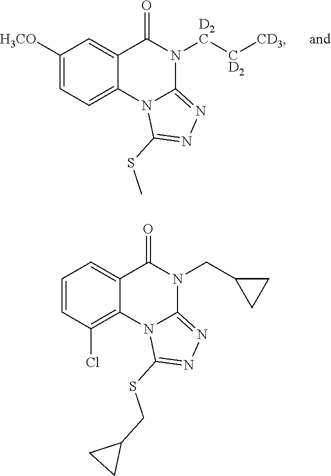

or a pharmaceutically acceptable salt thereof.

Aspect (29) A method of treating cancer comprising administering to a subject in need thereof a compound of formula (Ib-1) of aspect (27) or (28) or a pharmaceutically acceptable salt thereof, provided that the compound of formula (Ib-1) does not contain deuterium.

The following examples further illustrate the invention but, of course, should not be construed as in any way limiting its scope.

### EXAMPLES

Abbreviations. ADME, absorption distribution metabolism excretion; ATP, adenosine triphosphate; Boc, tert-butyloxycarbonyl; COMU, (1-cyano-2-ethoxy-2-oxoethylidenaminooxy)dimethylamino-morpholino-carbenium hexafluorophosphate; CYP, cytochrome P450; DCM, dichloromethane; DIPEA, N,N-diisopropylethylamine; DMF, N,N-dimethylformamide; DMSO, dimethylsulfoxide; EGFR, epidermal growth factor receptor; ELISA, enzyme-linked immunosorbent assay; FDA, Food and Drug Administration; FITC, fluorescein-5-isothiocyanate; FP, fluorescence polarization assay; HBA, hydrogen bond acceptor; HER2, human epidermal growth factor receptor 2; HPLC, high-performance liquid chromatography; HRMS, high-resolution mass spectrometry; IP, intraperitoneal; KD, kinase domain; LCMS, liquid chromatography-mass spectrometry; MLM, mouse liver microsome; MS/MS, tandem mass spectrometry; MT, microtubule; NAPDH, reduced nicotinamide adenine dinucleotide phosphate; PAMPA, parallel artificial membrane permeability assay; PBD, Polo-box domain; PBIP1, centromeric protein U; PEG, polyethylene glycol; PPI, protein-protein interaction; pyr, pyridine; RLM, rat liver microsome; RT, room temperature; SAR, structure-activity relationship; TFA, trifluoroacetic acid; UDPGA, uridine 5′-diphosphoglucuronic acid; UGT, uridine diphosphate glucuronosyltransferase

General Methods for Chemistry. All reactions were carried out under nitrogen atmosphere using anhydrous solvents. All moisture sensitive reactions were also performed with oven-dried glassware. Chemical reagents and anhydrous solvents were obtained from commercial sources and used as-is. Room temperature or rt refers to 25+2° C. Preparative purification was performed on a Waters semi-preparative HPLC. The column used was a Phenomenex Luna C18 (5 μm, 30×75 mm) at a flow rate of 45 m/min. The mobile phase consisted of acetonitrile and water (each containing 0.1% trifluoroacetic acid). A gradient of 10% to 50% acetonitrile over 8 min was used during the purification. Fraction collection was triggered by UV detection (220 nm). Initial analytical analysis during compound synthesis was performed on an Agilent 1200 LC-MS (Agilent Technologies) using a 3-min gradient of 4% to 100% acetonitrile (containing 0.025% trifluoroacetic acid) in water (containing 0.05% trifluoroacetic acid) was used with an 8-min run time at a flow rate of 1 mL/min. The purity of compounds newly synthesized was demonstrated on an Agilent 1200 LC-MS (Agilent Technologies) using a 7-min linear gradient of 4% to 100% acetonitrile (containing 0.025% trifluoroacetic acid) in water (containing 0.05% trifluoroacetic acid) followed by a 4.5-min run time at a flow rate of 1 m/min and a Phenomenex Luna C18 column (3 μm, 3×75 mm) at 50° C. Purity of purchased compounds was determined using an Agilent ZORBAX Eclipse XDB C18 column (5 mm, 4.6×250 mm) with a linear gradient of 5% to 95% acetonitrile in water (containing 10 mM triethylammonium acetate) for 20 min at a flow rate of 1.0 m/min. ¹H and ¹³C NMR spectra were recorded on either a Varian 400 (100) MHz spectrometer or a Bruker 400 MHz spectrometer. Chemical shifts are given in ppm (δ), calibrated to the residual solvent signals and frequency calibrated internally by solvent for ¹⁹F NMR (BrukerTopspin/MestReNova 10.0.2 or 14.1.0). High resolution mass spectrometry was recorded on either an Agilent 6210 Time-of-Flight LCMS system or a Waters Micromass spectrometer equipped with a standard electrospray ionization (ESI) and modular LockSpray™ interface. Purity of all the tested compounds (including both newly synthesized and purchased, active compounds) were demonstrated to be >95% pure at 254 nm, except commercially procured compound 58 which was 93.5% pure.

### General Procedure 1

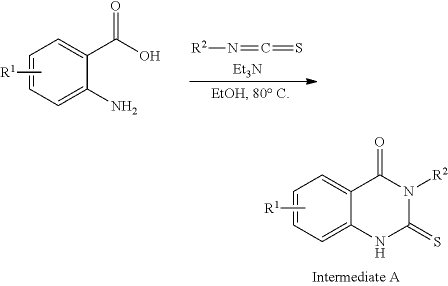

General Procedure A. To a solution of appropriate 2-aminobenzoic acid (1 eq.) and isothiocyanate (1.2 eq.) in EtOH (0.37 M) was added triethylamine (1.2 eq.). The reaction was stirred at 80° C. for 1-2 h. The reaction was cooled to room temperature (RT) and diluted with water. The solid (Intermediate A) was filtered and dried under vacuum and carried on without further purification.

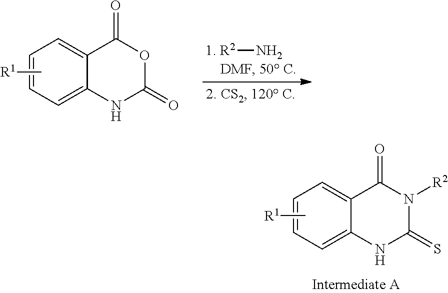

General Procedure B. A solution of the isatoic anhydride (1 eq.) in acetonitrile (0.67 M) was added the amine (1.5 eq.). In the case of amine salts, triethylamine (1.5 eq.) was added to a solution of the amine in acetonitrile (0.67 M), the salt was removed by filtration and the free based amine solution was added to the reaction. The reaction was heated at 50° C. for 30 min. The reaction was cooled to RT and carbon disulfide (7 eq.) was added cold. The reaction was heated to 120° C. for 45 min. The reaction was diluted with Et₂O, filtered, rinsed with Et₂O, and dried. The solid (Intermediate A) was dried under vacuum and carried on without further purification.

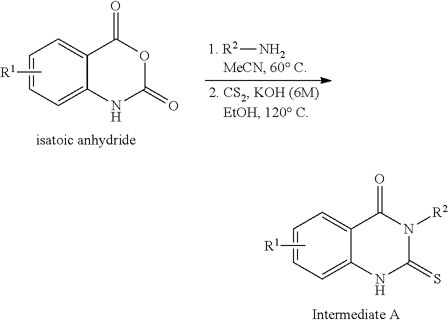

General Procedure C. A solution of the isatoic anhydride (1 eq.) in acetonitrile (0.67 M) was added the amine (1.5 eq.). In the case of amine salts, triethylamine (1.5 eq.) was added to a solution of the amine in acetonitrile (0.67 M), the salt was removed by filtration and the free based amine solution was added to the reaction. The reaction was heated at 60° C. for 3 h. The reaction was concentrated, and the residue dissolved in EtOH (0.67 M), and aq. KOH (1.2 eq.) was added followed by carbon disulfide (2 eq.). The reaction was heated to 120° C. for 2 h. The reaction was cooled to RT, diluted with water and washed with Et₂O. The solid (Intermediate A) was dried under vacuum and carried on without further purification.

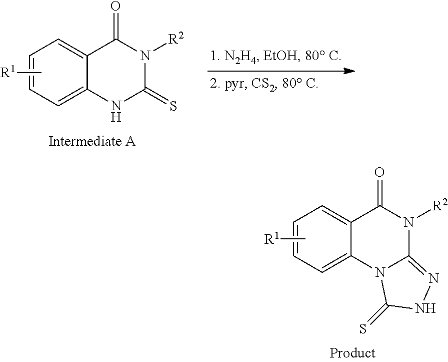

General Procedure D. To a solution of Intermediate A (1 eq.) in EtOH (0.25-0.35 M) was added hydrazine (7 eq.). The reaction was heated to 80° C. for 4 h. The reaction was cooled to RT and pyridine (10 eq.) and carbon disulfide (10 eq.) were added. The reaction was heated to 80° C. for 1-2 h. The reaction was poured into cold water, and the product filtered and washed with water or purified by HPLC.

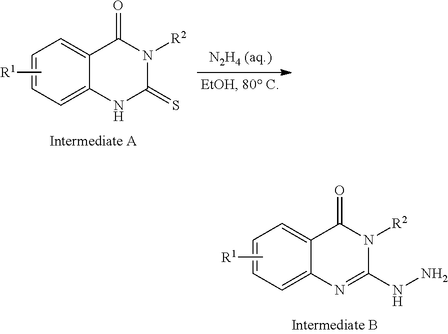

General Procedure E. To a solution of Intermediate A (1 eq.) in EtOH (0.35 M) was added water (2.8 eq.) followed by hydrazine (7 eq.). The reaction was heated to 80° C. for 4 h. The reaction was then poured into cold water and concentrated. The crude material was purified on a Teledyne ISCO CombiFlash® System by (dry-loading) (EtOAc/DCM: 0-3%) to give Intermediate B.

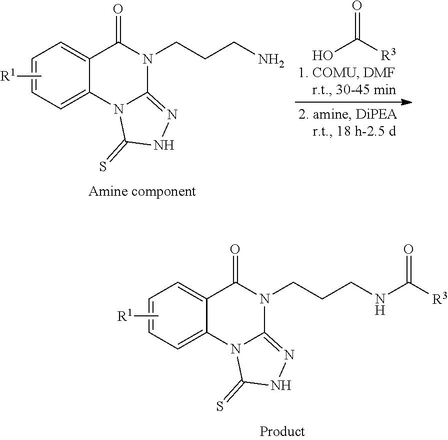

General Procedure F. To a solution of the carboxylic acid (1.5 eq.) in DMF (0.2 M) was added COMU (1.5 eq.) and the reaction stirred at RT for 30 to 45 min. Then the amine (1 eq.) was added followed by DIPEA (2.2 eq.). The reaction was then stirred for 18 h or 2.5 d at RT. The crude material was purified by HPLC.

### Example 1

This example demonstrates a synthesis of 4-phenethyl-1-thioxo-2,4-dihydro-[1,2,4]triazolo[4,3-a]quinazolin-5(1H)-one in an aspect of the invention.

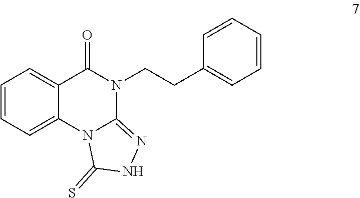 [SMILES: O=c1c2ccccc2n2c(=S)[nH]nc2n1CCc1ccccc1]

2-Amino-N-phenethylbenzamide: A mixture of methylanthranilate (0.856 mL, 6.615 mmol) and phenethylamine (1.3 mL, 10 mmol) was stirred in a round bottom flask at 190° C. for 5 h. The product was purified by silica-gel column chromatography (0.44 g, 28%). ¹H NMR (400 MHz, CDCl₃) δ 7.40-7.14 (m, 6H), 6.68 (dd, J=1.1, 8.1 Hz, 1H), 6.62 (ddd, J=1.2, 7.2, 8.1 Hz, 1H), 6.06 (s, 1H), 5.49 (s, 2H), 3.70 (td, J=5.8, 6.8 Hz, 2H), 2.94 (t, J=6.9 Hz, 2H); HRMS (M+H) for C₁₅H₁₆N₂O calculated 241.1341, found 241.1339.

3-Phenethyl-2-thioxo-2,3-dihydroquinazolin-4(1H)-one: Route 1: To a solution of 2-amino-N-phenethylbenzamide (100 mg, 0.416 mmol) in anhydrous DMF (2 mL) at room temperature was added carbon disulfide (72 μL, 1.248 mmol) and DBU (136 μL, 0.916 mmol) sequentially. After stirring the reaction mixture for 18 h at room temperature, cold TN Aq. HCl was added with vigorous stirring. The precipitate was collected by filtration, washed with cold water and hexanes, and dried to afford practically pure product (60 mg, 50%). Route 2: To a solution of 2-amino-N-phenethylbenzamide (100 mg, 0.416 mmol) in anhydrous ethanol (2 mL) at room temperature was added carbon disulfide (75 μL, 1.25 mmol) and solid KOH (32 mg, 0.916 mmol) sequentially. The reaction mixture was stirred at 80° C. for 18 h at room temperature, cooled, and was added to a cold TN Aq. HCl with vigorous stirring. The precipitate was collected by filtration, washed with cold water and hexanes, and dried to afford practically pure product (70 mg, 60%). The product was purified further by silica-gel column chromatography using 0-2% methanol in dichloromethane as an eluent. The purified product gave better yield in the next step. 1H NMR (400 MHz, CDCl3) δ 9.99 (s, 1H), 8.25-8.10 (m, 1H), 7.69 (ddd, J=1.5, 7.3, 8.4 Hz, 1H), 7.45-7.40 (m, 2H), 7.38-7.32 (m, 2H), 7.27 (d, J=5.6 Hz, 1H), 7.13 (dt, J=0.7, 8.2 Hz, 1H), 4.82-4.65 (m, 2H), 3.19-3.07 (m, 2H); HRMS (M+H) for C16H14N2OS calculated 283.0905, found 283.0909.

2-Hydrazineyl-3-phenethylquinazolin-4(3H)-one: To a suspension of 3-phenethyl-2-thioxo-2,3-dihydroquinazolin-4(1H)-one (200 mg, 0.708 mmol) in anhydrous ethanol (5 mL) was added anhydrous hydrazine (0.33 mL, 10.62 mmol), and heated at 85° C. for 18 h. The volatiles were evaporated under high vacuum and followed by co-evaporation with toluene (2×) gave 2-hydrazineyl-3-phenethylquinazolin-4(3H)-one as yellow solid and was used as such without further purification (200 mg, quantitative). 1H NMR (400 MHz, CDCl3) δ 8.16 (dd, J=1.6, 7.9 Hz, 1H), 7.68-7.56 (m, 1H), 7.45-7.14 (m, 5H), 4.21 (dd, J=6.7, 8.4 Hz, 2H), 3.09-2.99 (m, 1H); HRMS (M+H) for C16H16N4O calculated 281.1402, found 281.1405.

4-Phenethyl-1-thioxo-2,4-dihydro-[1,2,4]triazolo[4,3-a]quinazolin-5(1H)-one: 2-Hydrazineyl-3-phenethylquinazolin-4(3H)-one (200 mg, 0.713 mmol) was dissolved in anhydrous ethanol (10 mL). To the solution was added carbon disulfide (0.13 mL, 2.14 mmol) followed by solid KOH (120 mg, 2.14 mmol), and heated to 80° C. for 18 h. The reaction mixture was cooled, and poured into a cold 1N aq.HCl solution. The precipitates were collected and purified by silica-gel column chromatography using 0-2% methanol in dichloromethane as an eluent to afford pure product as a white solid (200 mg, 87%). ¹H NMR (400 MHz, DMSO-d₆) δ 14.10 (s, 1H), 10.23 (dd, J=1.1, 8.6 Hz, 1H), 8.22 (dd, J=1.6, 7.9 Hz, 1H), 7.91 (ddd, J=1.7, 7.3, 8.7 Hz, 1H), 7.62 (td, J=1.1, 7.6 Hz, 1H), 7.38-7.15 (m, 4H), 4.32-4.15 (m, 2H), 3.08-2.92 (m, 2H); HRMS (M+H) for C₁₇H₁₄N₄OS calculated 323.0967, found 323.0966.

### Example 2

This example demonstrates a synthesis of 4-phenethyl-2,4-dihydro-[1,2,4]triazolo[4,3-a]quinazoline-1,5-dione in an aspect of the invention.

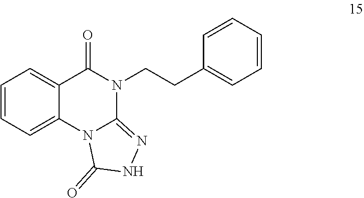 [SMILES: O=c1c2ccccc2n2c(=O)[nH]nc2n1CCc1ccccc1]

2-Hydrazineyl-3-phenethylquinazolin-4(3H)-one (35 mg, 0.117 mmol) was suspended in anhydrous toluene (2 mL), and was added carbonyl diimidazole (CDI, 22 mg, 0.135 mmol) and heated to reflux (115° C.) for 1.5 h. The volatiles were evaporated under vacuum and, the residue was treated with 1N aq. HCl and 5% isopropanol-dichloromethane. The organic layer was separated, dried over Na₂SO₄, evaporated, and the residue purified by silica-gel column chromatography to afford 7b as a white solid (13 mg, 34%). ¹H NMR (400 MHz, DMSO-d₆) δ 12.07 (s, 1H), 8.65 (dd, J=4.6, 9.1 Hz, 1H), 7.84 (dd, J=3.0, 8.6 Hz, 1H), 7.75 (td, J=3.1, 8.7 Hz, 1H), 7.35-7.19 (m, 4H), 4.26-4.05 (m, 2H), 3.08-2.92 (m, 2H); ¹⁹F NMR (376 MHz, DMSO-d₆) δ −113.33 (td, J=4.5, 8.1 Hz); HRMS (M+H) for C₁₇H₁₃N₄O₂F calculated 325.1101, found 325.1107.

### Example 3

This example demonstrates a synthesis of 1-amino-7-fluoro-4-phenethyl-[1,2,4]triazolo[4,3-a]quinazolin-5(4H)-one in an aspect of the invention.

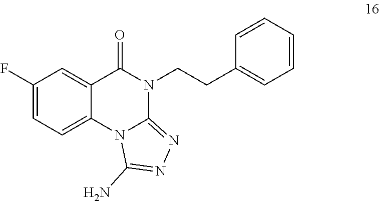 [SMILES: Nc1nnc2n(CCc3ccccc3)c(=O)c3cc(F)ccc3n12]

6-Fluoro-3-phenethyl-2-thioxo-2,3-dihydroquinazolin-4(1H)-one was synthesized according to General Procedure A. 6-Fluoro-2-hydrazineyl-3-phenethylquinazolin-4(3H)-one was synthesized according to General Procedure E. Then to a solution of 6-fluoro-2-hydrazinyl-3-phenethylquinazolin-4(3H)-one (0.1 g, 0.335 mmol) and cyanogen bromide (0.036 g, 0.335 mmol) in ethanol (16.8 ml) was added sodium hydroxide (0.067 g, 1.675 mmol) and the reaction was stirred at room temperature for 3 h. The reaction was neutralized with sat. NaHCO₃ and the solid filtered. The solid was redissolved and purified by HPLC to give 1-amino-7-fluoro-4-phenethyl-[1,2,4]triazolo[4,3-a]quinazolin-5(4H)-one, 2 TFA: ¹H NMR (400 MHz, DMSO-d₆) δ 8.29 (dd, J=9.2, 4.2 Hz, 1H), 7.92 (dd, J=8.5, 3.1 Hz, 1H), 7.82 (td, J=8.6, 3.1 Hz, 1H), 7.34-7.24 (m, 4H), 7.22 (d, J=7.0 Hz, 1H), 6.90 (s, 2H), 4.29 (dd, J=9.5, 6.5 Hz, 2H), 3.06-2.98 (m, 2H); ¹⁹F NMR (376 MHz, DMSO-d₆) δ −114.22; HRMS (ES-API): m/z calculated for C₁₇H₁₅FN₅O (M+H) 324.1255, found 324.1264.

### Example 4

This example demonstrates a synthesis of 7-fluoro-1-methyl-4-phenethyl-[1,2,4]triazolo[4,3-a]quinazolin-5(4H)-one in an aspect of the invention.

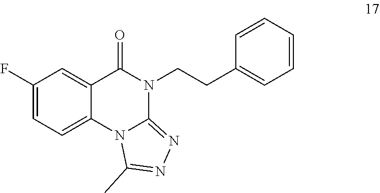 [SMILES: Cc1nnc2n(CCc3ccccc3)c(=O)c3cc(F)ccc3n12]

6-Fluoro-3-phenethyl-2-thioxo-2,3-dihydroquinazolin-4(1H)-one was synthesized according to General Procedure A. 6-Fluoro-2-hydrazineyl-3-phenethylquinazolin-4(3H)-one was synthesized according to General Procedure E. Then 6-fluoro-2-hydrazinyl-3-phenethylquinazolin-4(3H)-one (0.1 g, 0.335 mmol) was stirred in Ac₂O (16.8 ml) at 100° C. for 5 h. The reaction was concentrated, filtered, and washed with water. The solid was dried and crystallized from EtOH to give 7-fluoro-1-methyl-4-phenethyl-[1,2,4]triazolo[4,3-a]quinazolin-5(4H)-one (0.0453 g, 42%): ¹H NMR (400 MHz, DMSO-d₆) δ 8.11 (dd, J=9.2, 4.2 Hz, 1H), 7.95 (dd, J=8.5, 3.1 Hz, 1H), 7.80 (ddd, J=9.2, 8.0, 3.1 Hz, 1H), 7.29 (td, J=9.0, 8.4, 6.1 Hz, 4H), 7.21 (td, J=6.5, 2.2 Hz, 1H), 4.42-4.33 (m, 2H), 3.08-2.99 (m, 2H), 2.84 (s, 3H); ¹⁹F NMR (376 MHz, DMSO-d₆) δ −114.29 (td, J=8.2, 4.0 Hz).

### Example 5

This example demonstrates a synthesis of 7-fluoro-4-phenethyl-[1,2,4]triazolo[4,3-a]quinazolin-5(4H)-one in an aspect of the invention.

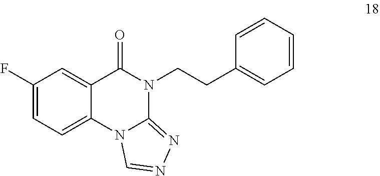 [SMILES: O=c1c2cc(F)ccc2n2cnnc2n1CCc1ccccc1]

6-Fluoro-3-phenethyl-2-thioxo-2,3-dihydroquinazolin-4(1H)-one was synthesized according to General Procedure A. 6-Fluoro-2-hydrazineyl-3-phenethylquinazolin-4(3H)-one was synthesized according to General Procedure E. Then 6-fluoro-2-hydrazinyl-3-phenethylquinazolin-4(3H)-one (0.1 g, 0.335 mmol) was stirred in formic acid (16.8 ml) at 100° C. for 5 h. The reaction was concentrated, redissolved, and purified by HPLC to give 7-fluoro-4-phenethyl-[1,2,4]triazolo[4,3-a]quinazolin-5(4H)-one, TFA: ¹H NMR (400 MHz, DMSO-d₆) δ 9.50 (s, 1H), 8.27 (dd, J=8.8, 4.3 Hz, 1H), 7.89 (ddd, J=11.0, 7.1, 3.9 Hz, 2H), 7.34-7.25 (m, 4H), 7.25-7.17 (m, 1H), 4.37 (dd, J=9.2, 6.8 Hz, 2H), 3.09-3.01 (m, 2H); ¹⁹F NMR (376 MHz, DMSO-d₆) δ −113.78 (td, J=8.3, 4.1 Hz); HRMS (ES-API): m/z calculated for C₁₇H₁₄FN₄O (M+H) 309.1146, found 309.1155.

### Example 6

This example demonstrates a synthesis of 1-(methylthio)-4-phenethyl-[1,2,4]triazolo[4,3-a]quinazolin-5(4H)-one in an aspect of the invention.

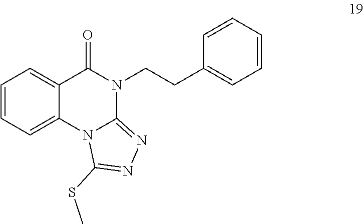 [SMILES: CSc1nnc2n(CCc3ccccc3)c(=O)c3ccccc3n12]

3-Phenethyl-2-thioxo-2,3-dihydroquinazolin-4(1H)-one was synthesized according to General Procedure A. Then 4-phenethyl-1-thioxo-2,4-dihydro-[1,2,4]triazolo[4,3-a]quinazolin-5(1H)-one was synthesized according to General Procedure D. Then 1-(methylthio)-4-phenethyl-[1,2,4]triazolo[4,3-a]quinazolin-5(4H)-one was synthesized by: to a solution of 4-phenethyl-1-thioxo-2,4-dihydro-[1,2,4]triazolo[4,3-a]quinazolin-5(1H)-one (0.092 g, 0.223 mmol) in DMF (1.113 ml) was added K₂CO₃ (0.037 g, 0.267 mmol) and MeI (0.017 ml, 0.267 mmol). The reaction was quenched with methanol and concentrated. The reaction was stirred for 3 hr and quenched with methanol. The crude mixture was concentrated and purified by ISCO to give 1-(methylthio)-4-phenethyl-[1,2,4]triazolo[4,3-a]quinazolin-5(4H)-one, TFA.

### Example 7

This example demonstrates a synthesis of 4-ethyl-1-thioxo-2,4-dihydro-[1,2,4]triazolo[4,3-a]quinazolin-5(1H)-one in an aspect of the invention.

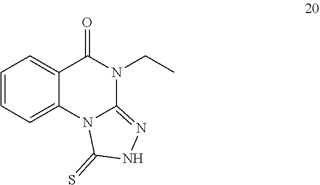 [SMILES: CCn1c(=O)c2ccccc2n2c(=S)[nH]nc12]

3-Ethyl-2-thioxo-2,3-dihydroquinazolin-4(1H)-one was synthesized according to General Procedure A. Then 4-ethyl-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.20 (d, J=8.5 Hz, 1H), 8.20 (d, J=7.8 Hz, 1H), 7.87 (t, J=8.0 Hz, 1H), 7.59 (t, J=7.7 Hz, 1H), 4.05 (q, J=7.1 Hz, 2H), 1.23 (t, J=7.2 Hz, 3H); HRMS (ES-API): m/z calculated for C₁₁H₁₀N₄NaOS (M+Na) 269.0468, found 269.0476.

### Example 8

This example exemplifies commercially available compounds of formula (I) in an aspect of the invention.

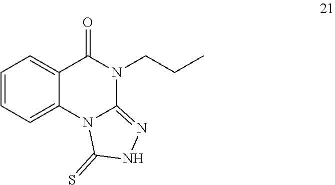 [SMILES: CCCn1c(=O)c2ccccc2n2c(=S)[nH]nc12]

4-Propyl-1-thioxo-2,4-dihydro-[1,2,4]triazolo[4,3-a]quinazolin-5(1H)-one was purchased from Enamine (Monmouth Jct., NJ).

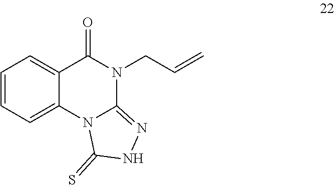 [SMILES: C=CCn1c(=O)c2ccccc2n2c(=S)[nH]nc12]

4-Allyl-1-thioxo-2,4-dihydro-[1,2,4]triazolo[4,3-a]quinazolin-5(1H)-one was purchased from Enamine (Monmouth Jct., NJ).

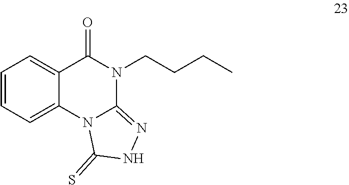 [SMILES: CCCCn1c(=O)c2ccccc2n2c(=S)[nH]nc12]

4-Butyl-1-thioxo-2,4-dihydro-[1,2,4]triazolo[4,3-a]quinazolin-5(1H)-one was purchased from Enamine (Monmouth Jet., NJ).

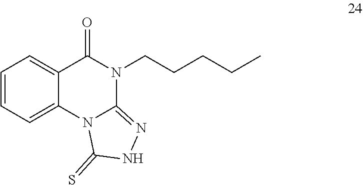 [SMILES: CCCCCn1c(=O)c2ccccc2n2c(=S)[nH]nc12]

4-Pentyl-1-thioxo-2,4-dihydro-[1,2,4]triazolo[4,3-a]quinazolin-5(1H)-one was purchased from Enamine (Monmouth Jet., NJ).

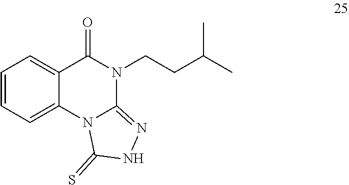 [SMILES: CC(C)CCn1c(=O)c2ccccc2n2c(=S)[nH]nc12]

4-Isopentyl-1-thioxo-2,4-dihydro-[1,2,4]triazolo[4,3-a]quinazolin-5(1H)-one was purchased from Enamine (Monmouth Jet., NJ).

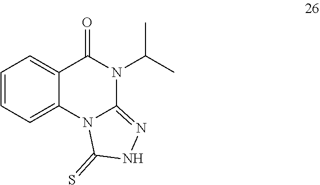 [SMILES: CC(C)n1c(=O)c2ccccc2n2c(=S)[nH]nc12]

4-Isopropyl-1-thioxo-2,4-dihydro-[1,2,4]triazolo[4,3-a]quinazolin-5(1H)-one was purchased from ChemDiv (San Diego, CA).

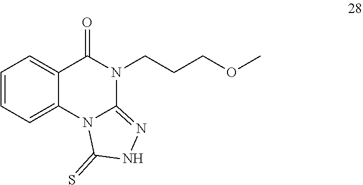 [SMILES: COCCCn1c(=O)c2ccccc2n2c(=S)[nH]nc12]

4-(3-Methoxypropyl)-1-thioxo-2,4-dihydro-[1,2,4]triazolo[4,3-a]quinazolin-5(1H)-one was purchased from Enamine (Monmouth Jet., NJ).

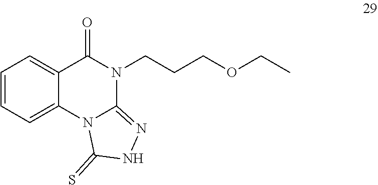 [SMILES: CCOCCCn1c(=O)c2ccccc2n2c(=S)[nH]nc12]

4-(3-Ethoxypropyl)-1-thioxo-2,4-dihydro-[1,2,4]triazolo[4,3-a]quinazolin-5(1H)-one was purchased from Enamine (Monmouth Jet., NJ).

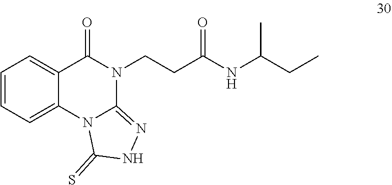 [SMILES: CCC(C)NC(=O)CCn1c(=O)c2ccccc2n2c(=S)[nH]nc12]

N-(sec-Butyl)-3-(5-oxo-1-thioxo-1,2-dihydro-[1,2,4]triazolo[4,3-a]quinazolin-4(5H)-yl)propenamide was purchased from ChemDiv (San Diego, CA).

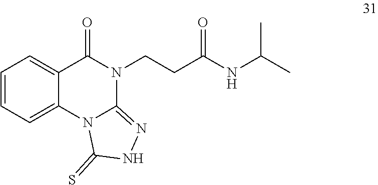 [SMILES: CC(C)NC(=O)CCn1c(=O)c2ccccc2n2c(=S)[nH]nc12]

N-Isopropyl-3-(5-oxo-1-thioxo-1,2-dihydro-[1,2,4]triazolo[4,3-a]quinazolin-4(5H)-yl)propenamide was purchased from ChemDiv (San Diego, CA).

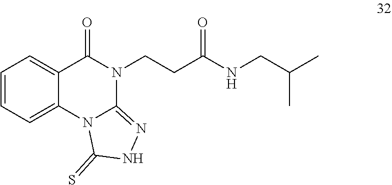 [SMILES: CC(C)CNC(=O)CCn1c(=O)c2ccccc2n2c(=S)[nH]nc12]

N-Isobutyl-3-(5-oxo-1-thioxo-1,2-dihydro-[1,2,4]triazolo[4,3-a]quinazolin-4(5H)-yl)propenamide was purchased from ChemDiv (San Diego, CA).

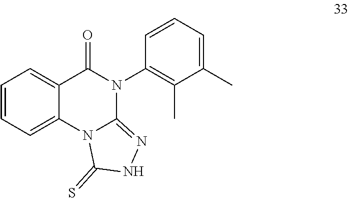 [SMILES: Cc1cccc(-n2c(=O)c3ccccc3n3c(=S)[nH]nc23)c1C]

4-(2,3-Dimethylphenyl)-1-thioxo-2,4-dihydro-[1,2,4]triazolo[4,3-a]quinazolin-5(11)-one was purchased from Enamine (Monmouth Jct., NJ).

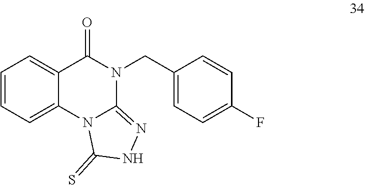 [SMILES: O=c1c2ccccc2n2c(=S)[nH]nc2n1Cc1ccc(F)cc1]

4-(4-Fluorobenzyl)-1-thioxo-2,4-dihydro-[1,2,4]triazolo[4,3-a]quinazolin-5(1H)-one was purchased from ChemDiv (San Diego, CA).

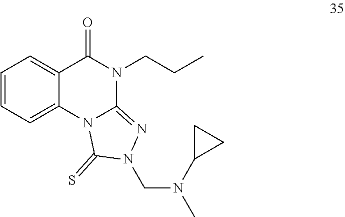 [SMILES: CCCn1c(=O)c2ccccc2n2c(=S)n(CN(C)C3CC3)nc12]

2-((Cyclopropyl(methyl)amino)methyl)-4-propyl-1-thioxo-2,4-dihydro-[1,2,4]triazolo[4,3-a]quinazolin-5(1H)-one was purchased from Enamine (Monmouth Jet., NJ).

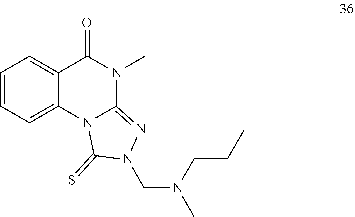 [SMILES: CCCN(C)Cn1nc2n(C)c(=O)c3ccccc3n2c1=S]

4-Methyl-2-((methyl(propyl)amino)methyl)-1-thioxo-2,4-dihydro-[1,2,4]triazolo[4,3-a]quinazolin-5(1H)-one was purchased from Enamine (Monmouth Jet., NJ).

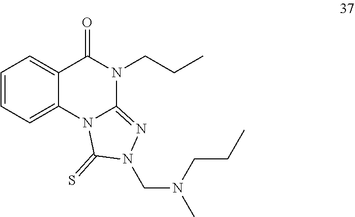 [SMILES: CCCN(C)Cn1nc2n(CCC)c(=O)c3ccccc3n2c1=S]

2-((Methyl(propyl)amino)methyl)-4-propyl-1-thioxo-2,4-dihydro-[1,2,4]triazolo[4,3-a]quinazolin-5(1H)-one was purchased from Enamine (Monmouth Jet., NJ).

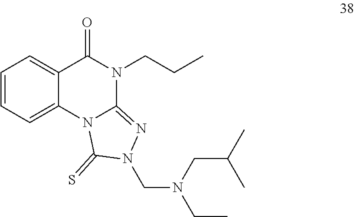 [SMILES: CCCn1c(=O)c2ccccc2n2c(=S)n(CN(CC)CC(C)C)nc12]

2-(((Cyclopropylmethyl)(ethyl)amino)methyl)-4-propyl-1-thioxo-2,4-dihydro-[1,2,4]triazolo[4,3-a]quinazolin-5(1H)-one was purchased from Enamine (Monmouth Jet., NJ).

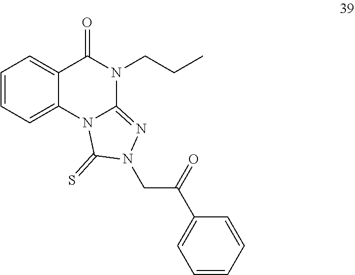 [SMILES: CCCn1c(=O)c2ccccc2n2c(=S)n(CC(=O)c3ccccc3)nc12]

2-(2-Oxo-2-phenylethyl)-4-propyl-1-thioxo-2,4-dihydro-[1,2,4]triazolo[4,3-a]quinazolin-5(1H)-one was purchased from ChemDiv (San Diego, CA).

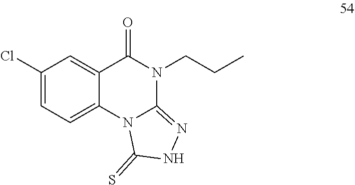 [SMILES: CCCn1c(=O)c2cc(Cl)ccc2n2c(=S)[nH]nc12]

7-Chloro-4-propyl-1-thioxo-2,4-dihydro-[1,2,4]triazolo[4,3-a]quinazolin-5(1H)-one one was purchased from ChemDiv (San Diego, CA).

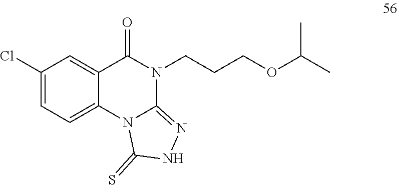 [SMILES: CC(C)OCCCn1c(=O)c2cc(Cl)ccc2n2c(=S)[nH]nc12]

7-Chloro-4-(3-isopropoxypropyl)-1-thioxo-2,4-dihydro-[1,2,4]triazolo[4,3-a]quinazolin-5(1H)-one was purchased from ChemDiv (San Diego, CA).

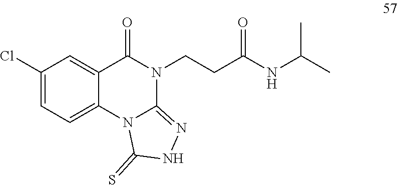 [SMILES: CC(C)NC(=O)CCn1c(=O)c2cc(Cl)ccc2n2c(=S)[nH]nc12]

3-(7-Chloro-5-oxo-1-thioxo-1,2-dihydro-[1,2,4]triazolo[4,3-a]quinazolin-4(5H)-yl)-N-isopropylpropanamide one was purchased from ChemDiv (San Diego, CA).

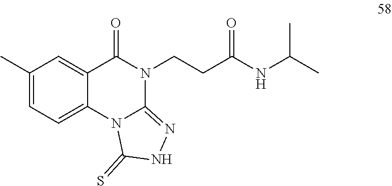 [SMILES: Cc1ccc2c(c1)c(=O)n(CCC(=O)NC(C)C)c1n[nH]c(=S)n21]

N-Isopropyl-3-(7-methyl-5-oxo-1-thioxo-1,2-dihydro-[1,2,4]triazolo[4,3-a]quinazolin-4(5H)-yl)propanamide one was purchased from ChemDiv (San Diego, CA). Purity (HPLC) 93.49%.

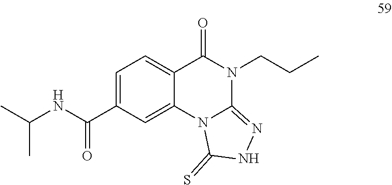 [SMILES: CCCn1c(=O)c2ccc(C(=O)NC(C)C)cc2n2c(=S)[nH]nc12]

N-Isopropyl-5-oxo-4-propyl-1-thioxo-1,2,4,5-tetrahydro-[1,2,4]triazolo[4,3-a]quinazoline-8-carboxamide one was purchased from ChemDiv (San Diego, CA).

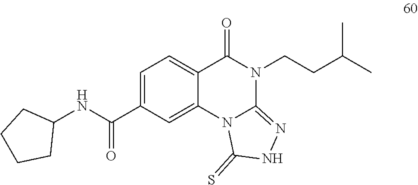 [SMILES: CC(C)CCn1c(=O)c2ccc(C(=O)NC3CCCC3)cc2n2c(=S)[nH]nc12]

N-Cyclopentyl-4-isopentyl-5-oxo-1-thioxo-1,2,4,5-tetrahydro-[1,2,4]triazolo[4,3-a]quinazoline-8-carboxamideone was purchased from ChemDiv (San Diego, CA).

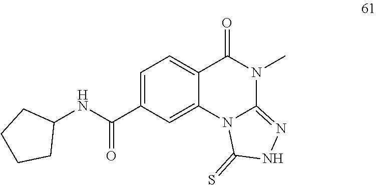 [SMILES: Cn1c(=O)c2ccc(C(=O)NC3CCCC3)cc2n2c(=S)[nH]nc12]

N-Cyclopentyl-4-methyl-5-oxo-1-thioxo-1,2,4,5-tetrahydro-[1,2,4]triazolo[4,3-a]quinazoline-8-carboxamide one was purchased from ChemDiv (San Diego, CA).

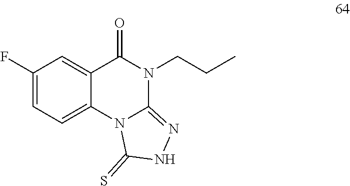 [SMILES: CCCn1c(=O)c2cc(F)ccc2n2c(=S)[nH]nc12]

7-Fluoro-4-propyl-1-thioxo-2,4-dihydro-[1,2,4]triazolo[4,3-a]quinazolin-5(1H)-one was purchased from ChemDiv (San Diego, CA).

### Example 9

This example demonstrates a synthesis of 1-thioxo-4-(3,3,3-trifluoropropyl)-2,4-dihydro-[1,2,4]triazolo[4,3-a]quinazolin-5(1H)-one in an aspect of the invention.

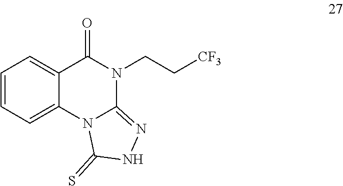 [SMILES: O=c1c2ccccc2n2c(=S)[nH]nc2n1CCC(F)(F)F]

2-Thioxo-3-(3,3,3-trifluoropropyl)-2,3-dihydroquinazolin-4(1H)-one was synthesized according to General Procedure B. Then 1-thioxo-4-(3,3,3-trifluoropropyl)-2,4-dihydro-[1,2,4]triazolo[4,3-a]quinazolin-5(1H)-one, TFA was synthesized according to General Procedure D: ¹H NMR (400 MHz, DMSO-d₆) δ 10.19 (d, J=8.5 Hz, 1H), 8.21 (d, J=7.8 Hz, 1H), 7.90 (t, J=8.1 Hz, 1H), 7.61 (t, J=7.7 Hz, 1H), 4.25 (t, J=7.3 Hz, 2H), 2.72 (dt, J=11.5, 7.4 Hz, 2H); ¹⁹F NMR (376 MHz, DMSO-d₆) δ −64.31 (t, J=11.3 Hz.

### Example 10

This example demonstrates a synthesis of 7-fluoro-4-phenethyl-1-thioxo-2,4-dihydro-[1,2,4]triazolo[4,3-a]quinazolin-5(1H)-one in an aspect of the invention.

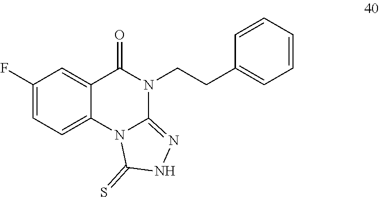 [SMILES: O=c1c2cc(F)ccc2n2c(=S)[nH]nc2n1CCc1ccccc1]

2-Amino-5-fluoro-N-phenethylbenzamide: To a solution of 2-amino-5-fluorobenzoic acid (250 mg, 1.61 mmol), phenethylamine (0.243 mL, 1.93 mmol), dimethylaminopyridine (DMAP, 100 mg, 0.81 mmol) and triethylamine (0.45 mL, 3.22 mmol) in anhydrous dichloromethane (5 mL) was added EDC·HCl (370 mg, 1.93 mmol), and stirred at room temperature for 18 h. Saturated Aq. NaHCO₃ solution and dichloromethane were added, stirred. The organic layer was separated, dried over Na₂SO₄, and evaporated under reduced pressure. The residue obtained was purified by silica-gel chromatography to afford the desired product as a white solid (200 mg, 48%). ¹H NMR (400 MHz, CDCl₃) δ 7.36 (t, J=7.3 Hz, 2H), 7.27 (q, J=6.7, 7.6 Hz, 3H), 6.96 (td, J=2.9, 8.4 Hz, 1H), 6.89 (dd, J=2.8, 9.2 Hz, 1H), 6.64 (dd, J=4.6, 9.0 Hz, 1H), 6.01 (s, 1H), 5.24 (s, 2H), 3.69 (q, J=6.6 Hz, 2H), 2.94 (t, J=6.9 Hz, 2H); ¹⁹F NMR (376 MHz, CDCl₃) δ −111.91; HRMS (M+H) for C₁₆H₁₅N₂OF calculated 259.1247, found 259.1247.

6-Fluoro-3-phenethyl-2-thioxo-2,3-dihydroquinazolin-4(1H)-one: To a solution of 2-amino-5-fluoro-N-phenethylbenzamide (100 mg, 0.387 mmol) in anhydrous DMF (2 mL) at room temperature was added carbon disulfide (70 μL, 1.16 mmol) and DBU (127 μL, 0.851 mmol) sequentially. After stirring the reaction mixture for 18 h at room temperature, cold 1N aq. HCl was added with vigorous stirring. The precipitate was collected by filtration, washed with cold water and hexanes, and dried to give a white solid (110 mg, 95%). ¹H NMR (400 MHz, CDCl₃) δ 9.94 (s, 1H), 7.82 (dd, J=2.9, 8.1 Hz, 1H), 7.40 (d, J=7.7 Hz, 3H), 7.35 (t, J=7.3 Hz, 1H), 7.27 (d, J=4.9 Hz, 2H), 7.19-7.03 (m, 1H), 4.74 (dd, J=6.4, 10.2 Hz, 2H), 3.11 (t, J=8.3 Hz, 2H); 19F NMR (376 MHz, CDCl3) δ −114.59; HRMS (M+H) for C16H13N2OSF calculated 301.0811, found 301.0813.

6-Fluoro-2-hydrazineyl-3-phenethylquinazolin-4(3H)-one: Following the procedure mentioned for the synthesis of 2-hydrazineyl-3-phenethylquinazolin-4(3H)-one, 6-fluoro-3-phenethyl-2-thioxo-2,3-dihydroquinazolin-4(1H)-one (110 mg, 0.366 mmol) gave product 6-fluoro-2-hydrazineyl-3-phenethylquinazolin-4(3H)-one as a white solid (80 mg, 73%). 1H NMR (400 MHz, CDCl3) δ 7.81 (dt, J=1.9, 8.5 Hz, 1H), 7.41-7.20 (m, 8H), 4.20 (t, J=7.4 Hz, 2H), 3.04 (t, J=7.4 Hz, 2H); 19F NMR (376 MHz, CDCl3) δ −117.99 (td, J=4.8, 8.3 Hz); HRMS (M+H) for C16H15N4OF calculated 299.1308, found 299.1307.

7-Fluoro-4-phenethyl-1-thioxo-2,4-dihydro-[1,2,4]triazolo[4,3-a]quinazolin-5(1H)-one: Following the procedure described for the synthesis of 4-phenethyl-1-thioxo-2,4-dihydro-[1,2,4]triazolo[4,3-a]quinazolin-5(1H)-one, 6-fluoro-2-hydrazineyl-3-phenethylquinazolin-4(3H)-one (50 mg, 0.168 mmol) gave product 7-fluoro-4-phenethyl-1-thioxo-2,4-dihydro-[1,2,4]triazolo[4,3-a]quinazolin-5(1H)-one as a white solid (20 mg, 35%). ¹H NMR (400 MHz, DMSO-d₆) δ 14.16 (s, 1H), 10.31 (dd, J=4.7, 9.3 Hz, 1H), 7.93 (dd, J=3.1, 8.6 Hz, 1H), 7.83 (td, J=3.1, 8.6 Hz, 1H), 7.27 (ddt, J=7.3, 14.2, 19.9 Hz, 4H), 4.34-4.15 (m, 2H), 3.09-2.93 (m, 2H); ¹⁹F NMR (376 MHz, DMSO-d₆) δ −112.99 (td, J=4.7, 8.3 Hz); HRMS (M+H) for C₁₇H₁₃N₄OSF calculated 341.0872, found 341.0871.

### Example 11

This example demonstrates a synthesis of 7-bromo-4-phenethyl-1-thioxo-2,4-dihydro-[1,2,4]triazolo[4,3-a]quinazolin-5(1H)-one in an aspect of the invention.

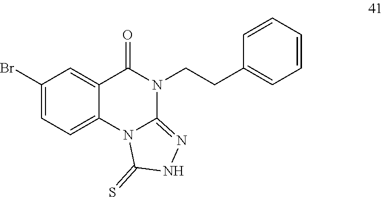 [SMILES: O=c1c2cc(Br)ccc2n2c(=S)[nH]nc2n1CCc1ccccc1]

6-Bromo-3-phenethyl-2-thioxo-2,3-dihydroquinazolin-4(1H)-one was synthesized according to General Procedure A. Then 7-bromo-4-phenethyl-1-thioxo-2,4-dihydro-[1,2,4]triazolo[4,3-a]quinazolin-5(1H)-one was synthesized according to General Procedure D: ¹H NMR (400 MHz, DMSO-d₆) δ 10.17 (d, J=9.1 Hz, 1H), 8.25 (d, J=2.4 Hz, 1H), 8.11 (dd, J=9.0, 2.5 Hz, 1H), 7.34-7.17 (m, 5H), 4.25-4.17 (m, 2H), 3.02-2.94 (m, 2H); HRMS (ES-API): m/z calculated for C₁₇H₁₄FN₄OS (M+H) 403.0046, found 403.0046.

### Example 12

This example demonstrates a synthesis of 7-iodo-4-phenethyl-1-thioxo-2,4-dihydro-[1,2,4]triazolo[4,3-a]quinazolin-5(1H)-one in an aspect of the invention.

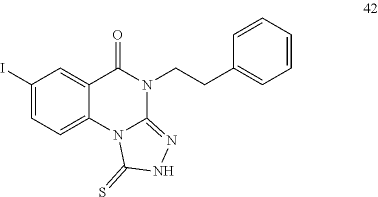 [SMILES: O=c1c2cc(I)ccc2n2c(=S)[nH]nc2n1CCc1ccccc1]

6-Iodo-3-phenethyl-2-thioxo-2,3-dihydroquinazolin-4(1H)-one was synthesized according to General Procedure A. Then 7-iodo-4-phenethyl-1-thioxo-2,4-dihydro-[1,2,4]triazolo[4,3-a]quinazolin-5(1H)-one was synthesized according to General Procedure D: ¹H NMR (400 MHz, DMSO-d₆) δ 10.00 (d, J=8.8 Hz, 1H), 8.41 (d, J=2.1 Hz, 1H), 8.24 (dd, J=8.9, 2.2 Hz, 1H), 7.32-7.18 (m, 5H), 4.24-4.16 (m, 2H), 3.02-2.93 (m, 2H).

### Example 13

This example demonstrates a synthesis of 7-methyl-4-phenethyl-1-thioxo-2,4-dihydro-[1,2,4]triazolo[4,3-a]quinazolin-5(1H)-one in an aspect of the invention.

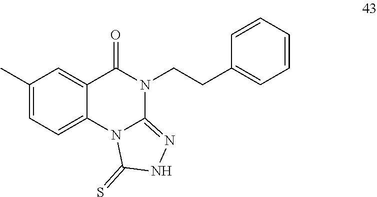 [SMILES: Cc1ccc2c(c1)c(=O)n(CCc1ccccc1)c1n[nH]c(=S)n21]

6-Methyl-3-phenethyl-2-thioxo-2,3-dihydroquinazolin-4(1H)-one was synthesized according to General Procedure A. Then 7-methyl-4-phenethyl-1-thioxo-2,4-dihydro-[1,2,4]triazolo[4,3-a]quinazolin-5(1H)-one was synthesized according to General Procedure D: ¹H NMR (400 MHz, DMSO-d₆) δ 10.06 (d, J=8.6 Hz, 1H), 8.02-7.97 (m, 1H), 7.71 (dd, J=8.8, 2.2 Hz, 1H), 7.33-7.16 (m, 5H), 4.26-4.18 (m, 2H), 3.03-2.94 (m, 2H), 2.44 (s, 3H).

### Example 14

This example demonstrates a synthesis of N-(5-oxo-4-phenethyl-1-thioxo-1,2,4,5-tetrahydro-[1,2,4]triazolo[4,3-a]quinazolin-7-yl)acetamide in an aspect of the invention.

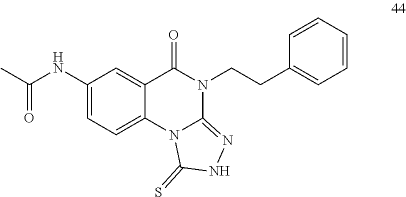 [SMILES: CC(=O)Nc1ccc2c(c1)c(=O)n(CCc1ccccc1)c1n[nH]c(=S)n21]

N-(4-Oxo-3-phenethyl-2-thioxo-1,2,3,4-tetrahydroquinazolin-6-yl)acetamide was synthesized according to General Procedure A. Then N-(5-oxo-4-phenethyl-1-thioxo-1,2,4,5-tetrahydro-[1,2,4]triazolo[4,3-a]quinazolin-7-yl)acetamide was synthesized according to General Procedure D: ¹H NMR (400 MHz, DMSO-d₆) δ 10.37 (s, 1H), 10.09 (d, J=9.1 Hz, 1H), 8.51 (d, J=2.6 Hz, 1H), 7.96 (dd, J=9.1, 2.7 Hz, 1H), 7.26 (ddd, J=19.6, 13.5, 7.3 Hz, 5H), 4.22 (dd, J=9.4, 6.5 Hz, 2H), 2.99 (t, J=7.9 Hz, 2H), 2.48 (t, J=3.0 Hz, 2H), 2.08 (s, 3H); HRMS (ES-API): m/z calculated for C₁₉H₁₈N₅O₂S (M+H) 380.1176, found 380.1174.

### Example 15

This example demonstrates a synthesis of 7-(dimethylamino)-4-phenethyl-1-thioxo-2,4-dihydro-[1,2,4]triazolo[4,3-a]quinazolin-5(1H)-one in an aspect of the invention.

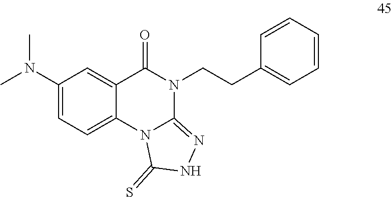 [SMILES: CN(C)c1ccc2c(c1)c(=O)n(CCc1ccccc1)c1n[nH]c(=S)n21]

6-(Dimethylamino)-3-phenethyl-2-thioxo-2,3-dihydroquinazolin-4(1H)-one was synthesized according to General Procedure A. Then 7-(dimethylamino)-4-phenethyl-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 13.92 (s, 1H), 9.98 (d, J=9.3 Hz, 1H), 7.33 (d, J=3.1 Hz, 1H), 7.32-7.16 (m, 6H), 4.26-4.18 (m, 2H), 2.99 (s, 6H), 3.00-2.94 (m, 2H); HRMS (ES-API): m/z calculated for C₁₉H₂₀N₅OS (M+H) 366.1383, found 366.1388; Purity (HPLC) 94.83%.

### Example 16

This example demonstrates a synthesis of 7-morpholino-4-phenethyl-1-thioxo-2,4-dihydro-[1,2,4]triazolo[4,3-a]quinazolin-5(1H)-one in an aspect of the invention.

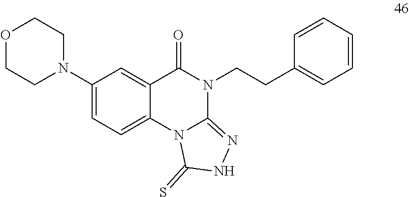 [SMILES: O=c1c2cc(N3CCOCC3)ccc2n2c(=S)[nH]nc2n1CCc1ccccc1]

6-Morpholino-3-phenethyl-2-thioxo-2,3-dihydroquinazolin-4(1H)-one was synthesized according to General Procedure A. Then 7-morpholino-4-phenethyl-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 13.97 (s, 1H), 10.02 (d, J=9.3 Hz, 1H), 7.57 (d, J=3.0 Hz, 1H), 7.53 (dd, J=9.3, 3.0 Hz, 1H), 7.31-7.17 (m, 5H), 4.26-4.18 (m, 2H), 3.76 (t, J=4.8 Hz, 4H), 3.22 (dd, J=6.1, 3.6 Hz, 4H), 3.02-2.94 (m, 2H); HRMS (ES-API): m/z calculated for C₂₁H₂₂N₅O₂S (M+H) 408.1489, found 408.1490.

### Example 17

This example demonstrates a synthesis of 8-fluoro-4-phenethyl-1-thioxo-2,4-dihydro-[1,2,4]triazolo[4,3-a]quinazolin-5(1H)-one in an aspect of the invention.

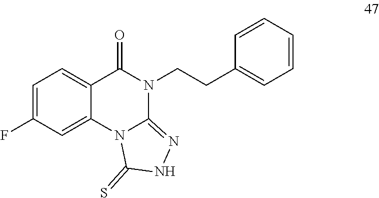 [SMILES: O=c1c2ccc(F)cc2n2c(=S)[nH]nc2n1CCc1ccccc1]

7-Fluoro-3-phenethyl-2-thioxo-2,3-dihydroquinazolin-4(1H)-one was synthesized according to General Procedure A. Then 8-fluoro-4-phenethyl-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.11 (dd, J=11.4, 2.5 Hz, 1H), 8.27 (dd, J=8.8, 6.3 Hz, 1H), 7.48 (td, J=8.4, 2.6 Hz, 1H), 7.26 (ddt, J=13.0, 11.5, 7.1 Hz, 5H), 4.25-4.16 (m, 2H), 3.02-2.94 (m, 2H); ¹⁹F NMR (376 MHz, DMSO-d₆) δ −101.36 (dt, J=11.9, 7.1 Hz); HRMS (ES-API): m/z calculated for C₁₇H₁₄FN₄OS (M+H) 341.0867, found 341.0882.

### Example 18

This example demonstrates a synthesis of 9-fluoro-4-phenethyl-1-thioxo-2,4-dihydro-[1,2,4]triazolo[4,3-a]quinazolin-5(1H)-one in an aspect of the invention.

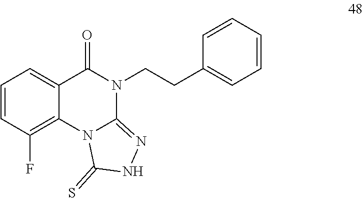 [SMILES: O=c1c2cccc(F)c2n2c(=S)[nH]nc2n1CCc1ccccc1]

8-Fluoro-3-phenethyl-2-thioxo-2,3-dihydroquinazolin-4(1H)-one was synthesized according to General Procedure A. Then 9-fluoro-4-phenethyl-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 13.88 (s, 1H), 8.01 (d, J=7.8 Hz, 1H), 7.83-7.73 (m, 1H), 7.62 (td, J=8.0, 4.0 Hz, 1H), 7.25 (ddd, J=18.6, 13.0, 7.3 Hz, 5H), 4.16 (dd, J=9.2, 6.7 Hz, 2H), 3.02-2.94 (m, 2H); ¹⁹F NMR (376 MHz, DMSO-d₆) δ −93.38 (dd, J=11.6, 4.1 Hz); HRMS (ES-API): m/z calculated for C₁₇H₁₄FN₄OS (M+H) 341.0867, found 341.0859.

### Example 19

This example demonstrates a synthesis of 4-phenethyl-1-thioxo-2,4-dihydropyrido[2,3-e][1,2,4]triazolo[4,3-a]pyrimidin-5(1H)-one in an aspect of the invention.

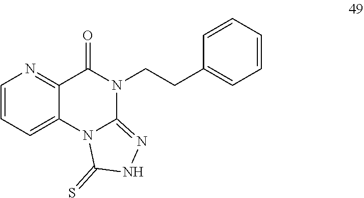 [SMILES: O=c1c2ncccc2n2c(=S)[nH]nc2n1CCc1ccccc1]

3-phenethyl-2-thioxo-2,3-dihydropyrido[3,2-d]pyrimidin-4(1H)-one was synthesized according to General Procedure A. Then 4-phenethyl-1-thioxo-2,4-dihydropyrido[2,3-e][1,2,4]triazolo[4,3-a]pyrimidin-5(1H)-one, TFA was synthesized according to General Procedure D.

### Example 20

This example demonstrates a synthesis of 4-phenethyl-1-thioxo-2,4-dihydropyrido[4,3-e][1,2,4]triazolo[4,3-a]pyrimidin-5(1H)-one in an aspect of the invention.

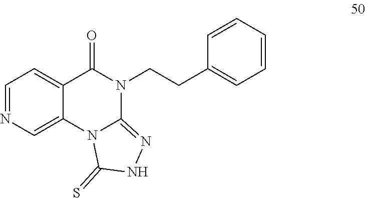 [SMILES: O=c1c2ccncc2n2c(=S)[nH]nc2n1CCc1ccccc1]

3-phenethyl-2-thioxo-2,3-dihydropyrido[3,4-d]pyrimidin-4(1H)-one was synthesized according to General Procedure A. Then 4-phenethyl-1-thioxo-2,4-dihydropyrido[4,3-e][1,2,4]triazolo[4,3-a]pyrimidin-5(1H)-one, TFA was synthesized according to General Procedure D: ¹H NMR (400 MHz, DMSO-d₆) δ 11.36 (s, 1H), 8.82 (d, J=5.0 Hz, 1H), 8.06 (d, J=5.0 Hz, 1H), 7.33-7.22 (m, 4H), 7.20 (t, J=7.1 Hz, 1H), 4.24-4.17 (m, 2H), 2.97 (t, J=8.0 Hz, 2H).

### Example 21

This example demonstrates a synthesis of 6-phenethyl-9-thioxo-8,9-dihydropyrido[3,2-e][1,2,4]triazolo[4,3-a]pyrimidin-5(6H)-one in an aspect of the invention.

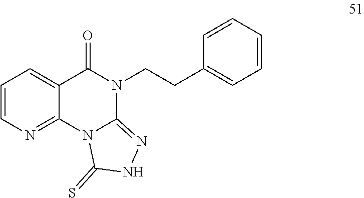 [SMILES: O=c1c2cccnc2n2c(=S)[nH]nc2n1CCc1ccccc1]

3-Phenethyl-2-thioxo-2,3-dihydropyrido[2,3-d]pyrimidin-4(1H)-one was synthesized according to General Procedure A. Then 6-phenethyl-9-thioxo-8,9-dihydropyrido[3,2-e][1,2,4]triazolo[4,3-a]pyrimidin-5(6H)-one, TFA was synthesized according to General Procedure D: ¹H NMR (400 MHz, DMSO-d₆) δ 13.84 (s, 1H), 8.83 (dd, J=4.7, 1.9 Hz, 1H), 8.52 (dd, J=7.8, 1.9 Hz, 1H), 7.63 (dd, J=7.8, 4.8 Hz, 1H), 7.24 (ddt, J=20.7, 14.0, 7.3 Hz, 5H), 4.23-4.14 (m, 2H), 2.97 (dd, J=9.4, 6.4 Hz, 2H).

### Example 22

This example demonstrates a synthesis of 4-benzyl-7-fluoro-1-thioxo-2,4-dihydro-[1,2,4]triazolo[4,3-a]quinazolin-5(1H)-one in an aspect of the invention.

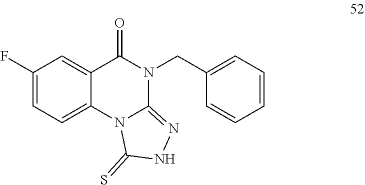 [SMILES: O=c1c2cc(F)ccc2n2c(=S)[nH]nc2n1Cc1ccccc1]

3-Benzyl-6-fluoro-2-thioxo-2,3-dihydroquinazolin-4(1H)-one was synthesized according to General Procedure A. Then 4-benzyl-7-fluoro-1-thioxo-2,4-dihydro-[1,2,4]triazolo[4,3-a]quinazolin-5(1H)-one was synthesized according to General Procedure D and purified by pouring into ice water and drops of acetic acid were added. The solid was filtered, and recrystallized from dioxane to give 4-benzyl-7-fluoro-1-thioxo-2,4-dihydro-[1,2,4]triazolo[4,3-a]quinazolin-5(1H)-one (0.0226 g, 25%): ¹H NMR (400 MHz, DMSO-d₆) δ 10.30 (dd, J=9.4, 4.7 Hz, 1H), 7.95 (dd, J=8.6, 3.1 Hz, 1H), 7.82 (ddd, J=9.3, 7.9, 3.1 Hz, 1H), 7.43-7.36 (m, 2H), 7.30 (dd, J=8.1, 6.2 Hz, 2H), 7.29-7.21 (m, 1H), 5.22 (s, 2H); ¹⁹F NMR (376 MHz, DMSO-d₆) δ −113.06 (td, J=8.4, 4.9 Hz); HRMS (ES-API): m/z calculated for C₁₆H₁₂FN₄OS (M+H) 327.0710, found 327.0716.

### Example 23

This example demonstrates a synthesis of 7-fluoro-4-(3-phenylpropyl)-1-thioxo-2,4-dihydro-[1,2,4]triazolo[4,3-a]quinazolin-5(1H)-one in an aspect of the invention.

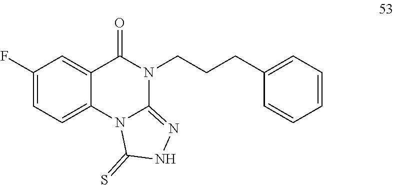 [SMILES: O=c1c2cc(F)ccc2n2c(=S)[nH]nc2n1CCCc1ccccc1]

6-Fluoro-3-(3-phenylpropyl)-2-thioxo-2,3-dihydroquinazolin-4(1H)-one was synthesized according to General Procedure A. Then 7-fluoro-4-(3-phenylpropyl)-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.27 (dd, J=9.3, 4.6 Hz, 1H), 7.90 (dd, J=8.6, 3.1 Hz, 1H), 7.79 (td, J=8.6, 3.1 Hz, 1H), 7.25-7.14 (m, 4H), 7.10 (t, J=7.0 Hz, 1H), 4.07 (t, J=7.2 Hz, 2H), 2.67 (t, J=7.7 Hz, 2H), 2.02 (p, J=7.4 Hz, 2H); ¹⁹F NMR (376 MHz, DMSO-d₆) δ −113.19 (td, J=8.4, 4.9 Hz).

### Example 24

This example demonstrates a synthesis of 7-chloro-1-thioxo-4-(3,3,3-trifluoropropyl)-2,4-dihydro-[1,2,4]triazolo[4,3-a]quinazolin-5(1H)-one in an aspect of the invention.

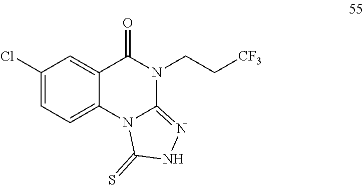 [SMILES: O=c1c2cc(Cl)ccc2n2c(=S)[nH]nc2n1CCC(F)(F)F]

6-Chloro-2-thioxo-3-(3,3,3-trifluoropropyl)-2,3-dihydroquinazolin-4(1H)-one was synthesized according to General Procedure B. Then 7-chloro-1-thioxo-4-(3,3,3-trifluoropropyl)-2,4-dihydro-[1,2,4]triazolo[4,3-a]quinazolin-5(H)-one, TFA was synthesized according to General Procedure D: ¹H NMR (400 MHz, DMSO-d₆) δ 10.23 (d, J=9.1 Hz, 1H), 8.14 (d, J=2.5 Hz, 1H), 8.00 (dd, J=9.1, 2.5 Hz, 1H), 4.25 (t, J=7.3 Hz, 2H), 2.72 (dt, J=17.7, 9.2 Hz, 2H); ¹⁹F NMR (376 MHz, DMSO-d₆) δ −64.33 (t, J=11.3 Hz); HRMS (ES-API): m/z calculated for C₁₂H₉ClF₃N₄OS (M+H) 349.0132, found 349.0141; Purity (HPLC) 91.99%.

### Example 25

This example demonstrates a synthesis of 6-fluoro-N-methyl-4-oxo-3-phenethyl-3,4-dihydroquinazoline-1(2H)-carbothioamide in an aspect of the invention.

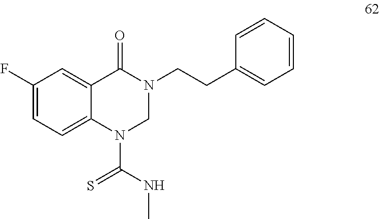 [SMILES: CNC(=S)N1CN(CCc2ccccc2)C(=O)c2cc(F)ccc21]

6-Fluoro-3-phenethyl-2-thioxo-2,3-dihydroquinazolin-4(1H)-one was synthesized according to General Procedure A. Then 6-fluoro-3-phenethyl-2,3-dihydroquinazolin-4(1H)-one was synthesized by: to a solution of 6-fluoro-3-phenethyl-2-thioxo-2,3-dihydroquinazolin-4(1H)-one (0.2 g, 0.666 mmol) and nickel(II)chloride (0.604 g, 4.66 mmol) in MeOH (5 ml) was added sodium borohydride (0.529 g, 13.98 mmol) carefully. The reaction was stirred at room temperature for 30 min. The reaction was then filtered through Celite and the solid washed with MeOH. The filtrate was taken up in EtOAc and washed with water. The organics were dried over MgSO₄, filtered, and concentrated. The product was recrystallized from EtOH.

6-Fluoro-N-methyl-4-oxo-3-phenethyl-3,4-dihydroquinazoline-1(2H)-carbothioamide, TFA was synthesized by: to a solution of 6-fluoro-3-phenethyl-2,3-dihydroquinazolin-4(1H)-one (0.05 g, 0.185 mmol) in DCM (0.462 ml) was added 1,1′-thiocarbonyldiimidazole (0.049 g, 0.277 mmol) and the reaction was stirred for 2 hr at room temperature. Methylamine hydrochloride (0.125 g, 1.850 mmol) and triethylamine (0.284 ml, 2.035 mmol) were added and the reaction stirred for 24 hr. The reaction was concentrated under a stream of N₂ and purified by HPLC: ¹H NMR (400 MHz, DMSO-d₆) δ 8.42 (s, 1H), 7.51 (d, J=8.8 Hz, 1H), 7.41 (d, J=6.3 Hz, 2H), 7.19 (dq, J=14.0, 7.2 Hz, 4H), 5.43 (s, 2H), 3.66 (t, J=7.3 Hz, 2H), 2.93 (d, J=4.2 Hz, 3H), 2.83 (t, J=7.4 Hz, 2H); ¹⁹F NMR (376 MHz, DMSO-d₆) δ −115.99; HRMS (ES-API): m/z calculated for C₁₈H₁₈F₃N₃NaOS (M+Na) 366.1047, found 366.1052.

### Example 26

This example demonstrates a synthesis of 4-ethyl-7-fluoro-1-thioxo-2,4-dihydro-[1,2,4]triazolo[4,3-a]quinazolin-5(1H)-one in an aspect of the invention.

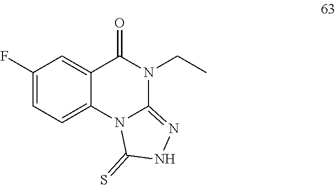 [SMILES: CCn1c(=O)c2cc(F)ccc2n2c(=S)[nH]nc12]

3-Ethyl-6-fluoro-2-thioxo-2,3-dihydroquinazolin-4(1H)-one was synthesized according to General Procedure A. Then 4-ethyl-7-fluoro-1-thioxo-2,4-dihydro-[1,2,4]triazolo[4,3-a]quinazolin-5(1H)-one was synthesized according to General Procedure D: ¹H NMR (400 MHz, DMSO-d₆) δ 10.28 (dd, J=9.3, 4.7 Hz, 1H), 7.91 (dd, J=8.6, 3.1 Hz, 1H), 7.79 (ddd, J=9.3, 8.0, 3.1 Hz, 1H), 4.06 (q, J=7.1 Hz, 2H), 1.24 (t, J=7.1 Hz, 3H); ¹⁹F NMR (376 MHz, DMSO-d₆) δ −113.15 (td, J=8.5, 4.8 Hz); HRMS (ES-API): m/z calculated for C₁₁H₁₀FN₄OS (M+H) 265.0554, found 265.0557; Purity (HPLC) 91.89%.

### Example 27

This example demonstrates a synthesis of 7-fluoro-1-thioxo-4-(3,3,3-trifluoropropyl)-2,4-dihydro-[1,2,4]triazolo[4,3-a]quinazolin-5(1H)-one in an aspect of the invention.

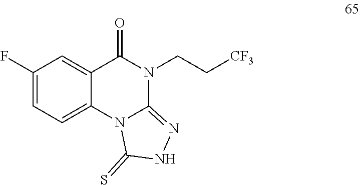 [SMILES: O=c1c2cc(F)ccc2n2c(=S)[nH]nc2n1CCC(F)(F)F]

6-Fluoro-2-thioxo-3-(3,3,3-trifluoropropyl)-2,3-dihydroquinazolin-4(1H)-one was synthesized according to General Procedure B. Then 7-fluoro-1-thioxo-4-(3,3,3-trifluoropropyl)-2,4-dihydro-[1,2,4]triazolo[4,3-a]quinazolin-5(H)-one, TFA was synthesized according to General Procedure D: ¹H NMR (400 MHz, DMSO-d₆) δ 10.27 (d, J=6.5 Hz, 1H), 7.93 (dd, J=8.6, 3.0 Hz, 1H), 7.82 (td, J=9.3, 8.7, 3.1 Hz, 1H), 4.26 (t, J=7.2 Hz, 2H), 2.72 (dtd, J=18.6, 11.3, 7.7 Hz, 2H); ¹⁹F NMR (376 MHz, DMSO-d₆) δ −64.33 (t, J=11.3 Hz), −112.81; HRMS (ES-API): m/z calculated for C₁₂H₈F₄N₄NaOS (M+Na) 355.0247, found 355.0245.

### Example 28

This example demonstrates a synthesis of 7-fluoro-4-(3-hydroxypropyl)-1-thioxo-2,4-dihydro-[1,2,4]triazolo[4,3-a]quinazolin-5(1H)-one in an aspect of the invention.

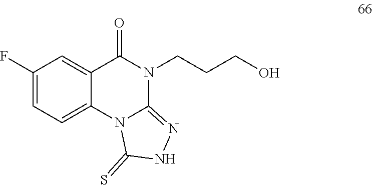 [SMILES: O=c1c2cc(F)ccc2n2c(=S)[nH]nc2n1CCCO]

6-Fluoro-3-(3-hydroxypropyl)-2-thioxo-2,3-dihydroquinazolin-4(1H)-one was synthesized according to General Procedure B. Then 7-fluoro-4-(3-hydroxypropyl)-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.28 (dd, J=9.4, 4.6 Hz, 1H), 7.90 (dd, J=8.6, 3.1 Hz, 1H), 7.78 (td, J=8.5, 3.0 Hz, 1H), 4.48 (t, J=5.1 Hz, 1H), 4.08 (t, J=7.4 Hz, 2H), 3.47 (q, J=5.8 Hz, 2H), 1.89-1.77 (m, 2H); ¹⁹F NMR (376 MHz, DMSO-d₆) δ −113.18 (q, J=7.4 Hz); HRMS (ES-API): m/z calculated for C₁₂H₁₂FN₄O₂S (M+H) 294.0660, found 294.0664.

### Example 29

This example demonstrates a synthesis of 4-(2-aminoethyl)-7-fluoro-1-thioxo-2,4-dihydro-[1,2,4]triazolo[4,3-a]quinazolin-5(1H)-one in an aspect of the invention.

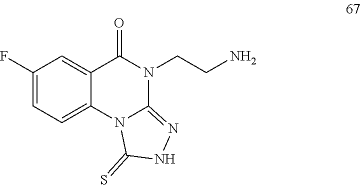 [SMILES: NCCn1c(=O)c2cc(F)ccc2n2c(=S)[nH]nc12]

tert-Butyl (2-(6-fluoro-4-oxo-2-thioxo-1,4-dihydroquinazolin-3(2H)-yl)ethyl)carbamate was synthesized according to General Procedure C. Then tert-butyl (2-(7-fluoro-5-oxo-1-thioxo-1,2-dihydro-[1,2,4]triazolo[4,3-a]quinazolin-4(5H)-yl)ethyl)carbamate was synthesized according to General Procedure D. Then 4-(2-aminoethyl)-7-fluoro-1-thioxo-2,4-dihydro-[1,2,4]triazolo[4,3-a]quinazolin-5(1H)-one, 2 TFA was synthesized by treating tert-butyl (2-(7-fluoro-5-oxo-1-thioxo-1,2-dihydro-[1,2,4]triazolo[4,3-a]quinazolin-4(5H)-yl)ethyl)carbamate (0.112 g, 0.295 mmol) in DCM (1.230 ml) and MeOH (0.246 ml) with TFA (0.989 ml, 12.84 mmol). The reaction was stirred at room temperature for 3 h, then blown down, redissolved, and purified by HPLC: ¹H NMR (400 MHz, DMSO-d₆) δ 10.31 (dd, J=9.3, 4.6 Hz, 1H), 7.95 (dd, J=8.5, 3.1 Hz, 1H), 7.85 (ddd, J=9.2, 7.6, 3.2 Hz, 3H), 4.32 (t, J=5.8 Hz, 2H), 3.20 (t, J=6.0 Hz, 2H); ¹⁹F NMR (376 MHz, DMSO-d₆) δ −112.75 (td, J=8.3, 4.7 Hz); HRMS (ES-API): m/z calculated for C₁₁H₁₀FN₅NaOS (M+Na) 302.0482, found 302.0493.

### Example 30

This example demonstrates a synthesis of 4-(3-aminopropyl)-7-fluoro-1-thioxo-2,4-dihydro-[1,2,4]triazolo[4,3-a]quinazolin-5(1H)-one in an aspect of the invention.

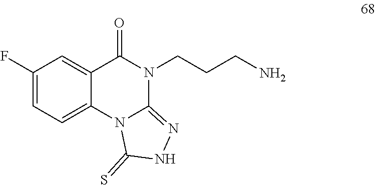 [SMILES: NCCCn1c(=O)c2cc(F)ccc2n2c(=S)[nH]nc12]

tert-Butyl (3-(6-fluoro-4-oxo-2-thioxo-1,4-dihydroquinazolin-3(2H)-yl)propyl)carbamate was synthesized according to General Procedure C. Then tert-butyl (3-(7-fluoro-5-oxo-1-thioxo-1,2-dihydro-[1,2,4]triazolo[4,3-a]quinazolin-4(5H)-yl)propyl)carbamate was synthesized according to General Procedure D. Then 4-(3-aminopropyl)-7-fluoro-1-thioxo-2,4-dihydro-[1,2,4]triazolo[4,3-a]quinazolin-5(1H)-one, 2 TFA was synthesized by treating a solution of tert-butyl (3-(7-fluoro-5-oxo-1-thioxo-1,2-dihydro-[1,2,4]triazolo[4,3-a]quinazolin-4(5H)-yl)propyl)carbamate (0.056 g, 0.142 mmol) in DCM (0.593 ml) and MeOH (0.119 ml) was added TFA (0.477 ml, 6.19 mmol) and the reaction stirred at room temperature for 3 h. The reaction was then concentrated and the product was purified by ISCO (DCM/MeOH+3.3% NH₄OH, 0-100%): ¹H NMR (400 MHz, DMSO-d₆) δ 10.30 (dd, J=9.4, 4.7 Hz, 1H), 7.92 (dd, J=8.5, 3.1 Hz, 1H), 7.83 (ddd, J=9.3, 7.9, 3.1 Hz, 1H), 7.65 (s, 3H), 4.10 (t, J=6.8 Hz, 2H), 2.89 (q, J=8.4, 7.3 Hz, 2H), 2.00 (p, J=7.0 Hz, 2H); ¹⁹F NMR (376 MHz, DMSO-d₆) δ −113.06 (td, J=8.3, 4.8 Hz); HRMS (ES-API): m/z calculated for C₁₂H₁₃FN₅OS (M+H) 294.0819, found 294.0825.

### Example 31

This example demonstrates a synthesis of 4-(3-aminopropyl)-9-fluoro-1-thioxo-2,4-dihydro-[1,2,4]triazolo[4,3-a]quinazolin-5(1H)-one in an aspect of the invention.

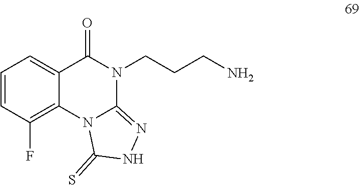 [SMILES: NCCCn1c(=O)c2cccc(F)c2n2c(=S)[nH]nc12]

tert-Butyl (3-(8-fluoro-4-oxo-2-thioxo-1,4-dihydroquinazolin-3(2H)-yl)propyl)carbamate was synthesized according to General Procedure A. Then tert-butyl (3-(9-fluoro-5-oxo-1-thioxo-1,2-dihydro-[1,2,4]triazolo[4,3-a]quinazolin-4(5H)-yl)propyl)carbamate was synthesized according to General Procedure D. Then 4-(3-aminopropyl)-9-fluoro-1-thioxo-2,4-dihydro-[1,2,4]triazolo[4,3-a]quinazolin-5(1H)-one was synthesized by: tert-butyl (3-(9-fluoro-5-oxo-1-thioxo-1,2-dihydro-[1,2,4]triazolo[4,3-a]quinazolin-4(5H)-yl)propyl)carbamate (0.170 g, 0.432 mmol) was dissolved in HCl in dioxane (1.512 ml, 6.05 mmol) and stirred at room temperature for 5 hr. The reaction was quenched with triethylamine (0.843 ml, 6.05 mmol), filtered, and concentrated. The product was purified by ISCO (conditions) to give 4-(3-aminopropyl)-9-fluoro-1-thioxo-2,4-dihydro-[1,2,4]triazolo[4,3-a]quinazolin-5(1H)-one, 2 TFA: ¹⁹F NMR (376 MHz, DMSO-d₆) δ −93.31 (dd, J=11.6, 4.2 Hz).

### Example 31

This example demonstrates a synthesis of 4-(2-(dimethylamino)ethyl)-7-fluoro-1-thioxo-2,4-dihydro-[1,2,4]triazolo[4,3-a]quinazolin-5(1H)-one in an aspect of the invention.

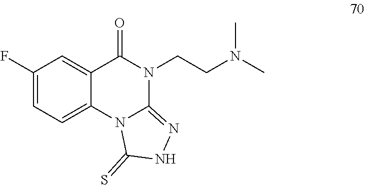 [SMILES: CN(C)CCn1c(=O)c2cc(F)ccc2n2c(=S)[nH]nc12]

3-(2-(Dimethylamino)ethyl)-6-fluoro-2-thioxo-2,3-dihydroquinazolin-4(1H)-one was synthesized according to General Procedure B. Then 4-(2-(dimethylamino)ethyl)-7-fluoro-1-thioxo-2,4-dihydro-[1,2,4]triazolo[4,3-a]quinazolin-5(1H)-one, 2 TFA was synthesized according to General Procedure D: ¹H NMR (400 MHz, DMSO-d₆) δ 10.30 (dd, J=9.3, 4.6 Hz, 1H), 9.10 (s, 1H), 7.95 (dd, J=8.5, 3.1 Hz, 1H), 7.86 (td, J=8.5, 3.1 Hz, 1H), 4.40 (t, J=5.6 Hz, 2H), 3.50-3.43 (m, 2H), 2.85 (s, 6H); ¹⁹F NMR (376 MHz, DMSO-d₆) δ −112.62 (q, J=7.5 Hz); HRMS (ES-API): m/z calculated for C₁₃H₁₅FN₅OS (M+H) 308.0976, found 308.0989.

### Example 32

This example demonstrates a synthesis of 4-(1-(dimethylamino)propan-2-yl)-7-fluoro-1-thioxo-2,4-dihydro-[1,2,4]triazolo[4,3-a]quinazolin-5(1H)-one in an aspect of the invention.

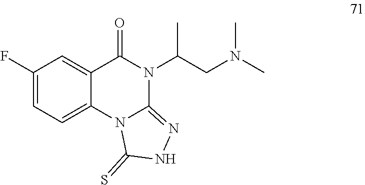 [SMILES: CC(CN(C)C)n1c(=O)c2cc(F)ccc2n2c(=S)[nH]nc12]

3-(1-(Dimethylamino)propan-2-yl)-6-fluoro-2-thioxo-2,3-dihydroquinazolin-4(1H)-one was synthesized according to General Procedure B. Then 4-(1-(dimethylamino)propan-2-yl)-7-fluoro-1-thioxo-2,4-dihydro-[1,2,4]triazolo[4,3-a]quinazolin-5(1H)-one, 2 TFA was synthesized according to General Procedure D: ¹H NMR (400 MHz, DMSO-d₆) δ 10.34 (dd, J=9.3, 4.6 Hz, 1H), 9.24 (s, 1H), 7.94 (dd, J=8.6, 3.1 Hz, 1H), 7.85 (td, J=9.2, 8.7, 3.1 Hz, 1H), 4.05 (t, J=12.3 Hz, 1H), 3.35 (s, 2H), 2.80 (d, J=17.7 Hz, 6H), 1.52 (d, J=6.8 Hz, 3H); ¹⁹F NMR (376 MHz, DMSO-d₆) δ −112.68 (q, J=7.8 Hz); HRMS (ES-API): m/z calculated for C₁₄H₁₇FN₅OS (M+H) 322.1132, found 322.1139.

### Example 33

This example demonstrates a synthesis of N-(2-(7-fluoro-5-oxo-1-thioxo-1,2-dihydro-[1,2,4]triazolo[4,3-a]quinazolin-4(5H)-yl)ethyl)acetamide in an aspect of the invention.

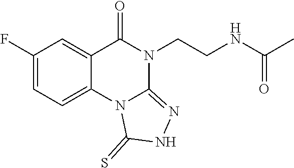 [SMILES: CC(=O)NCCn1c(=O)c2cc(F)ccc2n2c(=S)[nH]nc12]

N-(2-(6-Fluoro-4-oxo-2-thioxo-1,4-dihydroquinazolin-3(2H)-yl)ethyl)acetamide was synthesized according to General Procedure B. Then N-(2-(7-fluoro-5-oxo-1-thioxo-1,2-dihydro-[1,2,4]triazolo[4,3-a]quinazolin-4(5H)-yl)ethyl)acetamide, TFA was synthesized according to General Procedure D: ¹H NMR (400 MHz, DMSO-d₆) δ 10.30 (dd, J=9.3, 4.6 Hz, 1H), 7.92 (dd, J=8.6, 3.0 Hz, 1H), 7.88-7.76 (m, 2H), 4.08 (t, J=5.8 Hz, 2H), 3.39 (q, J=6.1 Hz, 2H), 1.64 (s, 3H); ¹⁹F NMR (376 MHz, DMSO-d₆) δ −113.00 (q, J=7.6, 6.7 Hz); HRMS (ES-API): m/z calculated for C₁₃H₁₃FN₅O₂S (M+H) 322.0769, found 322.0780.

### Example 34

This example demonstrates a synthesis of N-(3-(7-fluoro-5-oxo-1-thioxo-1,2-dihydro-[1,2,4]triazolo[4,3-a]quinazolin-4(5H)-yl)propyl)acetamide in an aspect of the invention.

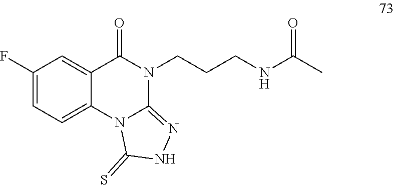 [SMILES: CC(=O)NCCCn1c(=O)c2cc(F)ccc2n2c(=S)[nH]nc12]

tert-Butyl (3-(6-fluoro-4-oxo-2-thioxo-1,4-dihydroquinazolin-3(2H)-yl)propyl)carbamate was synthesized according to General Procedure C.

3-(3-Aminopropyl)-6-fluoro-2-thioxo-2,3-dihydroquinazolin-4(1H)-one: to a solution of crude tert-butyl (3-(6-fluoro-4-oxo-2-thioxo-1,4-dihydroquinazolin-3(2H)-yl)propyl)carbamate (0.1556 g, 0.440 mmol) in DCM (2.201 ml) was added TFA (1.476 ml, 19.15 mmol) and the reaction stirred at room temperature for 2 hr. The reaction was then blown down, the residue dissolved in MeOH, gravity filtered through a PL-HCO₃ SPE cartridge, and concentrated. The crude product was carried on without further purification. N-(3-(6-fluoro-4-oxo-2-thioxo-1,4-dihydroquinazolin-3(2H)-yl)propyl)acetamide: to a solution of 3-(3-aminopropyl)-6-fluoro-2-thioxo-2,3-dihydroquinazolin-4(1H)-one (0.05 g, 0.197 mmol) in pyridine (0.6 ml) was added acetyl chloride (0.021 ml, 0.296 mmol) and the reaction stirred at 0° C. for 2 hr. The reaction was cooled to room temperature and poured into ice water, filtered, the solid washed with water and Et₂O, and dried. The crude product was carried on without further purification.

N-(3-(7-Fluoro-5-oxo-1-thioxo-1,2-dihydro-[1,2,4]triazolo[4,3-a]quinazolin-4(5H)-yl)propyl)acetamide, TFA was synthesized according to General Procedure D: ¹H NMR (400 MHz, DMSO-d₆) δ 10.29 (dd, J=9.4, 4.7 Hz, 1H), 7.91 (dd, J=8.6, 3.1 Hz, 1H), 7.86-7.75 (m, 2H), 4.02 (dd, J=8.2, 6.3 Hz, 2H), 3.09 (q, J=6.6 Hz, 2H), 1.82 (p, J=7.2 Hz, 2H), 1.76 (s, 3H); ¹⁹F NMR (376 MHz, DMSO-d₆) δ −113.17 (td, J=8.2, 4.5 Hz); HRMS (ES-API): m/z calculated for C₁₇H₁₈F₃N₄OS (M+H) 336.0925, found 336.0929; Purity (HPLC) 90.11%.

### Example 35

This example demonstrates a synthesis of N-(3-(7-fluoro-5-oxo-1-thioxo-1,2-dihydro-[1,2,4]triazolo[4,3-a]quinazolin-4(5H)-yl)propyl)benzamide in an aspect of the invention.

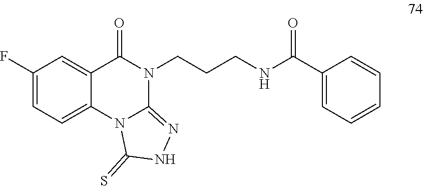 [SMILES: O=C(NCCCn1c(=O)c2cc(F)ccc2n2c(=S)[nH]nc12)c1ccccc1]

N-(3-(7-Fluoro-5-oxo-1-thioxo-1,2-dihydro-[1,2,4]triazolo[4,3-a]quinazolin-4(5H)-yl)propyl)benzamide, TFA was synthesized according to General Procedure F: ¹H NMR (400 MHz, DMSO-d₆) δ 10.31 (dd, J=9.3, 4.7 Hz, 1H), 8.46 (t, J=5.7 Hz, 1H), 7.91 (dd, J=8.6, 3.1 Hz, 1H), 7.85-7.78 (m, 3H), 7.54-7.49 (m, 1H), 7.48-7.42 (m, 2H), 4.11 (dd, J=8.1, 6.6 Hz, 2H), 3.36 (q, J=6.6 Hz, 2H), 1.99 (p, J=6.9 Hz, 2H); ¹⁹F NMR (376 MHz, DMSO-d₆) δ −113.15 (td, J=8.2, 4.6 Hz); HRMS (ES-API): m/z calculated for C₁₉H₁₇FN₅O₂S (M+H) 398.1082, found 398.1063.

### Example 36

This example demonstrates a synthesis of N-(2-(7-fluoro-5-oxo-1-thioxo-1,2-dihydro-[1,2,4]triazolo[4,3-a]quinazolin-4(5H)-yl)ethyl)methanesulfonamide in an aspect of the invention.

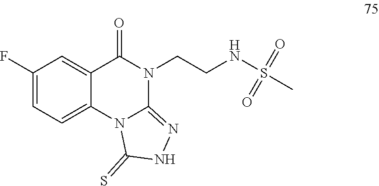 [SMILES: CS(=O)(=O)NCCn1c(=O)c2cc(F)ccc2n2c(=S)[nH]nc12]

N-(2-(6-Fluoro-4-oxo-2-thioxo-1,4-dihydroquinazolin-3(2H)-yl)ethyl)methanesulfonamide was synthesized according to General Procedure C. Then N-(2-(7-fluoro-5-oxo-1-thioxo-1,2-dihydro-[1,2,4]triazolo[4,3-a]quinazolin-4(5H)-yl)ethyl)methanesulfonamide, TFA was synthesized according to General Procedure D: ¹H NMR (400 MHz, DMSO-d₆) δ 10.30 (dd, J=9.3, 4.7 Hz, 1H), 7.93 (dd, J=8.6, 3.1 Hz, 1H), 7.81 (ddd, J=9.3, 8.0, 3.1 Hz, 1H), 7.17 (t, J=6.4 Hz, 1H), 4.18 (t, J=6.2 Hz, 2H), 3.36 (q, J=6.2 Hz, 2H), 2.88 (s, 3H); ¹⁹F NMR (376 MHz, DMSO-d₆) δ −112.99 (td, J=8.3, 4.7 Hz); HRMS (ES-API): m/z calculated for C₁₂H₁₃FN₅O₃S₂ (M+H) 358.0438, found 358.0441.

### Example 37

This example demonstrates a synthesis of N-(3-(7-fluoro-5-oxo-1-thioxo-1,2-dihydro-[1,2,4]triazolo[4,3-a]quinazolin-4(5H)-yl)propyl)methanesulfonamide in an aspect of the invention.

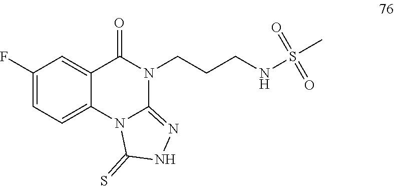 [SMILES: CS(=O)(=O)NCCCn1c(=O)c2cc(F)ccc2n2c(=S)[nH]nc12]

N-(3-(7-fluoro-5-oxo-1-thioxo-1,2-dihydro-[1,2,4]triazolo[4,3-a]quinazolin-4(5H)-yl)propyl)methanesulfonamide was synthesized by treating 4-(3-aminopropyl)-7-fluoro-1-thioxo-2,4-dihydro-[1,2,4]triazolo[4,3-a]quinazolin-5(1H)-one (0.05 g, 0.170 mmol) in DCM (0.852 ml) at room temperature with methanesulfonyl chloride (0.015 ml, 0.188 mmol) and triethylamine (0.029 ml, 0.205 mmol). The reaction was stirred at room temperature for 2 hr, concentrated, and purified by HPLC: ¹H NMR (400 MHz, DMSO-d₆) δ 10.29 (dd, J=9.3, 4.7 Hz, 1H), 7.92 (dd, J=8.6, 3.1 Hz, 1H), 7.81 (ddd, J=9.3, 8.0, 3.2 Hz, 1H), 7.00 (t, J=5.9 Hz, 1H), 4.07 (t, J=7.1 Hz, 2H), 3.02 (q, J=6.7 Hz, 2H), 2.86 (s, 3H), 1.89 (p, J=7.1 Hz, 2H); ¹⁹F NMR (376 MHz, DMSO-d₆) δ −113.18 (td, J=8.3, 5.0 Hz); HRMS (ES-API): m/z calculated for C₁₃H₁₅FN₅O₃S₂ (M+H) 372.0595, found 372.0601; Purity (HPLC) 94.02%.

### Example 38

This example demonstrates a synthesis of diethyl (2-(7-fluoro-5-oxo-1-thioxo-1,2-dihydro-[1,2,4]triazolo[4,3-a]quinazolin-4(5H)-yl)ethyl)phosphonate in an aspect of the invention.

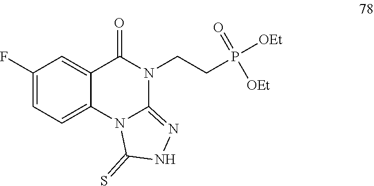 [SMILES: CCOP(=O)(CCn1c(=O)c2cc(F)ccc2n2c(=S)[nH]nc12)OCC]

Diethyl (2-(6-fluoro-4-oxo-2-thioxo-1,4-dihydroquinazolin-3(2H)-yl)ethyl)phosphonate was synthesized according to General Procedure B. Then diethyl (2-(7-fluoro-5-oxo-1-thioxo-1,2-dihydro-[1,2,4]triazolo[4,3-a]quinazolin-4(5H)-yl)ethyl)phosphonate, TFA was synthesized according to General Procedure D: ¹H NMR (400 MHz, DMSO-d₆) δ 10.32 (d, J=7.3 Hz, 1H), 7.92 (dd, J=8.5, 3.1 Hz, 1H), 7.80 (ddd, J=9.1, 7.9, 3.1 Hz, 1H), 4.25-4.15 (m, 2H), 4.00 (dqd, J=8.1, 7.0, 3.5 Hz, 4H), 3.26 (s, 1H), 2.28-2.15 (m, 2H), 1.22 (t, J=7.0 Hz, 6H); ¹⁹F NMR (376 MHz, DMSO-d₆) δ −113.07; ³¹P NMR (162 MHz, DMSO-d₆) δ 26.69; HRMS (ES-API): m/z calculated for C₁₅H₁₉FN₄O₄PS (M+H) 401.0843, found 401.0856.

### Example 39

This example demonstrates a synthesis of (2-(7-fluoro-5-oxo-1-thioxo-1,2-dihydro-[1,2,4]triazolo[4,3-a]quinazolin-4(5H)-yl)ethyl)phosphonic acid in an aspect of the invention.

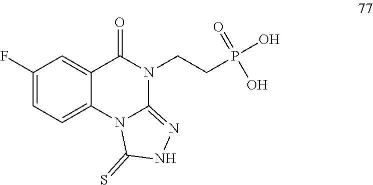 [SMILES: O=c1c2cc(F)ccc2n2c(=S)[nH]nc2n1CCP(=O)(O)O]

(2-(7-Fluoro-5-oxo-1-thioxo-1,2-dihydro-[1,2,4]triazolo[4,3-a]quinazolin-4(5H)-yl)ethyl)phosphonic acid was synthesized by treating a solution of diethyl (2-(7-fluoro-5-oxo-1-thioxo-1,2-dihydro-[1,2,4]triazolo[4,3-a]quinazolin-4(5H)-yl)ethyl)phosphonate (0.111 g, 0.277 mmol) in DCM (4.62 ml) at 0° C. with bromotrimethylsilane (0.216 ml, 1.663 mmol) dropwise (fast). The reaction was then warmed to room temperature and stirred for 8 hr. The reaction was concentrated, redissolved, and purified by HPLC: ¹H NMR (400 MHz, DMSO-d₆) δ 10.27 (dd, J=9.3, 4.6 Hz, 1H), 7.90 (dd, J=8.7, 3.1 Hz, 1H), 7.79 (td, J=8.5, 3.1 Hz, 1H), 4.24-4.14 (m, 2H), 2.06-1.93 (m, 2H); ¹⁹F NMR (376 MHz, DMSO-d₆) δ −113.10 (q, J=7.5 Hz); ³¹P NMR (162 MHz, DMSO-d₆) δ 20.70.

### Example 40

This example demonstrates a synthesis of 4-(cyclopropylmethyl)-7-fluoro-1-thioxo-2,4-dihydro-[1,2,4]triazolo[4,3-a]quinazolin-5(1H)-one in an aspect of the invention.

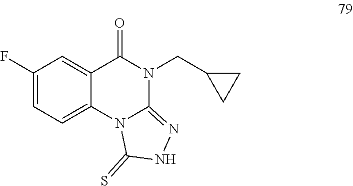 [SMILES: O=c1c2cc(F)ccc2n2c(=S)[nH]nc2n1CC1CC1]

3-(Cyclopropylmethyl)-6-fluoro-2-thioxo-2,3-dihydroquinazolin-4(1H)-one was synthesized according to General Procedure B. Then 4-(cyclopropylmethyl)-7-fluoro-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.29 (dd, J=9.3, 4.7 Hz, 1H), 7.92 (dd, J=8.6, 3.0 Hz, 1H), 7.80 (ddd, J=9.3, 7.9, 3.1 Hz, 1H), 3.92 (d, J=7.1 Hz, 2H), 1.27 (s, 1H), 0.49-0.37 (m, 4H); ¹⁹F NMR (376 MHz, DMSO-d₆) δ −113.02 (td, J=8.3, 4.7 Hz); HRMS (ES-API): m/z calculated for C₁₃H₁₂FN₄OS (M+H) 291.0710, found 291.0720.

### Example 41

This example demonstrates a synthesis of 7-fluoro-4-(trans-2-methylcyclopropyl)-1-thioxo-2,4-dihydro-[1,2,4]triazolo[4,3-a]quinazolin-5(1H)-one in an aspect of the invention.

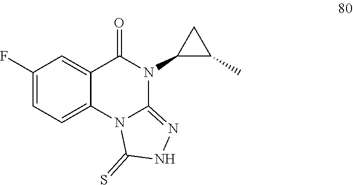 [SMILES: C[C@H]1C[C@@H]1n1c(=O)c2cc(F)ccc2n2c(=S)[nH]nc12]

6-Fluoro-3-(trans-2-methylcyclopropyl)-2-thioxo-2,3-dihydroquinazolin-4(1H)-one was synthesized according to General Procedure B. Then 7-fluoro-4-(trans-2-methylcyclopropyl)-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 13.99 (s, 1H), 10.26 (dd, J=9.3, 4.6 Hz, 1H), 7.86 (dd, J=8.8, 3.1 Hz, 1H), 7.76 (ddd, J=10.7, 8.2, 3.0 Hz, 1H), 2.56 (dt, J=7.3, 3.5 Hz, 1H), 1.27-1.19 (m, 1H), 1.15 (d, J=6.0 Hz, 3H), 1.07 (dt, J=9.6, 4.8 Hz, 1H), 0.90 (q, J=6.6 Hz, 1H); ¹⁹F NMR (376 MHz, DMSO-d₆) δ −113.28 (q, J=7.3, 6.8 Hz); HRMS (ES-API): m/z calculated for C₁₃H₁₂FN₄OS (M+H) 291.0710, found 291.0717.

### Example 42

This example demonstrates a synthesis of 4-(2-chlorophenethyl)-7-fluoro-1-thioxo-2,4-dihydro-[1,2,4]triazolo[4,3-a]quinazolin-5(1H)-one in an aspect of the invention.

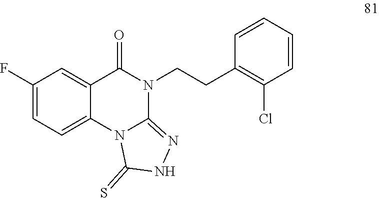 [SMILES: O=c1c2cc(F)ccc2n2c(=S)[nH]nc2n1CCc1ccccc1Cl]

3-(2-Chlorophenethyl)-6-fluoro-2-thioxo-2,3-dihydroquinazolin-4(1H)-one was synthesized according to General Procedure B. Then 4-(2-chlorophenethyl)-7-fluoro-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.28 (s, 1H), 7.88 (dd, J=8.6, 2.9 Hz, 1H), 7.80 (t, J=8.7 Hz, 1H), 7.39 (dd, J=5.6, 3.7 Hz, 1H), 7.36-7.29 (m, 1H), 7.23 (dd, J=5.9, 3.5 Hz, 2H), 4.27 (t, J=7.5 Hz, 2H), 3.13 (t, J=7.3 Hz, 2H); ¹⁹F NMR (376 MHz, DMSO-d₆) δ −112.98; HRMS (ES-API): m/z calculated for C₁₇H₁₃ClFN₄OS (M+H) 375.0477, found 375.0481.

### Example 43

This example demonstrates a synthesis of 4-(4-chlorophenethyl)-7-fluoro-1-thioxo-2,4-dihydro-[1,2,4]triazolo[4,3-a]quinazolin-5(1H)-one in an aspect of the invention.

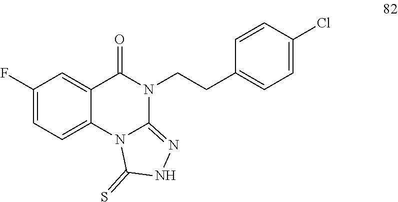 [SMILES: O=c1c2cc(F)ccc2n2c(=S)[nH]nc2n1CCc1ccc(Cl)cc1]

3-(4-Chlorophenethyl)-6-fluoro-2-thioxo-2,3-dihydroquinazolin-4(1H)-one was synthesized according to General Procedure B. Then 4-(4-chlorophenethyl)-7-fluoro-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.28 (dd, J=9.4, 4.6 Hz, 1H), 7.89 (dd, J=8.6, 3.1 Hz, 1H), 7.80 (ddd, J=9.3, 7.9, 3.1 Hz, 1H), 7.32 (d, J=8.5 Hz, 2H), 7.27 (d, J=8.5 Hz, 2H), 4.21 (t, J=7.7 Hz, 2H), 2.98 (t, J=7.6 Hz, 2H); ¹⁹F NMR (376 MHz, DMSO-d₆) δ −113.00 (td, J=8.4, 5.0 Hz); HRMS (ES-API): m/z calculated for C₁₇H₁₃ClFN₄OS (M+H) 375.0477, found 375.0480.

### Example 44

This example demonstrates a synthesis of 7-fluoro-4-(2-(pyridin-2-yl)ethyl)-1-thioxo-2,4-dihydro-[1,2,4]triazolo[4,3-a]quinazolin-5(1H)-one in an aspect of the invention.

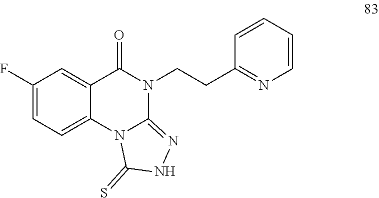 [SMILES: O=c1c2cc(F)ccc2n2c(=S)[nH]nc2n1CCc1ccccn1]

6-Fluoro-3-(2-(pyridin-2-yl)ethyl)-2-thioxo-2,3-dihydroquinazolin-4(1H)-one was synthesized according to General Procedure B. Then 7-fluoro-4-(2-(pyridin-2-yl)ethyl)-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.29 (dd, J=9.4, 4.6 Hz, 1H), 8.46 (d, J=5.0 Hz, 1H), 7.88 (dd, J=8.6, 3.0 Hz, 1H), 7.80 (td, J=8.6, 3.1 Hz, 1H), 7.76-7.73 (m, 1H), 7.36 (d, J=7.7 Hz, 1H), 7.29-7.24 (m, 1H), 4.38 (t, J=7.6 Hz, 2H), 3.20-3.11 (m, 2H); ¹⁹F NMR (376 MHz, DMSO-d₆) δ −112.97f-−113.04 (m); HRMS (ES-API): m/z calculated for C₁₆H₁₃FN₅OS (M+H) 342.0819, found 342.0818; Purity (HPLC) 94.92%.

### Example 45

This example demonstrates a synthesis of 7-fluoro-4-(2-(pyridin-3-yl)ethyl)-1-thioxo-2,4-dihydro-[1,2,4]triazolo[4,3-a]quinazolin-5(1H)-one in an aspect of the invention.

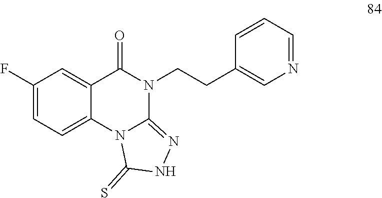 [SMILES: O=c1c2cc(F)ccc2n2c(=S)[nH]nc2n1CCc1cccnc1]

6-Fluoro-3-(2-(pyridin-3-yl)ethyl)-2-thioxo-2,3-dihydroquinazolin-4(1H)-one was synthesized according to General Procedure B. Then 7-fluoro-4-(2-(pyridin-3-yl)ethyl)-1-thioxo-2,4-dihydro-[1,2,4]triazolo[4,3-a]quinazolin-5(1H)-one was synthesized according to General Procedure D: ¹H NMR (400 MHz, DMSO-d₆) δ 10.30 (dd, J=9.3, 4.7 Hz, 1H), 8.56 (d, J=2.2 Hz, 1H), 8.50 (dd, J=4.9, 1.6 Hz, 1H), 7.92-7.85 (m, 2H), 7.82 (td, J=8.6, 3.1 Hz, 1H), 7.46 (dd, J=7.9, 5.0 Hz, 1H), 4.29 (t, J=7.2 Hz, 2H), 3.08 (t, J=7.2 Hz, 2H); ¹⁹F NMR (376 MHz, DMSO-d₆) δ −113.00 (td, J=8.2, 4.6 Hz); HRMS (ES-API): m/z calculated for C₁₆H₁₃FN₅OS (M+H) 342.0819, found 342.0811.

### Example 46

This example demonstrates a synthesis of 7-fluoro-4-(2-(pyridin-4-yl)ethyl)-1-thioxo-2,4-dihydro-[1,2,4]triazolo[4,3-a]quinazolin-5(1H)-one in an aspect of the invention.

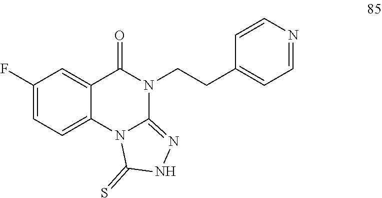 [SMILES: O=c1c2cc(F)ccc2n2c(=S)[nH]nc2n1CCc1ccncc1]

6-Fluoro-3-(2-(pyridin-4-yl)ethyl)-2-thioxo-2,3-dihydroquinazolin-4(1H)-one was synthesized according to General Procedure B. Then 7-fluoro-4-(2-(pyridin-4-yl)ethyl)-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.30 (dd, J=9.3, 4.7 Hz, 1H), 8.65-8.59 (m, 2H), 7.91-7.77 (m, 2H), 7.66 (d, J=5.6 Hz, 2H), 4.34 (t, J=7.1 Hz, 2H), 3.17 (t, J=7.1 Hz, 2H); ¹⁹F NMR (376 MHz, DMSO-d₆) δ −112.98 (td, J=8.2, 4.7 Hz); HRMS (ES-API): m/z calculated for C₁₆H₁₃FN₅OS (M+H) 342.0819, found 342.0825.

### Example 47

This example demonstrates a synthesis of tert-butyl 3-(2-(7-fluoro-5-oxo-1-thioxo-1,2-dihydro-[1,2,4]triazolo[4,3-a]quinazolin-4(5H)-yl)ethyl)azetidine-1-carboxylate in an aspect of the invention.

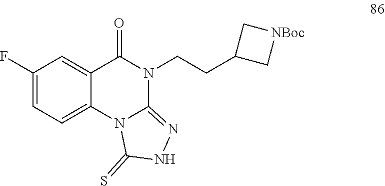 [SMILES: CC(C)(C)OC(=O)N1CC(CCn2c(=O)c3cc(F)ccc3n3c(=S)[nH]nc23)C1]

tert-Butyl 3-(2-(6-fluoro-4-oxo-2-thioxo-1,4-dihydroquinazolin-3(2H)-yl)ethyl)azetidine-1-carboxylate was synthesized according to General Procedure B. Then tert-butyl 3-(2-(7-fluoro-5-oxo-1-thioxo-1,2-dihydro-[1,2,4]triazolo[4,3-a]quinazolin-4(5H)-yl)ethyl)azetidine-1-carboxylate, TFA was synthesized according to General Procedure D: ¹H NMR (400 MHz, DMSO-d₆) δ 10.28 (dd, J=9.3, 4.6 Hz, 1H), 7.90 (dd, J=8.6, 3.1 Hz, 1H), 7.79 (ddd, J=9.3, 7.9, 3.1 Hz, 1H), 3.98 (t, J=6.8 Hz, 2H), 3.84 (s, 2H), 3.47 (s, 2H), 2.59-2.49 (m, 1H), 1.95 (q, J=7.1 Hz, 2H), 1.33 (s, 8H); ¹⁹F NMR (376 MHz, DMSO-d₆) δ −113.14 (q, J=7.5 Hz); HRMS (ES-API): m/z calculated for C₁₉H₂₂FN₅NaO₃S (M+Na) 442.1320, found 442.1332.

### Example 48

This example demonstrates a synthesis of 7-fluoro-4-(2-(1-methylpyrrolidin-2-yl)ethyl)-1-thioxo-2,4-dihydro-[1,2,4]triazolo[4,3-a]quinazolin-5(1H)-one in an aspect of the invention.

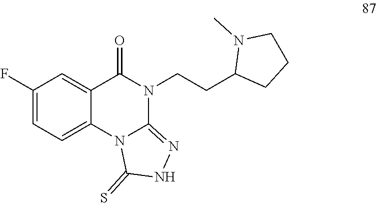 [SMILES: CN1CCCC1CCn1c(=O)c2cc(F)ccc2n2c(=S)[nH]nc12]

6-Fluoro-3-(2-(1-methylpyrrolidin-2-yl)ethyl)-2-thioxo-2,3-dihydroquinazolin-4(1H)-one was synthesized according to General Procedure B. Then 7-fluoro-4-(2-(1-methylpyrrolidin-2-yl)ethyl)-1-thioxo-2,4-dihydro-[1,2,4]triazolo[4,3-a]quinazolin-5(1H)-one, 2 TFA was synthesized according to General Procedure D: ¹H NMR (400 MHz, DMSO-d₆) δ 10.30 (dd, J=9.3, 4.6 Hz, 1H), 9.43 (s, 1H), 7.92 (dd, J=8.5, 3.1 Hz, 1H), 7.83 (td, J=8.5, 3.1 Hz, 1H), 4.11 (dp, J=28.3, 7.4 Hz, 2H), 3.51 (d, J=11.9 Hz, 1H), 3.01 (d, J=10.1 Hz, 1H), 2.73 (s, 3H), 2.65 (d, J=10.2 Hz, 1H), 2.43-2.25 (m, 1H), 2.04-1.79 (m, 3H), 1.72 (dq, J=16.0, 8.5 Hz, 1H); ¹⁹F NMR (376 MHz, DMSO-d₆) δ −112.96 (dt, J=12.5, 6.1 Hz); HRMS (ES-API): m/z calculated for C₁₆H₁₈FN₅NaOS (M+Na) 370.1108, found 370.1121.

### Example 49

This example demonstrates a synthesis of 7-fluoro-4-(2-(piperidin-1-yl)ethyl)-1-thioxo-2,4-dihydro-[1,2,4]triazolo[4,3-a]quinazolin-5(1H)-one in an aspect of the invention.

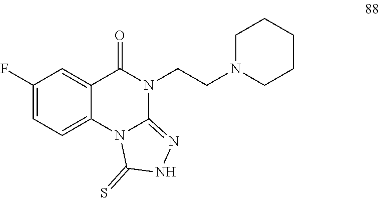 [SMILES: O=c1c2cc(F)ccc2n2c(=S)[nH]nc2n1CCN1CCCCC1]

6-Fluoro-3-(2-(piperidin-1-yl)ethyl)-2-thioxo-2,3-dihydroquinazolin-4(1H)-one was synthesized according to General Procedure B. Then 7-fluoro-4-(2-(piperidin-1-yl)ethyl)-1-thioxo-2,4-dihydro-[1,2,4]triazolo[4,3-a]quinazolin-5(1H)-one, 2 TFA was synthesized according to General Procedure D: ¹H NMR (400 MHz, DMSO-d₆) δ 10.31 (dd, J=9.3, 4.6 Hz, 1H), 8.78 (s, 1H), 7.96 (dd, J=8.5, 3.0 Hz, 1H), 7.86 (td, J=8.6, 3.0 Hz, 1H), 4.41 (t, J=6.1 Hz, 2H), 3.61 (s, 2H), 3.44 (s, 2H), 2.96 (s, 3H), 1.89-1.28 (m, 5H); ¹⁹F NMR (376 MHz, DMSO-d₆) δ −112.56 (q, J=7.4 Hz); HRMS (ES-API): m/z calculated for C₁₆H₁₉FN₅OS (M+H) 348.1289, found 348.1305.

### Example 50

This example demonstrates a synthesis of 4-(2-(4,4-difluoropiperidin-1-yl)ethyl)-7-fluoro-1-thioxo-2,4-dihydro-[1,2,4]triazolo[4,3-a]quinazolin-5(1H)-one in an aspect of the invention.

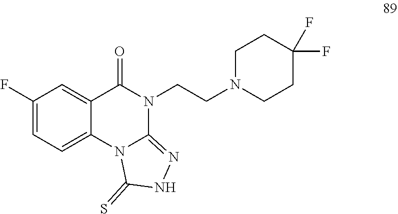 [SMILES: O=c1c2cc(F)ccc2n2c(=S)[nH]nc2n1CCN1CCC(F)(F)CC1]

3-(2-(4,4-Difluoropiperidin-1-yl)ethyl)-6-fluoro-2-thioxo-2,3-dihydroquinazolin-4(1H)-one was synthesized according to General Procedure B. Then 4-(2-(4,4-difluoropiperidin-1-yl)ethyl)-7-fluoro-1-thioxo-2,4-dihydro-[1,2,4]triazolo[4,3-a]quinazolin-5(1H)-one, 2 TFA was synthesized according to General Procedure D: ¹⁹F NMR (376 MHz, DMSO-d₆) δ −95.47 (d, J=213.5 Hz), −100.80 (d, J=228.4 Hz), −112.68; HRMS (ES-API): m/z calculated for C₁₆H₁₆F₃N₅NaOS (M+Na) 406.0920, found 406.0937.

### Example 51

This example demonstrates a synthesis of 7-fluoro-4-(2-morpholinoethyl)-1-thioxo-2,4-dihydro-[1,2,4]triazolo[4,3-a]quinazolin-5(1H)-one in an aspect of the invention.

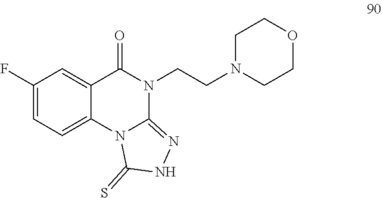 [SMILES: O=c1c2cc(F)ccc2n2c(=S)[nH]nc2n1CCN1CCOCC1]

6-Fluoro-3-(2-morpholinoethyl)-2-thioxo-2,3-dihydroquinazolin-4(1H)-one was synthesized according to General Procedure B. Then 7-fluoro-4-(2-morpholinoethyl)-1-thioxo-2,4-dihydro-[1,2,4]triazolo[4,3-a]quinazolin-5(1H)-one, 2 TFA was synthesized according to General Procedure D: ¹H NMR (400 MHz, DMSO-d₆) δ 10.31 (dd, J=9.4, 4.5 Hz, 1H), 9.48 (s, 1H), 7.95 (dd, J=8.6, 3.1 Hz, 1H), 7.86 (t, J=8.9 Hz, 1H), 4.41 (s, 2H), 3.98 (s, 2H), 3.57 (dd, J=25.5, 15.2 Hz, 2H), 3.35 (s, 4H), 3.18 (s, 2H); ¹⁹F NMR (376 MHz, DMSO-d₆) δ −112.55; HRMS (ES-API): m/z calculated for C₁₅H₁₇FN₅O₂S (M+H) 350.1082, found 350.1091.

### Example 52

This example demonstrates a synthesis of 7-fluoro-4-(2-(4-methylpiperazin-1-yl)ethyl)-1-thioxo-2,4-dihydro-[1,2,4]triazolo[4,3-a]quinazolin-5(1H)-one in an aspect of the invention.

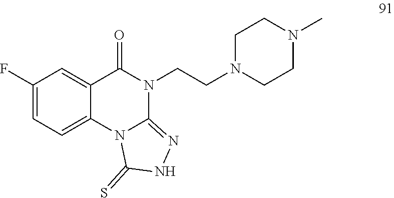 [SMILES: CN1CCN(CCn2c(=O)c3cc(F)ccc3n3c(=S)[nH]nc23)CC1]

6-Fluoro-3-(2-(4-methylpiperazin-1-yl)ethyl)-2-thioxo-2,3-dihydroquinazolin-4(1H)-one was synthesized according to General Procedure B. Then 7-fluoro-4-(2-(4-methylpiperazin-1-yl)ethyl)-1-thioxo-2,4-dihydro-[1,2,4]triazolo[4,3-a]quinazolin-5(1H)-one, 2 TFA was synthesized according to General Procedure D: HRMS (ES-API): m/z calculated for C₁₇H₁₈F₃N₄OS (M+H) 363.1398, found 363.1384.

### Example 53

This example demonstrates a synthesis of tert-butyl 4-(2-(7-fluoro-5-oxo-1-thioxo-1,2-dihydro-[1,2,4]triazolo[4,3-a]quinazolin-4(5H)-yl)ethyl)piperazine-1-carboxylate an aspect of the invention.

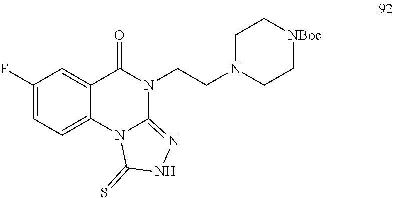 [SMILES: CC(C)(C)OC(=O)N1CCN(CCn2c(=O)c3cc(F)ccc3n3c(=S)[nH]nc23)CC1]

tert-Butyl 4-(2-(6-fluoro-4-oxo-2-thioxo-1,4-dihydroquinazolin-3(2H)-yl)ethyl)piperazine-1-carboxylate was synthesized according to General Procedure B. Then tert-butyl 4-(2-(7-fluoro-5-oxo-1-thioxo-1,2-dihydro-[1,2,4]triazolo[4,3-a]quinazolin-4(5H)-yl)ethyl)piperazine-1-carboxylate, 2 TFA was synthesized according to General Procedure D: HRMS (ES-API): m/z calculated for C₂₀H₂₆FN₆O₃S (M+H) 449.1766, found 449.1786.

### Example 54

This example demonstrates a synthesis of 4-(2-(4,4-difluorocyclohexyl)ethyl)-7-fluoro-1-thioxo-2,4-dihydro-[1,2,4]triazolo[4,3-a]quinazolin-5(1H)-one in an aspect of the invention.

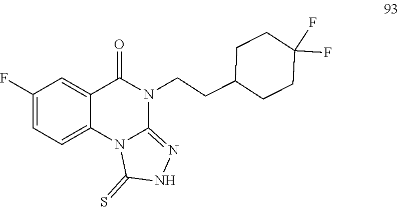 [SMILES: O=c1c2cc(F)ccc2n2c(=S)[nH]nc2n1CCC1CCC(F)(F)CC1]

3-(2-(4,4-Difluorocyclohexyl)ethyl)-6-fluoro-2-thioxo-2,3-dihydroquinazolin-4(1H)-one was synthesized according to General Procedure B. Then 4-(2-(4,4-difluorocyclohexyl)ethyl)-7-fluoro-1-thioxo-2,4-dihydro-[1,2,4]triazolo[4,3-a]quinazolin-5(1H)-one, TFA was synthesized according to General Procedure D: ¹H NMR (400 MHz, DMSO-d₆) δ 10.28 (dd, J=9.4, 4.6 Hz, 1H), 7.91 (dd, J=8.7, 3.0 Hz, 1H), 7.84-7.74 (m, 1H), 4.05 (t, J=7.4 Hz, 2H), 1.96 (d, J=10.9 Hz, 2H), 1.88-1.58 (m, 6H), 1.46 (s, 1H), 1.16 (q, J=12.3 Hz, 2H); ¹⁹F NMR (376 MHz, DMSO-d₆) δ −89.54 (d, J=231.6 Hz), −99.44 (d, J=231.4 Hz), −113.14 (q, J=7.1, 6.2 Hz); HRMS (ES-API): m/z calculated for C₁₇H₁₈F-3N₄OS (M+H) 383.1148, found 383.1166.

### Example 55

This example demonstrates a synthesis of tert-butyl (19-(7-fluoro-5-oxo-1-thioxo-1,2-dihydro-[1,2,4]triazolo[4,3-a]quinazolin-4(5H)-yl)-15-oxo-3,6,9,12-tetraoxa-16-azanonadecyl)carbamate in an aspect of the invention.

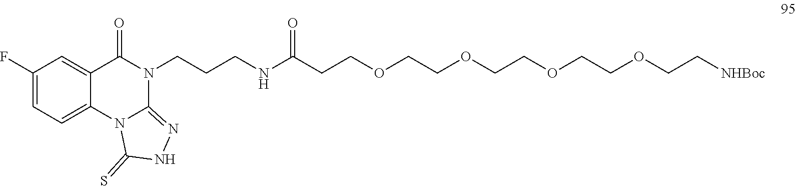 [SMILES: CC(C)(C)OC(=O)NCCOCCOCCOCCOCCC(=O)NCCCn1c(=O)c2cc(F)ccc2n2c(=S)[nH]nc12]

tert-Butyl (19-(7-fluoro-5-oxo-1-thioxo-1,2-dihydro-[1,2,4]triazolo[4,3-a]quinazolin-4(5H)-yl)-15-oxo-3,6,9,12-tetraoxa-16-azanonadecyl)carbamate, TFA was synthesized according to General Procedure F.

### Example 56

This example demonstrates a synthesis of 1-amino-N-(3-(7-fluoro-5-oxo-1-thioxo-1,2-dihydro-[1,2,4]triazolo[4,3-a]quinazolin-4(5H)-yl)propyl)-3,6,9,12-tetraoxapentadecan-15-amide in an aspect of the invention.

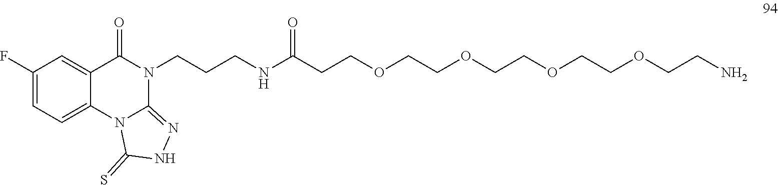 [SMILES: NCCOCCOCCOCCOCCC(=O)NCCCn1c(=O)c2cc(F)ccc2n2c(=S)[nH]nc12]

To a solution of 95 (0.025 g, 0.039 mmol) in DCM (0.195 ml) was added TFA (0.066 ml, 0.858 mmol) and the reaction stirred at room temperature overnight. The reaction was then blown down and redissolved. The crude material was purified by RP-ISCO (H₂O/MeCN+0.2% NH₄OH, 10-100%) to give 1-amino-N-(3-(7-fluoro-5-oxo-1-thioxo-1,2-dihydro-[1,2,4]triazolo[4,3-a]quinazolin-4(5H)-yl)propyl)-3,6,9,12-tetraoxapentadecan-15-amide, 2 TFA: ¹H NMR (400 MHz, CD₂Cl₂) δ 13.38 (s, 1H), 10.25 (dd, J=9.3, 4.6 Hz, 1H), 8.04 (s, 3H), 7.95 (dd, J=8.3, 3.0 Hz, 1H), 7.45 (ddd, J=9.4, 7.5, 3.1 Hz, 1H), 7.40 (s, 1H), 5.53 (s, 1H), 4.21 (t, J=6.7 Hz, 2H), 3.89-3.55 (m, 16H), 3.27 (d, J=22.8 Hz, 4H), 2.57 (s, 2H), 2.01 (s, 2H); ¹⁹F NMR (376 MHz, CD₂Cl₂) δ −112.54 (td, J=7.9, 4.4 Hz).

### Example 57

This example demonstrates a syntheses of (S)-22-amino-1-(7-fluoro-5-oxo-1-thioxo-1,2-dihydro-[1,2,4]triazolo[4,3-a]quinazolin-4(5H)-yl)-5,21-dioxo-8,11,14,17-tetraoxa-4,20-diazapentacosan-25-oic acid and tert-butyl (S)-22-amino-1-(7-fluoro-5-oxo-1-thioxo-1,2-dihydro-[1,2,4]triazolo[4,3-a]quinazolin-4(5H)-yl)-5,21-dioxo-8,11,14,17-tetraoxa-4,20-diazapentacosan-25-oate in an aspect of the invention.

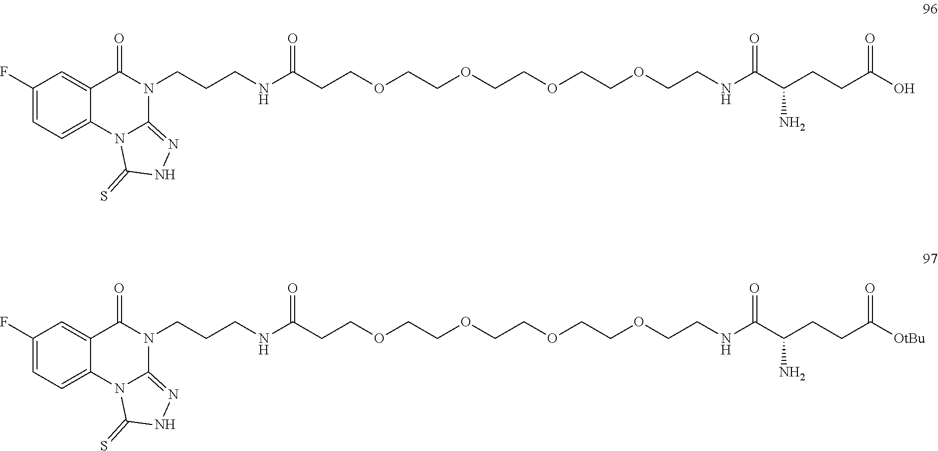 [SMILES: N[C@@H](CCC(=O)O)C(=O)NCCOCCOCCOCCOCCC(=O)NCCCn1c(=O)c2cc(F)ccc2n2c(=S)[nH]nc12 ; CC(C)(C)OC(=O)CC[C@H](N)C(=O)NCCOCCOCCOCCOCCC(=O)NCCCn1c(=O)c2cc(F)ccc2n2c(=S)[nH]nc12]

tert-Butyl (S)-22-((tert-butoxycarbonyl)amino)-1-(7-fluoro-5-oxo-1-thioxo-1,2-dihydro-[1,2,4]triazolo[4,3-a]quinazolin-4(51)-yl)-5,21-dioxo-8,11,14,17-tetraoxa-4,20-diazapentacosan-25-oate: To a solution of 1-amino-N-(3-(7-fluoro-5-oxo-1-thioxo-1,2-dihydro-[1,2,4]triazolo[4,3-a]quinazolin-4(5H)-yl)propyl)-3,6,9,12-tetraoxapentadecan-15-amide (0.04 g, 0.074 mmol) in DMF (0.190 ml) was added a solution of (S)-5-(tert-butoxy)-2-((tert-butoxycarbonyl)amino)-5-oxopentanoic acid (0.067 g, 0.222 mmol) and COMU (0.063 g, 0.148 mmol) followed by DIPEA (0.013 ml, 0.074 mmol). The reaction was then stirred overnight at room temperature. The reaction was quenched with water and concentrated. The product was purified by ISCO to give tert-butyl (S)-22-((tert-butoxycarbonyl)amino)-1-(7-fluoro-5-oxo-1-thioxo-1,2-dihydro-[1,2,4]triazolo[4,3-a]quinazolin-4(5H)-yl)-5,21-dioxo-8,11,14,17-tetraoxa-4,20-diazapentacosan-25-oate (0.0245 g, 40%): MS (ES-API) found (M+H) 826.2 and (M+Na) 848.1.

(S)-22-Amino-1-(7-fluoro-5-oxo-1-thioxo-1,2-dihydro-[1,2,4]triazolo[4,3-a]quinazolin-4(5H)-yl)-5,21-dioxo-8,11,14,17-tetraoxa-4,20-diazapentacosan-25-oic acid and tert-Butyl (S)-22-amino-1-(7-fluoro-5-oxo-1-thioxo-1,2-dihydro-[1,2,4]triazolo[4,3-a]quinazolin-4(5H)-yl)-5,21-dioxo-8,11,14,17-tetraoxa-4,20-diazapentacosan-25-oate: To a solution of tert-butyl (S)-22-((tert-butoxycarbonyl)amino)-1-(7-fluoro-5-oxo-1-thioxo-1,2-dihydro-[1,2,4]triazolo[4,3-a]quinazolin-4(5H)-yl)-5,21-dioxo-8,11,14,17-tetraoxa-4,20-diazapentacosan-25-oate (0.012 g, 0.015 mmol) in DCM (0.145 ml) was added TFA (0.022 ml, 0.291 mmol) and triisopropylsilane (5.95 μl, 0.029 mmol). The reaction was stirred at room temperature for 2 h and concentrated. The mixture of products was purified by HPLC to give (S)-22-amino-1-(7-fluoro-5-oxo-1-thioxo-1,2-dihydro-[1,2,4]triazolo[4,3-a]quinazolin-4(5H)-yl)-5,21-dioxo-8,11,14,17-tetraoxa-4,20-diazapentacosan-25-oic acid, 2 TFA and tert-butyl (S)-22-amino-1-(7-fluoro-5-oxo-1-thioxo-1,2-dihydro-[1,2,4]triazolo[4,3-a]quinazolin-4(5H)-yl)-5,21-dioxo-8,11,14,17-tetraoxa-4,20-diazapentacosan-25-oate, 2 TFA: 96: HRMS (ES-API): m/z calculated for C₂₈H₄₁FN₇O₉S (M+H) 670.2665, found 670.2672. 97: HRMS (ES-API): m/z calculated for C₃₂H₄₉FN₇O₉S (M+H) 726.3291, found 726.3284.

### Example 58

This example demonstrates a synthesis of tert-butyl (16-(7-fluoro-5-oxo-1-thioxo-1,2-dihydro-[1,2,4]triazolo[4,3-a]quinazolin-4(5H)-yl)-12-oxo-3,6,9-trioxa-13-azahexadecyl)carbamate in an aspect of the invention.

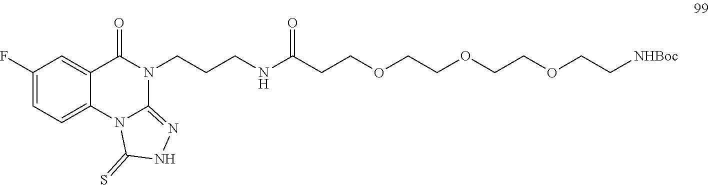 [SMILES: CC(C)(C)OC(=O)NCCOCCOCCOCCC(=O)NCCCn1c(=O)c2cc(F)ccc2n2c(=S)[nH]nc12]

tert-Butyl (16-(7-fluoro-5-oxo-1-thioxo-1,2-dihydro-[1,2,4]triazolo[4,3-a]quinazolin-4(5H)-yl)-12-oxo-3,6,9-trioxa-13-azahexadecyl)carbamate, TFA was synthesized according to General Procedure F: HRMS (ES-API): m/z calculated for C₂₆H₃₇F—N₆NaO₇S (M+Na) 619.2321, found 619.2330.

### Example 59

This example demonstrates a synthesis of 3-(2-(2-(2-aminoethoxy)ethoxy)ethoxy)-N-(3-(7-fluoro-5-oxo-1-thioxo-1,2-dihydro-[1,2,4]triazolo[4,3-a]quinazolin-4(5H)-yl)propyl)propenamide in an aspect of the invention.

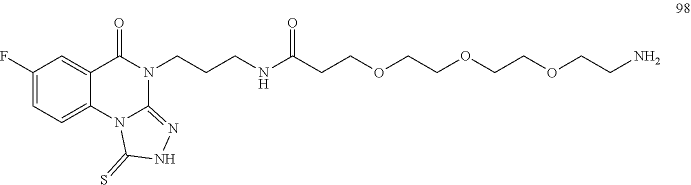 [SMILES: NCCOCCOCCOCCC(=O)NCCCn1c(=O)c2cc(F)ccc2n2c(=S)[nH]nc12]

To a solution of 99 (0.1496 g, 0.251 mmol) in HCl in dioxane (0.627 ml, 2.507 mmol) was added and triisopropylsilane (0.079 g, 0.501 mmol) and stirred at room temperature for 2 hr and the reaction was concentrated. The product was purified by HPLC to give 3-(2-(2-(2-aminoethoxy)ethoxy)ethoxy)-N-(3-(7-fluoro-5-oxo-1-thioxo-1,2-dihydro-[1,2,4]triazolo[4,3-a]quinazolin-4(5H)-yl)propyl)propenamide, 2 TFA: HRMS (ES-API): m/z calculated for C₂₁H₃₀FN₆O₅S (M+H) 497.1977, found 497.1973.

### Example 60

This example demonstrates a synthesis of tert-butyl (6-((3-(7-fluoro-5-oxo-1-thioxo-1,2-dihydro-[1,2,4]triazolo[4,3-a]quinazolin-4(5H)-yl)propyl)amino)-6-oxohexyl)carbamate in an aspect of the invention.

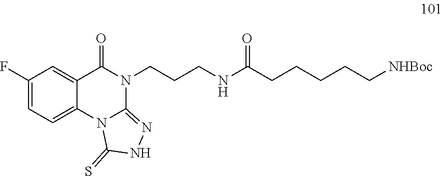 [SMILES: CC(C)(C)OC(=O)NCCCCCC(=O)NCCCn1c(=O)c2cc(F)ccc2n2c(=S)[nH]nc12]

tert-Butyl (6-((3-(7-fluoro-5-oxo-1-thioxo-1,2-dihydro-[1,2,4]triazolo[4,3-a]quinazolin-4(5H)-yl)propyl)amino)-6-oxohexyl)carbamate was synthesized according to General Procedure F: ¹H NMR (400 MHz, DMSO-d₆) δ 10.29 (dd, J=9.3, 4.7 Hz, 1H), 7.91 (dd, J=8.5, 3.1 Hz, 1H), 7.80 (ddd, J=9.1, 7.8, 2.8 Hz, 2H), 6.73 (t, J=5.6 Hz, 1H), 4.02 (dd, J=8.3, 6.3 Hz, 2H), 3.10 (q, J=6.6 Hz, 2H), 2.86 (q, J=6.6 Hz, 2H), 2.00 (t, J=7.4 Hz, 2H), 1.82 (p, J=7.0 Hz, 2H), 1.44 (p, J=7.4 Hz, 2H), 1.37-1.27 (m, 11H), 1.24-1.11 (m, 2H); ¹⁹F NMR (376 MHz, DMSO-d₆) δ −113.19; HRMS (ES-API): m/z calculated for C₂₃H₃₁FN₆NaO₄S (M+Na) 529.2004, found 529.1994.

### Example 61

This example demonstrates a synthesis of 6-amino-N-(3-(7-fluoro-5-oxo-1-thioxo-1,2-dihydro-[1,2,4]triazolo[4,3-a]quinazolin-4(5H)-yl)propyl)hexanamide in an aspect of the invention.

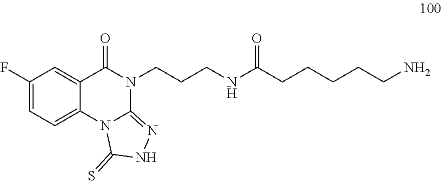 [SMILES: NCCCCCC(=O)NCCCn1c(=O)c2cc(F)ccc2n2c(=S)[nH]nc12]

To a solution of 101 (0.049 g, 0.097 mmol) in HCl in dioxane (0.242 ml, 0.967 mmol) was added triisopropylsilane (0.031 g, 0.193 mmol) and stirred at room temperature for 2 hr. The reaction was concentrated and purified by HPLC to give 6-amino-N-(3-(7-fluoro-5-oxo-1-thioxo-1,2-dihydro-[1,2,4]triazolo[4,3-a]quinazolin-4(5H)-yl)propyl)hexanamide, 2 TFA: HRMS (ES-API): m/z calculated for C₁₈H₂₃FN₆NaO₂S (M+Na) 430.1506, found 430.1512.

### Example 62

This example demonstrates a synthesis of tert-butyl (R)-4-((tert-butoxycarbonyl)amino)-5-((3-(7-fluoro-5-oxo-1-thioxo-1,2-dihydro-[1,2,4]triazolo[4,3-a]quinazolin-4(5H)-yl)propyl)amino)-5-oxopentanoate in an aspect of the invention.

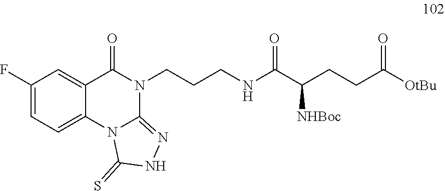 [SMILES: CC(C)(C)OC(=O)CC[C@@H](NC(=O)OC(C)(C)C)C(=O)NCCCn1c(=O)c2cc(F)ccc2n2c(=S)[nH]nc12]

tert-Butyl (R)-4-((tert-butoxycarbonyl)amino)-5-((3-(7-fluoro-5-oxo-1-thioxo-1,2-dihydro-[1,2,4]triazolo[4,3-a]quinazolin-4(5H)-yl)propyl)amino)-5-oxopentanoate, TFA was synthesized according to General Procedure F: ¹H NMR (400 MHz, DMSO-d₆) δ 10.29 (dd, J=9.3, 4.7 Hz, 1H), 7.90 (dd, J=8.6, 3.1 Hz, 1H), 7.85-7.76 (m, 2H), 6.82 (d, J=8.2 Hz, 1H), 4.03 (t, J=7.2 Hz, 2H), 3.90-3.80 (m, 1H), 3.12 (dp, J=20.2, 6.6 Hz, 2H), 2.18 (t, J=7.7 Hz, 2H), 1.83 (dt, J=13.7, 6.5 Hz, 3H), 1.66 (dt, J=14.3, 7.6 Hz, 1H), 1.36 (s, 9H), 1.35 (s, 9H); ¹⁹F NMR (376 MHz, DMSO-d₆) δ −113.21 (t, J=6.8 Hz); HRMS (ES-API): m/z calculated for C₂₆H₃₅FN₆NaO₆S (M+Na) 601.2215, found 601.2214.

### Example 63

This example demonstrates a synthesis of tert-butyl (S)-4-((tert-butoxycarbonyl)amino)-5-((3-(7-fluoro-5-oxo-1-thioxo-1,2-dihydro-[1,2,4]triazolo[4,3-a]quinazolin-4(5H)-yl)propyl)amino)-5-oxopentanoate in an aspect of the invention.

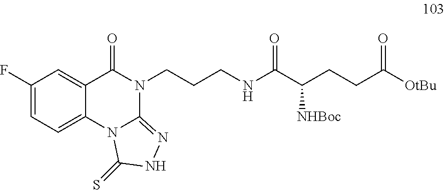 [SMILES: CC(C)(C)OC(=O)CC[C@H](NC(=O)OC(C)(C)C)C(=O)NCCCn1c(=O)c2cc(F)ccc2n2c(=S)[nH]nc12]

tert-Butyl (S)-4-((tert-butoxycarbonyl)amino)-5-((3-(7-fluoro-5-oxo-1-thioxo-1,2-dihydro-[1,2,4]triazolo[4,3-a]quinazolin-4(5H)-yl)propyl)amino)-5-oxopentanoate, TFA was synthesized according to General Procedure F: ¹⁹F NMR (376 MHz, DMSO-d₆) δ −113.15 (d, J=11.0 Hz); HRMS (ES-API): m/z calculated for C₂₆H₃₅FN₆NaO₆S (M+Na) 601.2215, found 601.2216.

### Example 64

This example demonstrates a syntheses of tert-butyl (S)-4-amino-5-((3-(7-fluoro-5-oxo-1-thioxo-1,2-dihydro-[1,2,4]triazolo[4,3-a]quinazolin-4(5H)-yl)propyl)amino)-5-oxopentanoate and (S)-4-amino-5-((3-(7-fluoro-5-oxo-1-thioxo-1,2-dihydro-[1,2,4]triazolo[4,3-a]quinazolin-4(5H)-yl)propyl)amino)-5-oxopentanoic acid in an aspect of the invention.

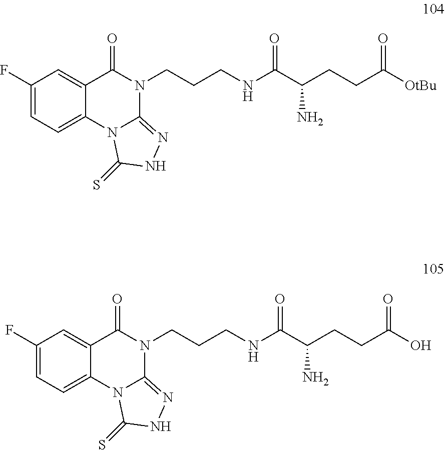 [SMILES: CC(C)(C)OC(=O)CC[C@H](N)C(=O)NCCCn1c(=O)c2cc(F)ccc2n2c(=S)[nH]nc12 ; N[C@@H](CCC(=O)O)C(=O)NCCCn1c(=O)c2cc(F)ccc2n2c(=S)[nH]nc12]

To a solution of 103 (0.0154 g, 0.027 mmol) in DCM (0.266 ml) was added TFA (0.041 ml, 0.532 mmol) and triisopropylsilane (10.90 μl, 0.053 mmol). The reaction was stirred at room temperature for 2 h and concentrated. The products were purified by HPLC to give tert-butyl (S)-4-amino-5-((3-(7-fluoro-5-oxo-1-thioxo-1,2-dihydro-[1,2,4]triazolo[4,3-a]quinazolin-4(5H)-yl)propyl)amino)-5-oxopentanoate, 2 TFA and (S)-4-amino-5-((3-(7-fluoro-5-oxo-1-thioxo-1,2-dihydro-[1,2,4]triazolo[4,3-a]quinazolin-4(5H)-yl)propyl)amino)-5-oxopentanoic acid; 104: ¹H NMR (400 MHz, DMSO-d₆) δ 10.30 (dd, J=9.3, 4.8 Hz, 1H), 8.25 (s, 3H), 8.01 (t, J=5.7 Hz, 1H), 7.91 (dd, J=8.6, 3.1 Hz, 1H), 7.82 (ddd, J=9.3, 8.0, 3.1 Hz, 1H), 4.07-3.99 (m, 2H), 3.92 (t, J=6.3 Hz, 1H), 3.13 (q, J=6.7 Hz, 2H), 2.22 (ddt, J=22.8, 15.3, 7.4 Hz, 2H), 1.95 (q, J=7.4 Hz, 2H), 1.85 (p, J=7.1 Hz, 2H), 1.45 (s, 9H); ¹⁹F NMR (376 MHz, DMSO-d₆) δ −113.12 (dt, J=7.8, 4.1 Hz); HRMS (ES-API): m/z calculated for C₂₁H₂₈FN₆O₄S (M+H) 479.1871, found 479.1866. 105: ¹H NMR (400 MHz, DMSO-d₆) δ 10.31 (dd, J=9.3, 4.7 Hz, 1H), 8.26 (s, 3H), 8.02 (t, J=5.7 Hz, 1H), 7.92 (dd, J=8.5, 3.1 Hz, 1H), 7.83 (ddd, J=9.3, 8.0, 3.1 Hz, 1H), 4.05 (dd, J=8.1, 6.4 Hz, 2H), 3.93 (t, J=6.6 Hz, 1H), 3.15 (q, J=6.7 Hz, 2H), 2.54 (s, OH), 2.24 (ddt, J=23.0, 15.4, 7.5 Hz, 2H), 1.97 (q, J=7.4 Hz, 2H), 1.86 (p, J=7.2 Hz, 2H), 1.46 (s, 9H); ¹⁹F NMR (376 MHz, DMSO-d₆) δ −113.10 (td, J=8.2, 4.8 Hz); HRMS (ES-API): m/z calculated for C₁₇H₂₀FN₆O₄S (M+H) 423.1245, found 423.1232.

### Example 65

This example demonstrates a synthesis of 4-((3-(7-fluoro-5-oxo-1-thioxo-1,2-dihydro-[1,2,4]triazolo[4,3-a]quinazolin-4(5H)-yl)propyl)amino)-4-oxobutanoic acid in an aspect of the invention.

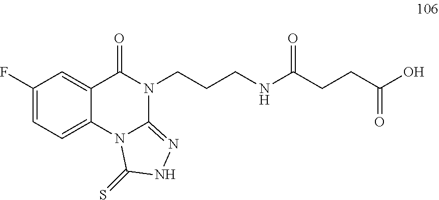 [SMILES: O=C(O)CCC(=O)NCCCn1c(=O)c2cc(F)ccc2n2c(=S)[nH]nc12]

4-((3-(7-Fluoro-5-oxo-1-thioxo-1,2-dihydro-[1,2,4]triazolo[4,3-a]quinazolin-4(5H)-yl)propyl)amino)-4-oxobutanoic acid, TFA was synthesized according to General Procedure F: ¹H NMR (400 MHz, DMSO-d₆) δ 10.34 (s, 1H), 7.92 (dd, J=8.6, 3.1 Hz, 1H), 7.86 (t, J=5.6 Hz, 1H), 7.80 (ddd, J=9.3, 8.0, 3.1 Hz, 1H), 4.05 (t, J=7.3 Hz, 2H), 3.12 (q, J=6.7 Hz, 2H), 2.45-2.37 (m, 2H), 2.32-2.24 (m, 3H), 1.84 (p, J=7.1 Hz, 2H); ¹⁹F NMR (376 MHz, DMSO-d₆) δ −113.28; HRMS (ES-API): m/z calculated for C₁₆H₁₇FN₅O₄S (M+H) 394.0980, found 394.0979.

### Example 66

This example demonstrates a synthesis of N-(3-(7-fluoro-5-oxo-1-thioxo-1,2-dihydro-[1,2,4]triazolo[4,3-a]quinazolin-4(5H)-yl)propyl)-4-sulfamoylbutanamide in an aspect of the invention.

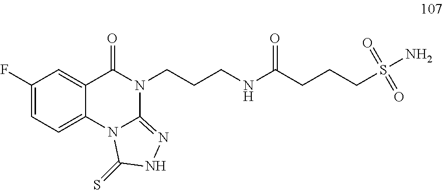 [SMILES: NS(=O)(=O)CCCC(=O)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-4-sulfamoylbutanamide, TFA was synthesized according to General Procedure F: ¹H NMR (400 MHz, DMSO-d₆) δ 10.31 (dd, J=9.3, 4.7 Hz, 1H), 7.93 (dd, J=8.6, 3.1 Hz, 1H), 7.87 (t, J=5.6 Hz, 1H), 7.85-7.78 (m, 1H), 6.75 (s, 2H), 4.05 (dd, J=8.1, 6.3 Hz, 2H), 3.13 (q, J=6.6 Hz, 2H), 3.00-2.92 (m, 2H), 2.20 (t, J=7.4 Hz, 2H), 1.94-1.81 (m, 4H); ¹⁹F NMR (376 MHz, DMSO-d₆) δ −113.14; HRMS (ES-API): m/z calculated for C₁₆H₂₀FN₆O₄S₂ (M+H) 443.0966, found 443.0977.

### Example 67

This example demonstrates a synthesis of N-(3-(7-fluoro-5-oxo-1-thioxo-1,2-dihydro-[1,2,4]triazolo[4,3-a]quinazolin-4(5H)-yl)propyl)cyclopentanecarboxamide in an aspect of the invention.

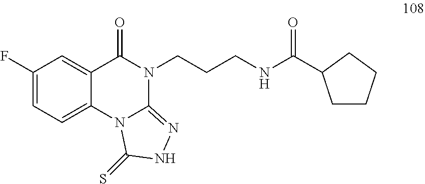 [SMILES: O=C(NCCCn1c(=O)c2cc(F)ccc2n2c(=S)[nH]nc12)C1CCCC1]

N-(3-(7-Fluoro-5-oxo-1-thioxo-1,2-dihydro-[1,2,4]triazolo[4,3-a]quinazolin-4(5H)-yl)propyl)cyclopentanecarboxamide, TFA was synthesized according to General Procedure F: ¹H NMR (400 MHz, DMSO-d₆) δ 10.29 (dd, J=9.3, 4.7 Hz, 1H), 7.91 (dd, J=8.6, 3.1 Hz, 1H), 7.80 (ddd, J=9.3, 8.0, 3.1 Hz, 1H), 7.75 (t, J=5.6 Hz, 1H), 4.02 (dd, J=8.2, 6.5 Hz, 2H), 3.10 (q, J=6.6 Hz, 2H), 1.82 (p, J=7.0 Hz, 2H), 1.73-1.63 (m, 2H), 1.63-1.51 (m, 4H), 1.50-1.40 (m, 2H); ¹⁹F NMR (376 MHz, DMSO-d₆) δ −113.19 (td, J=8.2, 4.6 Hz); HRMS (ES-API): m/z calculated for C₁₈H₂₁FN₅O₂S (M+H) 390.1395, found 390.1387.

### Example 68

This example demonstrates a synthesis of trans-4-(tert-butyl)-N-(3-(7-fluoro-5-oxo-1-thioxo-1,2-dihydro-[1,2,4]triazolo[4,3-a]quinazolin-4(5H)-yl)propyl)cyclohexane-1-carboxamide in an aspect of the invention.

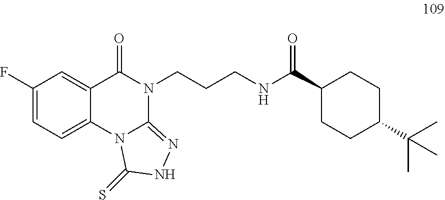 [SMILES: CC(C)(C)[C@H]1CC[C@H](C(=O)NCCCn2c(=O)c3cc(F)ccc3n3c(=S)[nH]nc23)CC1]

trans-4-(tert-Butyl)-N-(3-(7-fluoro-5-oxo-1-thioxo-1,2-dihydro-[1,2,4]triazolo[4,3-a]quinazolin-4(5H)-yl)propyl)cyclohexane-1-carboxamide, TFA was synthesized according to General Procedure F: ¹H NMR (400 MHz, DMSO-d₆) δ 10.29 (dd, J=9.3, 4.7 Hz, 1H), 7.91 (dd, J=8.6, 3.1 Hz, 1H), 7.80 (ddd, J=9.3, 8.0, 3.1 Hz, 1H), 7.67 (t, J=5.7 Hz, 1H), 4.01 (dd, J=8.4, 6.4 Hz, 2H), 3.09 (q, J=6.5 Hz, 2H), 1.93 (td, J=12.2, 2.9 Hz, 1H), 1.81 (p, J=6.9 Hz, 2H), 1.73 (d, J=9.8 Hz, 4H), 1.26 (q, J=12.2 Hz, 2H), 0.97-0.87 (m, 3H), 0.80 (s, 9H); ¹⁹F NMR (376 MHz, DMSO-d₆) δ −113.21 (td, J=8.3, 4.6 Hz); HRMS (ES-API): m/z calculated for C₂₃H₃₁FN₅O₂S (M+H) 460.2177, found 460.2177.

### Example 69

This example demonstrates a synthesis of tert-butyl (4-((3-(7-fluoro-5-oxo-1-thioxo-1,2-dihydro-[1,2,4]triazolo[4,3-a]quinazolin-4(5H)-yl)propyl)carbamoyl)cyclohexyl)carbamate in an aspect of the invention.

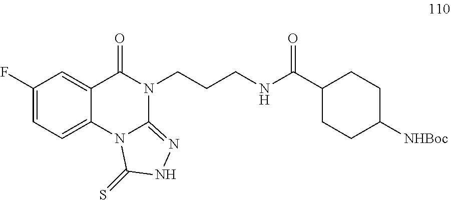 [SMILES: CC(C)(C)OC(=O)NC1CCC(C(=O)NCCCn2c(=O)c3cc(F)ccc3n3c(=S)[nH]nc23)CC1]

tert-Butyl (4-((3-(7-fluoro-5-oxo-1-thioxo-1,2-dihydro-[1,2,4]triazolo[4,3-a]quinazolin-4(5H)-yl)propyl)carbamoyl)cyclohexyl)carbamate, 2 TFA was synthesized according to General Procedure F: ¹H NMR (400 MHz, DMSO-d₆) δ 10.29 (dd, J=9.3, 4.7 Hz, 1H), 7.91 (dd, J=8.6, 3.1 Hz, 1H), 7.80 (ddd, J=9.4, 8.0, 3.2 Hz, 1H), 7.67 (t, J=5.7 Hz, 1H), 6.65 (d, J=6.5 Hz, 1H), 4.06-3.98 (m, 2H), 3.43 (s, 1H), 3.11 (q, J=6.5 Hz, 2H), 2.10 (dq, J=12.2, 3.8 Hz, 1H), 1.82 (p, J=7.7, 7.3 Hz, 2H), 1.77-1.68 (m, 1H), 1.64-1.53 (m, 2H), 1.47-1.37 (m, 5H), 1.36 (s, 9H); ¹⁹F NMR (376 MHz, DMSO-d₆) δ −113.17 (td, J=8.3, 4.8 Hz); HRMS (ES-API): m/z calculated for C₂₄H₃₁FN₆NaO₄S (M+Na) 541.2004, found 541.2011.

### Example 70

This example demonstrates a synthesis of 4-amino-N-(3-(7-fluoro-5-oxo-1-thioxo-1,2-dihydro-[1,2,4]triazolo[4,3-a]quinazolin-4(5H)-yl)propyl)cyclohexane-1-carboxamide in an aspect of the invention.

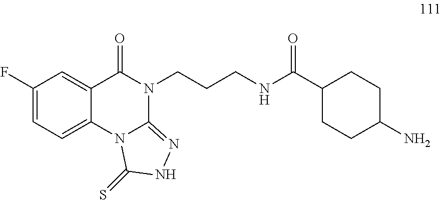 [SMILES: NC1CCC(C(=O)NCCCn2c(=O)c3cc(F)ccc3n3c(=S)[nH]nc23)CC1]

tert-Butyl (4-((3-(7-fluoro-5-oxo-1-thioxo-1,2-dihydro-[1,2,4]triazolo[4,3-a]quinazolin-4(5H)-yl)propyl)carbamoyl)cyclohexyl)carbamate (0.0282 g, 0.054 mmol) was dissolved in HCl in dioxane (0.136 ml, 0.544 mmol) and stirred at room temperature for 2 hr. The reaction was concentrated and purified by HPLC (conditions) to give 4-amino-N-(3-(7-fluoro-5-oxo-1-thioxo-1,2-dihydro-[1,2,4]triazolo[4,3-a]quinazolin-4(5H)-yl)propyl)cyclohexane-1-carboxamide, 2 TFA: ¹H NMR (400 MHz, DMSO-d₆) δ 10.31 (dd, J=9.3, 4.7 Hz, 1H), 7.93 (dd, J=8.5, 3.1 Hz, 1H), 7.88-7.76 (m, 2H), 7.71 (s, 3H), 4.04 (dd, J=8.3, 6.4 Hz, 2H), 3.14 (q, J=6.6 Hz, 2H), 2.33-2.23 (m, OH), 1.91-1.78 (m, 5H), 1.72-1.63 (m, 6H), 1.58-1.47 (m, 1H); ¹⁹F NMR (376 MHz, DMSO-d₆) δ −113.09 (td, J=8.4, 4.8 Hz); HRMS (ES-API): m/z calculated for C₁₉H₂₄FN₆O₂S (M+H) 419.1660, found 419.1642.

### Example 71

This example demonstrates a synthesis of N-(3-(7-fluoro-5-oxo-1-thioxo-1,2-dihydro-[1,2,4]triazolo[4,3-a]quinazolin-4(5H)-yl)propyl)piperidine-2-carboxamide in an aspect of the invention.

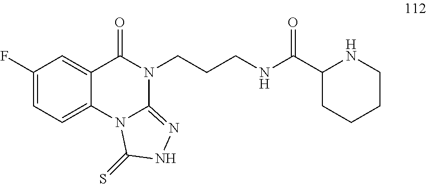 [SMILES: O=C(NCCCn1c(=O)c2cc(F)ccc2n2c(=S)[nH]nc12)C1CCCCN1]

tert-Butyl 2-((3-(7-fluoro-5-oxo-1-thioxo-1,2-dihydro-[1,2,4]triazolo[4,3-a]quinazolin-4(5H)-yl)propyl)carbamoyl)piperidine-1-carboxylate was synthesized according to General Procedure F. Then N-(3-(7-fluoro-5-oxo-1-thioxo-1,2-dihydro-[1,2,4]triazolo[4,3-a]quinazolin-4(5H)-yl)propyl)piperidine-2-carboxamide was synthesized by: tert-butyl 2-((3-(7-fluoro-5-oxo-1-thioxo-1,2-dihydro-[1,2,4]triazolo[4,3-a]quinazolin-4(5H)-yl)propyl)carbamoyl)piperidine-1-carboxylate (0.172 g, 0.341 mmol) was dissolved in HCl in dioxane (0.852 ml, 3.41 mmol) and stirred at room temperature for 2 hr. The reaction was concentrated and purified by HPLC (basic conditions) to give N-(3-(7-fluoro-5-oxo-1-thioxo-1,2-dihydro-[1,2,4]triazolo[4,3-a]quinazolin-4(5H)-yl)propyl)piperidine-2-carboxamide: HRMS (ES-API): m/z calculated for C₁₈H₂₂FN₆O₂S (M+H) 405.1503, found 405.1517.

### Example 72

This example demonstrates a synthesis of N-(3-(7-fluoro-5-oxo-1-thioxo-1,2-dihydro-[1,2,4]triazolo[4,3-a]quinazolin-4(5H)-yl)propyl)piperidine-3-carboxamide in an aspect of the invention.

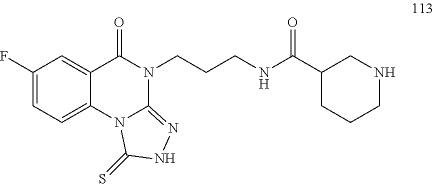 [SMILES: O=C(NCCCn1c(=O)c2cc(F)ccc2n2c(=S)[nH]nc12)C1CCCNC1]

tert-Butyl 2-((3-(7-fluoro-5-oxo-1-thioxo-1,2-dihydro-[1,2,4]triazolo[4,3-a]quinazolin-4(5H)-yl)propyl)carbamoyl)piperidine-1-carboxylate was synthesized according to General Procedure F. Then N-(3-(7-fluoro-5-oxo-1-thioxo-1,2-dihydro-[1,2,4]triazolo[4,3-a]quinazolin-4(5H)-yl)propyl)piperidine-3-carboxamide was synthesized by: tert-butyl 3-((3-(7-fluoro-5-oxo-1-thioxo-1,2-dihydro-[1,2,4]triazolo[4,3-a]quinazolin-4(5H)-yl)propyl)carbamoyl)piperidine-1-carboxylate (0.172 g, 0.341 mmol) was dissolved in HCl in dioxane (0.852 ml, 3.41 mmol) and stirred at room temperature for 2 hr. The reaction was concentrated and purified by HPLC (basic conditions) to give N-(3-(7-fluoro-5-oxo-1-thioxo-1,2-dihydro-[1,2,4]triazolo[4,3-a]quinazolin-4(5H)-yl)propyl)piperidine-3-carboxamide: HRMS (ES-API): m/z calculated for C₁₈H₂₂FN₆O₂S (M+H) 405.1503, found 405.1491.

### Example 73

This example demonstrates a synthesis of N-(3-(7-fluoro-5-oxo-1-thioxo-1,2-dihydro-[1,2,4]triazolo[4,3-a]quinazolin-4(5H)-yl)propyl)piperidine-4-carboxamide in an aspect of the invention.

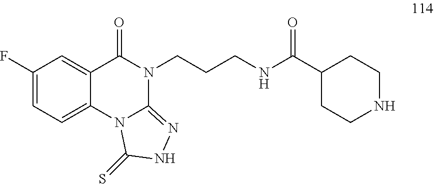 [SMILES: O=C(NCCCn1c(=O)c2cc(F)ccc2n2c(=S)[nH]nc12)C1CCNCC1]

tert-Butyl 4-((3-(7-fluoro-5-oxo-1-thioxo-1,2-dihydro-[1,2,4]triazolo[4,3-a]quinazolin-4(5H)-yl)propyl)carbamoyl)piperidine-1-carboxylate was synthesized according to General Procedure F. Then N-(3-(7-fluoro-5-oxo-1-thioxo-1,2-dihydro-[1,2,4]triazolo[4,3-a]quinazolin-4(5H)-yl)propyl)piperidine-4-carboxamide was synthesized by: tert-butyl 4-((3-(7-fluoro-5-oxo-1-thioxo-1,2-dihydro-[1,2,4]triazolo[4,3-a]quinazolin-4(5H)-yl)propyl)carbamoyl)piperidine-1-carboxylate (0.172 g, 0.341 mmol) was dissolved in HCl in dioxane (0.852 ml, 3.41 mmol) and stirred at room temperature for 2 hr. The reaction was concentrated and purified by HPLC (basic conditions) to give N-(3-(7-fluoro-5-oxo-1-thioxo-1,2-dihydro-[1,2,4]triazolo[4,3-a]quinazolin-4(5H)-yl)propyl)piperidine-4-carboxamide: HRMS (ES-API): m/z calculated for C₁₈H₂₂FN₆O₂S (M+H) 405.1503, found 405.1511; Purity (HPLC) 93.09%.

### Example 74

This example demonstrates a synthesis of N-(3-(7-fluoro-5-oxo-1-thioxo-1,2-dihydro-[1,2,4]triazolo[4,3-a]quinazolin-4(5H)-yl)propyl)tetrahydro-2H-pyran-4-carboxamide in an aspect of the invention.

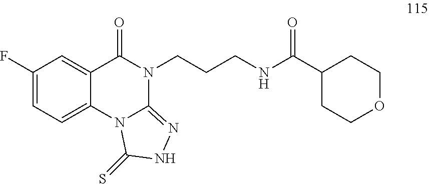 [SMILES: O=C(NCCCn1c(=O)c2cc(F)ccc2n2c(=S)[nH]nc12)C1CCOCC1]

N-(3-(7-Fluoro-5-oxo-1-thioxo-1,2-dihydro-[1,2,4]triazolo[4,3-a]quinazolin-4(5H)-yl)propyl)tetrahydro-2H-pyran-4-carboxamide, TFA was synthesized according to General Procedure F: HRMS (ES-API): m/z calculated for C₁₈H₂₁FN₅O₃S (M+H) 406.1344, found 406.1355.

### Example 75

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(piperidin-4-yl)acetamide in an aspect of the invention.

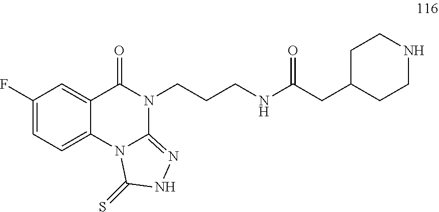 [SMILES: O=C(CC1CCNCC1)NCCCn1c(=O)c2cc(F)ccc2n2c(=S)[nH]nc12]

tert-Butyl 4-(2-((3-(7-fluoro-5-oxo-1-thioxo-1,2-dihydro-[1,2,4]triazolo[4,3-a]quinazolin-4(5H)-yl)propyl)amino)-2-oxoethyl)piperidine-1-carboxylate was synthesized according to General Procedure F. Then N-(3-(7-fluoro-5-oxo-1-thioxo-1,2-dihydro-[1,2,4]triazolo[4,3-a]quinazolin-4(5H)-yl)propyl)-2-(piperidin-4-yl)acetamide was synthesized by: tert-butyl 4-(2-((3-(7-fluoro-5-oxo-1-thioxo-1,2-dihydro-[1,2,4]triazolo[4,3-a]quinazolin-4(5H)-yl)propyl)amino)-2-oxoethyl)piperidine-1-carboxylate (0.0209 g, 0.040 mmol) was dissolved in HCl in dioxane (0.101 ml, 0.403 mmol) and stirred at room temperature for 2 hr. The reaction was concentrated and purified by HPLC (conditions) to give N-(3-(7-fluoro-5-oxo-1-thioxo-1,2-dihydro-[1,2,4]triazolo[4,3-a]quinazolin-4(5H)-yl)propyl)-2-(piperidin-4-yl)acetamide, TFA: ¹H NMR (400 MHz, DMSO-d₆) δ 10.31 (dd, J=9.3, 4.7 Hz, 1H), 8.43 (s, 1H), 8.12 (s, 1H), 7.96-7.89 (m, 2H), 7.83 (ddd, J=9.3, 8.0, 3.1 Hz, 1H), 4.08-4.00 (m, 2H), 3.24 (d, J=13.1 Hz, 2H), 3.13 (q, J=6.6 Hz, 2H), 2.86 (dd, J=21.5, 10.8 Hz, 3H), 2.02 (d, J=7.0 Hz, 2H), 1.97-1.89 (m, 1H), 1.84 (p, J=7.0 Hz, 2H), 1.77 (d, J=14.0 Hz, 2H), 1.35-1.22 (m, 2H); ¹⁹F NMR (376 MHz, DMSO-d₆) δ −113.06-−113.17 (m).

### Example 76

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(4-methylpiperazin-1-yl)acetamide in an aspect of the invention.

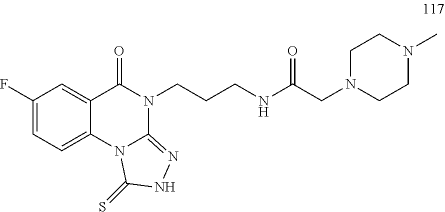 [SMILES: CN1CCN(CC(=O)NCCCn2c(=O)c3cc(F)ccc3n3c(=S)[nH]nc23)CC1]

N-(3-(7-Fluoro-5-oxo-1-thioxo-1,2-dihydro-[1,2,4]triazolo[4,3-a]quinazolin-4(5H)-yl)propyl)-2-(4-methylpiperazin-1-yl)acetamide, 2 TFA was synthesized according to General Procedure F: ¹H NMR (400 MHz, DMSO-d₆) δ 10.30 (dd, J=9.3, 4.7 Hz, 1H), 9.49 (s, 1H), 7.97 (t, J=5.5 Hz, 1H), 7.93 (dd, J=8.5, 3.1 Hz, 1H), 7.82 (ddd, J=9.3, 8.0, 3.2 Hz, 1H), 4.02 (t, J=7.2 Hz, 2H), 3.40 (d, J=10.2 Hz, 1H), 3.21-3.11 (m, 2H), 3.08 (s, 4H), 2.99 (d, J=13.1 Hz, 3H), 2.78 (s, 3H), 1.84 (p, J=6.8 Hz, 2H); ¹⁹F NMR (376 MHz, DMSO-d₆) δ −113.07 (td, J=8.2, 4.6 Hz); HRMS (ES-API): m/z calculated for C₁₉H₂₅FN₇O₂S (M+H) 434.1769, found 434.1763.

### Example 77

This example demonstrates a synthesis of N-(3-(7-fluoro-5-oxo-1-thioxo-1,2-dihydro-[1,2,4]triazolo[4,3-a]quinazolin-4(5H)-yl)propyl)-2-morpholinoacetamide in an aspect of the invention.

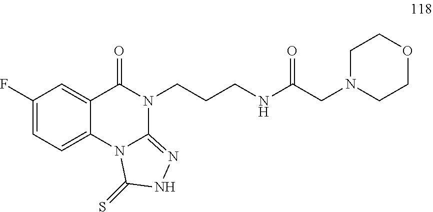 [SMILES: O=C(CN1CCOCC1)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-morpholinoacetamide, 2 TFA was synthesized according to General Procedure F: ¹H NMR (400 MHz, DMSO-d₆) δ 10.29 (dd, J=9.3, 4.7 Hz, 1H), 10.18 (s, 1H), 8.55 (s, 1H), 7.92 (dd, J=8.6, 3.1 Hz, 1H), 7.82 (ddd, J=9.3, 8.0, 3.1 Hz, 1H), 4.05 (t, J=7.1 Hz, 2H), 3.97-3.67 (m, 8H), 3.22 (q, J=6.6 Hz, 2H), 1.89 (p, J=7.1 Hz, 2H); ¹⁹F NMR (376 MHz, DMSO-d₆) δ −113.07 (td, J=8.3, 4.6 Hz); HRMS (ES-API): m/z calculated for C₁₈H₂₂FN₆O₃S (M+H) 421.1453, found 421.1449.

### Example 78

This example demonstrates a synthesis of 4-((1H-imidazol-1-yl)methyl)-N-(3-(7-fluoro-5-oxo-1-thioxo-1,2-dihydro-[1,2,4]triazolo[4,3-a]quinazolin-4(5H)-yl)propyl)benzamide in an aspect of the invention.

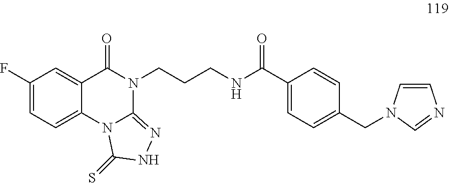 [SMILES: O=C(NCCCn1c(=O)c2cc(F)ccc2n2c(=S)[nH]nc12)c1ccc(Cn2ccnc2)cc1]

4-((1H-imidazol-1-yl)methyl)-N-(3-(7-fluoro-5-oxo-1-thioxo-1,2-dihydro-[1,2,4]triazolo[4,3-a]quinazolin-4(5H)-yl)propyl)benzamide, TFA was synthesized according to General Procedure F: ¹H NMR (400 MHz, DMSO-d₆) δ 10.30 (dd, J=9.3, 4.7 Hz, 1H), 9.13 (t, J=1.5 Hz, 1H), 8.52 (t, J=5.7 Hz, 1H), 7.91 (dd, J=8.5, 3.1 Hz, 1H), 7.86-7.77 (m, 3H), 7.75 (t, J=1.7 Hz, 1H), 7.64 (t, J=1.6 Hz, 1H), 7.57-7.45 (m, 2H), 5.46 (s, 2H), 4.11 (dd, J=8.3, 6.3 Hz, 2H), 3.37-3.33 (m, 2H), 1.99 (p, J=7.0 Hz, 2H); ¹⁹F NMR (376 MHz, DMSO-d₆) δ −113.10 (td, J=8.3, 4.7 Hz); HRMS (ES-API): m/z calculated for C₂₃H₂₁FN₇O₂S (M+H) 478.1456, found 478.1454.

### Example 79

This example demonstrates a synthesis of N-(3-(7-fluoro-5-oxo-1-thioxo-1,2-dihydro-[1,2,4]triazolo[4,3-a]quinazolin-4(5H)-yl)propyl)-2-phenylacetamide in an aspect of the invention.

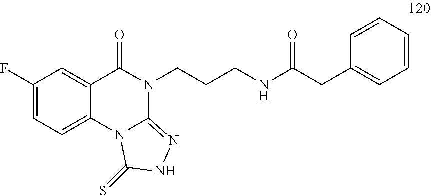 [SMILES: O=C(Cc1ccccc1)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-phenylacetamide was synthesized according to General Procedure F: ¹H NMR (400 MHz, DMSO-d₆) δ 10.31 (dd, J=9.3, 4.7 Hz, 1H), 8.05 (t, J=5.7 Hz, 1H), 7.92 (dd, J=8.6, 3.1 Hz, 1H), 7.81 (ddd, J=9.3, 8.0, 3.1 Hz, 1H), 7.33-7.15 (m, 5H), 4.04 (dd, J=8.2, 6.4 Hz, 2H), 3.38 (s, 2H), 3.14 (q, J=6.7 Hz, 2H), 1.86 (p, J=7.2 Hz, 2H); ¹⁹F NMR (376 MHz, DMSO-d₆) δ −113.17 (td, J=8.3, 4.8 Hz); HRMS (ES-API): m/z calculated for C₁₉H₁₇FN₅O₂S (M+H) 398.1082, found 398.1063.

### Example 80

This example demonstrates a synthesis of 3-(3,5-dimethylisoxazol-4-yl)-N-(3-(7-fluoro-5-oxo-1-thioxo-1,2-dihydro-[1,2,4]triazolo[4,3-a]quinazolin-4(5H)-yl)propyl)propenamide in an aspect of the invention.

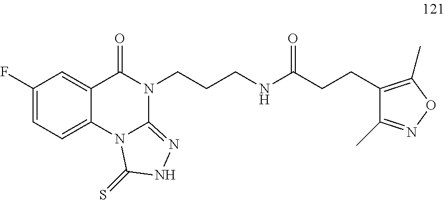 [SMILES: Cc1noc(C)c1CCC(=O)NCCCn1c(=O)c2cc(F)ccc2n2c(=S)[nH]nc12]

3-(3,5-Dimethylisoxazol-4-yl)-N-(3-(7-fluoro-5-oxo-1-thioxo-1,2-dihydro-[1,2,4]triazolo[4,3-a]quinazolin-4(5H)-yl)propyl)propenamide, TFA was synthesized according to General Procedure F: ¹H NMR (400 MHz, DMSO-d₆) δ 10.31 (dd, J=9.3, 4.7 Hz, 1H), 7.92 (dd, J=8.6, 3.0 Hz, 1H), 7.85-7.78 (m, 2H), 4.01 (t, J=7.2 Hz, 2H), 3.32-3.23 (m, 2H), 3.10 (q, J=6.7 Hz, 2H), 2.25 (s, 3H), 2.19 (dd, J=7.9, 7.0 Hz, 2H), 2.13 (s, 3H), 1.81 (p, J=7.0 Hz, 2H); ¹⁹F NMR (376 MHz, DMSO-d₆) δ −113.14 (dd, J=8.3, 5.0 Hz); HRMS (ES-API): m/z calculated for C₂₀H₂₂FN₆O₃S (M+H) 445.1453, found 445.1475.

### Example 81

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(3-fluorophenyl)acetamide in an aspect of the invention.

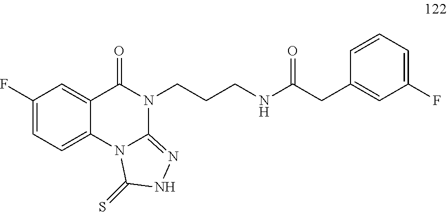 [SMILES: O=C(Cc1cccc(F)c1)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-(3-fluorophenyl)acetamide, TFA was synthesized according to General Procedure F: ¹H NMR (400 MHz, DMSO-d₆) δ 10.29 (dd, J=9.3, 4.7 Hz, 1H), 8.08 (t, J=5.6 Hz, 1H), 7.90 (dd, J=8.6, 3.1 Hz, 1H), 7.80 (ddd, J=9.3, 8.0, 3.1 Hz, 1H), 7.31 (td, J=8.1, 6.5 Hz, 1H), 7.08-6.99 (m, 3H), 4.03 (t, J=7.3 Hz, 2H), 3.40 (s, 2H), 3.13 (q, J=6.6 Hz, 2H), 1.84 (p, J=7.1 Hz, 2H); ¹⁹F NMR (376 MHz, DMSO-d₆) δ −113.19 (q, J=7.7, 7.1 Hz), −113.86-−113.98 (m); HRMS (ES-API): m/z calculated for C₂₀H₁₈F₂N₅O₂S (M+H) 430.1144, found 430.1153.

### Example 82

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(4-fluorophenyl)acetamide in an aspect of the invention.

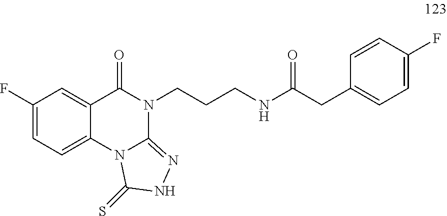 [SMILES: O=C(Cc1ccc(F)cc1)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-(4-fluorophenyl)acetamide, TFA was synthesized according to General Procedure F: ¹H NMR (400 MHz, DMSO-d₆) δ 10.29 (dd, J=9.4, 4.7 Hz, 1H), 8.05 (t, J=5.7 Hz, 1H), 7.91 (dd, J=8.6, 3.1 Hz, 1H), 7.80 (ddd, J=9.3, 8.0, 3.1 Hz, 1H), 7.30-7.22 (m, 2H), 7.14-7.05 (m, 2H), 4.02 (t, J=7.3 Hz, 2H), 3.36 (s, 2H), 3.12 (q, J=6.6 Hz, 2H), 1.84 (p, J=7.1 Hz, 2H); ¹⁹F NMR (376 MHz, DMSO-d₆) δ −113.18 (td, J=8.3, 4.8 Hz), −116.95 (ddd, J=14.6, 9.1, 5.5 Hz); HRMS (ES-API): m/z calculated for C₂₀H₁₈F₂N₅O₂S (M+H) 430.1144, found 430.1141; Purity (HPLC) 93.64%.

### Example 83

This example demonstrates a synthesis of 2-(3-chlorophenyl)-N-(3-(7-fluoro-5-oxo-1-thioxo-1,2-dihydro-[1,2,4]triazolo[4,3-a]quinazolin-4(5H)-yl)propyl)acetamide in an aspect of the invention.

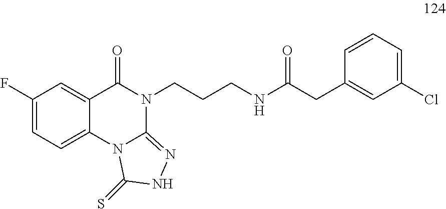 [SMILES: O=C(Cc1cccc(Cl)c1)NCCCn1c(=O)c2cc(F)ccc2n2c(=S)[nH]nc12]

2-(3-Chlorophenyl)-N-(3-(7-fluoro-5-oxo-1-thioxo-1,2-dihydro-[1,2,4]triazolo[4,3-a]quinazolin-4(5H)-yl)propyl)acetamide, TFA was synthesized according to General Procedure F: ¹H NMR (400 MHz, DMSO-d₆) δ 10.29 (dd, J=9.3, 4.7 Hz, 1H), 8.09 (t, J=5.7 Hz, 1H), 7.91 (dd, J=8.6, 3.1 Hz, 1H), 7.80 (ddd, J=9.3, 8.0, 3.1 Hz, 1H), 7.33-7.24 (m, 3H), 7.19 (dt, J=7.5, 1.5 Hz, 1H), 4.03 (t, J=7.2 Hz, 2H), 3.39 (s, 2H), 3.27 (s, 1H), 3.12 (q, J=6.7 Hz, 2H), 1.85 (p, J=7.2 Hz, 2H); ¹⁹F NMR (376 MHz, DMSO-d₆) δ −113.19 (d, J=10.0 Hz); HRMS (ES-API): m/z calculated for C₂₀H₁₈ClFN₅O₂S (M+H) 446.0848, found 446.0867; Purity (HPLC) 94.66%.

### Example 84

This example demonstrates a synthesis of 2-(4-bromophenyl)-N-(3-(7-fluoro-5-oxo-1-thioxo-1,2-dihydro-[1,2,4]triazolo[4,3-a]quinazolin-4(5H)-yl)propyl)acetamide in an aspect of the invention.

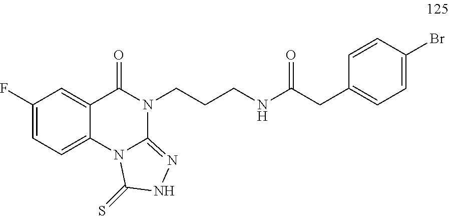 [SMILES: O=C(Cc1ccc(Br)cc1)NCCCn1c(=O)c2cc(F)ccc2n2c(=S)[nH]nc12]

2-(4-Bromophenyl)-N-(3-(7-fluoro-5-oxo-1-thioxo-1,2-dihydro-[1,2,4]triazolo[4,3-a]quinazolin-4(5H)-yl)propyl)acetamide, TFA was synthesized according to General Procedure F: ¹H NMR (400 MHz, DMSO-d₆) δ 10.29 (dd, J=9.3, 4.6 Hz, 1H), 8.08 (t, J=5.7 Hz, 1H), 7.91 (dd, J=8.6, 3.1 Hz, 1H), 7.80 (ddd, J=9.2, 7.9, 3.1 Hz, 1H), 7.50-7.42 (m, 2H), 7.22-7.15 (m, 2H), 4.02 (t, J=7.3 Hz, 2H), 3.36 (s, 2H), 3.12 (q, J=6.6 Hz, 2H), 1.84 (p, J=7.0 Hz, 2H); ¹⁹F NMR (376 MHz, DMSO-d₆) δ −113.19; Purity (HPLC) 88.26%.

### Example 85

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(p-tolyl)acetamide in an aspect of the invention.

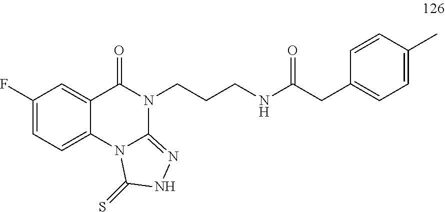 [SMILES: Cc1ccc(CC(=O)NCCCn2c(=O)c3cc(F)ccc3n3c(=S)[nH]nc23)cc1]

N-(3-(7-Fluoro-5-oxo-1-thioxo-1,2-dihydro-[1,2,4]triazolo[4,3-a]quinazolin-4(5H)-yl)propyl)-2-(p-tolyl)acetamide, TFA was synthesized according to General Procedure F: ¹H NMR (400 MHz, DMSO-d₆) δ 10.29 (dd, J=9.4, 4.6 Hz, 1H), 7.99 (t, J=5.8 Hz, 1H), 7.91 (dd, J=8.6, 3.1 Hz, 1H), 7.80 (ddd, J=10.9, 8.0, 3.1 Hz, 1H), 7.09 (q, J=8.0 Hz, 4H), 4.02 (t, J=7.3 Hz, 2H), 3.11 (q, J=6.6 Hz, 2H), 2.24 (s, 3H), 1.83 (p, J=7.2 Hz, 2H); ¹⁹F NMR (376 MHz, DMSO-d₆) δ −113.20; Purity (HPLC) 89.66%.

### Example 86

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(4-hydroxyphenyl)acetamide in an aspect of the invention.

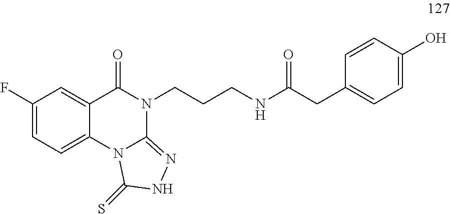 [SMILES: O=C(Cc1ccc(O)cc1)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-(4-hydroxyphenyl)acetamide, TFA was synthesized according to General Procedure F: ¹H NMR (400 MHz, DMSO-d₆) δ 10.29 (dd, J=9.3, 4.7 Hz, 1H), 9.16 (s, 1H), 7.94-7.87 (m, 2H), 7.80 (ddd, J=9.3, 8.0, 3.1 Hz, 1H), 7.04-6.98 (m, 2H), 6.69-6.60 (m, 2H), 4.06-3.98 (m, 2H), 3.23 (s, 2H), 3.10 (q, J=6.6 Hz, 2H), 1.83 (p, J=7.2 Hz, 2H); ¹⁹F NMR (376 MHz, DMSO-d₆) δ −113.19; Purity (HPLC) 90.60%.

### Example 87

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(2-methoxyphenyl)acetamide in an aspect of the invention.

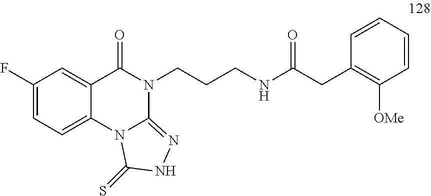 [SMILES: COc1ccccc1CC(=O)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-(2-methoxyphenyl)acetamide, TFA was synthesized according to General Procedure F: ¹H NMR (400 MHz, DMSO-d₆) δ 10.29 (dd, J=9.3, 4.7 Hz, 1H), 7.91 (dd, J=8.6, 3.1 Hz, 1H), 7.80 (ddd, J=9.2, 7.8, 3.0 Hz, 2H), 7.22-7.16 (m, 1H), 7.13 (dd, J=7.5, 1.7 Hz, 1H), 6.92 (dd, J=8.2, 1.1 Hz, 1H), 6.86 (td, J=7.4, 1.1 Hz, 1H), 4.03 (t, J=7.3 Hz, 2H), 3.74 (s, 3H), 3.34 (s, 2H), 3.13 (q, J=6.6 Hz, 2H), 1.84 (p, J=7.0 Hz, 2H); ¹⁹F NMR (376 MHz, DMSO-d₆) δ −113.18 (td, J=8.3, 4.7 Hz); HRMS (ES-API): m/z calculated for C₂₁H₂₁FN₅O₃S (M+H) 442.1344, found 442.1360.

### Example 88

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(3-methoxyphenyl)acetamide in an aspect of the invention.

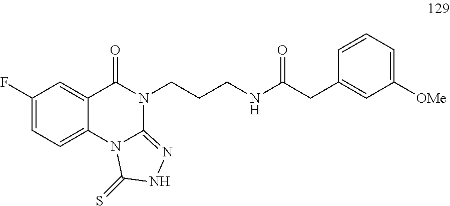 [SMILES: COc1cccc(CC(=O)NCCCn2c(=O)c3cc(F)ccc3n3c(=S)[nH]nc23)c1]

N-(3-(7-Fluoro-5-oxo-1-thioxo-1,2-dihydro-[1,2,4]triazolo[4,3-a]quinazolin-4(5H)-yl)propyl)-2-(3-methoxyphenyl)acetamide, TFA was synthesized according to General Procedure F: ¹H NMR (400 MHz, DMSO-d₆) δ 10.29 (dd, J=9.3, 4.6 Hz, 1H), 8.01 (t, J=5.7 Hz, 1H), 7.91 (dd, J=8.6, 3.1 Hz, 1H), 7.80 (ddd, J=9.3, 8.0, 3.1 Hz, 1H), 7.22-7.13 (m, 1H), 6.82-6.73 (m, 3H), 4.03 (t, J=7.2 Hz, 2H), 3.71 (s, 3H), 3.33 (s, 2H), 3.12 (q, J=6.7 Hz, 2H), 1.84 (p, J=7.2 Hz, 2H); ¹⁹F NMR (376 MHz, DMSO-d₆) δ −113.19.

### Example 89

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(4-methoxyphenyl)acetamide in an aspect of the invention.

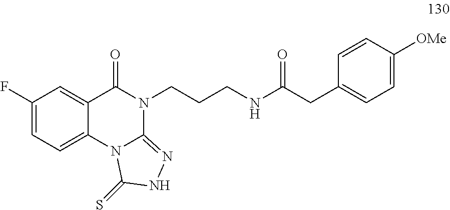 [SMILES: COc1ccc(CC(=O)NCCCn2c(=O)c3cc(F)ccc3n3c(=S)[nH]nc23)cc1]

N-(3-(7-Fluoro-5-oxo-1-thioxo-1,2-dihydro-[1,2,4]triazolo[4,3-a]quinazolin-4(5H)-yl)propyl)-2-(4-methoxyphenyl)acetamide, TFA was synthesized according to General Procedure F: ¹H NMR (400 MHz, DMSO-d₆) δ 10.29 (dd, J=9.3, 4.7 Hz, 1H), 7.96 (t, J=5.8 Hz, 1H), 7.91 (dd, J=8.6, 3.1 Hz, 1H), 7.80 (ddd, J=9.3, 8.0, 3.1 Hz, 1H), 7.17-7.09 (m, 2H), 6.87-6.79 (m, 2H), 4.02 (t, J=7.3 Hz, 2H), 3.69 (s, 3H), 3.29 (s, 2H), 3.11 (q, J=6.6 Hz, 2H), 1.83 (p, J=7.1 Hz, 2H); ¹⁹F NMR (376 MHz, DMSO-d₆) δ −113.19 (td, J=8.2, 4.7 Hz).

### Example 90

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(2-(trifluoromethyl)phenyl)acetamide in an aspect of the invention.

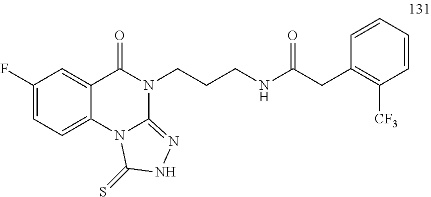 [SMILES: O=C(Cc1ccccc1C(F)(F)F)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-(2-(trifluoromethyl)phenyl)acetamide, TFA was synthesized according to General Procedure F: ¹H NMR (400 MHz, DMSO-d₆) δ 10.29 (dd, J=9.3, 4.7 Hz, 1H), 8.05 (t, J=5.7 Hz, 1H), 7.91 (dd, J=8.6, 3.1 Hz, 1H), 7.80 (ddd, J=9.2, 7.9, 3.1 Hz, 1H), 7.65 (d, J=7.7 Hz, 1H), 7.60 (t, J=7.6 Hz, 1H), 7.44 (d, J=7.8 Hz, 2H), 4.04 (t, J=7.3 Hz, 2H), 3.61 (s, 2H), 3.15 (q, J=6.7 Hz, 2H), 1.85 (p, J=7.1 Hz, 2H); ¹⁹F NMR (376 MHz, DMSO-d₆) δ −58.53, −113.20 (td, J=8.3, 4.8 Hz).

### Example 91

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(3-(trifluoromethyl)phenyl)acetamide in an aspect of the invention.

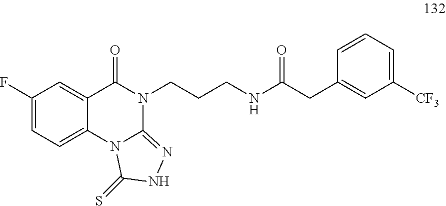 [SMILES: O=C(Cc1cccc(C(F)(F)F)c1)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-(3-(trifluoromethyl)phenyl)acetamide, TFA was synthesized according to General Procedure F: ¹H NMR (400 MHz, DMSO-d₆) δ 10.29 (dd, J=9.3, 4.7 Hz, 1H), 8.14 (t, J=5.6 Hz, 1H), 7.90 (dd, J=8.6, 3.1 Hz, 1H), 7.80 (ddd, J=9.3, 8.0, 3.1 Hz, 1H), 7.61-7.49 (m, 4H), 4.03 (dd, J=8.1, 6.3 Hz, 2H), 3.50 (s, 2H), 3.13 (q, J=6.8 Hz, 2H), 1.85 (p, J=7.1 Hz, 2H); ¹⁹F NMR (376 MHz, DMSO-d₆) δ −61.01, −113.20 (td, J=8.2, 4.7 Hz); HRMS (ES-API): m/z calculated for C₂₁H₁₈F₄N₅O₂S (M+H) 480.1112, found 480.1126.

### Example 100

This example demonstrates a synthesis of 2-(2-cyanophenyl)-N-(3-(7-fluoro-5-oxo-1-thioxo-1,2-dihydro-[1,2,4]triazolo[4,3-a]quinazolin-4(5H)-yl)propyl)acetamide in an aspect of the invention.

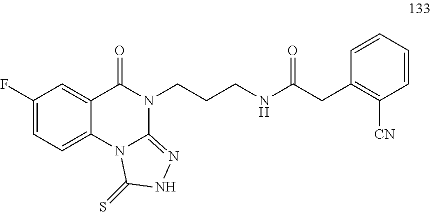 [SMILES: N#Cc1ccccc1CC(=O)NCCCn1c(=O)c2cc(F)ccc2n2c(=S)[nH]nc12]

2-(2-Cyanophenyl)-N-(3-(7-fluoro-5-oxo-1-thioxo-1,2-dihydro-[1,2,4]triazolo[4,3-a]quinazolin-4(5H)-yl)propyl)acetamide, TFA was synthesized according to General Procedure F: ¹H NMR (400 MHz, DMSO-d₆) δ 10.29 (dd, J=9.3, 4.7 Hz, 1H), 8.20 (t, J=5.6 Hz, 1H), 7.91 (dd, J=8.6, 3.1 Hz, 1H), 7.85-7.73 (m, 2H), 7.63 (td, J=7.7, 1.4 Hz, 1H), 7.46 (d, J=7.8 Hz, 1H), 7.42 (td, J=7.6, 1.2 Hz, 1H), 4.05 (t, J=7.2 Hz, 2H), 3.65 (s, 2H), 3.16 (q, J=6.7 Hz, 2H), 1.87 (p, J=7.2 Hz, 2H); ¹⁹F NMR (376 MHz, DMSO-d₆) δ −113.18 (td, J=8.4, 4.8 Hz); HRMS (ES-API): m/z calculated for C₂₁H₁₈FN₆O₂S (M+H) 437.1190, found 437.1194.

### Example 101

This example demonstrates a synthesis of 2-(4-cyanophenyl)-N-(3-(7-fluoro-5-oxo-1-thioxo-1,2-dihydro-[1,2,4]triazolo[4,3-a]quinazolin-4(5H)-yl)propyl)acetamide in an aspect of the invention.

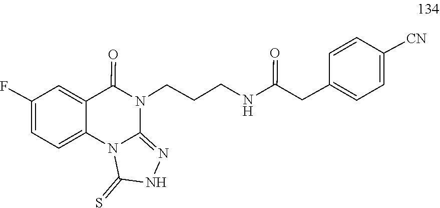 [SMILES: N#Cc1ccc(CC(=O)NCCCn2c(=O)c3cc(F)ccc3n3c(=S)[nH]nc23)cc1]

2-(4-Cyanophenyl)-N-(3-(7-fluoro-5-oxo-1-thioxo-1,2-dihydro-[1,2,4]triazolo[4,3-a]quinazolin-4(5H)-yl)propyl)acetamide, TFA was synthesized according to General Procedure F: ¹H NMR (400 MHz, DMSO-d₆) δ 10.29 (dd, J=9.3, 4.7 Hz, 1H), 8.16 (t, J=5.6 Hz, 1H), 7.91 (dd, J=8.6, 3.1 Hz, 1H), 7.80 (ddd, J=9.3, 8.0, 3.1 Hz, 1H), 7.77-7.73 (m, 2H), 7.47-7.40 (m, 2H), 4.02 (t, J=7.3 Hz, 2H), 3.50 (s, 2H), 3.18-3.09 (m, 2H), 1.84 (p, J=7.1 Hz, 2H); ¹⁹F NMR (376 MHz, DMSO-d₆) δ −113.17.

### Example 102

This example demonstrates a synthesis of tert-butyl (4-(2-((3-(7-fluoro-5-oxo-1-thioxo-1,2-dihydro-[1,2,4]triazolo[4,3-a]quinazolin-4(5H)-yl)propyl)amino)-2-oxoethyl)phenyl)carbamate in an aspect of the invention.

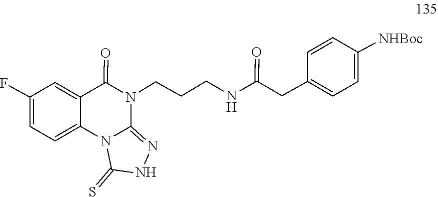 [SMILES: CC(C)(C)OC(=O)Nc1ccc(CC(=O)NCCCn2c(=O)c3cc(F)ccc3n3c(=S)[nH]nc23)cc1]

tert-Butyl (4-(2-((3-(7-fluoro-5-oxo-1-thioxo-1,2-dihydro-[1,2,4]triazolo[4,3-a]quinazolin-4(5H)-yl)propyl)amino)-2-oxoethyl)phenyl)carbamate, TFA was synthesized according to General Procedure F: ¹H NMR (400 MHz, DMSO-d₆) δ 10.30 (dd, J=9.3, 4.7 Hz, 1H), 9.23 (s, 1H), 7.98 (t, J=5.7 Hz, 1H), 7.92 (dd, J=8.6, 3.1 Hz, 1H), 7.81 (ddd, J=9.3, 8.0, 3.1 Hz, 1H), 7.34 (d, J=8.5 Hz, 2H), 7.15-7.05 (m, 2H), 4.03 (dd, J=8.1, 6.5 Hz, 2H), 3.30 (s, 2H), 3.12 (q, J=6.7 Hz, 2H), 1.84 (p, J=7.1 Hz, 2H), 1.46 (s, 9H); ¹⁹F NMR (376 MHz, DMSO-d₆) δ −113.16 (td, J=8.2, 4.8 Hz).

### Example 103

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(129yridine-2-yl)acetamide in an aspect of the invention.

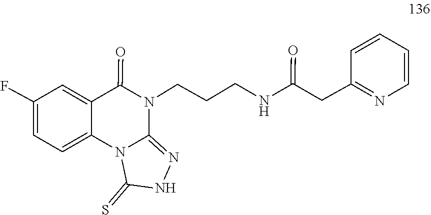 [SMILES: O=C(Cc1ccccn1)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-(pyridine-2-yl)acetamide was synthesized according to General Procedure F.

### Example 104

This example demonstrates a synthesis of N-(3-(7-Fluoro-5-oxo-1-thioxo-1,2-dihydro-[1,2,4]triazolo[4,3-a]quinazolin-4(5H)-yl)propyl)-2-(pyridine-3-yl)acetamide in an aspect of the invention.

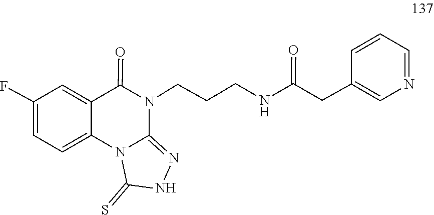 [SMILES: O=C(Cc1cccnc1)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-(pyridine-3-yl)acetamide was synthesized according to General Procedure F: ¹H NMR (400 MHz, DMSO-d₆) δ 10.29 (dd, J=9.3, 4.7 Hz, 1H), 8.44-8.37 (m, 2H), 8.14 (t, J=5.7 Hz, 1H), 7.90 (dd, J=8.6, 3.1 Hz, 1H), 7.80 (ddd, J=9.3, 8.0, 3.1 Hz, 1H), 7.64 (dt, J=7.8, 2.0 Hz, 1H), 7.31 (dd, J=7.8, 4.8 Hz, 1H), 4.03 (dd, J=8.3, 6.3 Hz, 2H), 3.42 (s, 2H), 3.13 (q, J=6.5 Hz, 2H), 1.85 (p, J=7.1 Hz, 2H); ¹⁹F NMR (376 MHz, DMSO-d₆) δ −113.20 (d, J=9.2 Hz).

### Example 105

This example demonstrates a synthesis of N-(3-(7-fluoro-5-oxo-1-thioxo-1,2-dihydro-[1,2,4]triazolo[4,3-a]quinazolin-4(5H)-yl)propyl)-2-(pyridine-4-yl)acetamide in an aspect of the invention.

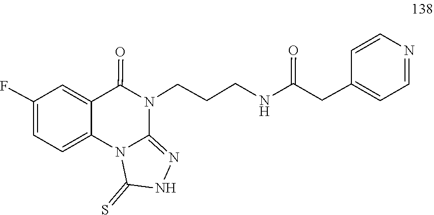 [SMILES: O=C(Cc1ccncc1)NCCCn1c(=O)c2cc(F)ccc2n2c(=S)[nH]nc12]

N-(3-(7-Fluoro-5-oxo-1-thioxo-1,2-dihydro-[1,2,4]triazolo[4,3-a]quinazolin-4(5H)-yl)propyl)-2-(pyridine-4-yl)acetamide was synthesized according to General Procedure F.

### Example 106

This example demonstrates a synthesis of 9-fluoro-4-propyl-1-thioxo-2,4-dihydro-[1,2,4]triazolo[4,3-a]quinazolin-5(1H)-one in an aspect of the invention.

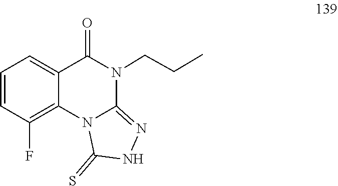 [SMILES: CCCn1c(=O)c2cccc(F)c2n2c(=S)[nH]nc12]

8-Fluoro-3-propyl-2-thioxo-2,3-dihydroquinazolin-4(TH)-one, TFA was synthesized according to General Procedure A. Then 9-fluoro-4-propyl-1-thioxo-2,4-dihydro-[1,2,4]triazolo[4,3-a]quinazolin-5(1H)-one was synthesized according to General Procedure D: ¹H NMR (400 MHz, DMSO-d₆) δ 13.83 (s, 1H), 8.02 (dd, J=7.9, 1.5 Hz, 1H), 7.77 (ddd, J=11.5, 8.3, 1.5 Hz, 1H), 7.61 (td, J=8.0, 4.1 Hz, 1H), 3.97-3.89 (m, 2H), 1.70 (sext, J=7.5 Hz, 2H), 0.89 (t, J=7.5 Hz, 3H); ¹⁹F NMR (376 MHz, DMSO-d₆) δ −93.30 (dd, J=11.5, 4.2 Hz).

### Example 107

This example demonstrates a synthesis of 9-chloro-4-propyl-1-thioxo-2,4-dihydro-[1,2,4]triazolo[4,3-a]quinazolin-5(11H)-one in an aspect of the invention.

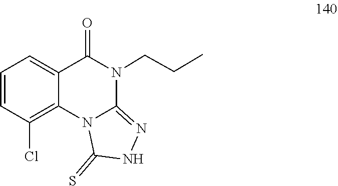 [SMILES: CCCn1c(=O)c2cccc(Cl)c2n2c(=S)[nH]nc12]

8-Chloro-3-propyl-2-thioxo-2,3-dihydroquinazolin-4(1H)-one, TFA was synthesized according to General Procedure A. Then 9-chloro-4-propyl-1-thioxo-2,4-dihydro-[1,2,4]triazolo[4,3-a]quinazolin-5(1H)-one was synthesized according to General Procedure D: ¹H NMR (400 MHz, DMSO-d₆) δ 13.78 (s, 1H), 8.10 (dd, J=7.7, 1.5 Hz, 1H), 7.91 (dd, J=8.0, 1.5 Hz, 1H), 7.60 (t, J=7.9 Hz, 1H), 3.94-3.86 (m, 2H), 1.70 (sext, J=7.5 Hz, 2H), 0.89 (t, J=7.4 Hz, 3H).

### Example 108

This example demonstrates a synthesis of 9-methoxy-4-propyl-1-thioxo-2,4-dihydro-[1,2,4]triazolo[4,3-a]quinazolin-5(1H)-one in an aspect of the invention.

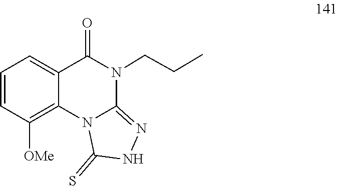 [SMILES: CCCn1c(=O)c2cccc(OC)c2n2c(=S)[nH]nc12]

8-Methoxy-3-propyl-2-thioxo-2,3-dihydroquinazolin-4(1H)-one was synthesized according to General Procedure A. Then 9-methoxy-4-propyl-1-thioxo-2,4-dihydro-[1,2,4]triazolo[4,3-a]quinazolin-5(1H)-one was synthesized according to General Procedure D.

### Example 109

This example demonstrates a synthesis of S-(5-oxo-4-propyl-4,5-dihydro-[1,2,4]triazolo[4,3-a]quinazolin-1-yl) ethanethioate in an aspect of the invention.

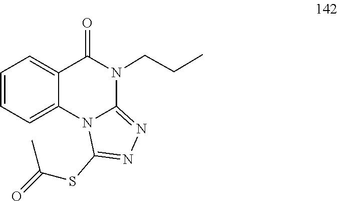 [SMILES: CCCn1c(=O)c2ccccc2n2c(SC(C)=O)nnc12]

3-Propyl-2-thioxo-2,3-dihydroquinazolin-4(1H)-one was synthesized according to General Procedure A. Then 4-propyl-1-thioxo-2,4-dihydro-[1,2,4]triazolo[4,3-a]quinazolin-5(1H)-one, TFA was synthesized according to General Procedure D. Then S-(5-oxo-4-propyl-4,5-dihydro-[1,2,4]triazolo[4,3-a]quinazolin-1-yl) ethanethioate was synthesized by: to a solution of 4-propyl-1-thioxo-2,4-dihydro-[1,2,4]triazolo[4,3-a]quinazolin-5(1H)-one (0.05 g, 0.192 mmol) in acetonitrile (0.549 ml) was added triethylamine (0.054 ml, 0.384 mmol) and acetic anhydride (0.022 ml, 0.230 mmol). The reaction was stirred for 2 hr and quenched with water. The crude mixture was concentrated and purified by HPLC to give S-(5-oxo-4-propyl-4,5-dihydro-[1,2,4]triazolo[4,3-a]quinazolin-1-yl) ethanethioate, TFA: ¹H NMR (400 MHz, DMSO-d₆) δ 10.32 (dd, J=8.6, 1.0 Hz, 1H), 8.24 (dd, J=7.9, 1.6 Hz, 1H), 7.90 (ddd, J=8.8, 7.3, 1.7 Hz, 1H), 7.64 (td, J=7.6, 1.1 Hz, 1H), 4.06-4.00 (m, 2H), 2.65 (s, 3H), 1.77 (sext, J=7.4 Hz, 2H), 0.93 (t, J=7.5 Hz, 3H).

### Example 110

This example demonstrates a synthesis of 1-(methylthio)-4-propyl-[1,2,4]triazolo[4,3-a]quinazolin-5(4H)-one in an aspect of the invention.

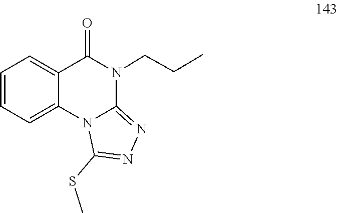 [SMILES: CCCn1c(=O)c2ccccc2n2c(SC)nnc12]

4-Propyl-1-thioxo-2,4-dihydro-[1,2,4]triazolo[4,3-a]quinazolin-5(1H)-one was synthesized as described above. Then 1-(methylthio)-4-propyl-[1,2,4]triazolo[4,3-a]quinazolin-5(4H)-one was synthesized by: to a solution of 4-propyl-1-thioxo-2,4-dihydro-[1,2,4]triazolo[4,3-a]quinazolin-5(1H)-one (0.05 g, 0.192 mmol) in DMF (0.549 ml) was added K₂CO₃ (0.032 g, 0.230 mmol) and MeI (0.012 ml, 0.192 mmol). The reaction was stirred for 2 hr and quenched with methanol. The crude mixture was concentrated and purified by HPLC (conditions) to give 1-(methylthio)-4-propyl-[1,2,4]triazolo[4,3-a]quinazolin-5(4H)-one, TFA.

### Example 111

This example demonstrates a synthesis of 7-fluoro-1-(methylthio)-4-propyl-[1,2,4]triazolo[4,3-a]quinazolin-5(4H)-one in an aspect of the invention.

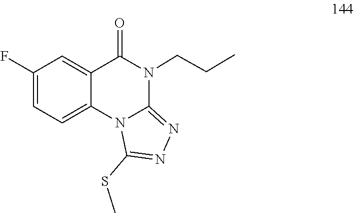 [SMILES: CCCn1c(=O)c2cc(F)ccc2n2c(SC)nnc12]

To a solution of 7-fluoro-4-propyl-1-thioxo-2,4-dihydro-[1,2,4]triazolo[4,3-a]quinazolin-5(1H)-one (0.05 g, 0.180 mmol) in DMF (0.513 ml) was added K₂CO₃ (0.030 g, 0.216 mmol) and MeI (0.011 ml, 0.180 mmol). The reaction was stirred for 2 hr and quenched with methanol. The crude mixture was concentrated and purified by HPLC (conditions) to give 7-fluoro-1-(methylthio)-4-propyl-[1,2,4]triazolo[4,3-a]quinazolin-5(4H)-one, TFA.

### Example 112

This example demonstrates a synthesis of 9-fluoro-1-(methylthio)-4-propyl-[1,2,4]triazolo[4,3-a]quinazolin-5(4H)-one in an aspect of the invention.

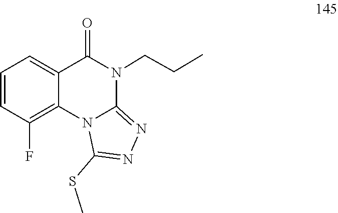 [SMILES: CCCn1c(=O)c2cccc(F)c2n2c(SC)nnc12]

To a solution of 9-fluoro-4-propyl-1-thioxo-2,4-dihydro-[1,2,4]triazolo[4,3-a]quinazolin-5(1H)-one (0.0461 g, 0.166 mmol) in DMF (0.473 ml) was added K₂CO₃ (0.027 g, 0.199 mmol) and MeI (10.36 μl, 0.166 mmol). The reaction was stirred for 3 hr and quenched with methanol. The crude mixture was concentrated and purified by HPLC to give 9-fluoro-1-(methylthio)-4-propyl-[1,2,4]triazolo[4,3-a]quinazolin-5(4H)-one, TFA.

### Example 113

This example demonstrates a synthesis of 9-chloro-1-(methylthio)-4-propyl-[1,2,4]triazolo[4,3-a]quinazolin-5(4H)-one in an aspect of the invention.

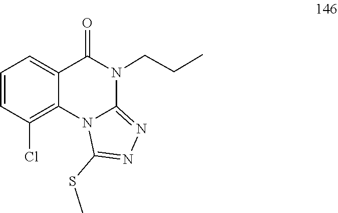 [SMILES: CCCn1c(=O)c2cccc(Cl)c2n2c(SC)nnc12]

To a solution of 9-chloro-4-propyl-1-thioxo-2,4-dihydro-[1,2,4]triazolo[4,3-a]quinazolin-5(1H)-one (0.1179 g, 0.400 mmol) in DMF (2.000 ml) was added K₂CO₃ (0.066 g, 0.480 mmol) and MeI (0.030 ml, 0.480 mmol). The reaction was stirred for 3 hr and quenched with methanol. The crude mixture was concentrated and purified by ISCO to give 9-chloro-1-(methylthio)-4-propyl-[1,2,4]triazolo[4,3-a]quinazolin-5(4H)-one.

### Example 114

This example demonstrates a synthesis of 9-methoxy-1-(methylthio)-4-propyl-[1,2,4]triazolo[4,3-a]quinazolin-5(4H)-one in an aspect of the invention.

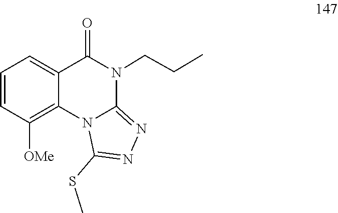 [SMILES: CCCn1c(=O)c2cccc(OC)c2n2c(SC)nnc12]

To a solution of 9-methoxy-4-propyl-1-thioxo-2,4-dihydro-[1,2,4]triazolo[4,3-a]quinazolin-5(1H)-one (0.103 g, 0.355 mmol) in DMF (1.774 ml) was added K₂CO₃ (0.059 g, 0.426 mmol) and MeI (0.027 ml, 0.426 mmol). The reaction was stirred for 3 hr and quenched with methanol. The crude mixture was concentrated and purified by ISCO to give 9-methoxy-1-(methylthio)-4-propyl-[1,2,4]triazolo[4,3-a]quinazolin-5(4H)-one.

### Example 115

This example describes a first Structure Activity Relationship (SAR) study performed for the compound of formula (I) in an aspect of the invention.

The SAR of the 1-thioxo-2,4-dihydro-[1,2,4]triazolo[4,3-a]quinazolin-5(1H)-one scaffold as PBD inhibitors was explored (Compounds 15-138, Tables 1-9). All compounds were evaluated for their efficacy against the full-length human Plk1 in an ELISA assay and for their in vitro physiochemical properties (half-life, permeability, and solubility).

An ELISA-based PBD-binding inhibition assay was performed essentially as described previously. Briefly, a biotinylated PBIP1 p-T78 peptide (i.e., Biotin-Ahx-C-ETFDPPLHSpTAI-NH₂) was first diluted with 1× coating solution (SeraCare, Gaithersburg, MD) to the final concentration of 0.3 μM, and then 100 μl of the resulting solution was immobilized onto a 96-well streptavidin-coated plate (Nalgene Nunc, Rochester, NY). The wells were washed once with PBS+0.05% Tween20 (PBST) and incubated with 200 μl of PBS plus 1% BSA (blocking buffer) for 1 h to prevent non-specific binding. HEK293A lysates expressing HA-EGFP-Plk1 were prepared in a TBSN [20 mM Tris-HCl (pH 8.0), 150 mM NaCl, 0.5% NP-40, 5 mM EGTA, 2 mM MgCl₂, 1.5 mM EDTA, and protease inhibitor cocktail (Roche)]+20% DMSO buffer (˜20 μg total lysates in 100 μl buffer), mixed with the indicated amount of the competitors (p-T78 peptide and its derivative compounds) for 30 min, provided onto the biotinylated peptide-coated ELISA wells, and then incubated with constant rocking for 1 h at 25° C. Following the incubation, ELISA plates were washed 5 times with PBST. To detect bound HA-EGFP-Plk1, the plates were probed for 2 h with 100 μl/well of anti-HA antibody at a concentration of 0.5 μg/ml in the blocking buffer and then washed 5 times. The plates were further probed for 1 h with 100 μl/well of HRP-conjugated secondary antibody (GE Healthcare) at a 1:1,000 dilution in the blocking buffer. The plates were washed 5 times with PBST and incubated with 100 μl/well of 3,3′,5,5′-tetramethylbenzidine solution (Sigma-Aldrich, St. Louis, MO) as substrate until a desired absorbance was reached. The reactions were stopped by the addition of 100 μl/well of stop solution (Cell Signaling Technology, Danvers, MA). The optical density (O.D.) was measured at 450 nm by using a Perkin-Elmer Enspire Multimode Plate reader (PerkinElmer, Inc., Boston, MA). Data obtained from more than three independent experiments were analyzed by GraphPad (San Diego, CA) Prism software version 7.

Microsomal metabolic stability assays were performed as reported previously (Urban et al., Sci. Rep. 2017, 7 (1), 12758). PAMPA permeability was measured using a high throughput protocol, as reported (Sun et al., Bioorg. Med. Chem. 2017, 25 (3), 1266-1276). Aqueous solubility was determined by a published procedure (Sun et al., Bioorg. Med. Chem. 2019, 27 (14), 3110-3114).

Due to the lack of structural information for the binding of 7 to the PBD domain of Plk1, early SAR studies began by exploring the 2,4-dihydro-3H-1,2,4-triazole-3-thione moiety to determine empirically the requirements for binding. Table 1 shows the inhibitory activity of exemplary triazoloquinazolinones modified in zones 5 and 6 at the Plk1 PBD, microsomal half-life, PAMPA assays, and aqueous solubility.

TABLE 1

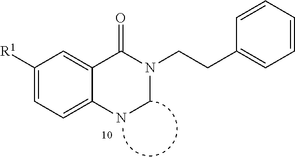

| | | | IC₅₀ (μM, | t_{1/2} (min, | PAMPA | Solub. |
|---|---|---|---|---|---|---|
| Cmpd | Triazole | R¹ = | ELISA) | RLM) | (1e-6 cm/s) | (μg/ml) |
| PLHSpT | — | — | 14.74 ± 0.48 (12) | ND | ND | ND |
| 6a | | | | | | |
| (prior art) | | | | | | |
| 7 | 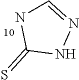 | H | 4.38 ± 0.41 (6) | ND | ND | ND |
| 15 | 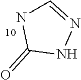 | H | >50 | ND | ND | <1 |
| 16 | 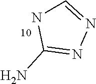 | F | >50 | 29.4 | 885 | 2.2 |
| 17 | 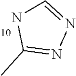 | F | >50 | 10.7 | 1084 | 11.0 |
| 18 | 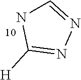 | F | >50 | 11.8 | 924 | 14.8 |
| 19 | 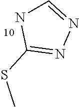 | H | >50 | ND | ND | ND |

IC₅₀ values are n = 3, unless noted in parentheses
ND, not determined.

The SAR of the side chain, R², was explored by systematically modifying the length and composition of the linker as well as the end group of the side chain. Table 2 shows the inhibitory activity of early triazoloquinazolinones modified in zones 3 and 4 at the Plk1 PBD, microsomal half-life, PAMPA assays, and aqueous solubility.

TABLE 2

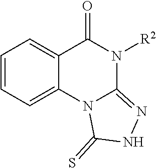

| | | IC₅₀ (μM, | t_{1/2} (min, | PAMPA | Solub. |
|---|---|---|---|---|---|
| Cmpd | R² = | ELISA) | RLM) | (1e-6 cm/s) | (μg/ml) |
| 7 | 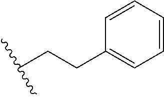 [SMILES: *CCc1ccccc1] | 4.38 ± 0.41 (6) | ND | ND | ND |
| 20 | Et | 1.49 ± 0.22 (6) | >30 | 427 | >36 |
| 21 | Pr | 1.03 ± 0.08 (9) | >30 | 37.0 | 21.3 |
| 22 | 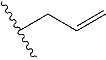 [SMILES: *CC=C] | 1.31 ± 0.03 | ND | ND | 19.4 |
| 23 | Bu | 1.03 ± 0.14 (5) | >30 | 77.5 | 8.5 |
| 24 | Pent | 1.97 ± 0.11 (6) | ND | ND | 4.2 |
| 25 | 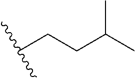 [SMILES: *CCC(C)C] | 1.73 ± 0.08 (6) | ND | ND | 8.0 |
| 26 | i-Pr | 1.30 ± 0.06 | ND | ND | 19.5 |
| 27 | 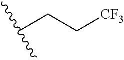 [SMILES: *CCC(F)(F)F] | 1.25 ± 0.11 (5) | >30, | 82 | 23.3 |
| 28 | 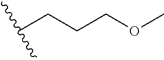 [SMILES: *CCCOC] | 1.75 ± 0.19 | ND | ND | 31.2 |
| 29 | 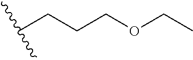 [SMILES: *CCCOCC] | 1.68 ± 0.11 | ND | ND | 15.5 |
| 30^{a} | 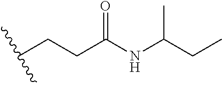 [SMILES: *CCC(=O)NC(C)CC] | 3.23 ± 0.44 | ND | ND | ND |
| 31 | 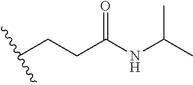 [SMILES: *CCC(=O)NC(C)C] | 3.90 ± 0.43 | ND | ND | ND |
| 32 | 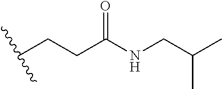 [SMILES: *CCC(=O)NCC(C)C] | 2.92 ± 0.56 | ND | ND | 30.8 |
| 33 | 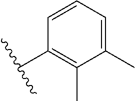 [SMILES: *c1cccc(C)c1C] | 1.85 ± 0.12 | ND | ND | ND |
| 34 | 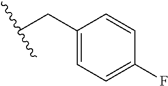 [SMILES: *Cc1ccc(F)cc1] | 3.54 ± 0.57 | ND | ND | ND |

IC₅₀ values are n = 3, unless noted in parentheses
aRacemic
ND, not determined.

Table 3 shows the inhibitory activity of zone 5-modified triazoloquinazolinone derivatives at the Plk1 PBD, microsomal half-life, PAMPA assays, and aqueous solubility.

TABLE 3

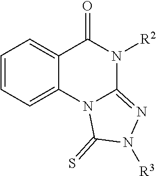

| | | | IC₅₀ (μM, | t_{1/2} (min, | PAMPA | Solub. |
|---|---|---|---|---|---|---|
| Cmpd | R² = | R³ = | ELISA) | RLM) | (1e-6 cm/s) | (μg/ml) |
| 21 | Pr | H | 1.03 ± 0.08 (9) | >30 | 37 | 21.3 |
| 35 | Pr | 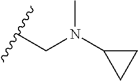 [SMILES: *CN(C)C1CC1] | 1.63 ± 0.05 | ND | ND | Insol. |
| 36 | CH₃ | 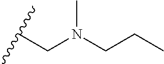 [SMILES: *CN(C)CCC] | 1.20 ± 0.03 | ND | ND | ND |
| 37 | Pr | 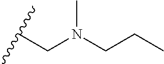 [SMILES: *CN(C)CCC] | 1.83 ± 0.04 | ND | ND | ND |
| 38 | Pr | 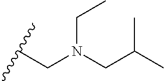 [SMILES: *CN(CC)CC(C)C] | 1.67 ± 0.06 | ND | ND | 8.7 |
| 39 | Pr | 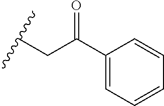 [SMILES: *CC(=O)c1ccccc1] | >50 | ND | ND | ND |

IC₅₀ values are n = 3, unless noted in parentheses
ND, not determined.

The fused phenyl ring of the core was explored to determine whether substitution would be tolerated in this region. Table 4 shows the inhibitory activity of triazoloquinazolinones modified in zones 1 and 2 at the Plk1 PBD, microsomal half-life, PAMPA assays, and aqueous solubility.

TABLE 4

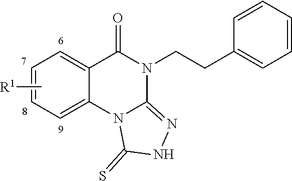

| | | | t_{1/2} | PAMPA | |
|---|---|---|---|---|---|
| | | IC₅₀ (μM, | (min, | (1e-6 | Solub. |
| Cmpd | R¹ = | ELISA) | RLM) | cm/s) | (μg/ml) |
| 7 | H | 4.38 ± 0.41 (6) | ND | ND | ND |
| 40 | 7-F | 12.92 ± 1.85 | >30 | 77.0 | ND |
| 41 | 7-Br | 14.74 ± 0.81 | >30 | 162 | <1 |
| 42 | 7-I | 30.71 ± 1.31 | >30 | 1025 | <1 |
| 43 | 7-Me | 11.29 ± 0.92 | >30 | ND | <1 |
| 44 | 7-NHAc | 2.19 ± 0.10 | >30 | 253 | 2.2 |
| 45 | 7-N(CH₃)₂ | 2.77 ± 0.33 | 13.3 | 356 | <1 |
| 46 | 7-(1- | 1.54 ± 0.21 | >30 | 1.2 | 3.4 |
| | morpholino) | | | | |
| 47 | 8-F | 8.29 ± 0.59 | 22.7 | 91.0 | <1 |
| 48 | 9-F | 2.58 ± 0.12 | >30 | 141 | 3.4 |
| 49 | H (6-aza) | 6.16 ± 0.44 | >30 | 16.8 | 26.9 |
| 50 | H (8-aza) | 27.78 ± 4.07 | 15.8 | 202 | 17.3 |
| 51 | H (9-aza) | 7.55 ± 0.44 | >30 | 51.8 | 4.9 |

IC₅₀ values are n = 3, unless noted in parentheses
ND, not determined

The next stage of the SAR exploration involved combining features from each of the regions explored so far. Table 5 shows the inhibitory activity of triazoloquinazolinones modified in combined zones 1, 2, 3, and 4 at the Plk1 PBD, microsomal half-life, PAMPA assays, and aqueous solubility.

TABLE 5

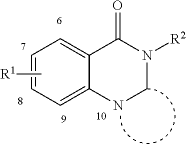

| | | | | IC₅₀ | t_{1/2} | PAMPA | |
|---|---|---|---|---|---|---|---|
| | | | | (μM, | (min, | (1e-6 | Solub. |
| Cmpd | R¹ = | R² = | Triazole | ELISA) | RLM) | cm/s) | (μg/ml) |
| 52 | 7-F | 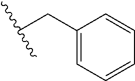 [SMILES: *Cc1ccccc1] | 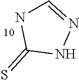 | 6.42 ± 0.44 | >30 | 841 | 8.3 |
| 53 | 7-F | 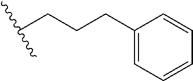 [SMILES: *CCCc1ccccc1] | 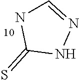 | 6.63 ± 0.39 | 8.8 | 1269 | <1 |
| 54 | 7-Cl | 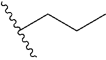 [SMILES: *CCC] | 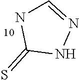 | 1.77 ± 0.15 (6) | ND | ND | ND |
| 55 | 7-Cl | 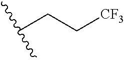 [SMILES: *CCC(F)(F)F] | 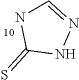 | 2.14 ± 0.12 | >30 | 257 | 5.1 |
| 56 | 7-Cl | 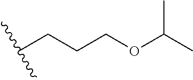 [SMILES: *CCCOC(C)C] | 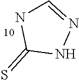 | 1.62 ± 0.17 | ND | ND | 8.6 |
| 57 | 7-Cl | 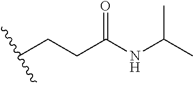 [SMILES: *CCC(=O)NC(C)C] | 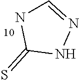 | 1.49 ± 0.09 | ND | ND | 8.1 |
| 58 | 7-CH₃ | 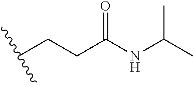 [SMILES: *CCC(=O)NC(C)C] | 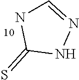 | 2.01 ± 0.21 | ND | ND | ND |
| 59 | 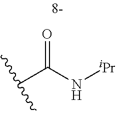 [SMILES: *C(=O)NC(C)C] | 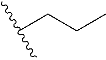 [SMILES: *CCC] | 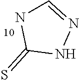 | 3.01 ± 0.10 | ND | ND | ND |
| 60 | 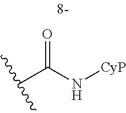 | 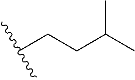 [SMILES: *CCC(C)C] | 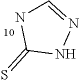 | 11.11 ± 0.90 | ND | ND | ND |
| 61 | 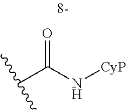 | CH₃ | 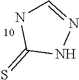 | 2.75 ± 0.17 | ND | ND | ND |
| 62 | 7-F | 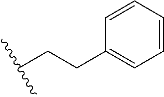 [SMILES: *CCc1ccccc1] | 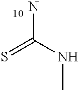 | >50 | 2.2 | 161 | 4.5 |

IC₅₀ values are n = 3, unless noted in parentheses
CyP, cyclopentyl
ND, not determined

Based on the observed results thus far, it became clear that the most significant positive modifications to the chemotype are in the R^{Z} side chain. Therefore, a large number of analogs on the 7-fluoro core with varied side chains were prepared. Table 6 shows the inhibitory activity at the Plk1 PBD of exemplary triazoloquinazolinones modified in zones 3 and 4 with a preferred 7-fluoro substitution, except for 69 with 9-fluoro, microsomal half-life, PAMPA assays, and aqueous solubility.

TABLE 6

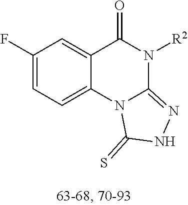

| | | IC₅₀ | t_{1/2} | | |
|---|---|---|---|---|---|
| | | (μM, | (min, | PAMPA | Solub. |
| Cmpd | R² = | ELISA) | RLM) | (1e-6 cm/s) | (μg/ml) |
| 63 | Et | 1.98 ± 0.07 | >30 | 39.5 | >39 |
| 64 | Pr | 1.16 ± 0.06 (12) | >30 | 195 | >28 |
| 65 | 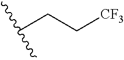 [SMILES: *CCC(F)(F)F] | 0.75 ± 0.06 (5) | >30 | 117 | 17.3 |
| 66 | 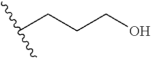 [SMILES: *CCCO] | 1.44 ± 0.10 (9) | >30 | 222 | >43 |
| 67 | 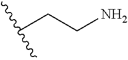 [SMILES: *CCN] | 4.33 ± 0.18 | >30 | 8.2 | >41 |
| 68 | 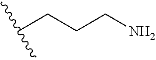 [SMILES: *CCCN] | 2.26 ± 0.07 (4) | >30^{c} >30^{d} | <1^{c} 16.8^{d} | >43^{c} 27^{d} |
| 69 | 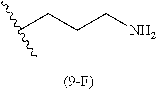 [SMILES: *CCCN] | 5.07 ± 0.16 | >30 | 4.2 | 15.9 |
| 70 | 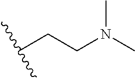 [SMILES: *CCN(C)C] | 2.45 ± 0.25 | >30 | 177 | >45 |
| 71^{a} | 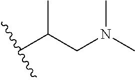 [SMILES: *C(C)CN(C)C] | 6.12 ± 0.17 | >30 | 186 | >47 |
| 72 | 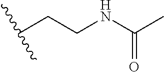 [SMILES: *CCNC(C)=O] | 2.48 ± 0.20 | >30 | 12.9 | >47 |
| 73 | 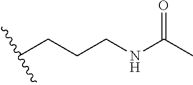 [SMILES: *CCCNC(C)=O] | 2.48 ± 0.08 (6) | >30 | 18.0 | >50 |
| 74 | 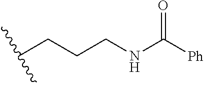 [SMILES: *CCCNC(=O)c1ccccc1] | 1.60 ± 0.19 (4) | >30 | 267 | 21.9 |
| 75 | 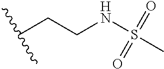 [SMILES: *CCNS(C)(=O)=O] | 2.77 ± 0.31 | >30 | 8.9 | >53 |
| 76 | 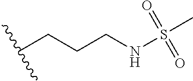 [SMILES: *CCCNS(C)(=O)=O] | 1.87 ± 0.06 | >30 | 10.8 | 34 |
| 77 | 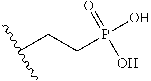 [SMILES: *CCP(=O)(O)O] | 21.70 ± 1.73 | ND | 9.3 | >51 |
| 78 | 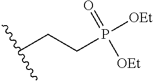 [SMILES: *CCP(=O)(OCC)OCC] | 13.89 ± 0.59 | >30 | 16.7 | >59 |
| 79 | 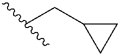 [SMILES: *CC1CC1] | 0.77 ± 0.08 (8) | >30 | 46.0 | 16.2 |
| 80^{b} | 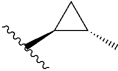 [SMILES: *[C@H]1C[C@@H]1C] | 1.19 ± 0.05 (4) | >30 | 188 | 13.2 |
| 81 | 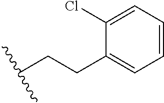 [SMILES: *CCc1ccccc1Cl] | 14.37 ± 0.33 | 22.8 | 56.3 | 3.0 |
| 82 | 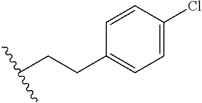 [SMILES: *CCc1ccc(Cl)cc1] | 5.71 ± 0.44 | >30 | <1 | 1.2 |
| 83 | 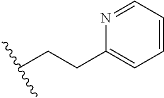 [SMILES: *CCc1ccccn1] | 0.87 ± 0.07 (4) | >30 | 355 | 13.0 |
| 84 | 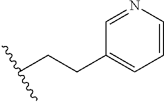 [SMILES: *CCc1cccnc1] | 1.07 ± 0.13 (9) | >30 | <1 | 25.1 |
| 85 | 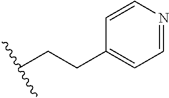 [SMILES: *CCc1ccncc1] | 0.89 ± 0.05 (4) | >30 | 32.7 | 10.9 |
| 86 | 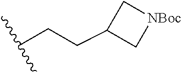 [SMILES: *CCC1CN(C(=O)OC(C)(C)C)C1] | 5.67 ± 0.30 | ND | 229 | 15 |
| 87^{a} | 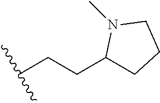 [SMILES: *CCC1CCCN1C] | 5.67 ± 0.42 | >30 | 53.8 | 17.8 |
| 88 | 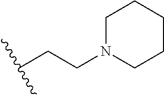 [SMILES: *CCN1CCCCC1] | 1.51 ± 0.19 (4) | >30 | 358 | 2.4 |
| 89 | 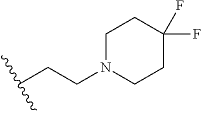 [SMILES: *CCN1CCC(F)(F)CC1] | 1.37 ± 0.08 (5) | >30 | 234 | 20.2 |
| 90 | 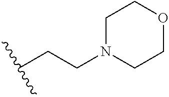 [SMILES: *CCN1CCOCC1] | 1.63 ± 0.22 (5) | >30 | 67.0 | >52 |
| 91 | 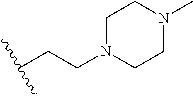 [SMILES: *CCN1CCN(C)CC1] | 6.72 ± 0.17 | >30 | 12.7 | >54 |
| 92 | 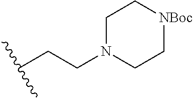 [SMILES: *CCN1CCN(C(=O)OC(C)(C)C)CC1] | 1.84 ± 0.01 | 26.3 | 33.1 | 20.5 |
| 93 | 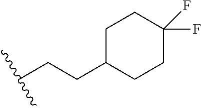 [SMILES: *CCC1CCC(F)(F)CC1] | 2.83 ± 0.36 | >30 | 201 | <1 |

IC₅₀ values are n = 3, unless noted in parentheses
^{a}Racemic.
^{b}Relative stereochemistry, trans.
^{c}TFA salt.
^{d}Free base
ND, not determined

Expanding upon the SAR established above, the side chain region continued to be explored by focusing on the propyl amide motif, given that this serves as a good handle for further modification to probe the binding pocket and that compound 74 with the benzamide was one of the more potent analogs thus far. Table 7 shows the inhibitory activity of exemplary amide-containing triazoloquinazolinones modified in zones 3 and 4 with preferred 7-fluorine at the Plk1 PBD, microsomal half-life, PAMPA assays, and aqueous solubility.

TABLE 7

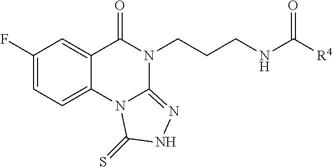

| | | IC₅₀ | t_{1/2} | PAMPA | |
|---|---|---|---|---|---|
| | | (μM, | (min, | (1e-6 | Solub. |
| Cmpd | R² = | ELISA) | RLM) | cm/s) | (μg/ml) |
| 94 | 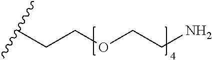 | 5.36 ± 0.40 | ND | ND | ND |
| 95 | 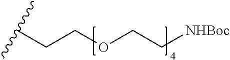 | 3.22 ± 0.12 | ND | ND | ND |
| 96 | 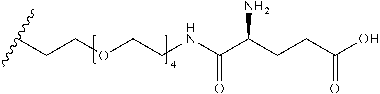 | 13.13 ± 0.36 | >30 | 13.4 | >99 |
| 97 | 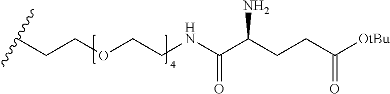 | 9.40 ± 0.35 | >30 | 18.1 | >107 |
| 98 | 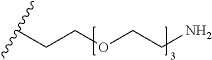 | 5.02 ± 0.29 | >30 | 8.8 | 36.1 |
| 99 | 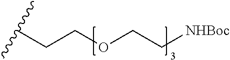 | 2.88 ± 0.21 | 27.9 | 44.3 | >88 |
| 100 | 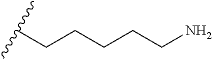 [SMILES: *CCCCCN] | 3.04 ± 0.16 | >30 | 3.5 | 31.8 |
| 101 | 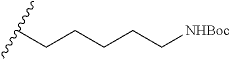 [SMILES: *CCCCCNC(=O)OC(C)(C)C] | 3.23 ± 0.15 | >30 | 405 | >75 |
| 102 | 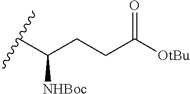 [SMILES: *[C@@H](CCC(=O)OC(C)(C)C)NC(=O)OC(C)(C)C] | 8.19 ± 0.26 | 25.1 | 1214 | 20.4 |
| 103 | 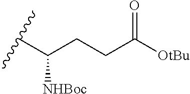 [SMILES: *[C@H](CCC(=O)OC(C)(C)C)NC(=O)OC(C)(C)C] | 3.31 ± 0.14 | 3.8 | 744 | 41.6 |
| 104 | 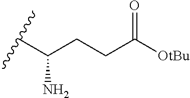 [SMILES: *[C@@H](N)CCC(=O)OC(C)(C)C] | 2.33 ± 0.13 | >30 | 8.7 | 37.5 |
| 105 | 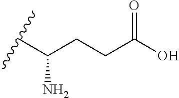 [SMILES: *[C@@H](N)CCC(=O)O] | 12.65 ± 0.44 | >30 | 20.1 | 32.0 |
| 106 | 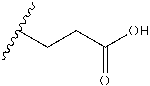 [SMILES: *CCC(=O)O] | 5.05 ± 0.22 | >30 | <1 | >58 |
| 107 | 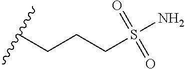 [SMILES: *CCCS(N)(=O)=O] | 2.18 ± 0.19 | >30 | 3.0 | 42.4 |
| 108 | 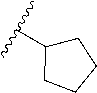 [SMILES: *C1CCCC1] | 1.47 ± 0.18 | >30 | 155 | >58 |
| 109^{a} | 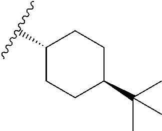 [SMILES: *[C@H]1CC[C@H](C(C)(C)C)CC1] | >50 | >30 | 66.9 | 1.1 |
| 110^{b} | 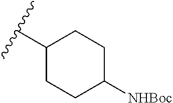 [SMILES: *C1CCC(NC(=O)OC(C)(C)C)CC1] | 2.38 ± 0.74 | 24.8 | 330 | >77 |
| 111^{b} | 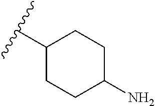 [SMILES: *C1CCC(N)CC1] | 3.97 ± 0.26 | >30 | 1.6 | 38.8 |
| 112^{b} | 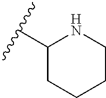 [SMILES: *C1CCCCN1] | 27.66 ± 1.25 | >30 | ND | >60 |
| 113^{b} | 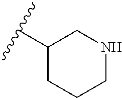 [SMILES: *C1CCCNC1] | 30.20 ± 1.09 | >30 | ND | 29.6 |
| 114 | 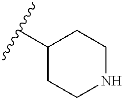 [SMILES: *C1CCNCC1] | 44.01 ± 2.32 | >30 | ND | >60 |
| 115 | 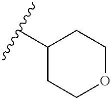 [SMILES: *C1CCOCC1] | 1.79 ± 0.11 (4) | ND | 8.0 | <1 |
| 116 | 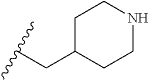 [SMILES: *CC1CCNCC1] | 3.96 ± 0.37 | >30 | 4.4 | 37.5 |
| 117 | 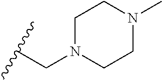 [SMILES: *CN1CCN(C)CC1] | 4.66 ± 0.23 | >30 | <1 | >64 |
| 118 | 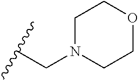 [SMILES: *CN1CCOCC1] | 3.77 ± 0.19 | >30 | <1 | >62 |
| 119 | 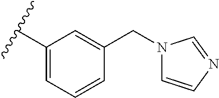 [SMILES: *c1cccc(Cn2ccnc2)c1] | 1.48 ± 0.12 (4) | >30 | 46.8 | 13.7 |
| 120 | 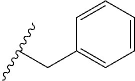 [SMILES: *Cc1ccccc1] | 1.23 ± 0.09 (6) | 18.7 | 248 | 31.7 |
| 121 | 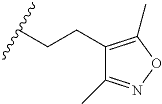 [SMILES: *CCc1c(C)noc1C] | 1.78 ± 0.13 (4) | 6.3 | 27.6 | 37.6 |

IC₅₀ values are n = 3, unless noted in parentheses
^{a}Relative stereochemistry, trans.
^{b}Racemic.
ND, not determined

Given that compound 120 showed the best activity of the amide side chains, substitution around the phenylacetamide portion was explored. Table 8 shows the inhibitory activity of exemplary phenylacetic acid triazoloquinazolinone derivatives modified in zones 3 and 4 with preferred 7-fluorine at the P(k PBD, microsomal half-life, PAMPA assays, and aqueous solubility.

TABLE 8

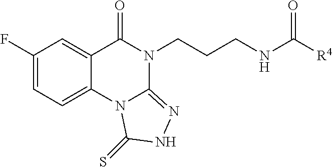

| | | IC₅₀ (μM, | t_{1/2} (min, | PAMPA | Solub. |
|---|---|---|---|---|---|
| Cmpd | R⁴ = | ELISA) | RLM) | (1e-6 cm/s) | (μg/ml) |
| 122 | 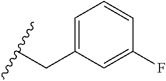 [SMILES: *Cc1cccc(F)c1] | 1.42 ± 0.18 (4) | 26.5 | 364 | >63 |
| 123 | 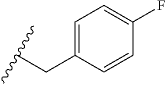 [SMILES: *Cc1ccc(F)cc1] | 1.35 ± 0.17 (4) | 26.3 | 8.5 | 8.5 |
| 124 | 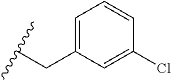 [SMILES: *Cc1cccc(Cl)c1] | 1.15 ± 0.03 (4) | 8.4 | 861 | >66 |
| 125 | 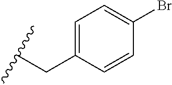 [SMILES: *Cc1ccc(Br)cc1] | 1.59 ± 0.12 | 20.6 | 767 | <1 |
| 126 | 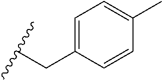 [SMILES: *Cc1ccc(C)cc1] | 1.18 ± 0.01 | 23.9 | 621 | 12.9 |
| 127 | 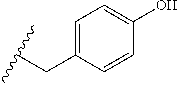 [SMILES: *Cc1ccc(O)cc1] | 1.16 ± 0.06 | >30 | 3.7 | 31.0 |
| 128 | 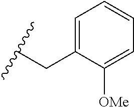 [SMILES: *Cc1ccccc1OC] | 1.15 ± 0.11 (5) | 16.1 | 379 | >65 |
| 129 | 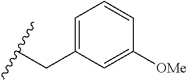 [SMILES: *Cc1cccc(OC)c1] | 0.96 ± 0.08 (4) | 20.1 | 300 | >65 |
| 130 | 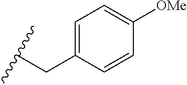 [SMILES: *Cc1ccc(OC)cc1] | 1.06 ± 0.08 (5) | >30 | 233 | 3.2 |
| 131 | 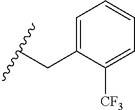 [SMILES: *Cc1ccccc1C(F)(F)F] | 1.40 ± 0.07 (4) | 3.7 | 825 | 40.3 |
| 132 | 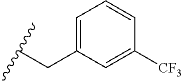 [SMILES: *Cc1cccc(C(F)(F)F)c1] | 2.23 ± 0.33 | 17.3 | 1088 | 26.8 |
| 133 | 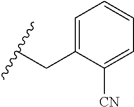 [SMILES: *Cc1ccccc1C#N] | 1.24 ± 0.14 (4) | >30 | 156 | 20.9 |
| 134 | 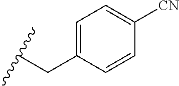 [SMILES: *Cc1ccc(C#N)cc1] | 0.99 ± 0.14 | >30 | 172 | 2.0 |
| 135 | 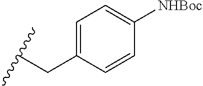 [SMILES: *Cc1ccc(NC(=O)OC(C)(C)C)cc1] | 2.72 ± 0.63 | >30 | 910 | 17.4 |
| 136 | 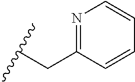 [SMILES: *Cc1ccccn1] | 5.34 ± 0.64 | >30 | 68.0 | 41.4 |
| 137 | 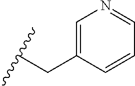 [SMILES: *Cc1cccnc1] | 2.50 ± 0.28 | >30 | 21.0 | 31.8 |
| 138 | 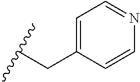 [SMILES: *Cc1ccncc1] | 3.01 ± 0.26 | >30 | <1 | 9.6 |

IC₅₀ values are n = 3, unless noted in parentheses
ND, not determined

ADME data was compared for the six most potent analogs (65, 79, 83, 85, 129, and 134). Five of the six analogs had half-lives >30 min in RLMs, and the sixth (129) had a fairly good (20.1 min) half-life (Tables 6 and 8). Notably, the PAMPA permeability at pH 7.4 was favorable for six compounds (65, 83, 129, and 134) with moderate to good permeability (100 to >200×10⁻⁶ cm/s). Three analogs (65, 79, and 129) showed moderate to good solubility (10 to >60 μg/mL). Compound 129 displayed both good permeability and solubility (P=311×10⁻⁶ cm/s, S >65 μg/mL) (Table 8). Compound 129 may represent the best balance of PLK1 PBD affinity and ADME properties.

Compounds 139-144 are S-methyl and S-acetyl derivatives that were also explored as potential prodrugs of the corresponding active 1-thioxo derivatives. By decreasing the polarity of the molecules, it was expected to provide better intracellular levels of the active species, with the expectation that the masking moiety on the S could be labile prior to reaching the site of action. Table 9 shows the inhibitory activity of prodrug triazoloquinazolinones and their corresponding parent drugs at the Plk1 PBD. The corresponding parent drugs were unsubstituted 21 for 142 and 143, 7-F analog 64 for 144, 9-F analog 139 for 145, 9-Cl analog 140 for 146 and 9-OMe analog 141 for 147.

TABLE 9

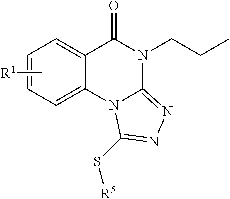

| Cmpd | R¹ = | R⁵ = | IC₅₀ (μM) |
|---|---|---|---|
| 21 | H | H^{a} | 1.03 ± 0.08 (9) |
| 64 | 7-F | H^{a} | 1.16 ± 0.06 (12) |
| 139 | 9-F | H^{a} | 2.06 ± 0.07 (4) |
| 140 | 9-Cl | H^{a} | 2.49 ± 0.02 |
| 141 | 9-OMe | H^{a} | 2.43 ± 0.01 |
| 142 | H | Ac | 1.33 ± 0.08 |
| 143 | H | Me | >50 |
| 144 | 7-F | Me | >50 |
| 145 | 9-F | Me | >50 |
| 146 | 9-Cl | Me | >50 |
| 147 | 9-OMe | Me | >50 |

IC₅₀ values are n = 3, unless noted in parentheses
^{a}Shown as the enol tautomers, while this series is shown in the thiocarbonyl form elsewhere.
ND, not determined

In HeLa cells, compounds 143, 144, and 145 were compared for their ability to induce mitotic arrest and an antiproliferation effect, which are characteristics of Plk1 PBD inhibition (Liu et al., Nat. Chem. Biol. 2011, 7 (9), 595-601; and Yun et al., Nat. Struct. Mol. Biol. 2009, 16 (8), 876-88). HeLa cells were cultured as recommended by the American Type Culture Collection (ATCC). Asynchronously growing cells were treated with control DMSO or the indicated concentration of 143. To reveal chromosome morphologies, cells were treated with 4′,6-diamidino-2-phenylindole (DAPI), fixed, and subjected to confocal microscopy. Immunostaining was carried out using anti-Plk1 (Santa Cruz Biotechnology, Santa Cruz, CA), anti-Cep63 (MilliporeSigma), anti-CREST (Antibodies Incorp, Davis, CA), and anti-α-tubulin (Sigma, St. Louis, MO) antibodies. Confocal images were acquired using a Zeiss LSM780 equipped with a plan-apochromat 63× (N.A. 1.4) oil-immersion objective lens, 34-channel GaAsP spectral detector (Carl Zeiss Microscopy, LLC.), and 8-bit, 0.5-μm z-steps.

To quantify fluorescence signal intensities, images were acquired under the same settings and the images obtained after the maximum-intensity projection of z-stacks were analyzed using the Zeiss ZEN v2.1 software (Carl Zeiss Microscopy, LLC). All the quantifications were performed with randomly chosen fields from at least three independent experiments. All values are given as mean±s. D. P values were calculated by unpaired t test from the mean data of each group in Graph Pad Prism (***P<0.001). The statistical details including the definitions and value of n (e.g., number of experimental replicates, cells, etc.) And standard deviations are provided herein.

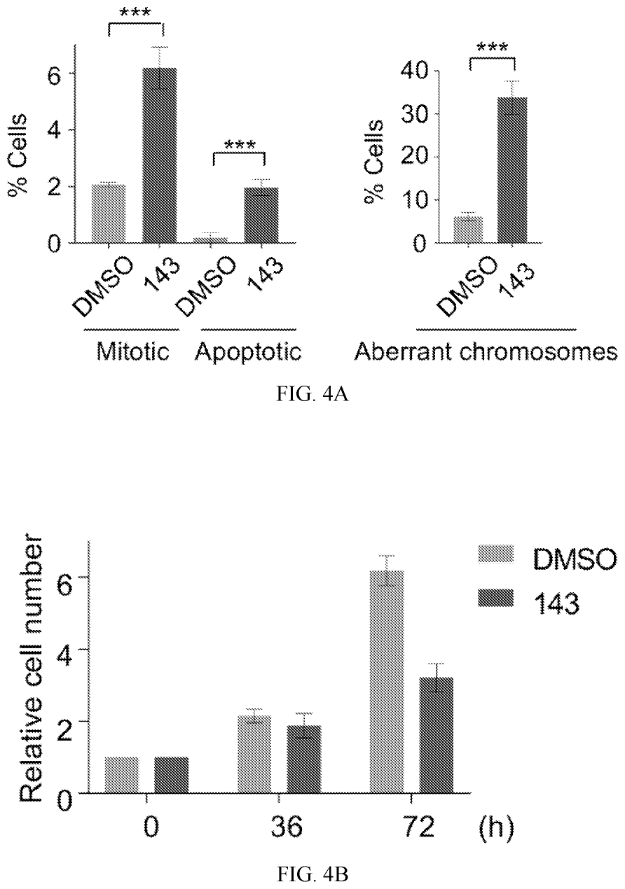

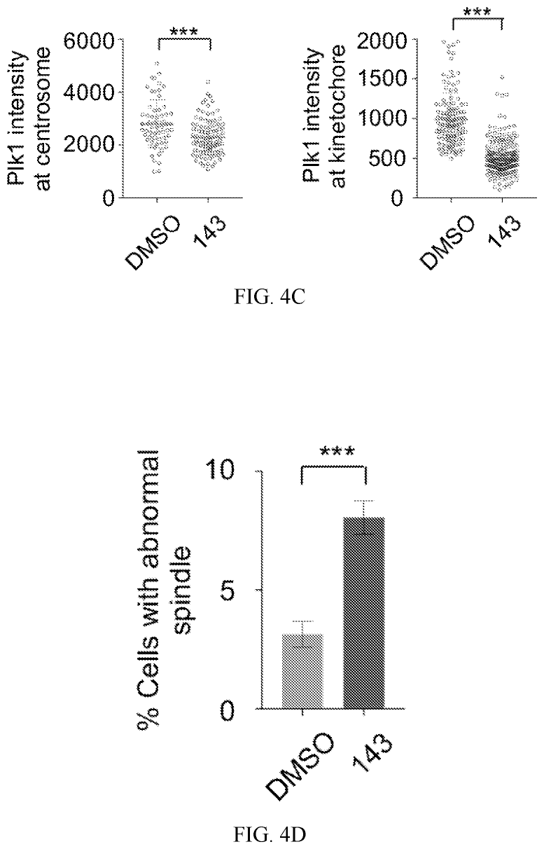

Results showed that treatment of cells with 100 μM of either compound 143 or 145 effectively induced rounded cells with apoptotic or aberrant chromosome morphologies (FIGS. 4A and 4B), indicative of a sustained mitotic arrest. Consequently, both compounds inhibited cell proliferation, decreasing the total cell population nearly by 50% three days after treatment. Considering that blocking the function of Plk1 PBD-dependent PPI is destined to be less drastic than that of Plk1 KD (Lee et al., Trends Pharmacol. Sci. 2015, 36 (12), 858-877; and Park et al., F1000Res. 2017, 6, 1024) and that several purported inhibitors reported to date show a low level (IC₅₀ of 50-1000 μM) of cellular activities (see, e.g., Huggins et al., ACS Omega 2020, 5 (1), 822-831; Sharma et al., Sci. Rep. 2019, 9 (1), 15930; and Rubner et al., Angew. Chem. Int. Ed. 2018, 57 (52), 17043-17047), the activity of 143 or 145 is a clear improvement. Treatment with compound 144 also induced mitotic arrest but at a somewhat reduced level, whereas control DMSO failed to induce any detectable phenotype under the same conditions. As expected if the arrest was caused by interfering with the PBD-dependent Plk1 function, compound 143 significantly inhibited Plk1 localization to centrosomes (approximately 20% inhibition) and kinetochores (approximately 50% inhibition) frequently yielding cytosolic aggregates of delocalized Plk1 as observed previously (Kang et al., Mol. Cell 2006, 24 (3), 409-422). See FIG. 4C. A weaker blocking of Plk1 localization to the centrosome is likely due to the presence of PBD-independent Plk1-binding targets at this structure. Consistent with impaired Plk1 localization, spindle bipolarity was significantly compromised in 143-treated cells (FIG. 4D). Under the same conditions, the parent drugs, i.e. 21, 64, and 139 lacked a detectable level of cellular response. These findings suggest that, although the prodrugs themselves failed to inhibit Plk1 PBD (Table 9) due to the absence of a free 1-thioxy group, the S-methyl thioethers had a more pronounced response, presumably by promoting cell membrane permeability while regenerating the parent drug intracellularly.

### Example 116

This example demonstrates the Plk1 PBD specificity of exemplary compounds of formula (I) in an aspect of the invention.

To determine whether the compounds of formula (I) show Plk1 PBD-binding specificity, fluorescence polarization (FP)-based inhibition assays were carried out using fluorescein isothiocyanate (FITC)-labeled peptides that specifically bind to each of the PBDs from Plk1, Plk2, and Plk3 (Qian et al., Biopolymers 2014, 102 (6), 444-455).

FP assays were carried out essentially as described previously (Qian, ibid). Briefly, an appropriate 5-carboxyfluorescein-labeled PBD-binding peptide for Plk1, Plk2, or Plk3, was incubated at a final concentration of 5 nM with various concentrations of the bacterially-expressed and purified PBD of Plk1, Plk2 or Plk3, respectively, in a binding buffer [10 mM Tris (pH 8.0), 1 mM EDTA, 50 mM NaCl, and 0.01% Nonidet P-40]. Samples were analyzed 30 min after mixing all components in a 384-well format using the Molecular Devices (San Jose, CA) SpectraMax Paradigm multi-mode microplate detection platform. To eliminate the possibility that the apparent anti-Plk1 PBD activity is not the result of compounds' ability to inhibit to the ligand peptide used for the Plk1 FP assay above (i.e., FITC-Ahx-DPPLHSpTAI-NH₂), a second Plk1 PBD-binding peptide (FITC-Ahx-GPMQSpTPLNG-NH₂) was used to confirm the activity (Liu et al., Nat. Chem. Biol. 2011, 7 (9), 595-601; and Yun et al., Nat. Struct. Mol. Biol. 2009, 16 (8), 876-88). All experiments were performed in triplicate for each sample. Obtained data were plotted using GraphPad Prism software version 7.

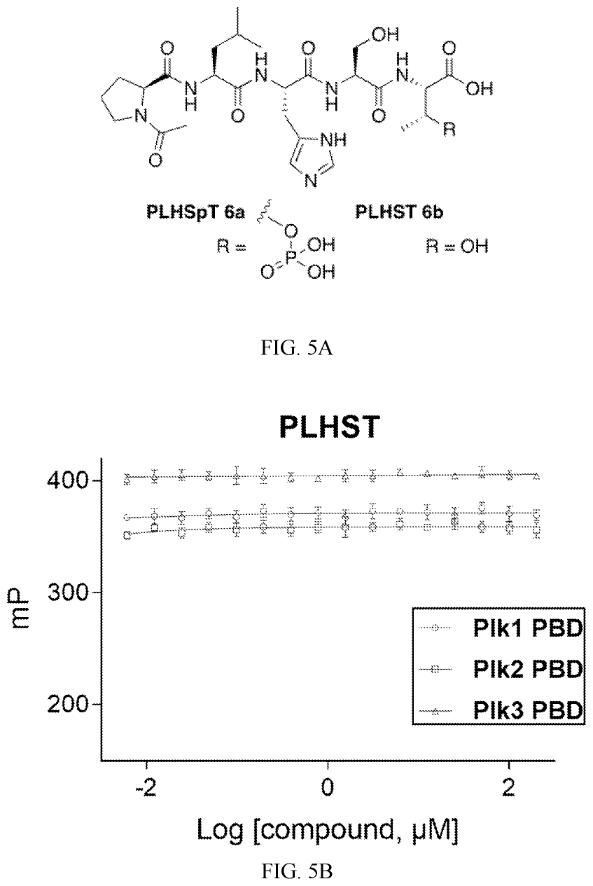

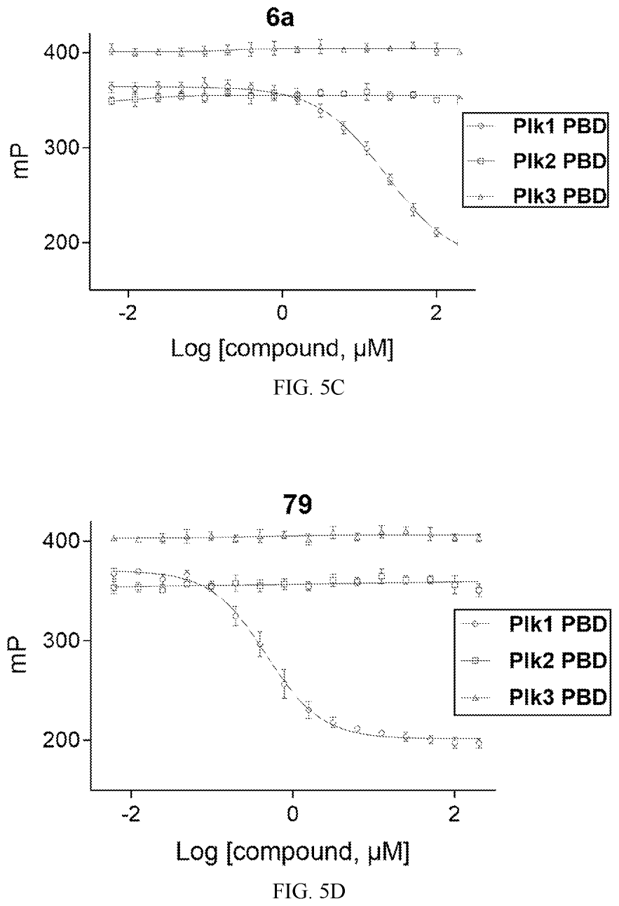

Under these experimental settings, PLHSpT 6a (FIG. 5B) but not its respective nonphosphorylated peptide (FIG. 5A), specifically inhibited Plk1 PBD with an IC₅₀ of 22 μM. Under the same conditions, representative, potent compound 79 inhibited Plk1 PBD with an IC₅₀ of 0.47 μM and failed to exhibit any detectable level of inhibition against the PBDs from Plk2 and Plk3 (FIG. 5C). An approximately 40-fold increased potency for compound 79 over PLHSpT 6a are largely in good agreement with the data obtained with ELISA-based assays described above. Similar to this observation, expanded FP assays showed that several additional compounds, i.e., compounds 20, 27, 89, and 90, tested all showed approximately the same degree of Plk1 PBD specificity. The all-or-none selectivity for Plk1 PBD is significant, given that the well-characterized Plk1 kinase domain inhibitor BI2536 (prior art) exhibits Plk1 selectivity only around four folds (Lenart et al., Curr. Biol. 2007, 17 (4), 304-315).

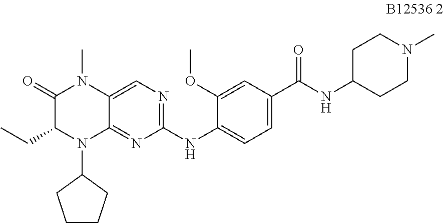 [SMILES: CC[C@@H]1C(=O)N(C)c2cnc(Nc3ccc(C(=O)NC4CCN(C)CC4)cc3OC)nc2N1C1CCCC1]

### Example 117

This example demonstrates the stability in mice of exemplary compounds of formula (I) in an aspect of the invention.

Despite the in vitro data suggesting that most analogs were stable in RLMs up to 30 min (Tables 1-8), further assays were required to fully understand the stability in both in vitro and in vivo systems. Therefore, for selected compounds, additional microsome-based stability assays were performed to determine the major metabolites and class of enzymes responsible for the modifications.

Compounds 23, 46, 65, 79, 83, and 134 were subjected to in vitro mouse liver microsome (MLM) assay. In the in vitro MLM assay, the incubation system (200 μL) contained 100 mM phosphate buffer solution (pH=7.4), 1.0 mg·mL⁻¹ MLMs, 5 mM MgCl₂, and 20 μM compound. The mixture was preincubated at 37° C. for 3 min and initiated with 2 mM NADPH or 2 mM UDPGA. The reaction was stopped by an aliquot of 200 μL of ice-cold acetonitrile after 60 min. After centrifugation at 14 000×g for 10 min, a 5 μL aliquot of the supernatant was injected into a UHPLC-ESIQTOFMS.

For UHPLC-ESI-QTOFMS and UHPLC-ESI-TQMS analysis, metabolite profiling and identification were performed on an Acquity UPLC/Synapt HDMS Q-TOF MS system (Waters Corp., Milford, MA) with an electrospray ionization source. Separation was achieved on an Acquity C18 BEH UPLC column (1.7 mm, 2.1×50 mm; Waters Corp.). The mobile phase consisted of water containing 0.1% formic acid (A) and acetonitrile containing 0.1% formic acid (B). Initial condition of 2% B was held for 0.5 min, with the following linear gradient employed: 20% B at 4 min, 95% B at 8 min, to 99% B at 9 min, held for 1 min for column flushing, then returning to initial conditions for 2 min for column equilibration before the next injection. The flow rate of the mobile phase was set 0.4 mL/min. Column temperature was maintained at 40° C. throughout the run. Data were collected in positive ion mode, which was operated in full-scan mode at 50-950 m/z. Nitrogen was used as both cone gas (50 L/h) and desolvation gas (950 L/h). Source temperature and desolvation temperature were set at 150° C. and 400° C., respectively. The capillary voltage and cone voltage were 3000 and 30 V, respectively. The structures of metabolites of interest were elucidated by tandem MS fragmentography with collision energies ramping from 15 to 40 eV, employing the same chromatographic conditions described above.

LC-MS/MS analysis revealed that glucuronidation is the major metabolite. Note that, in vitro glucuronidation resulted in two products, S- and N-glucuronides in varying amounts, which resulted in two glucuronidation peaks in LC-traces for some compounds. To enhance the stability of prodrugs, S-methyl thioether analogues (143-145) were prepared, which exhibited anti-Plk1 PBD activity in HeLa cells (FIGS. 4A-4D). The analyses with two of these analogues (143 and 144) revealed that although they were stable in human, mouse, and rat cytosol with t_{1/2} values up to 120 min, unlike their parental 21 and 64, they exhibited much shorter t_{1/2} values when reacted with the microsomes from the corresponding three species (Table 10). These findings suggest that the membrane-bound enzymes in the microsomes are mainly responsible for the metabolism of these prodrugs, rather than the soluble cytosolic enzymes.

TABLE 10

| | t_{1/2} Human LM | t_{1/2} Mouse LM | t_{1/2} Rat LM |
|---|---|---|---|
| Cmpd | (min) | (min) | (min) |
| 21 | >120 | >120 | >120 |
| 64 | >120 | >120 | 119.71 |
| 68 | >120 | >120 | 106.37 |
| 69 | >120 | 88.35 | >120 |
| 80 | 50.83 | 105.09 | >120 |
| 130 | >120 | 78.19 | 54.81 |
| 134 | >120 | >120 | >120 |
| 143 | 16.80 | 3.77 | 9.41 |
| 144 | 14.45 | 10.58 | 26.36 |
| | t_{1/2} Human cytosol | t_{1/2} Mouse cytosol | t_{1/2} Rat cytosol |
| | (min) | (min) | (min) |
| 21 | >120 | >120 | >120 |
| 64 | >120 | >120 | >120 |
| 68 | >120 | >120 | >120 |
| 69 | 118.48 | 97.58 | >120 |
| 80 | >120 | >120 | >120 |
| 130 | >120 | >120 | >120 |
| 134 | >120 | 103.84 | >120 |
| 143 | >120 | >120 | >120 |
| 144 | >120 | >120 | >120 |

Next, to achieve a comprehensive understanding of the metabolic stability of the prodrugs, an in vivo experiment was conducted by injecting 143 in mice under a protocol approved by the National Cancer Institute Animal Care and Use Committee. Male C57BL/6 mice from Charles River Laboratories (Wilmington, MA) were housed in the National Cancer Institute animal facility that is a pathogen-free environment controlled for temperature, light (25° C., 12-h light/dark cycle) and humidity (45-65%) with free access to food and water. The National Cancer Institute Animal Care and Use Committee approved all animal experiments conducted in this study.

In the in vivo mouse pharmacokinetics study, 6-8-week-old male C57BL/6 mice were selected for metabolite profiling. In brief, 200 μL/20 g mice of WA (20 mg/kg dissolved in 5% DMSO and 5% Tween 80-contained saline) was administered intraperitoneally to mice. Control mice were treated with saline containing 5% DMSO and 5% Tween 80 alone. Each group consisted of 3 mice. Blood samples were collected by retro-orbital bleeding at 4 h postdose. After centrifugation for 10 min at 14 000×g, serum was obtained and prepared by mixing 20 μL serum with 180 μL 50% aqueous acetonitrile. After centrifugation at 14 000×g for 20 min, a 5 μL aliquot of the supernatants was injected into a Waters UPLC-ESI-QTOFMS system (Waters Corporation, Milford, MA).

Serum was collected at 15, 30, 60, 120 and 240 min after injection. In close agreement with in vitro results, both the hydroxylated and demethylated products were detected in the serum ≥15 min post-injection. While both metabolites decreased time-dependently, demethylated product markedly increased in the serum ≥240 min post-injection. A trace amount of the subsequent glucuronide of the demethylated metabolite was also seen in the serum, as it could be promptly excreted from the liver to the bile, once generated, and can then undergo enterohepatic circulation through which the demethylated product is reabsorbed to the circulation. This could also serve as an explanation for the increased demethylated metabolite at the last time point. The in vitro and in vivo stability of compounds 143 and 145 in mice are shown in Table 11.

TABLE 11

| Cmpd | t_{1/2} in vitro (min) | t_{1/2} in vivo (min) |
|---|---|---|
| 143 | 11.37 ± 3.49 | 28.74 ± 8.30 |
| 145 | 21.80 ± 6.03 | 51.26 ± 11.23 |

Compound 145 yielded similar metabolites with a somewhat improved t_{1/2} value of 51.26 min. These results illustrated that the prodrugs could release the corresponding parent drugs enzymatically, and the released parent drugs levels could be longer lasting due to an enterohepatic circulation.

### General Procedure 2

The following syntheses are directed to the compounds set forth in FIGS. 11A-11I.

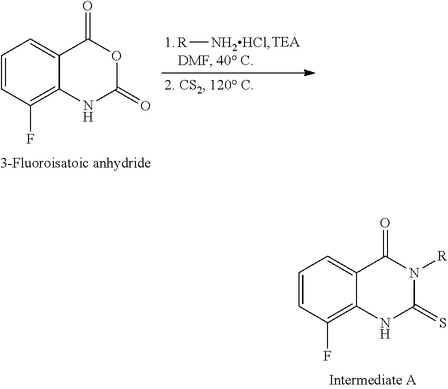

General Procedure A. The amine salt (1.5 mmol) in dimethylformamide (1 mL) was treated with triethylamine (1.5 mmol) with stirring. After the filtration, the amine as free base was added to 3-fluoroisatoic anhydride (1.0 mmol). The reaction mixture was heated at 40° C. for 45 min. The mixture was cooled to room temperature, and carbon disulfide (7.0 mmol, kept at 4° C. until addition) was added. The reaction mixture was heated to 120° C. for 1 h, followed by cooling to room temperature and quenching with water. The reaction mixture was extracted three times with ethyl acetate, the organic layer dried with MgSO₄, filtered and concentrated. The product was isolated from the reaction mixture by column chromatography.

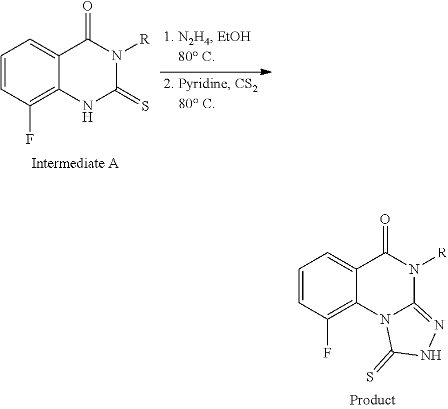

General Procedure B. The intermediate A (1.0 mmol) was dissolved in ethanol or 1,4-dioxane (3 mL) and the Hydrazine anhydrous (7.0 mmol) was added. The reaction mixture was heated to 80° C. for 4-18 h. The reaction mixture was cooled to room temperature and volume reduced under a stream of nitrogen. Pyridine (10.0 mmol) and carbon disulfide (10.0 mmol) were added, and the reaction mixture was heated to 80° C. for 15 h. The reaction mixture was concentrated, and the product was purified by column chromatography.

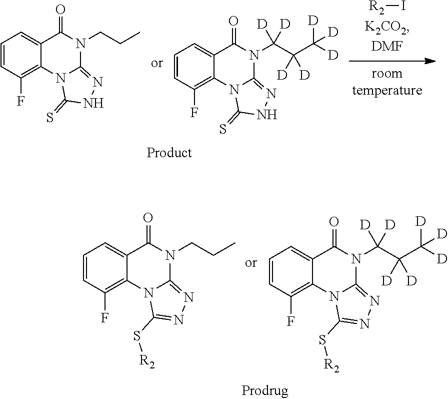

General Procedure C. The Product (1.0 mmol) dissolved in dimethylformamide (5 mL) was add potassium carbonate (1.2 mmol) and the desired alkyl iodide (1.2 mmol). The reaction mixture was stirred at room temperature for 2 h. After the completion, the reaction mixture was quenched with water and extracted with ethyl acetate, and the organic layer dried with MgSO₄ and filtered. The filtrate was concentrated. The product was purified by column chromatography.

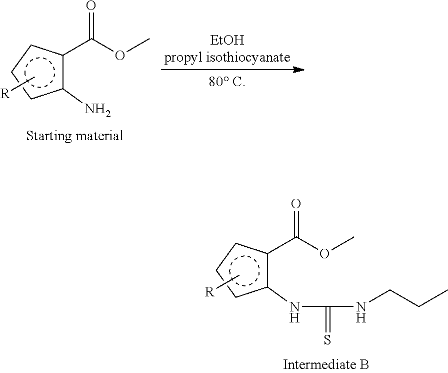

General Procedure D (intermediate B synthesis). The starting material (1.0 mmol) dissolved in ethanol or DMF (2 mL) was treated with propyl isothiocyanate (3.0 mmol) and heat to 80° C. for 18 h. The reaction mixture was diluted with water and extracted with ethyl acetate, the organic layer dried with MgSO₄ and filtered. The filtrate was concentrated. The crude product (intermediate B) was used for the next reaction without further purification.

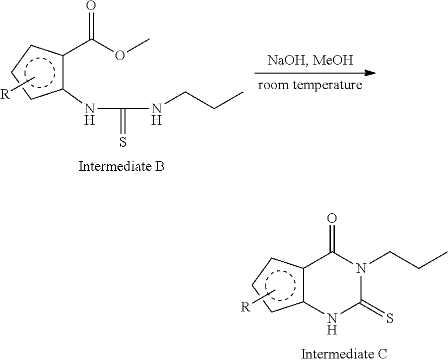

General Procedure E (intermediate C synthesis-1). The intermediate B (1.0 mmol) was dissolved in methanol (5 mL) and sodium hydroxide (3.0 mmol) was added as solid. The reaction mixture was stirred at room temperature to 50° C. for 4-18 h. After the completion, the mixture was treated dropwise with 1 N HCl to pH 1, extracted with ethyl acetate, the organic layer dried with MgSO₄ and filtered. The filtrate was concentrated. The residue was stirred under diethyl ether and filtered to get intermediate C as a solid.

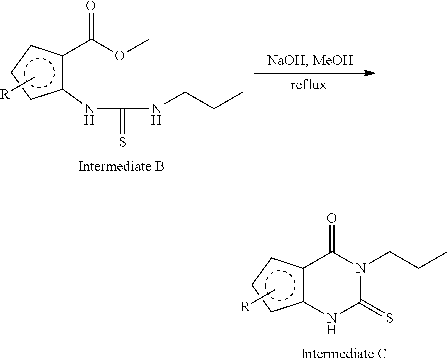

General Procedure F (intermediate C synthesis-2). To a stirred solution of 20% NaOMe in MeOH (15 mL), intermediate B (1.0 mmol) was added. The reaction mixture was refluxed overnight. After the completion, the reaction mixture was cooled and poured into water, which was extracted with ethyl acetate, and the organic layer dried with MgSO₄ and filtered. The filtrate was concentrated. The residue was stirred under diethyl ether and the solid that formed separated by filtration to get intermediate C.

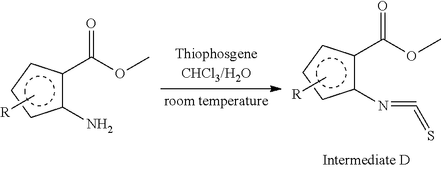

General Procedure G (Intermediate D synthesis-1) (Il'chenko et al., Synthetic communications 37, 2559-2568 (2007)). Starting material (1.0 mmol) was added to a stirred mixture of chloroform (1.0 mL) and water (0.5 mL). A solution of thiophosgene (1.1 mmol) in chloroform (0.5 mL) was added dropwise. The reaction mixture was stirred at room temperature for 3 h. A solution of potassium carbonate (2.5 mmol) in water (0.5 mL) was added. The reaction mixture was diluted with water and extracted with ethyl acetate, dried with MgSO₄ and filtered. The filtrate was concentrated. The reaction mixture (Intermediate D) was used for the next reaction without further purification.

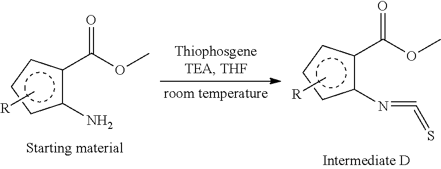

General Procedure H (Intermediate D synthesis-2). (Kawai et al., J Med. Chem. 57, 9844-9854 (2014)). A solution of the starting material (1.0 mmol) in THF (5 mL) was treated with triethylamine (3.0 mmol) and cooled to 0° C. in an ice bath and followed by thiophosgene (1.1 mmol). The reaction mixture was slowly warmed to room temperature and stirred for 15 min to overnight. Water was added to quench the reaction, and the mixture was extracted with ether. The organic layer was dried with MgSO₄ and filtered, and the filtrate was concentrated. The reaction mixture (Intermediate D) was used for the next reaction without further purification.

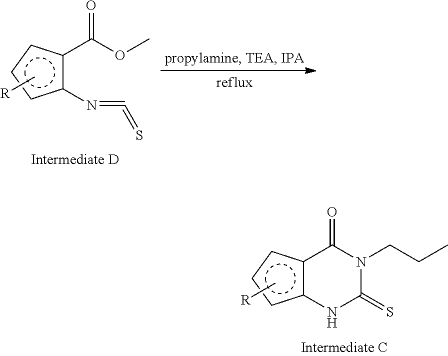

General Procedure I (intermediate C synthesis-3). Intermediate D (1.0 mmol) in 2-propanol (0.3 mL) was treated with propylamine (1.1 mmol) and triethylamine (1.1 mmol). The reaction mixture was refluxed for 2 h and cooled to room temperature. Acetic acid was added until pH 4-5 and the mixture stirred for 15 min. The precipitate was filtered and washed with water. The crystal was recrystallized from 2-propanol.

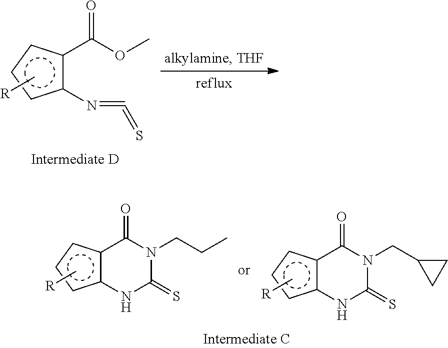

General Procedure J (Intermediate C synthesis-4). To the intermediate D (1.0 mmol) in THF (1.5 mL) was added the appropriate alkylamine (1.1 mmol) and the mixture refluxed for 18 h. After cooling and dilution with water, the mixture was extracted with ethyl acetate, the organic layer dried with MgSO₄ and filtered, and the filtrate was concentrated. The residue was purified by column chromatography.

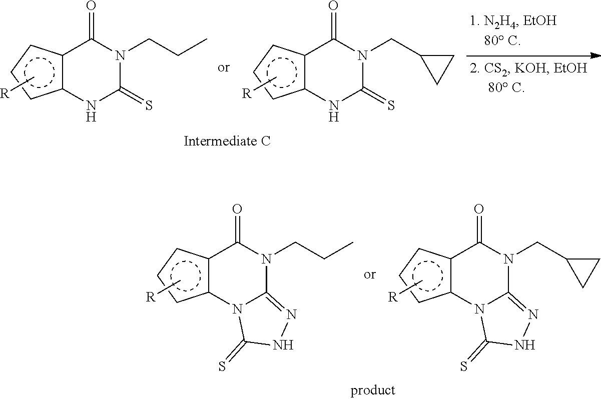

General Procedure K. The intermediate C (1.0 mmol) was dissolved in ethanol (3 mL), and anhydrous hydrazine (7.0 mmol) was added. The reaction mixture was heated to 80° C. for 18 h. The reaction mixture was cooled to room temperature and volume reduced under a stream of nitrogen. Ethanol (3 mL), potassium hydroxide (3 mmol) and carbon disulfide (3 mmol) were added, and the reaction mixture was heated to 80° C. for 18 h. The reaction mixture was cooled and treated dropwise with 1 N HCl to pH 1 with vigorous stirring, and the mixture extracted with ethyl acetate. The organic layer was dried with MgSO₄ and filtered. The filtrate was concentrated. The product was isolated from the reaction mixture by column chromatography.

General procedure L. A mixture of intermediate (0.2 g, 0.72 mmol, 1.0 equiv.), water (2.3 mL) and sodium hydroxide (0,032 g, 0.79 mmol, 1.1 equiv.) was stirred until a solution formed. The corresponding-1-methyl-4-nitroimidazole (0.72 mmol, 1.0 equiv.) was added to the reaction mixture and the mixture stirred for 3 h at room temperature. After completion, the suspension was neutralized with acetic acid and a solid precipitated. The precipitate was isolated by filtration and the solid recrystallized from ethanol. The product was purified by column chromatography (45% yield).

### Example 118

This example demonstrates a synthesis of 9-fluoro-4-(2-(pyridin-4-yl)ethyl)-1-thioxo-2,4-dihydro-[1,2,4]triazolo[4,3-a]quinazolin-5(1H)-one, 10 (NCK131) in an aspect of the invention.

To a solution of 8-fluoro-3-(2-(pyridin-4-yl)ethyl)-2-thioxo-2,3-dihydroquinazolin-4(1H)-one (0.3 g, 0.996 mmol) in EtOH (2.84 mL) was added hydrazine (0.219 mL, 6.97 mmol). The reaction mixture was heated to 80° C. for 18 h. The reaction mixture was cooled to room temperature, volume reduced under a stream of nitrogen, the residue redissolved in EtOH (2.84 mL), and pyridine (0.805 mL, 9.96 mmol) and carbon disulfide (0.600 mL, 9.96 mmol) were added. The reaction mixture was heated to 80° C. for 18 h. When cool, the volume was reduced under a stream of nitrogen and the residue purified by RP-ISCO (H₂O/MeCN+0.1% TFA, 10-100%).

### Example 119

This example demonstrates a synthesis of 4-ethyl-9-fluoro-1-thioxo-2,4-dihydro-[1,2,4]triazolo[4,3-a]quinazolin-5(1H)-one, 12 (NCK138) in an aspect of the invention.

To a solution of 3-ethyl-8-fluoro-2-thioxo-2,3-dihydroquinazolin-4(1H)-one (0.217 g, 0.968 mmol) in EtOH (2.76 mL) was added hydrazine (0.213 mL, 6.77 mmol). The mixture reaction was heated to 80° C. for 18 h. The mixture was cooled to room temperature, volume reduced under a stream of nitrogen, and the residue redissolved in EtOH (2.76 mL). Pyridine (0.783 mL, 9.68 mmol) and carbon disulfide (0.583 mL, 9.68 mmol) were added. The reaction mixture was heated to 80° C. for 18 h. The reaction mixture was reduced in volume under a stream of nitrogen and the product purified by ISCO (EtOAc/hexanes, 0-100%).

### Example 120

This example demonstrates a synthesis of 7-methoxy-4-propyl-1-thioxo-2,4-dihydro-[1,2,4]triazolo[4,3-a]quinazolin-5(1H)-one, 16 (NCK137) in an aspect of the invention.

To a solution of 6-methoxy-3-propyl-2-thioxo-2,3-dihydroquinazolin-4(1H)-one (0.55 g, 2.197 mmol) in EtOH (6.28 mL) was added hydrazine (0.483 mL, 15.38 mmol). The reaction mixture was heated to 80° C. for 18 h. The reaction mixture was cooled to room temperature, reduced in volume under a stream of nitrogen, and the residue redissolved in EtOH (6.28 mL) and pyridine (1.78 mL, 22.0 mmol) and carbon disulfide (1.32 mL, 22.0 mmol) were added. The reaction mixture was heated to 80° C. for 18 h. The reaction mixture was volume reduced under a stream of nitrogen with air and the product purified by RP-ISCO (H₂O/MeCN+0.1% TFA, 10-100%) followed by ISCO (EtOAc/hexanes, 0-100%).

### Example 121

This example demonstrates a synthesis of 9-hydroxy-4-propyl-1-thioxo-2,4-dihydro-[1,2,4]triazolo[4,3-a]quinazolin-5(1H)-one, 18 (NCK144) in an aspect of the invention.

To a solution of 9-methoxy-4-propyl-1-thioxo-2,4-dihydro-[1,2,4]triazolo[4,3-a]quinazolin-5(1H)-one (0.035 g, 0.121 mmol) in DCM (2.41 mL) at 0° C. was added BBr₃ (0.013 mL, 0.133 mmol). The reaction mixture was warmed to room temperature over 2 h. The reaction mixture was quenched, and the mixture gradually warmed to room temperature over 2 h. Following an extraction workup with EtOAc, the organic layer was separated and dried (MgSO₄) and the product purified by ISCO (EtOAc/hexanes, 0-100%); ¹H NMR (400 MHz, DMSO-d₆) δ 11.33 (s, 1H), 7.79 (dd, J=7.7, 1.6 Hz, 1H), 7.51 (t, J=7.9 Hz, 1H), 7.34 (dd, J=8.1, 1.6 Hz, 1H), 4.00-3.92 (m, 2H), 1.76-1.63 (m, 2H), 0.89 (t, J=7.4 Hz, 3H).

### Example 122

This example demonstrates a synthesis of 9-fluoro-1-thioxo-4-(3,3,3-trifluoropropyl)-2,4-dihydro-[1,2,4]triazolo[4,3-a]quinazolin-5(1H)-one, 23 (NCK108) in an aspect of the invention.

8-Fluoro-2-thioxo-3-(3,3,3-trifluoropropyl)-2,3-dihydroquinazolin-4(1H)-one x was synthesized according to General Procedure A. Compound x was converted to 9-fluoro-1-thioxo-4-(3,3,3-trifluoropropyl)-2,4-dihydro-[1,2,4]triazolo[4,3-a]quinazolin-5(1H)-one 23 according to General Procedure B. (51% yield). ¹H NMR (400 MHz, DMSO-d₆): δ 13.93 (s, 1H), 8.06 (d, J=7.2 Hz, 1H), 7.78-7.85 (m, 1H), 7.62-7.69 (td, J=4.0, 7.9 Hz, 1H), 4.22 (t, J=7.0 Hz, 2H), 1.89 (m, 2H); HRMS (ES-API) m/z: calcd. for C₁₂H₈F₄N₄OS (M+H), 333.0433; found, 333.0436.

### Example 123

This example demonstrates a synthesis of 4-(2,2-dconmpifluoropropyl)-9-fluoro-1-thioxo-2,4-dihydro-[1,2,4]triazolo[4,3-a]quinazolin-5(1H)-one, 24 (NCK110) in an aspect of the invention.

3-(2,2-Difluoropropyl)-8-fluoro-2-thioxo-2,3-dihydroquinazolin-4(1H)-one x was synthesized according to General Procedure A. Compound x was converted to 4-(2,2-difluoropropyl)-9-fluoro-1-thioxo-2,4-dihydro-[1,2,4]triazolo[4,3-a]quinazolin-5(1H)-one according to General Procedure B. (9% yield). ¹H NMR (400 MHz, DMSO-d₆): δ 13.91 (s, 1H), 8.04 (dd, 1H, J=0.8, 7.8 Hz), 7.80-7.85 (m, 1H), 7.64-7.67 (m, 1H), 4.46-4.53 (t, 2H, J=13.4 Hz), 1.65-1.75 (t, 3H, J=19.3 Hz); HRMS (ES-API) m/z: calcd. for C₁₂H₉F₃N₄OS (M+H), 315.0527; found, 315.0523.

### Example 124

This example demonstrates a synthesis of 4-(3,3-difluoropropyl)-9-fluoro-1-thioxo-2,4-dihydro-[1,2,4]triazolo[4,3-a]quinazolin-5(1H)-one, 25 (NCK121) in an aspect of the invention.

3-(3,3-Difluoropropyl)-8-fluoro-2-thioxo-2,3-dihydroquinazolin-4(1H)-one x was synthesized according to General Procedure A. Compound x was converted to 4-(3,3-difluoropropyl)-9-fluoro-1-thioxo-2,4-dihydro-[1,2,4]triazolo[4,3-a]quinazolin-5(1H)-one according to General Procedure B. (22% yield). ¹H NMR (400 MHz, DMSO-d₆): δ 13.91 (s, 1H), 8.04 (dd, 1H, J=0.8, 7.8 Hz), 7.80-7.85 (m, 1H), 7.64-7.68 (m, 1H), 4.46-4.58 (t, 2H, J=13.4 Hz), 1.65-1.75 (t, 3H, J=19.3 Hz); HRMS (ES-API) m/z: calcd. for C₁₂H₉F₃N₄OS (M+H), 315.0527; found, 315.0531.

### Example 125

This example demonstrates a synthesis of 9-fluoro-4-(2,2,3,3,3-pentafluoropropyl)-1-thioxo-2,4-dihydro-[1,2,4]triazolo[4,3-a]quinazolin-5(1H)-one, 26 (NCK125) in an aspect of the invention.

8-Fluoro-3-(2,2,3,3,3-pentafluoropropyl)-2-thioxo-2,3-dihydroquinazolin-4(1H)-one x was synthesized according to General Procedure A. Compound x was converted to 9-fluoro-4-(2,2,3,3,3-pentafluoropropyl)-1-thioxo-2,4-dihydro-[1,2,4]triazolo[4,3-a]quinazolin-5(1H)-one according to General Procedure B. (9% yield). ¹H NMR (400 MHz, DMSO-d₆): δ 14.02 (s, 1H), 8.06 (d, 1H, J=7.8 Hz), 7.84-7.90 (m, 1H), 7.65-7.70 (td, 1H, J=4.0, 7.9 Hz), 4.83-4.91 (t, 2H, J=15.2 Hz); ¹⁹F NMR (376 MHz, DMSO-d₆): δ −83.55, −93.46, −119.03; HRMS (ES-API) m/z: calcd. for C₁₂H₆F₆N₄OS (M+H), 369.0245; found, 369.0252.

### Example 126

This example demonstrates a synthesis of 9-fluoro-4-(propyl-d₇)-1-thioxo-2,4-dihydro-[1,2,4]triazolo[4,3-a]quinazolin-5(1H)-one, 27 (NCK112) in an aspect of the invention.

8-Fluoro-3-(propyl-d7)-2-thioxo-2,3-dihydroquinazolin-4(1H)-one x was synthesized according to General Procedure A. Compound x was converted to 9-fluoro-4-(propyl-d7)-1-thioxo-2,4-dihydro-[1,2,4]triazolo[4,3-a]quinazolin-5(1H)-one according to General Procedure B. (27% yield). ¹H NMR (400 MHz, DMSO-d₆): δ 13.86 (s, 1H), 8.03 (dd, 1H, J=0.8, 7.8 Hz), 7.77-7.82 (m, 1H), 7.60-7.65 (td, 1H, J=4.2, 7.9 Hz); ¹⁹F NMR (376 MHz, DMSO-d₆): δ −93.47; HRMS (ES-API) m/z: calcd. for C₁₂H₄D₇FN₄OS (M+H), 286.1155; found, 286.1159.

### Example 127

This example demonstrates a synthesis of 4-(cyclopropylmethyl)-9-fluoro-1-thioxo-2,4-dihydro-[1,2,4]triazolo[4,3-a]quinazolin-5(1H)-one, 29 (NCK143) in an aspect of the invention.

To a solution of 3-(cyclopropylmethyl)-8-fluoro-2-thioxo-2,3-dihydroquinazolin-4(1H)-one (0.155 g, 0.619 mmol) in EtOH (1.769 mL) was added hydrazine (0.136 mL, 4.33 mmol). The reaction mixture was heated to 80° C. for 18 h. The reaction mixture was cooled to room temperature, volume reduced under a stream of nitrogen, and the residue redissolved in EtOH (1.769 mL), and pyridine (0.501 mL, 6.19 mmol) and carbon disulfide (0.373 mL, 6.19 mmol) were added. The reaction mixture was heated to 80° C. for 18 h. The reaction mixture was volume reduced under a stream of nitrogen with air and the product purified by ISCO (EtOAc/hexanes, 0-100%); ¹H NMR (400 MHz, DMSO-d₆) δ 13.87 (s, 1H), 8.03 (d, J=7.8 Hz, 1H), 7.83-7.73 (m, 1H), 7.62 (td, J=8.1, 4.1 Hz, 1H), 3.87 (d, J=7.1 Hz, 2H), 1.26 (dd, J=9.3, 4.5 Hz, 1H), 0.51-0.34 (m, 4H); ¹⁹F NMR (376 MHz, DMSO-d₆) δ −93.22 (dd, J=11.5, 4.1 Hz).

### Example 128

This example demonstrates a synthesis of N-(3-(9-fluoro-5-oxo-1-thioxo-1,2-dihydro-[1,2,4]triazolo[4,3-a]quinazolin-4(5H)-yl)propyl)-2-(3-methoxyphenyl)acetamide, 32 (NCK133) in an aspect of the invention.

A solution of COMU (0.131 g, 0.307 mmol) in DMF (0.511 mL) was added 2-(3-methoxyphenyl)acetic acid (0.051 g, 0.307 mmol) and the reaction stirred at room temperature for 30 min. A solution of 4-(3-aminopropyl)-9-fluoro-1-thioxo-2,4-dihydro-[1,2,4]triazolo[4,3-a]quinazolin-5(1H)-one, 20 (0.06 g, 0.205 mmol, Alverez et al. (18)) in DMF (0.511 mL) was added followed by DIPEA (0.079 mL, 0.450 mmol). The reaction mixture was stirred overnight at room temperature. The reaction mixture was concentrated and the product purified by RP-ISCO (H₂O/MeCN+0.1% TFA, 10-100%).

### Example 129

This example demonstrates a synthesis of 2-(4-cyanophenyl)-N-(3-(9-fluoro-5-oxo-1-thioxo-1,2-dihydro-[1,2,4]triazolo[4,3-a]quinazolin-4(5H)-yl)propyl)acetamide, 34 (NCK135) in an aspect of the invention.

A solution of COMU (0.131 g, 0.307 mmol) in DMF (0.511 mL) was added 2-(4-cyanophenyl)acetic acid (0.049 g, 0.307 mmol) and the reaction stirred at room temperature for 30 min. A solution of 4-(3-aminopropyl)-9-fluoro-1-thioxo-2,4-dihydro-[1,2,4]triazolo[4,3-a]quinazolin-5(1H)-one, 20 (0.06 g, 0.205 mmol) in DMF (0.511 mL) was added followed by DIPEA (0.079 mL, 0.450 mmol). The reaction mixture was then stirred overnight at room temperature. The reaction mixture was concentrated and the product purified by RP-ISCO (H₂O/MeCN+0.1% TFA, 10-100%).

### Example 130

This example demonstrates a synthesis of methyl 4-(2-((3-(9-fluoro-5-oxo-1-thioxo-1,2-dihydro-[1,2,4]triazolo[4,3-a]quinazolin-4(5H)-yl)propyl)amino)-2-oxoethyl)benzoate, 36 (NCK160) in an aspect of the invention.

A solution of COMU (0.110 g, 0.256 mmol) in DMF (0.426 mL) was added 2-(4-(methoxycarbonyl)phenyl)acetic acid (0.050 g, 0.256 mmol) and the reaction stirred at room temperature for 30 min. A solution of 4-(3-aminopropyl)-9-fluoro-1-thioxo-2,4-dihydro-[1,2,4]triazolo[4,3-a]quinazolin-5(1H)-one, 20 (0.05 g, 0.170 mmol) in DMF (0.426 mL) was added followed by DIPEA (0.065 mL, 0.375 mmol). The reaction mixture was stirred overnight at room temperature. The reaction mixture was concentrated and the product purified by RP-ISCO (H₂O/MeCN+0.1% TFA, 10-100%); ¹H NMR (400 MHz, DMSO-d₆) δ 13.85 (s, 1H), 8.13 (t, J=5.7 Hz, 1H), 8.01 (dd, J=7.8, 1.5 Hz, 1H), 7.87 (d, J=8.1 Hz, 2H), 7.77 (ddd, J=11.4, 8.3, 1.5 Hz, 1H), 7.61 (td, J=8.0, 4.1 Hz, 1H), 7.37 (d, J=8.0 Hz, 2H), 4.69 (s, 4H), 3.97 (t, J=7.3 Hz, 2H), 3.82 (s, 2H), 3.17-3.07 (m, 2H), 1.83 (p, J=7.1 Hz, 2H); ¹⁹F NMR (376 MHz, DMSO-d₆) δ −93.40 (dd, J=11.5, 4.2 Hz).

### Example 131

This example demonstrates a synthesis of N-(3-(9-fluoro-5-oxo-1-thioxo-1,2-dihydro-[1,2,4]triazolo[4,3-a]quinazolin-4(5H)-yl)propyl)-2-(4-(morpholinomethyl)phenyl) acetamide, 37 (NCK159) in an aspect of the invention.

A solution of COMU (0.110 g, 0.256 mmol) in DMF (0.86 mL) was added 2-(4-(morpholinomethyl)phenyl)acetic acid (0.060 g, 0.256 mmol) and the reaction stirred at room temperature for 2 h. A solution of 4-(3-aminopropyl)-9-fluoro-1-thioxo-2,4-dihydro-[1,2,4]triazolo[4,3-a]quinazolin-5(1H)-one, 20 (0.05 g, 0.170 mmol) in DMF (0.86 mL) was added followed by DIPEA (0.065 mL, 0.375 mmol). The reaction mixture was then stirred overnight at room temperature. The reaction mixture was concentrated and the product purified by RP-ISCO (H₂O/MeCN+0.1% TFA, 10-100%) followed by RP-ISCO (H₂O/MeCN+0.1% NH₄OH, 10-100%); ¹H NMR (400 MHz, DMSO-d₆) δ 13.84 (s, 1H), 8.06-7.97 (m, 2H), 7.77 (dd, J=11.4, 8.2 Hz, 1H), 7.61 (td, J=8.1, 4.1 Hz, 1H), 7.23-7.14 (m, 4H), 3.96 (t, J=7.3 Hz, 2H), 3.53 (t, J=4.7 Hz, 4H), 3.40 (s, 2H), 3.33 (s, 2H), 3.10 (q, J=6.6 Hz, 2H), 2.31 (s, 4H), 1.83 (p, J=7.2 Hz, 2H); ¹⁹F NMR (376 MHz, DMSO-d₆) δ −93.40 (dd, J=11.4, 4.2 Hz).

### Example 132

This example demonstrates a synthesis of 4-propyl-1-thioxo-2,4-dihydrofuro[2,3-e][1,2,4]triazolo[4,3-a]pyrimidin-5(1H)-one, 39 (NCK147) in an aspect of the invention.

Methyl 3-(3-propylthioureido)furan-2-carboxylate x was synthesized according to General Procedure D. Compound x was converted to 3-propyl-2-thioxo-2,3-dihydrofuro[3,2-d]pyrimidin-4(1H)-one y according to General Procedure E. Compound y was converted to 4-propyl-1-thioxo-2,4-dihydrofuro[2,3-e][1,2,4]triazolo[4,3-a]pyrimidin-5(1H)-one 39 according to General Procedure K. (5% yield). ¹H NMR (400 MHz, DMSO-d₆): δ 14.09 (s, 1H), 8.90 (d, J=2.0 Hz, 1H), 8.31 (d, J=1.9 Hz, 1H), 3.95 (t, J=7.4 Hz, 2H), 1.73 (m, 2H), 0.92 (t, J=7.5 Hz, 3H); HRMS (ES-API) m/z: calcd. for C₁₀H₁₀N₄O₂S (M+H), 251.0603; found, 251.0607.

### Example 133

This example demonstrates a synthesis of 4-propyl-1-thioxo-2,4-dihydrobenzofuro[2,3-e][1,2,4]triazolo[4,3-a]pyrimidin-5(1H)-one, 40 (NCK148) in an aspect of the invention.

Ethyl 3-isothiocyanatobenzofuran-2-carboxylate x was synthesized according to General Procedure G. Compound x was converted to 3-propyl-2-thioxo-2,3-dihydrobenzofuro[3,2-d]pyrimidin-4(1H)-one y according to General Procedure I. Compound y was converted to 4-propyl-1-thioxo-2,4-dihydrobenzofuro[2,3-e][1,2,4]triazolo[4,3-a]pyrimidin-5(1H)-one 40 according to General Procedure K. (3% yield). ¹H NMR (400 MHz, DMSO-d₆): δ 13.89 (s, 1H), 8.21 (d, J=7.9 Hz, 1H), 7.82 (d, J=8.5 Hz, 1H), 7.70 (t, J=7.4 Hz, 1H), 7.50 (t, J=7.5 Hz, 1H), 4.38 (t, J=7.6 Hz, 2H), 1.73 (m, 2H), 0.93 (t, J=7.4 Hz, 3H); HRMS (ES-API) m/z: calcd. for C14H12N4O2S (M+H), 301.0759; found, 301.0764.

### Example 134

This example demonstrates a synthesis of 10-fluoro-4-propyl-1-thioxo-2,4-dihydrobenzofuro[2,3-e][1,2,4]triazolo[4,3-a]pyrimidin-5(1H)-one, 41 (NCK164) in an aspect of the invention.

Ethyl 4-fluoro-3-isothiocyanatobenzofuran-2-carboxylate x was synthesized according to General Procedure G. Compound x was converted to 9-fluoro-3-propyl-2-thioxo-2,3-dihydrobenzofuro[3,2-d]pyrimidin-4(1H)-one y according to General Procedure I. Compound y was converted to 10-fluoro-4-propyl-1-thioxo-2,4-dihydrobenzofuro[2,3-e][1,2,4]triazolo[4,3-a]pyrimidin-5(1H)-one 41 according to General Procedure K. (4% yield). ¹H NMR (400 MHz, DMSO-d₆): δ 13.99 (s, 1H), 7.77 (d, J=7.8 Hz, 1H), 7.72 (td, J=4.5, 8.0 Hz, 1H), 7.34 (t, J=9.5 Hz, 1H), 4.00 (t, J=7.3 Hz, 2H), 1.74 (m, 2H), 0.93 (t, J=7.4 Hz, 3H); HRMS (ES-API) m/z: calcd. for C14H11FN4O2S (M+H), 319.0665; found, 319.0669.

### Example 135

This example demonstrates a synthesis of 4-propyl-1-thioxo-2,4-dihydrothieno[2,3-e][1,2,4]triazolo[4,3-a]pyrimidin-5(1H)-one, 42 (NCK156) in an aspect of the invention.

Methyl 3-isothiocyanatothiophene-2-carboxylate was synthesized according to General Procedure H. Compound x was converted to 3-propyl-2-thioxo-2,3-dihydrothieno[3,2-d]pyrimidin-4(1H)-one y according to General Procedure J. Compound y was converted to 4-propyl-1-thioxo-2,4-dihydrothieno[2,3-e][1,2,4]triazolo[4,3-a]pyrimidin-5(1H)-one 42 according to General Procedure K. (2% yield)¹H NMR (400 MHz, DMSO-d₆): δ 14.06 (s, 1H), 8.67 (s, 1H), 3.96 (t, J=7.0 Hz, 2H), 2.67 (s, 3H), 1.71 (m, 2H), 0.91 (t, J=7.4 Hz, 3H); HRMS (ES-API) m/z: calcd. for C₁₀H₁₀N₄OS₂ (M+H), 267.0374; found, 267.0369.

### Example 136

This example demonstrates a synthesis of 7-methyl-4-propyl-1-thioxo-2,4-dihydrothieno[2,3-e][1,2,4]triazolo[4,3-a]pyrimidin-5(1H)-one, 43 (NCK149) in an aspect of the invention.

Methyl 3-isothiocyanato-5-methylthiophene-2-carboxylate x was synthesized according to General Procedure H. Compound x was converted to 6-methyl-3-propyl-2-thioxo-2,3-dihydrothieno[3,2-d]pyrimidin-4(1H)-one y according to General Procedure J. Compound y was converted to 7-methyl-4-propyl-1-thioxo-2,4-dihydrothieno[2,3-e][1,2,4]triazolo[4,3-a]pyrimidin-5(1H)-one 43 according to General Procedure K (66% yield). ¹H NMR (400 MHz, DMSO-d₆): δ 14.09 (s, 1H), 8.90 (d, J=5.5 Hz, 1H), 8.31 (d, J=5.3 Hz, 1H), 3.98 (t, J=7.3 Hz, 2H), 1.73 (m, 2H), 0.92 (t, J=7.3 Hz, 3H); HRMS (ES-API) m/z: calcd. for C₁₁H₁₂N₄OS₂ (M+H), 281.0531; found, 281.0531.

### Example 137

This example demonstrates a synthesis of 8-methyl-4-propyl-1-thioxo-2,4-dihydrothieno[2,3-e][1,2,4]triazolo[4,3-a]pyrimidin-5(1H)-one, 44 (NCK157) in an aspect of the invention.

Methyl 3-isothiocyanato-4-methylthiophene-2-carboxylate was synthesized according to General Procedure H. Then, 7-methyl-3-propyl-2-thioxo-2,3-dihydrothieno[3,2-d]pyrimidin-4(1H)-one was synthesized according to General Procedure J. Then, 8-methyl-4-propyl-1-thioxo-2,4-dihydrothieno[2,3-e][1,2,4]triazolo[4,3-a]pyrimidin-5(1H)-one was synthesized according to General Procedure K. (27% yield). ¹H NMR (400 MHz, DMSO-d₆): δ 13.91 (s, 1H), 7.89 (s, 1H), 3.96 (t, J=1.8 Hz, 2H), 2.98 (s, 3H), 1.70 (m, 2H), 0.90 (t, J=7.4 Hz, 3H); HRMS (ES-API) m/z: calcd. for C₁₁H₁₂N₄OS₂ (M+H), 281.0531; found, 281.0528.

### Example 138

This example demonstrates a synthesis of 7-chloro-4-propyl-1-thioxo-2,4-dihydrothieno[2,3-e][1,2,4]triazolo[4,3-a]pyrimidin-5(1H)-one, 45 (NCK158) in an aspect of the invention.

Methyl 5-chloro-3-isothiocyanatothiophene-2-carboxylate was synthesized according to General Procedure H. Then, 6-chloro-3-propyl-2-thioxo-2,3-dihydrothieno[3,2-d]pyrimidin-4(1H)-one was synthesized according to General Procedure J. Then, 7-chloro-4-propyl-1-thioxo-2,4-dihydrothieno[2,3-e][1,2,4]triazolo[4,3-a]pyrimidin-5(1H)-one was synthesized according to General Procedure K. (5% yield). ¹H NMR (400 MHz, DMSO-d₆): δ 14.16 (s, 1H), 8.89 (s, 1H), 3.97 (t, J=7.3 Hz, 2H), 1.71 (m, 2H), 0.91 (t, J=7.4 Hz, 3H); HRMS (ES-API) m/z: calcd. for C₁₀H₉ClN₄OS₂ (M+H), 300.9985; found, 300.9982.

### Example 139

This example demonstrates a synthesis of 7-(tert-butyl)-4-propyl-1-thioxo-2,4-dihydrothieno[2,3-e][1,2,4]triazolo[4,3-a]pyrimidin-5(1H)-one, 46 (NCK165) in an aspect of the invention.

Methyl 5-(tert-butyl)-3-isothiocyanatothiophene-2-carboxylate was synthesized according to General Procedure H. Then, 6-(tert-butyl)-3-propyl-2-thioxo-2,3-dihydrothieno[3,2-d]pyrimidin-4(1H)-one was synthesized according to General Procedure J. Then, 7-(tert-butyl)-4-propyl-1-thioxo-2,4-dihydrothieno[2,3-e][1,2,4]triazolo[4,3-a]pyrimidin-5(1H)-one was synthesized according to General Procedure K. (70% yield). ¹H NMR (400 MHz, DMSO-d₆): δ 14.05 (s, 1H), 8.78 (s, 1H), 3.97 (t, J=7.2 Hz, 2H), 1.71 (m, 2H), 1.43 (s, 9H), 0.91 (t, J=7.4 Hz, 3H); HRMS (ES-API) m/z: calcd. for C₁₄H₁₈N₄OS₂ (M+H), 323.1000; found, 323.1003.

### Example 140

This example demonstrates a synthesis of 7-phenyl-4-propyl-1-thioxo-2,4-dihydrothieno[2,3-e][1,2,4]triazolo[4,3-a]pyrimidin-5(1H)-one, 47 (NCK166) in an aspect of the invention.

Methyl 3-isothiocyanato-5-phenylthiophene-2-carboxylate was synthesized according to General Procedure H. Then, 6-phenyl-3-propyl-2-thioxo-2,3-dihydrothieno[3,2-d]pyrimidin-4(1H)-one was synthesized according to General Procedure J. Then, 7-phenyl-4-propyl-1-thioxo-2,4-dihydrothieno[2,3-e][1,2,4]triazolo[4,3-a]pyrimidin-5(1H)-one was synthesized according to General Procedure K. (5% yield). ¹H NMR (400 MHz, DMSO-d₆): δ 14.12 (s, 1H), 9.21 (s, 1H), 7.80 (m, 2H), 7.51-7.58 (m, 3H), 3.99 (t, J=6.7 Hz, 2H), 1.73 (m, 2H), 0.93 (t, J=7.4 Hz, 3H); HRMS (ES-API) m/z: calcd. for C₁₆H₁₄N₄OS₂ (M+H), 343.0687; found, 343.0683.

### Example 141

This example demonstrates a synthesis of 4-propyl-1-thioxo-2,4-dihydrobenzo[4,5]thieno[2,3-e][1,2,4]triazolo[4,3-a]pyrimidin-5(1H)-one, 48 (NCK162) in an aspect of the invention.

Methyl 3-(3-propylthioureido)benzo[b]thiophene-2-carboxylate was synthesized according to General Procedure D. Then, 3-propyl-2-thioxo-2,3-dihydrobenzo[4,5]thieno[3,2-d]pyrimidin-4(1H)-one was synthesized according to General Procedure E under condition of 50° C. Then, 4-propyl-1-thioxo-2,4-dihydrobenzo[4,5]thieno[2,3-e][1,2,4]triazolo[4,3-a]pyrimidin-5(1H)-one was synthesized according to General Procedure K. (21% yield). ¹H NMR (400 MHz, DMSO-d₆): δ 14.14 (s, 1H), 10.66 (s, 1H), 8.17 (d, J=8.3 Hz, 1H), 7.62 (t, J=7.2 Hz, 1H),), 4.02 (t, J=7.0 Hz, 2H), 1.75 (m, 2H), 0.93 (t, J=7.4 Hz, 3H); HRMS (ES-API) m/z: calcd. for C₁₄H₁₂N₄OS₂ (M+H), 317.0531; found, 317.0534.

### Example 142

This example demonstrates a synthesis of 5-propyl-8-thioxo-7,8-dihydrothieno[3,2-e][1,2,4]triazolo[4,3-a]pyrimidin-4(5H)-one, 49a (NCK163) an aspect of the invention.

Methyl 2-(3-propylthioureido)thiophene-3-carboxylate was synthesized according to General Procedure D using DMF as solvent. Then, 3-propyl-2-thioxo-2,3-dihydrothieno[2,3-d]pyrimidin-4(1H)-one was synthesized according to General Procedure F. Then, 5-propyl-8-thioxo-7,8-dihydrothieno[3,2-e][1,2,4]triazolo[4,3-a]pyrimidin-4(5H)-one was synthesized according to General Procedure K. (20% yield). ¹H NMR (400 MHz, DMSO-d₆): δ 14.16 (s, 1H), 7.52 (d, J=5.5 Hz, 1H), 7.45 (t, J=5.5 Hz, 1H), 3.97 (t, J=7.0 Hz, 2H), 1.72 (m, 2H), 0.92 (t, J=7.5 Hz, 3H); HRMS (ES-API) m/z: calcd. for C₁₀H₁₀N₄OS₂ (M+H), 267.0374; found, 267.0373.

### Example 143

This example demonstrates a synthesis of 8-fluoro-4-propyl-1-thioxo-2,4-dihydrobenzofuro[2,3-e][1,2,4]triazolo[4,3-a]pyrimidin-5(1H)-one, 49d (NCK178) in an aspect of the invention.

Methyl 6-fluoro-3-isothiocyanatobenzofuran-2-carboxylate x was synthesized according to General Procedure G. Compound x was converted to 7-fluoro-3-propyl-2-thioxo-2,3-dihydrobenzofuro[3,2-d]pyrimidin-4(1H)-one carboxylate y according to General Procedure I (64% yield). Then, 8-fluoro-4-propyl-1-thioxo-2,4-dihydrobenzofuro[2,3-e][1,2,4]triazolo[4,3-a]pyrimidin-5(1H)-one was synthesized according to General Procedure K (13% yield).

¹H NMR (400 MHz, DMSO-d₆) δ: 10.10 (bs, 1H), 7.84 (d, J=7.3 Hz, 1H), 7.36-7.40 (m, 1H), 6.51 (s, 1H), 4.00 (t, J=6.7 Hz, 2H), 1.71 (m, 2H), 0.90 (t, J=7.3 Hz, 3H). ¹⁹F NMR (376 MHz, DMSO-d₆): δ 109.75

### Example 144

This example demonstrates a synthesis of 4-propyl-1-thioxo-2,4-dihydro-1H-pyrrolo[2,3-e][1,2,4]triazolo[4,3-a]pyrimidin-5(6H)-one, 49b (NCK179) in an aspect of the invention.

Methyl 3-isothiocyanato-1H-pyrrole-2-carboxylate x was synthesized according to General Procedure H. Compound x was converted to 3-propyl-2-thioxo-1,2,3,5-tetrahydro-4H-pyrrolo[3,2-d]pyrimidin-4-one y according to General Procedure J. Compound y was converted to 4-propyl-1-thioxo-2,4-dihydro-1H-pyrrolo[2,3-e][1,2,4]triazolo[4,3-a]pyrimidin-5(6H)-one z according to General Procedure K (71% yield). ¹H NMR (400 MHz, DMSO-d₆) δ: 12.79 (s, 1H), 12.21 (s, 1H), 7.30 (s, 1H), 5.95 (s, 1H), 4.33 (t, J=6.7 Hz, 2H), 1.66 (m, 2H), 0.89 (t, J=7.3 Hz, 3H).

### Example 145

This example demonstrates a synthesis of 4-(cyclopropylmethyl)-7-methyl-1-thioxo-2,4-dihydrothieno[2,3-e][1,2,4]triazolo[4,3-a]pyrimidin-5(1H)-one, 49e (NCK181) in an aspect of the invention.

Methyl 3-isothiocyanato-5-methylthiophene-2-carboxylate x was synthesized according to General Procedure H. Compound x was converted to 3-(cyclopropylmethyl)-6-methyl-2-thioxo-2,3-dihydrothieno[3,2-d]pyrimidin-4(1H)-one y according to General Procedure J. Compound y was converted to 4-(cyclopropylmethyl)-7-methyl-1-thioxo-2,4-dihydrothieno[2,3-e][1,2,4]triazolo[4,3-a]pyrimidin-5(1H)-one 49e according to General Procedure K (77% yield). ¹H NMR (400 MHz, DMSO-d₆): δ 14.09 (s, 1H), 8.68 (s, 1H), 3.90 (t, J=7.1 Hz, 2H), 2.67 (s, 3H), 1.27 (m, 1H), 0.41-0.49 (m, 4H); HRMS (ES-API) m/z: calcd. for C₁₂H₁₃N₄OS₂ (M+H), 293.0531; found, 293.0527.

### Example 146

This example demonstrates a synthesis of 4-ethyl-1-(ethylthio)-9-fluoro-[1,2,4]triazolo[4,3-a]quinazolin-5(4H)-one, 60 (NCK139) in an aspect of the invention.

1-(Ethylthio)-9-fluoro-4-propyl-[1,2,4]triazolo[4,3-a]quinazolin-5(4H)-one was synthesized according to General Procedure C using 9-fluoro-4-(propyl-d7)-1-thioxo-2,4-dihydro-[1,2,4]triazolo[4,3-a]quinazolin-5(1H)-one (27) as starting material (86% yield). ¹H NMR (400 MHz, DMSO-d₆): δ 8.10 (d, J=6.4 Hz, 1H), 7.86 (m, 1H), 7.63 (td, J=4.5, 8.0 Hz, 1H), 4.14 (t, J=7.4 Hz, 2H), 3.24 (q, J=7.3 Hz, 2H), 1.77 (m, 2H), 1.35 (t, J=7.2 Hz, 3H) 0.94 (t, J=7.49 Hz, 3H); ¹⁹F NMR (376 MHz, DMSO-d₆): δ −112.06 (dd, J=4.4, 12.8 Hz); HRMS (ES-API) m/z: calcd. for C₁₄H₁₅FN₄OS (M+H), 307.1029; found, 307.1025.

### Example 147

This example demonstrates a synthesis of 9-fluoro-1-(methylthio)-4-(propyl-d₇)-[1,2,4]triazolo[4,3-a]quinazolin-5(4H)-one, 61 (NCK111) in an aspect of the invention.

9-Fluoro-1-(methylthio)-4-(propyl-d7)-[1,2,4]triazolo[4,3-a]quinazolin-5(4H)-one was synthesized according to General Procedure C using 9-fluoro-4-(propyl-d7)-1-thioxo-2,4-dihydro-[1,2,4]triazolo[4,3-a]quinazolin-5(1H)-one (27) as starting material (57% yield). ¹H NMR (400 MHz, DMSO-d₆): δ 8.11 (d, J=7.1 Hz, 1H), 7.88 (m, 1H), 7.63 (td, J=4.6, 8.2 Hz, 1H), 2.68 (s, 3H); ¹⁹F NMR (376 MHz, DMSO-d₆): δ −113.07 (dd, J=4.2, 13.3 Hz); HRMS (ES-API) m/z: calcd. for C₁₃H₆D₇FN₄OS (M+H), 300.1312; found, 300.1307.

### Example 148

This example demonstrates a synthesis of 1-(ethylthio)-9-fluoro-4-(propyl-d7)-[1,2,4]triazolo[4,3-a]quinazolin-5(4H)-one, 62 (NCK146) in an aspect of the invention.

1-(Ethylthio)-9-fluoro-4-(propyl-d7)-[1,2,4]triazolo[4,3-a]quinazolin-5(4H)-one was synthesized according to General Procedure C using 9-fluoro-4-(propyl-d7)-1-thioxo-2,4-dihydro-[1,2,4]triazolo[4,3-a]quinazolin-5(1H)-one (27) as starting material (91% yield). ¹H NMR (400 MHz, DMSO-d₆): δ 8.10 (d, J=7.8 Hz, 1H), 7.87 (m, 1H), 7.63 (td, J=4.6, 8.2 Hz, 1H), 3.24 (q, J=7.2 Hz), 1.34 (t, J=7.3 Hz, 3H); HRMS (ES-API) m/z: calcd. for C₁₄H₈D₇FN₄OS (M+H), 314.1468; found, 314.1467.

### Example 149

This example demonstrates a synthesis of 9-fluoro-1-((methyl-d₃)thio)-4-(propyl-d₇)-[1,2,4]triazolo[4,3-a]quinazolin-5(4H)-one, 63 (NCK141) in an aspect of the invention.

9-Fluoro-1-((methyl-d₃)thio)-4-(propyl-d7)-[1,2,4]triazolo[4,3-a]quinazolin-5(4H)-one was synthesized according to General Procedure C using 9-fluoro-4-(propyl-d7)-1-thioxo-2,4-dihydro-[1,2,4]triazolo[4,3-a]quinazolin-5(1H)-one (27) as starting material (59% yield). ¹H NMR (400 MHz, DMSO-d₆): δ 8.11 (d, J=6.9 Hz, 1H), 7.87 (m, 1H), 7.63 (td, J=4.6, 8.0 Hz, 1H); ¹⁹F NMR (376 MHz, DMSO-d₆): δ −113.01 (dd, J=4.8, 13.1 Hz); HRMS (ES-API) m/z: calcd. for C₁₃H₃D₁₀FN₄OS (M+H), 303.1500; found, 303.1497.

### Example 150

This example demonstrates a synthesis of 9-fluoro-1-((methyl-d₃)thio)-4-propyl-[1,2,4]triazolo[4,3-a]quinazolin-5(4H)-one, 64 (NCK140) in an aspect of the invention.

9-Fluoro-1-((methyl-d₃)thio)-4-propyl-[1,2,4]triazolo[4,3-a]quinazolin-5(4H)-one was synthesized according to General Procedure C using 9-fluoro-4-(propyl-d7)-1-thioxo-2,4-dihydro-[1,2,4]triazolo[4,3-a]quinazolin-5(1H)-one (27) as starting material (71% yield). ¹H NMR (400 MHz, DMSO-d₆): δ 8.11 (d, J=7.2 Hz, 1H), 7.87 (m, 1H), 7.63 (td, J=4.5, 8.0 Hz, 1H), 4.14 (t, J=7.3 Hz, 2H), 1.77 (m, 2H), 0.94 (t, J=7.4 Hz, 3H); ¹⁹F NMR (376 MHz, DMSO-d₆): δ −113.01 (dd, J=4.9, 13.2 Hz); HRMS (ES-API) m/z: calcd. for C₁₃H₁₀D₃FN₄OS (M+H), 296.1061; found, 296.1058.

### Example 151

This example demonstrates a synthesis of N-(3-(1-(Ethylthio)-9-fluoro-5-oxo-[1,2,4]triazolo[4,3-a]quinazolin-4(5H)-yl)propyl)-2-(3-methoxyphenyl)acetamide, 65 (NCK153) in an aspect of the invention.

To a solution of N-(3-(9-fluoro-5-oxo-1-thioxo-1,2-dihydro-[1,2,4]triazolo[4,3-a]quinazolin-4(5H)-yl)propyl)-2-(3-methoxyphenyl)acetamide, 32 (0.04 g, 0.091 mmol) in DMF (0.453 mL) were added K₂CO₃ (0.015 g, 0.109 mmol) and iodoethane (8.79 μl, 0.109 mmol). The reaction mixture was quenched with methanol and concentrated. The product was then purified by RP-ISCO (H₂O/MeCN+0.1% TFA, 10-100%); ¹H NMR (400 MHz, DMSO-d₆) δ 8.07 (d, J=7.8 Hz, 1H), 8.02 (t, J=6.1 Hz, 1H), 7.84 (ddd, J=12.8, 8.3, 1.5 Hz, 1H), 7.61 (td, J=8.1, 4.5 Hz, 1H), 7.17 (t, J=8.0 Hz, 1H), 6.84-6.78 (m, 2H), 6.75 (dd, J=7.9, 2.6 Hz, 1H), 4.17 (t, J=7.2 Hz, 2H), 3.71 (s, 3H), 3.22 (q, J=7.3 Hz, 2H), 3.13 (q, J=6.7 Hz, 2H), 1.88 (p, J=7.0 Hz, 2H), 1.32 (t, J=7.3 Hz, 3H); ¹⁹F NMR (376 MHz, DMSO-d₆) δ −112.66 (dd, J=12.6, 4.4 Hz).

### Example 152

This example demonstrates a synthesis of 2-(4-cyanophenyl)-N-(3-(1-(ethylthio)-9-fluoro-5-oxo-[1,2,4]triazolo[4,3-a]quinazolin-4(5H)-yl)propyl)acetamide, 66 (NCK154) in an aspect of the invention.

To a solution of 2-(4-cyanophenyl)-N-(3-(9-fluoro-5-oxo-1-thioxo-1,2-dihydro-[1,2,4]triazolo[4,3-a]quinazolin-4(5H)-yl)propyl)acetamide, 34 (0.03 g, 0.069 mmol) in DMF (0.344 mL) were added K₂CO₃ (0.011 g, 0.082 mmol) and iodoethane (6.67 μl, 0.082 mmol). The reaction mixture was quenched with methanol and concentrated. The product was then purified by RP-ISCO (H₂O/MeCN+0.1% TFA, 10-100%); ¹H NMR (400 MHz, DMSO-d₆) δ 8.19 (d, J=5.3 Hz, 1H), 8.08 (d, J=7.8 Hz, 1H), 7.84 (dd, J=12.7, 8.2 Hz, 1H), 7.75 (d, J=8.2 Hz, 2H), 7.61 (td, J=8.1, 4.5 Hz, 1H), 7.46 (d, J=8.0 Hz, 2H), 4.17 (t, J=7.3 Hz, 2H), 3.50 (s, 2H), 3.28-3.09 (m, 4H), 1.89 (q, J=7.1 Hz, 2H), 1.33 (t, J=7.3 Hz, 3H); ¹⁹F NMR (376 MHz, DMSO-d₆) δ −112.62-−112.73 (m).

### Example 153

This example demonstrates a synthesis of 1-(ethylthio)-9-hydroxy-4-propyl-[1,2,4]triazolo[4,3-a]quinazolin-5(4H)-one, 67 (NCK145) in an aspect of the invention.

To a solution of 9-hydroxy-4-propyl-1-thioxo-2,4-dihydro-[1,2,4]triazolo[4,3-a]quinazolin-5(1H)-one, 18 (0.03 g, 0.109 mmol) in DMF (0.543 mL) were added K₂CO₃ (0.018 g, 0.130 mmol) and iodoethane (10.53 μl, 0.130 mmol). The reaction mixture was quenched with methanol and concentrated. The product was then purified by RP-ISCO (C18, H₂O/MeCN+0.1% NH₄OH, 10-100%); ¹H NMR (400 MHz, DMSO-d₆) δ 11.06 (s, 1H), 7.80 (dd, J=7.7, 1.6 Hz, 1H), 7.52 (t, J=7.9 Hz, 1H), 7.36 (dd, J=8.1, 1.6 Hz, 1H), 4.26 (q, J=7.2 Hz, 2H), 4.01-3.93 (m, 2H), 1.71 (h, J=7.5 Hz, 2H), 1.35 (t, J=7.2 Hz, 3H), 0.90 (t, J=7.4 Hz, 3H).

### Example 154

This example demonstrates a synthesis of 9-fluoro-4-propyl-1-(propylthio)-[1,2,4]triazolo[4,3-a]quinazolin-5(4H)-one, 68 (NCK150) in an aspect of the invention.

9-Fluoro-4-propyl-1-(propylthio)-[1,2,4]triazolo[4,3-a]quinazolin-5(4H)-one was synthesized according to General Procedure C using 9-fluoro-4-propyl-1-thioxo-2,4-dihydro-[1,2,4]triazolo[4,3-a]quinazolin-5(1H)-one (14) as starting material (87% yield). ¹H NMR (400 MHz, DMSO-d₆): δ 8.11 (d, J=8.2 Hz, 1H), 7.86 (m, 1H), 7.64 (td, J=4.6, 8.0 Hz, 1H), 4.14 (t, J=7.0 Hz, 2H), 3.21 (t, J=6.9 Hz, 2H), 1.68-1.80 (m, 4H), 0.92-1.00 (m, 6H); ¹⁹F NMR (376 MHz, DMSO-d₆): δ −112.60 (dd, J=4.4, 12.8 Hz); HRMS (ES-API) m/z: calcd. for C₁₅H₁₇FN₄OS (M+H), 321.1185; found, 321.1184.

### Example 155

This example demonstrates a synthesis of 1-(butylthio)-9-fluoro-4-propyl-[1,2,4]triazolo[4,3-a]quinazolin-5(4H)-one, 69 (NCK151) in an aspect of the invention.

1-(Butylthio)-9-fluoro-4-propyl-[1,2,4]triazolo[4,3-a]quinazolin-5(4H)-one (NCK151). 1-(butylthio)-9-fluoro-4-propyl-[1,2,4]triazolo[4,3-a]quinazolin-5(4H)-one was synthesized according to General Procedure C using 9-fluoro-4-propyl-1-thioxo-2,4-dihydro-[1,2,4]triazolo[4,3-a]quinazolin-5(1H)-one (14) as starting material (92% yield). ¹H NMR (400 MHz, DMSO-d₆): δ 8.11 (d, J=7.9 Hz, 1H), 7.86 (m, 1H), 7.64 (td, J=4.8, 8.3 Hz, 1H), 4.14 (t, J=6.9 Hz, 2H), 3.23 (t, J=7.1 Hz, 2H), 1.75 (m, 2H), 1.69 (m, 2H), 1.38 (m, 2H), 0.88-0.95 (m, 6H); HRMS (ES-API) m/z: calcd. for C₁₆H₁₉FN₄OS (M+H), 335.1342; found, 335.1344.

### Example 156

This example demonstrates a synthesis of 9-fluoro-1-(isopropylthio)-4-propyl-[1,2,4]triazolo[4,3-a]quinazolin-5(4H)-one, 70 (NCK152) in an aspect of the invention.

9-Fluoro-1-(isopropylthio)-4-propyl-[1,2,4]triazolo[4,3-a]quinazolin-5(4H)-one was synthesized according to General Procedure C using 9-fluoro-4-propyl-1-thioxo-2,4-dihydro-[1,2,4]triazolo[4,3-a]quinazolin-5(1H)-one (14) as starting material (78% yield). ¹H NMR (400 MHz, DMSO-d₆): δ 8.10 (d, J=7.8 Hz, 1H), 7.86 (m, 1H), 7.63 (td, J=4.6, 8.0 Hz, 1H), 4.15 (t, J=7.2 Hz, 2H), 3.87 (m, 1H), 1.76 (m, 2H), 1.36 (d, J=6.7 Hz, 2H), 0.93 (t, J=7.4 Hz, 3H); ¹⁹F NMR (376 MHz, DMSO-d₆): δ −111.88 (d, J=12.9 Hz); HRMS (ES-API) m/z: calcd. for C₁₅H₁₇FN₄OS (M+H), 321.1186; found, 321.1185.

### Example 157

This example demonstrates a synthesis of 1,1′-disulfanediylbis(9-fluoro-4-propyl-[1,2,4]triazolo[4,3-a]quinazolin-5(4H)-one), 71 (NCK155) in an aspect of the invention.

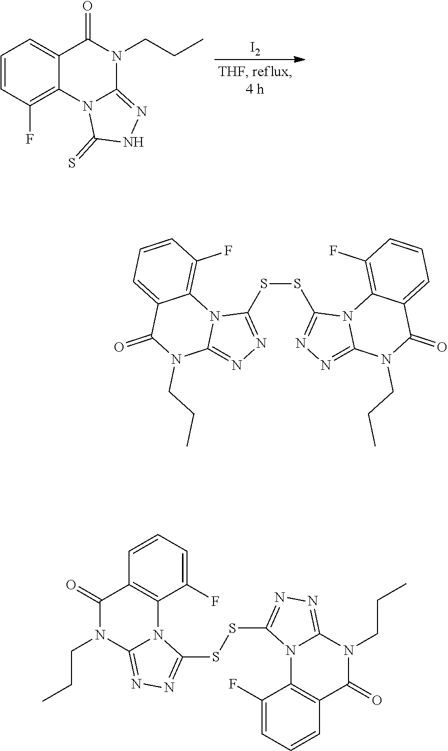

To a solution of 9-fluoro-4-propyl-1-thioxo-2,4-dihydro-[1,2,4]triazolo[4,3-a]quinazolin-5(1H)-one x (0.1 g, 0.359 mmol) in THF (35.9 mL) was added iodine (9.12 mg, 0.036 mmol), the reaction heated to 65° C. for 5 h, and the volume was reduced. The residue was then purified by ISCO (EtOAc/hexanes, 0-100%) followed by a wash with 0.01M Na₂S₂O₅, then repurified by ISCO (EtOAc/hexanes 0-100%); ¹H NMR (400 MHz, DMSO-d₆) δ 7.99 (d, J=7.8 Hz, 2H), 7.74-7.64 (m, 2H), 7.58 (td, J=8.0, 4.3 Hz, 2H), 4.08 (t, J=7.4 Hz, 4H), 1.71 (h, J=7.5 Hz, 4H), 0.91 (t, J=7.4 Hz, 7H); ¹⁹F NMR (376 MHz, DMSO-d₆) δ −107.82 (dd, J=12.4, 4.3 Hz).

### Example 158

This example demonstrates a synthesis of 1-(ethyldisulfaneyl)-9-fluoro-4-propyl-[1,2,4]triazolo[4,3-a]quinazolin-5(4H)-one, 72 (NCK161) in an aspect of the invention.

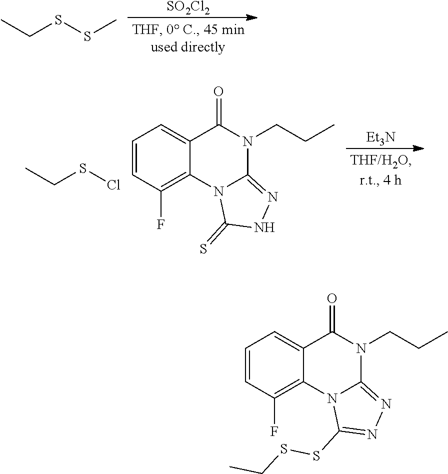

To a solution of 1,2-diethyldisulfane (0.01 mL, 0.081 mmol) in THF (0.812 mL) at 0° C. was added sulfuryl chloride (7.26 μl, 0.089 mmol) dropwise. The reaction mixture was stirred at 0° C. for 30 min and used directly in the next reaction.

To a solution of 9-fluoro-4-propyl-1-thioxo-2,4-dihydro-[1,2,4]triazolo[4,3-a]quinazolin-5(1H)-one (0.04 g, 0.144 mmol), triethylamine (0.044 mL, 0.316 mmol), and triethylamine (0.044 mL, 0.316 mmol) in THF (0.575 mL) was added a solution of ethanesulfenyl chloride (0.014 g, 0.144 mmol) (CNA011-022). The reaction mixture was stirred at room temperature for 1-6 h. The reaction mixture was diluted with DCM and washed with sat. NaHCO₃ then water. The organic layer was dried over MgSO₄, filtered, and concentrated. The product was purified by RP-ISCO (C18, H₂O/MeCN+0.1% NH₄OH, 10-100%); ¹H NMR (400 MHz, DMSO-d₆) δ 8.11 (d, J=7.9 Hz, 1H), 7.94-7.84 (m, 1H), 7.71-7.61 (m, 1H), 4.15 (dd, J=8.3, 6.5 Hz, 2H), 2.93 (q, J=7.3 Hz, 2H), 1.83-1.69 (m, 2H), 1.29 (td, J=7.3, 1.5 Hz, 3H), 0.92 (td, J=7.4, 1.5 Hz, 3H); ¹⁹F NMR (376 MHz, DMSO-d₆) δ −110.08 (dd, J=12.5, 4.5 Hz).

### Example 159

This example demonstrates a synthesis of 9-Fluoro-1-((1-methyl-4-nitro-1H-imidazol-5-yl)thio)-4-propyl-[1,2,4]triazolo[4,3-a]quinazolin-5(4H)-one, 73 in an aspect of the invention.

A mixture of 9-fluoro-4-propyl-1-thioxo-2,4-dihydro-[1,2,4]triazolo[4,3-a]quinazolin-5(1H)-one (14) (1.0 equiv.), water (0.23 mL) and sodium hydroxide (1.1 equiv.) was stirred until a solution formed. 5-Chloro-1-methyl-4-nitroimidazole (1.0 equiv.) was add to the reaction mixture and the mixture stirred for 1 h at room temperature. After completion, the suspension was neutralized with acetic acid and a solid precipitated. The precipitate was filtered and washed with water and recrystallized from ethanol. (45% yield). ¹H NMR (400 MHz, DMSO-d₆): δ 8.18 (s, 1H), 8.16 (d, J=9.7 Hz, 1H), 7.99 (m, 1H), 7.71 (m, 1H), 4.11 (t, J=6.7 Hz, 2H), 3.74 (s, 3H), 1.73 (m, 2H), 0.89 (t, J=7.3 Hz, 3H); ¹⁹F NMR (376 MHz, DMSO-d₆): δ −115.03 (dd, J=4.8, 13.1 Hz); HRMS (ES-API) m/z: calcd. for C₁₆H₁₄FN₇O₃S (M+H), 404.0941; found, 404.0941.

### Example 160

This example demonstrates a synthesis of 9-fluoro-1-((1-methyl-4-nitro-1H-imidazol-5-yl)thio)-4-propyl-[1,2,4]triazolo[4,3-a]quinazolin-5(4H)-one 73 (NCK167) in an aspect of the invention.

9-fluoro-1-((1-methyl-4-nitro-1H-imidazol-5-yl)thio)-4-propyl-[1,2,4]triazolo[4,3-a]quinazolin-5(4H)-one was synthesized according to General Procedure L, as a beige solid (45% yield).

¹H NMR (400 MHz, DMSO-d₆) δ: 8.18 (s, 1H), 8.16 (d, J=9.7 Hz, 1H), 7.99 (m, 1H), 7.71 (m, 1H), 4.11 (t, J=6.7 Hz, 2H), 3.74 (s, 3H), 1.73 (m, 2H), 0.89 (t, J=7.3 Hz, 3H). HRMS (ES-API) m/z: for C₁₆H₁₄FN₇O₃S calcd. 404.0941; found, 404.0941.

### Example 161

This example demonstrates a synthesis of 8-methyl-1-((1-methyl-4-nitro-1H-imidazol-5-yl)thio)-4-propylthieno[2,3-e][1,2,4]triazolo[4,3-a]pyrimidin-5(4H)-one 74 (NCK170) in an aspect of the invention.

8-Methyl-1-((1-methyl-4-nitro-1H-imidazol-5-yl)thio)-4-propylthieno[2,3-e][1,2,4]triazolo[4,3-a]pyrimidin-5(4H)-one was synthesized according to General Procedure L, and isolated as a beige solid (40% yield).

¹H NMR (400 MHz, DMSO-d₆) δ: 8.13 (s, 1H), 8.03 (S, 1H), 4.11 (t, J=6.7 Hz, 2H), 3.83 (s, 3H), 2.90 (s, 3H) 1.71 (m, 2H), 0.87 (t, J=7.3 Hz, 3H). ¹⁹F NMR (376 MHz, DMSO-d₆): δ −115.03 (dd, J=4.8, 13.1 Hz). HRMS (ES-API) m/z: for C₁₅H₁₅N₇O₃OS₂ calcd. 406.0756; found, 406.0753.

### Example 162

This example demonstrates a synthesis of 7-methyl-1-((1-methyl-4-nitro-1H-imidazol-5-yl)thio)-4-propylthieno[2,3-e][1,2,4]triazolo[4,3-a]pyrimidin-5(4H)-one 75 (NCK173) in an aspect of the invention.

7-Methyl-1-((1-methyl-4-nitro-1H-imidazol-5-yl)thio)-4-propylthieno[2,3-e][1,2,4]triazolo[4,3-a]pyrimidin-5(4H)-one was synthesized according to General Procedure L, and isolated as a white solid (75% yield).

¹H NMR (400 MHz, DMSO-d₆) δ: 6 8.12 (s, 1H), 7.95 (s, 1H), 4.11 (t, J=7.3 Hz, 2H), 3.83 (s, 3H), 2.62 (s, 3H), 1.70-1.76 (m, 2H), 0.89 (t, J=7.3 Hz, 3H). HRMS (ES-API) m/z: for C₁₅H₁₅N₇O₃S₂ calcd. 406.0756; found, 406.0753.

### Example 163

This example demonstrates a synthesis of 7-methyl-1-((1-methyl-4-(trifluoromethyl)-1H-imidazol-5-yl)thio)-4-propylthieno[2,3-e][1,2,4]triazolo[4,3-a]pyrimidin-5(4H)-one 76 (NCK174) in an aspect of the invention.

7-Methyl-1-((1-methyl-4-(trifluoromethyl)-1H-imidazol-5-yl)thio)-4-propylthieno[2,3-e][1,2,4]triazolo[4,3-a]pyrimidin-5(4H)-one was synthesized according to General Procedure L, and isolated as a white solid (52% yield).

¹H NMR (400 MHz, DMSO-d₆) δ: 8.67 (s, 1H), 6.78 (s, 1H), 3.95 (t, J=7.3 Hz, 2H), 3.29 (s, 3H), 2.66 (s, 3H), 1.71 (t, J=7.3 Hz, 2H), 0.90 (J=7.3 Hz, 3H). ¹⁹F NMR (376 MHz, DMSO-d₆): δ: −60.17.

### Example 164

This example demonstrates a synthesis of 1-methyl-5-((7-methyl-5-oxo-4-propyl-4,5-dihydrothieno[2,3-e][1,2,4]triazolo[4,3-a]pyrimidin-1-yl)thio)-1H-imidazole-4-sulfonamide 77 (NCK175) in an aspect of the invention.

1-Methyl-5-((7-methyl-5-oxo-4-propyl-4,5-dihydrothieno[2,3-e][1,2,4]triazolo[4,3-a]pyrimidin-1-yl)thio)-1H-imidazole-4-sulfonamide was synthesized according to General Procedure L, and isolated as a white solid (34% yield).

¹H NMR (400 MHz, DMSO-d₆) δ: 8.66 (s, 1H), 6.77 (s, 1H), 3.95 (t, J=7.3 Hz, 2H), 3.32 (s, 3H), 2.66 (s, 3H), 1.69 (t, J=7.3 Hz, 2H), 0.90 (J=7.3 Hz, 3H).

### Example 165

This example demonstrates a synthesis of 4-(cyclopropylmethyl)-7-methyl-1-((1-methyl-4-nitro-1H-imidazol-5-yl)thio)thieno[2,3-e][1,2,4]triazolo[4,3-a]pyrimidin-5(4H)-one, 85 (NCK182) in an aspect of the invention.

4-(cyclopropylmethyl)-7-methyl-1-((1-methyl-4-nitro-TH-imidazol-5-yl)thio)thieno[2,3-e][1,2,4]triazolo[4,3-a]pyrimidin-5(4H)-one was synthesized according to General Procedure L using 4-(cyclopropylmethyl)-7-methyl-1-thioxo-2,4-dihydrothieno[2,3-e][1,2,4]triazolo[4,3-a]pyrimidin-5(1H)-one (49e) as starting material (74% yield). ¹H NMR (400 MHz, DMSO-d₆): δ 8.13 (s, 1H), 7.97 (s, 1H), 4.05 (d, J=7.2 Hz, 2H), 3.85 (s, 3H), 2.70 (s, 3H), 1.32 (m, 1H), 0.46 (m, 4H); HRMS (ES-API) m/z: calcd. for C₁₆H₁₆N₇O₃S₂ (M+H), 418.0756; found, 418.0757.

### Example 166

This example describes a second Structure Activity Relationship (SAR) study performed for the compound of formula (I) in an aspect of the invention.

ELISA-based PBD-binding Inhibition Assay. The assay was performed essentially as described previously (Yun et al., Nat. Struct. Mol. Biol. 16, 876-882. (2009)), using a highly specific Biotin-Ahx-C-ETFDPPLHS-pT-AI-NH₂) peptide (Kang et al., Mol. Cell 24, 409-422. (2006)) and the full-length human Plk1 expressed in HEK293A cells. The reaction products were measured at 450 nm by using a Perkin-Elmer Enspire Multimode Plate reader (PerkinElmer, Inc., Boston, MA). Data obtained from more than three independent experiments were analyzed by GraphPad (San Diego, CA) Prism software version 7.

PBD Fluorescence Polarization (FP) Binding Assays for Plk1 specificity. FP assays were carried out essentially as described previously (Liu et al., Serendipitous alkylation of a Plk1 ligand uncovers a new binding channel. Nat. Chem. Biol. 7, 595-601 (2011); and Alverez et al., Identification of a New Heterocyclic Scaffold for Inhibitors of the Polo-Box Domain of Polo-like Kinase 1. J. Med. Chem. 63, 14087-14117 (2020)). Samples were analyzed approximately 30 min after mixing all components in a 384-well format using the Molecular Devices (San Jose, CA) SpectraMax Paradigm multi-mode microplate detection platform. All experiments were performed in triplicate for each sample. Obtained data were plotted using GraphPad Prism software version 7.

MTS Assay. A tetrazolium compound [3-(4,5-dimethylthiazol-2-yl)-5-(3-carboxymethoxyphenyl)-2-(4-sulfophenyl)-2H-tetrazolium, inner salt; MTS]-based cell proliferation assay was carried out using the CELLTITER 96™ AQᵤₑₒᵤₛ One Solution Cell Proliferation Assay (MTS) kit (Promega, WI). Briefly, asynchronously growing L363 cells cultured in a 96-well plate were treated with the indicated compounds for 48 h. The cells in 100 μL of culture medium were then treated with 20 μl of the MTS solution, incubated at 37° C. for 1-4 h in a humidified 5% CO₂ atmosphere, and the absorbance was measured at 490 nm using a Perkin-Elmer EnSpire Multimode 96-well plate reader.

Metabolic Study

Animals. Male C57BL/6 mice from Charles River Laboratories (Wilmington, MA) were housed in the National Cancer Institute animal facility that is a pathogen-free environment controlled for temperature, light (25° C., 12-h light/dark cycle) and humidity (45-65%) with free access to food and water. The National Cancer Institute Animal Care and Use Committee approved all animal experiments conducted in this study.

In Vivo Mouse Pharmacokinetics (PK) Study. 6-8 week-old male C57Bl/6 mice were selected for PK study of 51 (NCK 106) and 73 (NCK 167). The compounds 51 and 73 were dissolved in 5% DMSO plus 95% corn oil (Sigma, St. Louis, MO) and 20% Labrasol (a polyethyleneglycerol derivative) (Sigma, St. Louis, MO). Mice were divided into 4 groups of 3 each and orally gavaged with 100 mg/kg of 51 and 20 or 50 mg/kg of 73. Blood samples were collected by retro-orbital bleeding at 0, 0.25, 0.5, 1, 2, 4, 24, and 48 h. Serum was obtained by centrifugation for 10 min, at 14,000×g and prepared by mixing 10 μL serum with 40 μL of 90% acetonitrile including 4 μM newly synthesized reference compound 4 as internal standard. After centrifugation at 14,000×g for 20 min, 30 μL of supernatant was diluted with 30 μL of 10% acetonitrile, and a 5 μL aliquot was injected into Waters UPLC-QTOFMS system (Waters Corporation, Milford, MA).

In Vitro MLM Assay. Incubations of the mouse liver microsome (MLM) samples were performed in duplicate. Reactions (200 μL final volume) were conducted in 50 mM potassium phosphate buffer, 3 mM MgCl₂ (pH 7.4), 1 mg/mL MLM protein, and 2.5 μM test compound with or without 50 μM inhibitor. The mixture was preincubated at 37° C. for 3 min and initiated with 4 mM NADPH. At the serial time of the incubation, 20 μL of the mixture was added to ice-cold 80 μL of 90% acetonitrile including 4 μM of 4 followed by a 25 min incubation on ice and centrifugation at 14,000×g for 10 min. 40 μL of supernatant was diluted with 20 μL of water, and a 5 μL aliquot was injected into the UPLC-QTOF system.

UHPLC-ESI-QTOFMS and UHPLC-ESI-TQMS Analysis. Metabolite profiling and identification were performed on an Acquity UPLC/Synapt G2S Q-TOF MS system (Waters Corp., Milford, MA). Separation was achieved on an Acquity C18 BEH column (1.7 mm, 2.1×50 mm; Waters Corp.). The mobile phase consisted of water containing 0.1% formic acid (A) and acetonitrile containing 0.1% formic acid (B). Initial condition of 5% B was held for 0.5 min, with the following linear gradient employed: 60% B at 4 min, 95% B at 8 min, to 99% B at 8.1 min, held for 0.9 min for column flushing, then returning to initial conditions for 0.9 min for column equilibration before the next injection. The flow rate of the mobile phase was set 0.4 mL/min. Column temperature was maintained at 50° C. throughout the run. Data were collected in positive ion mode, which was operated in full-scan mode at 50-950 m/z. Nitrogen was used as both cone gas (151 L/h) and desolvation gas (950 L/h). Source temperature and desolvation temperature were set at 150° C. and 500° C., respectively. The capillary voltage and cone voltage were 2.82 kV and 41 V, respectively. Q-TOF MS was operated in positive ionization mode with MSE data collection.

Based on the initial report by Alverez et al. (J. Med. Chem. 63, 14087-14117 (2020)), scaffold changes, functional group substitution and prodrug masking were explored to enhance the efficacy of Plk1 PBD inhibitors, starting with the 1-thioxo-2,4-dihydro-[1,2,4]triazolo[4,3-a]quinazolin-5(1H)-one scaffold as a lead structure. The reported inhibitors are selective for inhibition of Plk1 PBD but not Plk2 and Plk3 PBDs. Cell efficacy was achieved with S-methylation to make prodrugs that are unmasked intracellularly. However, even for the best S-methyl derivatives, including 6, concentrations in excess of ˜100 μM of these compounds were required to induce mitotic arrest and proliferation in cancer cells in vitro (Alverez et al., J. Med. Chem. 63, 14087-14117 (2020)). Therefore, additional modified structures were sought to increase the inhibitory potency in binding to the PBD, as determined in an ELISA assay, and to increase the cellular anticancer efficacy, by structurally modifying either the active drug scaffold or the masking group present in the prodrugs.

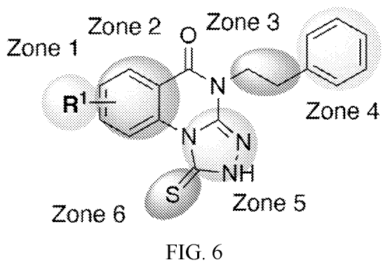

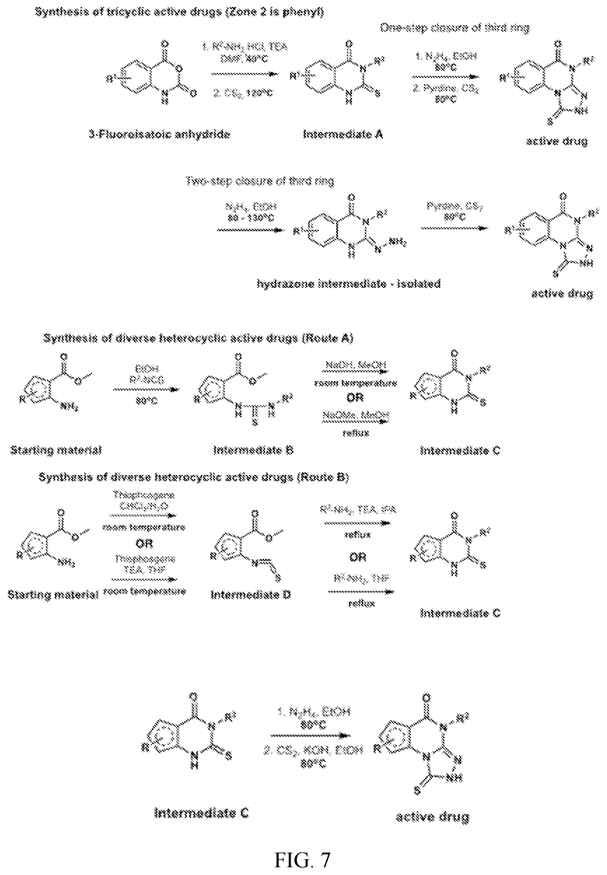

Substitution of the N-alkyl group on Zone 3 (FIG. 6) with fluoro or deutero atoms to decrease potential oxidation by CYP enzymes in vivo. Multiple substitutions of the N-propyl group are shown in Table 12, prepared as shown in Scheme 3 (FIG. 7). Fluoroalkanes are known to protect alkyl groups against oxidation by CYP enzymes and in some cases increase affinity at a target protein (N. A. Meanwell, J Med. Chem. 61, 5822-5880 (2018)). However, such substitution can also have major detrimental effects on the binding affinity. Thus, alkylfluoro substitution was cautiously proceeded with by measuring the ELISA binding of each modification. Furthermore, the deutero substitution of alkyl groups can decrease metabolism by the deuterium isotope effect (Knutson et al., J Med. Chem. 61, 2422-2446 (2018); and Pirali et al., J. Med. Chem. 62, 5276-5297 (2019)), without altering the interaction at the target binding site. Consequently, for these analogues, similar or identical ELISA affinity as the nondeutero lead compound are expected. Table 12 shows the inhibitory activity of triazoloquinazolinones substituted on the phenyl ring and N⁴-alkyl group at the Plk1 PBD (IC₅₀ values are n=3, unless noted in parentheses) and rat liver microsomal half-life, PAMPA permeability assays and kinetic aqueous solubility.

TABLE 12

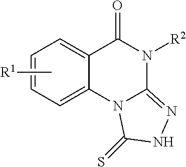

| | | | IC₅₀ ± sd | t_{1/2} | PAMPA | |
|---|---|---|---|---|---|---|
| | | | (μM, | (min, | (1e-6 | Solub. |
| Cmpd | R¹ = | R² = | ELISA)^{a} | RLM) | cm/s) | (μg/mL) |
| 4^{a} | H | 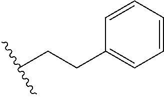 [SMILES: *CCc1ccccc1] | 4.38 ± 0.41 (6) | ND | ND | ND |
| 7^{b} (40) | 7-F | 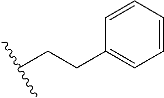 [SMILES: *CCc1ccccc1] | 12.92 ± 1.85 | >30 | 77.0 | ND |
| 8^{b} (48) | 9-F | 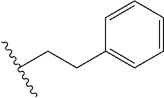 [SMILES: *CCc1ccccc1] | 2.58 ± 0.12 | >30 | 141 | 3.4 |
| 9^{b} (85) | H | 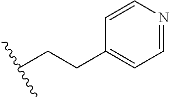 [SMILES: *CCc1ccncc1] | 0.89 ± 0.05 (4) | >30 | 32.7 | 10.9 |
| 10 (NCK131) | 9-F | 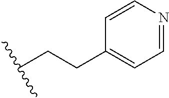 [SMILES: *CCc1ccncc1] | 4.97 ± 0.52 | ND | ND | ND |
| 11^{b} (63) | H | Et | 1.98 ± 0.07 | >30 | 39.5 | >39 |
| 12 (NCK138) | 9-F | Et | 2.96 ± 0.17 | ND | ND | ND |
| 5^{b} (21KJ129) | H | Pr | 1.03 ± 0.08 (9) | >30 | 37.0 | 21.3 |
| 13^{b} (64) | 7-F | Pr | 1.16 ± 0.06 (12) | >30 | 195 | >28 |
| 14 (NCK103) | 9-F | Pr | 2.12 ± 0.05 | ND | ND | ND |
| 15 (NCK104) | 9-Cl | Pr | 2.49 ± 0.02 | ND | ND | ND |
| (comp140) | | | | | | |
| 16 (NCK137) | 7-OMe | Pr | 1.82 ± 0.01 | ND | ND | ND |
| 17 (NCK119) | 9-OMe | Pr | 2.43 ± 0.01 | ND | ND | ND |
| 18 (NCK144) | 9-OH | Pr | 38.56 | ND | ND | ND |
| 19^{b} (68) (NCK44) | 7-F | 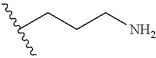 [SMILES: *CCCN] | 2.26 ± 0.07 (4) | >30^{c} >30^{d} | <1^{c} 16.8^{d} | >43^{c} 27^{d} |
| 20^{b} (69) | 9-F | 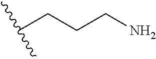 [SMILES: *CCCN] | 5.07 ± 0.16 | >30 | 4.2 | 15.9 |
| 21^{b} (27) | H | 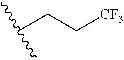 [SMILES: *CCC(F)(F)F] | 1.25 ± 0.11 (5) | >30, | 82 | 23.3 |
| 22^{b} (65) | 7-F | 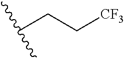 [SMILES: *CCC(F)(F)F] | 0.75 ± 0.06 (5) | >30 | 117 | 17.3 |
| 23 (NCK108; NCK142JE) | 9-F | 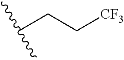 [SMILES: *CCC(F)(F)F] | 2.01 ± 0.16 | ND | ND | ND |
| 24 (NCK110) | 9-F | 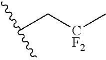 [SMILES: *CC(C)(F)F] | 3.27 ± 0.28 | ND | ND | ND |
| 25 (NCK121) | 9-F | 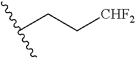 [SMILES: *CCC(F)F] | 2.45 ± 0.16 | ND | ND | ND |
| 26 (NCK125) | 9-F | 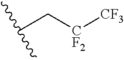 [SMILES: *CC(F)(F)C(F)(F)F] | 2.59 ± 0.1 | ND | ND | ND |
| 27 (NCK112) | 9-F | 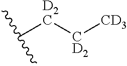 | 2.08 ± 0.02 | ND | ND | ND |
| 28^{b} (79) (NCK33) | 7-F | 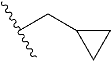 [SMILES: *CC1CC1] | 0.77 ± 0.08 (8) | >30 | 46.0 | 16.2 |
| 29 (NCK143) | 9-F | 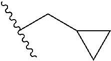 [SMILES: *CC1CC1] | 1.41 ± 0.02 | ND | ND | ND |

^{a}IC₅₀ values were determined from at least three independent experiments.
^{b}Data from Alverez et al., 2020 (18).
^{c}TFA salt.
^{d}Free base.

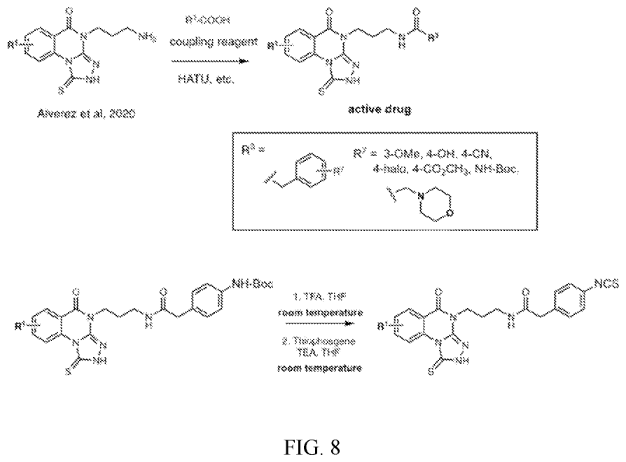

The functional group substitution of the phenylacetyl amide group (i.e. amide formation with a N-(3-aminopropyl) substituent, already shown to enhance binding affinity at the target protein was explored (Scheme 4) (FIG. 8). In some cases, already reported substitution of this phenyl ring with identical substitution already reported in Alverez et al. (Alverez et al., J. Med. Chem. 63, 14087-14117 (2020)) was combined with the favorable 9-fluoro substitution (Zone 1) as shown in Table 13. In other cases, new substitution of the Zone 4 phenyl ring was prepared. With these modifications, distal interactions from the binding region of the core 1-thioxo-2,4-dihydro-[1,2,4]triazolo[4,3-a]quinazolin-5(1H)-one scaffold were sought. One of the novel substitutions of the terminal phenyl ring was a 4-boronate ester, which could serve as a substrate for a Suzuki or other Pd-catalyzed reaction to extend the chain. The inclusion of a borate ester resulted in the widely used anticancer (multiple myeloma) drug, bortezomib, in which it reacts covalently with the target protein at a Thr residue (Bandyopadhyay et al., Curr. Opin. Chem. Biol. 34, 110-116 (2016)). Table 13 shows the inhibitory activity of 4-phenylacetic acid amide triazoloquinazolinone derivatives modified on the phenyl ring and phenylacetyl group at the Plk1 PBD (IC₅₀ values are n=3, unless noted in parentheses) and microsomal half-life, PAMPA assays and aqueous solubility.

TABLE 13

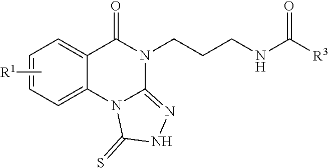

| | | | IC₅₀ ± sd (μM, | t_{1/2} (min, | PAMPA | Solub. |
|---|---|---|---|---|---|---|
| Cmpd | R¹ = | R³ = | ELISA)^{a} | RLM) | (1e-6 cm/s) | (μg/mL) |
| 30^{b} (127) (NCK97) | 7-F | 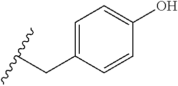 [SMILES: *Cc1ccc(O)cc1] | 1.16 ± 0.06 | >30 | 3.7 | 31.0 |
| 31^{b} (129) | H | 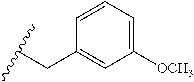 [SMILES: *Cc1cccc(OC)c1] | 0.96 ± 0.08 (4) | 20.1 | 300 | >65 |
| 32 (NCK133) | 9-F | 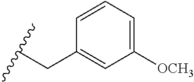 [SMILES: *Cc1cccc(OC)c1] | 1.94 ± 0.02 | | | |
| 33^{b} (134) | H | 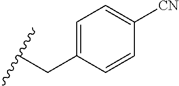 [SMILES: *Cc1ccc(C#N)cc1] | 0.99 ± 0.14 | >30 | 172 | 2.0 |
| 34 (NCK135) | 9-F | 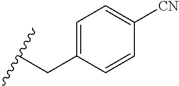 [SMILES: *Cc1ccc(C#N)cc1] | 1.52 ± 0.1 (4) | | | |
| 35 (NCK105) | 7-F | 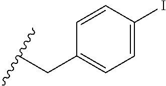 [SMILES: *Cc1ccc(I)cc1] | 2.4 ± 0.12 | | | |
| 36 (NCK160) | 9-F | 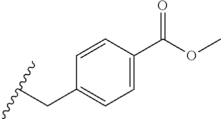 [SMILES: *Cc1ccc(C(=O)OC)cc1] | 1.38 ± 0.03 (4) | | | |
| 37 (NCK159) | 9-F | 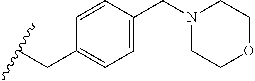 [SMILES: *Cc1ccc(CN2CCOCC2)cc1] | 2.44 ± 0.37 (4) | | | |
| 38 (NCK102) | 7-F | 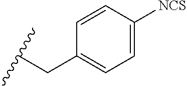 | 1.4 ± 0.09 | | | |

^{a}IC₅₀ values were determined from at least three independent experiments.
^{b}Data from Alverez et al., 2020.

Substitution of the Zone 2 ring (FIG. 6) with non-phenyl rings to try to increase the Plk1 PBD affinity. Monoheterocyclic aromatic rings in place of the phenyl ring of Zone 2 are included (thiophene, furan, pyrrole, etc.), and bicyclic aromatic systems (benzothiophene, benzofuran, etc.), as well (Table 14). The inclusion of a 5-Cl on the thiophene protects it against in vivo reactivity, as was shown in the approved anticoagulant drug rivaroxaban (Lang et al., Drug Metab. Dispos. 37, 1046-1055 (2009)) and antithrombotic P2Y₁₂ receptor antagonist elinogrel³ and other experimental drugs (Fang et al., Biochem. Pharmacol. 97, 215-223 (2015)). Table 14 shows the inhibitory activity of 4-phenylacetic acid amide triazoloquinazolinone derivatives modified on the aryl or heteroaryl Zone 2 ring at the Plk1 PBD (IC₅₀ values are n=3, unless noted in parentheses) and microsomal half-life, PAMPA assays and aqueous solubility. R² ═(CH₂)₂CH₃, unless noted.

TABLE 14

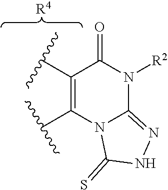

| | | IC₅₀ ± sd (μM, | t_{1/2} (min, | PAMPA | Solub. |
|---|---|---|---|---|---|
| Cmpd | R⁴ = | ELISA)^{a} | RLM) | (1e-6 cm/s) | (μg/mL) |
| 39 (NCK147) | 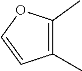 | 7.67 ± 0.3 | | | |
| 40 (NCK148) | 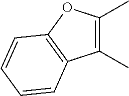 | 1.56 ± 0.13 (7) | | | |
| 41 (NCK164) | 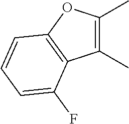 | 3.36 ± 0.39 (4) | | | |
| 42 (NCK156) | 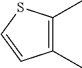 | 2.84 ± 0.14 (6) | | | |
| 43 (NCK149) | 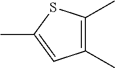 | 2.15 ± 0.13 (6) | | | |
| 44 (NCK157) | 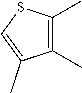 | 1.07 ± 0.04 (4) | | | |
| 45 (NCK158) | 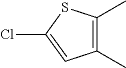 | 1.46 ± 0.17 | | | |
| 46 (NCK165) | 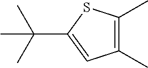 | 2.1 ± 0.26 | | | |
| 47 (NCK166) | 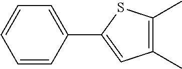 | 4.66 ± 0.54 | | | |
| 48 (NCK162) | 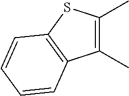 | 1.72 ± 0.06 (2) | | | |
| 49a (NCK163) | 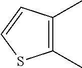 | 3.6 ± 0.19 (2) | | | |
| 49b (NCK179) | 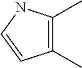 | 15.28 ± 0.36 (4) | | | |
| 49c (NCK180) | 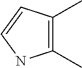 | | | | |
| 49d (NCK178) | 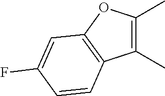 | 3.38 ± 0.14 (4) | | | |
| 49e (NCK181) | 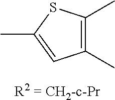 | 1.66 ± 0.04 (4) | | | |
| 18 (NCK144) | 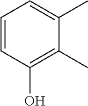 | 39.71 ± 1.23 | | | |
| NCK171 | 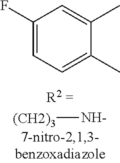 | 5.77 ± 0.33 | | | |
| NCK172 | 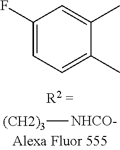 | 9.72 ± 0.52 | | | |

^{a}IC₅₀ values were determined from at least three independent experiments.

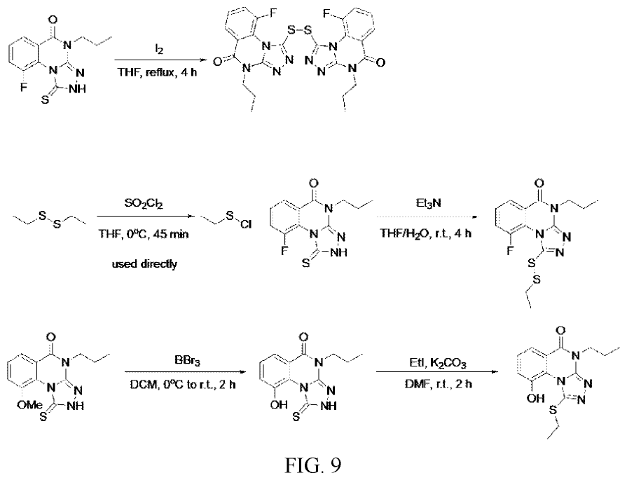

Alternative prodrug masking of the thiocarbonyl group at Zone 6 (FIG. 6) was tested in HeLa cell-based assays of efficacy in disruption of the cell cycle. Alverez et al. reported only S-methyl prodrugs as a feasible approach to protected compounds for in vivo unmasking (Alverez et al., J. Med. Chem. 63, 14087-14117 (2020)). Various S-alkyl compounds have been noted to be oxidized in vivo to S-O derivatives or to liberate a thio group (Bandyopadhyay et al., Curr. Opin. Chem. Biol., 34, 110-116 (2016); Mazel et al., J. Pharmacol. Exp. Ther., 143, 1-6 (1964); and Larsen et al., Xenobiotica 18, 313-322 (1988)), which itself is susceptible to further oxidation. A similar S-acetyl prodrug was shown to be unstable. Therefore, other homologated S-alkyl groups: S-ethyl, S-propyl, S-butyl, S-isopropyl, etc. were explored (Scheme 5) (FIG. 9). Furthermore, some extended S-alkyl derivatives, shown to be ineffective in the ELISA binding assay, were tested in cell-based assays of efficacy (Table 15). Table 15 shows prodrug derivatives of triazoloquinazolinones with efficacy in inducing death of leukemia cells (IC50 values are n=3, unless noted in parentheses).

TABLE 15

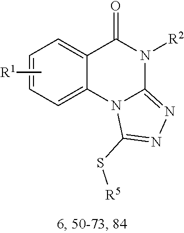

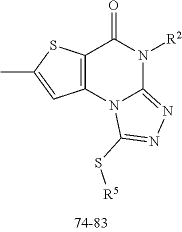

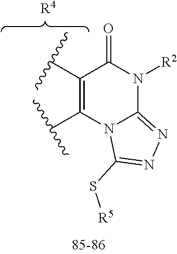

| | | | | Cell efficacy, GI₅₀ ± |
|---|---|---|---|---|
| Cmpd | R¹ or R⁴ = | R² = | R⁵ = | sd (μM)^{a} |
| 6 (143) | H | (CH₂)₂CH₃ | CH₃ | 164.87 ± 16.4 |
| (NCK100) | | | | |
| 50 (144) | 7-F | (CH₂)₂CH₃ | CH₃ | 243.46 ± 3.65 |
| (NCK101) | | | | |
| 51 (145) | 9-F | (CH₂)₂CH₃ | CH₃ | 335.53 ± 52.52 |
| (NCK106) | | | | |
| 52 (146) | 9-Cl | (CH₂)₂CH₃ | CH₃ | 348.30 ± 1.90 |
| 53 (147) | 9-OMe | (CH₂)₂CH₃ | CH₃ | >400 |
| 54 (KJ110) | H | (CH₂)₂CH₃ | CH(CH₃)₂ | 342.23 ± 68.61 |
| 55 (KJ175) | H | (CH₂)₂CH₃ | (CH₂)₂OH | >400 |
| 56 (KJ151) | 7-F | (CH₂)₂CH₃ | CH₂CN | >400 |
| 57 (KJ159) | 7-F | (CH₂)₂CH₃ | CH₂CONH₂ | >400 |
| 58 KJ163 | 7-F | (CH₂)₂CH₃ | CH₂Ph | >400 |
| 59 (KJ160) | 7-F | (CH₂)₂CH₃ | CH₂Ph(4-Cl) | >400 |
| 60 (NCK139) | 9-F | CH₂CH₃ | CH₂CH₃ | 157.03 ± 17.76 |
| 61 (NCK111) | 9-F | (CD₂)₂CD₃ | CH₃ | 254.67 ± 34.07 |
| 62 (NCK146) | 9-F | (CD₂)₂CD₃ | CH₂CH₃ | 150.13 ± 16.06 |
| 63 (NCK141) | 9-F | (CD₂)₂CD₃ | CD₃ | 360.60 ± 26.20 |
| 64 (NCK140) | 9-F | (CH₂)₂CH₃ | CD₃ | >400 |
| 65 (NCK153) | 9-F | (CH₂)₃—NHCO-3-MeOPh | CH₂CH₃ | 103.55 ± 7.48 |
| 66 (NCK154) | N—Pr—NHCO-4-CN—Ph, | (CH₂)₃—NHCO-4-CN—Ph | CH₂CH₃ | >400 |
| | 9-F | | | |
| 67 (NCK145) | 9-OH | (CH₂)₂CH₃ | CH₂CH₃ | >400 |
| 68 (NCK150) | 9-F | (CH₂)₂CH₃ | (CH₂)₃CH₃ | >400 |
| 69 (NCK151) | 9-F | (CH₂)₂CH₃ | (CH₂)₃CH₃ | >400 |
| 70 (NCK152) | 9-F | (CH₂)₂CH₃ | CH(CH₃)₂ | >400 |
| 71 (NCK155) | 9-F | (CH₂)₂CH₃ | [dimer S-S] | >400 |
| 72 (NCK161) | 9-F | (CH₂)₂CH₃ | SCH₂CH₃ | 21.40 ± 0.24 |
| 73 (NCK167) | 9-F | (CH₂)₂CH₃ | 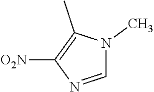 | 14.01 ± 0.39 |
| 74 (NCK170) | 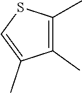 | (CH₂)₂CH₃ | 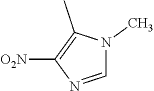 | 25.83 ± 0.26 |
| 75 (NCK173) | 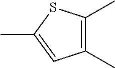 | (CH₂)₂CH₃ | 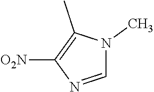 | 4.14 ± 0.31 |
| 76 (NCK174) | 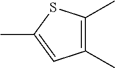 | (CH₂)₂CH₃ | 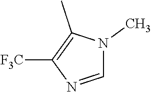 | >400 |
| 77 (NCK175) | 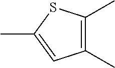 | (CH₂)₂CH₃ | 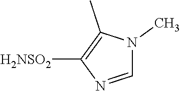 | >400 |
| 78 (NCK183) | 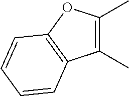 | (CH₂)₂CH₃ | 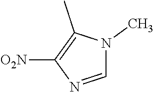 | 8.31 ± 0.56 |
| 79 (NCK184) | 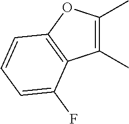 | (CH₂)₂CH₃ | 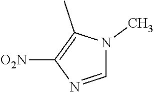 | 7.53 ± 0.43 |
| 80 (NCK185) | 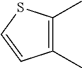 | (CH₂)₂CH₃ | 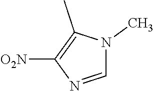 | 8.40 ± 0.74 |
| 81 (NCK186) | 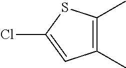 | (CH₂)₂CH₃ | 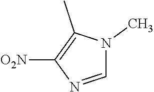 | 6.04 ± 0.17 |
| 82 (NCK187) (HBL-207) | 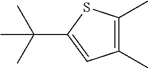 | (CH₂)₂CH₃ | 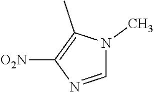 | 4.19 ± 0.15 |
| 83 (NCK188) | 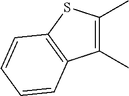 | (CH₂)₂CH₃ | 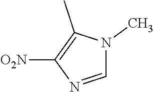 | 7.05 ± 0.27 |
| 84 | 9-F | 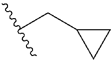 [SMILES: *CC1CC1] | 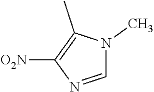 | TBD |
| 85 (NCK182) | 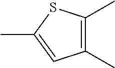 | 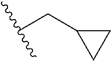 [SMILES: *CC1CC1] | 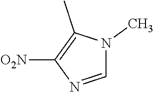 | 3.11 ± 0.17 |
| 86 (NCK190) | 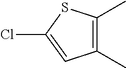 | 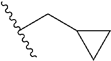 [SMILES: *CC1CC1] | 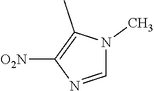 | |
| 87 (NCK192) | 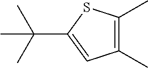 | 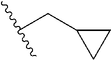 [SMILES: *CC1CC1] | 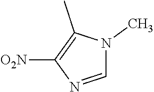 | |
| 88 (NCK194) | 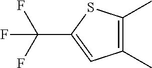 | 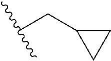 [SMILES: *CC1CC1] | 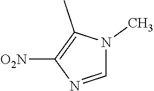 | |

^{a}A colorimetric MTS cell proliferation assay was carried out using multiple myeloma-derived L363 cells, as described in the
Materials and Methods. GI₅₀ values were determined from at least three independent experiments.

Since the mechanism of the unmasking of the thioethers was not identified, it was sought to first explore the parameters of the S-alkyl SAR empirically to identify which groups might be subject to intracellular unmasking. S-ethyl was found to be more efficient in the cell-based assay compared to S-methyl (nearly as efficacious as S-ethyl) and other S-alkyl analogues that were much less efficacious. Another prodrug scheme that was explored was the formation of disulfide derivatives of the thiocarbonyl moiety. Disulfides are known to be readily cleaved in the intracellular reducing environment (Sun et al., Nat. Commun. 10, 3211 (2019); Zhang et al., Drug Metab. Dispos. 47, 1156-1163 (2019); and Ochi et al., Curr. Protoc. Nucleic Acid Chem. 62, 4 63 61-64 63 20 (2015)).

A known aryl thioether prodrug moiety was adapted to the Plk1-PBD inhibitors (G. B. Elion, Annals of the New York Academy of Sciences 685, 401-407 (1993); and Van Scoik et al., Drug Metab. Rev. 16, 157-174 (1985)). The immunosuppressant drug 6-mercaptopurine was converted to a prodrug, [6-(1-methyl-4-nitro-5-imidazolyl)thiopurine, i.e. azathioprine, which displayed improved absorption over the parent drug (G. B. Elion, In Vitro Cell Dev. Biol. 25, 321-330 (1989)). The prodrug can react with a sulfhydryl, such as glutathione, or an amino group to regenerate the parent 6-mercaptopurine along with a 1-methyl-4-nitroimidazole 5-thioether. The same group was also applied recently to an experimental antiviral drug. Azathioprine is also used as a dermatological drug (Patel et al., J. Am. Acad. Dermatol. 55, 369-389 (2006)). Furthermore, substitutions, including trifluoromethyl and primary sulfonamide, for the aryl nitro group (Robello et al., Nipamovir: synthesis and preclinical evaluation of an anti-HIV thiobenzamide prodrug. International Society for Antiviral Research, 34th International Conference on Antiviral Research Poster #121 (2021)), are known to be associated with toxicity. The synthetic approach is shown in Scheme 6 (FIG. 10).

The most favorable combinations of the abovementioned changes, gleaned in parallel for the active and prodrug derivatives, were combined into a small number of analogues that are to be considered as potential candidates for therapeutic development. The in vitro and in vivo pharmacokinetics of these agents was explored. Inhibitors of CYP enzymes were compared for their ability to impede the in vivo degradation of key molecules. Among these CYP inhibitors, the most effective at prolonging the in vivo half-life was ketoconazole, suggesting that metabolism is CYP 3A4-dependent (Granvil et al., Drug Metab. Dispos. 31, 548-558 (2003)). Thus, coadministration of ketoconazole in understood to prolong the in vivo efficacy of this series of PBD inhibitors.

### Example 167

This example describes the Plk1 PBD inhibition activity and binding specificity of exemplary compounds of formula (I) in an aspect of the invention.

The ELISA-based Plk1 PBD inhibition assay (Yun et al., Nat. Struct. Mol. Biol. 16, 876-882 (2009)), which utilizes the highly specific interaction between the full-length human Plk1 expressed in HEK293T cells and a specific phospho-Thr (pT)-containing peptide (Biotin-Ahx-C-ETFDPPLHSpTAI-NH₂) (Kang et al., Mol. Cell 24, 409-422 (2006)), was used to determine the Plk1 PBD-binding activity for all the compounds reported here. Representative results for some of selected compounds in Tables 12-13 were provided in FIG. 12. These compounds inhibited PBD with IC₅₀ values of 1.49-2.94 μM under these experimental conditions. When compared to the previously characterized Plk1 PBD-binding control phosphopeptide, PLHSpT 2 (IC₅₀ of 14.74 μM) (Alverez et al., J. Med. Chem. 63, 14087-14117 (2020)), showing a K_{d} of ˜450 nM (Yun et al., Nat. Struct. Mol. Biol. 16, 876-882 (2009)), the affinity of some of the best inhibitors is expected to be approximately an order of magnitude higher than PLHSpT 2.

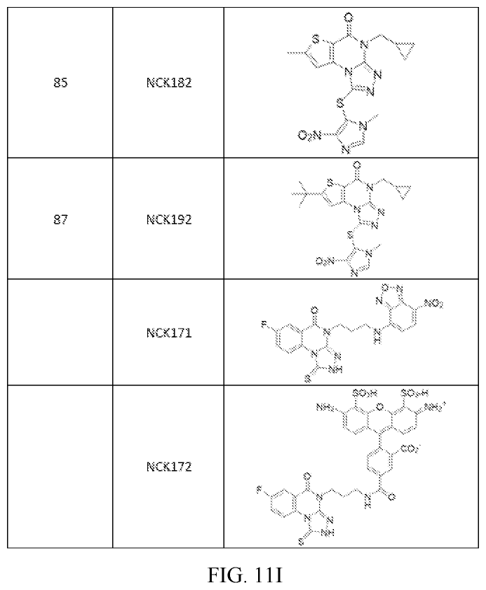

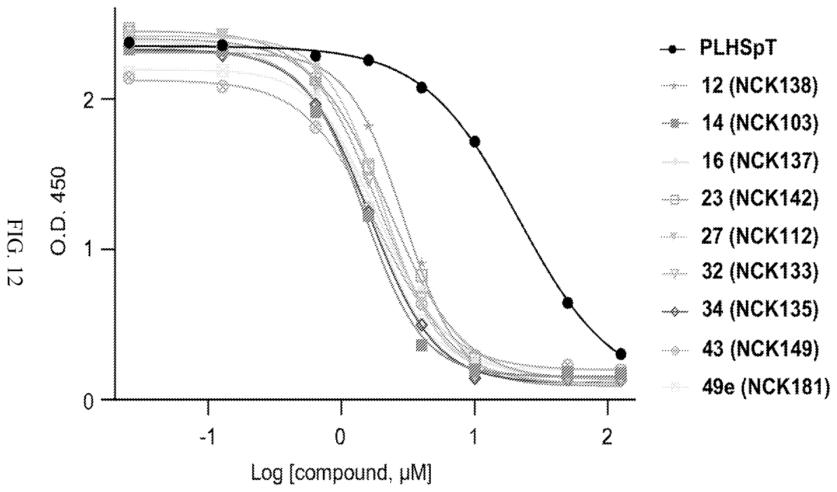

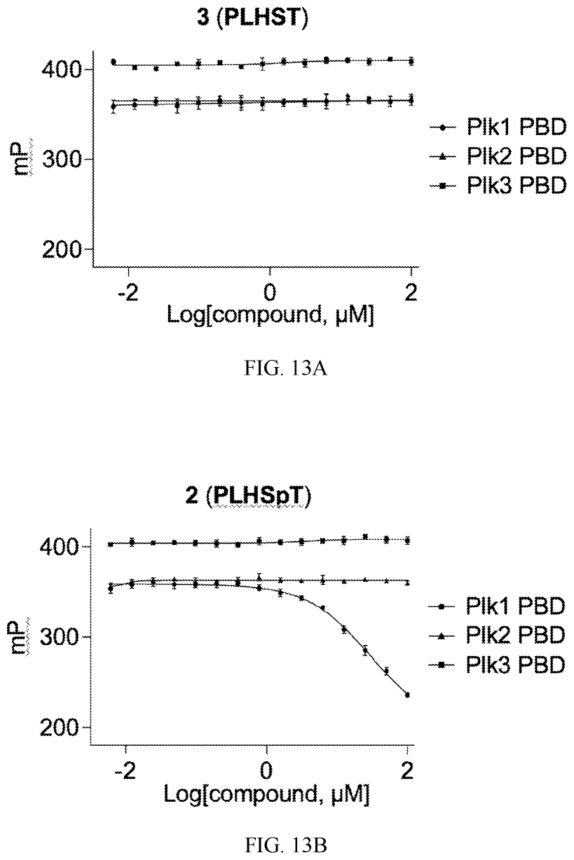

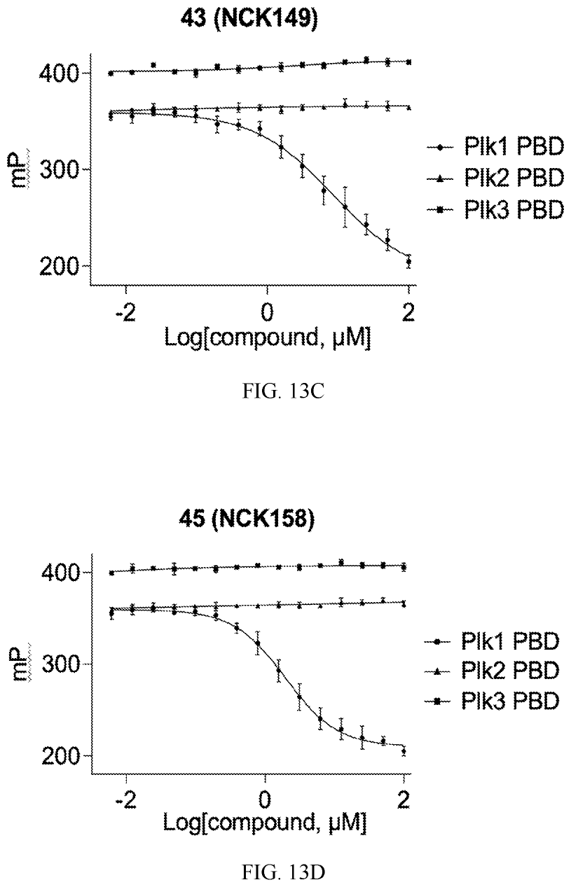

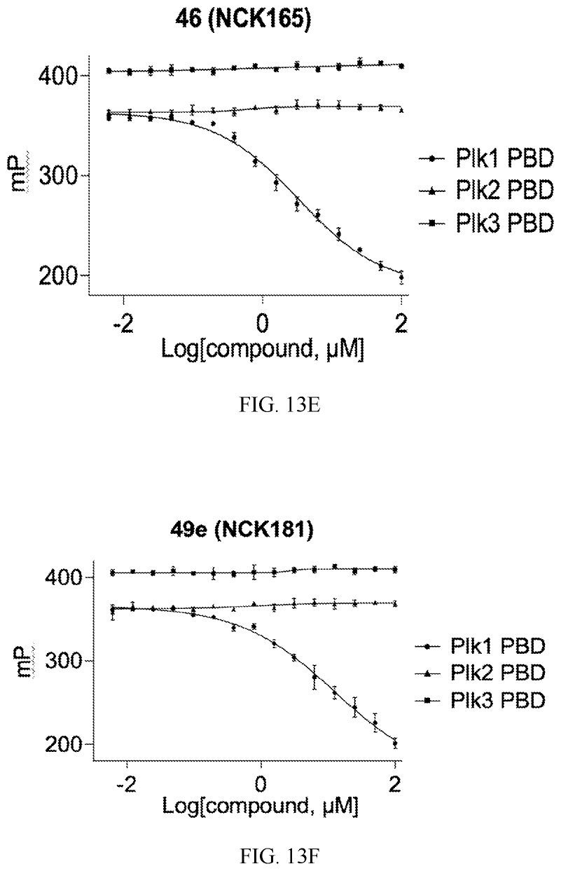

While targeting Plk1 PBD offers a great opportunity to achieve superior binding specificity over targeting the ATP-binding motif because of the nature of specific protein-protein interaction, Plk1 PBD still exhibits a high level of homology (approximately 39%) with the two closely related Plk2 and Plk3 PBDs (Lee et al., Trends Pharmacol. Sci. 36, 858-877 (2015)). To determine whether the above compounds exhibit Plk1 PBD-binding specificity, fluorescence polarization (FP)-based assays developed to determine their affinities to each PBD of Plk1-3 were carried out as described previously (Liu et al., Nat. Chem. Biol. 7, 595-601 (2011); and Alverez et al., J. Med. Chem. 63, 14087-14117 (2020)). As expected, PLHSpT 2, but not its respective nonphosphorylated peptide 3, specifically inhibited Plk1 PBD with an IC₅₀ of 37.7 μM, a value comparable to that with the ELISA data shown in FIG. 12. Under these experimental settings, all the compounds tested (e.g., compounds 43, 45, 46, and 49e) exhibited a high level of specificity against Plk1 PBD, even though their Plk1 PBD-binding affinities were increased approximately 10-fold (FIGS. 13A-13F). FIGS. 13C-13F show that all the compounds of formula (I) tested potently inhibited the PBD of Plk1 but not Plk2 and Plk3. Under the same experimental conditions, PLHSpT 2 (FIG. 13B) but not its non-phosphorylated form, PLHST 3 (FIG. 13A), exhibited Plk1 PBD-specific inhibition. These observations draw contrast to the recently described Plk1 PBD inhibitor, KBJK557, that shows considerable cross-reactivities among Plk1-3 PBDs (Gunasekaran et al., J. Med. Chem. 63, 14905-14920 (2020)).

### Example 168

This example describes the cellular efficacy of certain prodrugs of the compound of formula (I) in an aspect of the invention.

To evaluate the cellular efficacy of the prodrug compounds listed in Table 15, multiple myeloma-derived L363 cells optimized for mouse xenograft tumor assays were used (Gabrea et al., Genes Chromosomes Cancer 47, 573-590 (2008); and Simmons et al., Mol. Oncol. 8, 261-272 (2014)). These prodrugs are expected to achieve a higher level of their intracellular active species by promoting cell membrane permeability. To assess their anti-Plk1 PBD activity, the prodrugs were compared for their ability to induce mitotic arrest and an antiproliferation effect, which are characteristics of Plk1 PBD inhibition regardless of the degrees of cell transformation (Park et al., Cell Cycle 14, 3624-3634 (2015); and Liu et al., Nat. Chem. Biol. 7, 595-601 (2011)). Results showed that several of the prodrugs in Table 15 (namely, 60, 61, 63, 72-75, 78-83, and 85) inhibited cell proliferation substantially more potently than the previously reported methylthio prodrug, 51 (Alverez et al., J. Med. Chem. 63, 14087-14117 (2020)) (Table 15). See FIG. 14.

Initially, the S-methyl group of 50 (7-F, GI₅₀ 243 μM) and 51 (9-F, GI₅₀ 334 μM) was extended to larger alkyl groups. Among the S-alkyl derivatives, S-ethyl prodrugs, e.g. 60 and 62 (GI₅₀ 150-157 μM), generally displayed higher potency in the L363 cell assay than the corresponding S-methyl derivatives. The 3-methoxyphenylacetyl derivative 65 (9-F, GI₅₀ 104 μM), despite having a larger molecular weight, displayed enhanced cellular efficacy, but the related CN derivative 66 was poorly active. However, elongation beyond S-ethyl, including S-propyl 68, S-butyl 69 and the branched alkyl (70) derivatives or a variety of other functional groups (55-59) greatly reduced the cell efficacy or completely abolished it. However, S-isopropyl analogue 54 in the acyclic fluorinated series had only 2-fold lower potency than the corresponding S-methyl reference compound 6. Several disulfide derivatives, e.g. 71, 72, were compared to the S-alkyl derivatives in the whole cell assays. The dimeric 71 (disulfide form of 14) was inactive. However, the ethyl disulfide 72 (N-propyl) showed considerable efficacy (GI₅₀ 21.4 μM), i.e. 7-fold more potent than the S-ethyl prodrug 60, although 60 was the N-ethyl homologue rather than N-propyl. Effects of deuterium substitution in 61-64 were probed in the cell assay. The heptadeuterated S-ethyl prodrug 62 was slightly more potent in the cell assay than the corresponding S-methyl derivative 61. Deuteration in 64 of the S-methyl group of 51 reduced cell efficacy, suggesting a possible deuterium isotope effect in the intracellular demethylation reaction, and the slight reduction with S-CD₃ in compound 63 compared to 61 supported the same hypothesis. Moreover, S-ethyl prodrugs (65, 66) that were elongated beyond N-propyl had variable efficacies in cell assays.

Among the imidazole prodrug derivatives, 73 induced a readily discernable anti-proliferative effect with a GI₅₀ value (i.e., inhibition of cell growth by 50% of the DMSO control) of 14.0 μM. 75 (GI₅₀ of 4.1 μM), which contains a monoheterocyclic aromatic ring in substitution of the phenyl ring in 73, exhibited an anti-proliferation activity several-fold improved from 73. Under the same conditions, their parental compounds, 14 and 43, respectively, failed to induce any a detectable level of cellular response, confirming the previous observation (Alverez et al., J. Med. Chem. 63, 14087-14117 (2020)) that prodrug moieties are critically required to induce cellular effects.

Based on the favorable cellular efficacy results with 73 and 75, other heterocyclic substitutions of the zone 2 ring instead of 9-fluorophenyl were compared, i.e. compounds 78-85. All contained an N-propyl group, and the prodrug moiety was the same S-1-methyl-4-nitroimidazol-5-yl moiety as in 73 and 75. Compounds 84-85 contained an N-cyclopropylmethyl group, similar to potent active drugs 28 and 29. 2-Methyl-thienyl prodrug derivative 85 proved to be the most potent analogue in the cell assay with a GI₅₀ of 3.11 μM (Table 15). Thus, the substitution of the N-propyl group of prodrug 75 with N-cyclopropylmethyl decreased the GI₅₀ by 25%. This is consistent with the increased affinity (29% lower IC₅₀) of N-cyclopropylmethyl active drug 29 compared to 14 in the ELISA assay. Also, 2-methyl-thienyl active drug 49e compared to the equivalent N-propyl derivative 43.

To investigate whether the effect of 73 or 75 on L363 cell proliferation could be attributable to their capacity to inhibit PBD-dependent Plk1 function, adherent HeLa cells, which are well suited for cytological analyses, were treated with either of these inhibitors at 25 μM concentration, incubated for 12 h (a minimum period of time necessary to observe any cell cycle effect), fixed, and stained with 4′,6-diamidino-2-phenylindole (DAPI) to reveal chromosomal DNA morphologies. For comparison, their parental compounds 14 and 43 were included for analyses. Under these conditions, 73 effectively induced rounded cells with mitotically arrested or apoptotic chromosome morphologies (judging from the DAPI stains) in greater than 30% of the population. Consistent with the potent inhibition of cell proliferation in FIG. 14, 75 induced mitotically arrested or apoptotic cells in an approximately 60% of the population. As expected, parent drugs, 14 and 43, failed to show any of these phenotypes. In line with these findings, the N-cyclopropylmethyl-containing compound 85 also induced Plk1 delocalization and mitotic arrest at a level similar to those of compound 75. These observations assure that the mitotic block induced by 75 and 85 are likely the consequence of inhibiting the mitotic functions of Plk1, as well documented previously (Strebhardt et al., Nat. Rev. Cancer. 6, 321-330 (2006)).

To directly examine whether the observed mitotic arrest was the consequence of inhibiting Plk1 PBD function, immunostaining analysis was carried out to determine the ability of intracellular Plk1 to localize to centrosomes and kinetochores, an event that requires PBD-mediated protein-protein interaction (Lee et al., Trends Pharmacol. Sci. 36, 858-877 (2015); and Elia et al., Cell 115, 83-95 (2003)). As expected, HeLa cells treated with 25 μM of 75 for 2 h exhibited greatly diminished Plk1 signal intensities at both centrosomes and kinetochores. 73 crippled Plk1 localization somewhat less effectively than 75. A short treatment time (i.e., 2 h), which was intended to minimize a potential indirect effect of these inhibitors, was sufficient to delocalize greater than 70% of Plk1 signals from centrosomes (marked by anti-Cep63 signals) and 90% of those from kinetochores (marked by anti-CREST signals). Less effective Plk1 delocalization from centrosomes is likely due to the presence of PBD-independent Plk1 localization to this site, as reported previously (Park et al., Cell. Mol. Life Sci. 67, 1957-1970 (2010)). As a result of Plk1 delocalization, these cells showed a prometa/meta-arrested DNA morphology, a phenotype that has been observed by expressing a dominant-negative Plk1 PBD (Hanisch et al., Mol. Biol. Cell 17, 448-459 (2006); and Seong et al., J. Biol. Chem. 277, 32282-32293. (2002)).

### Example 169

This example describes the pharmacokinetic analysis of exemplary compounds of formula (I) in an aspect of the invention.

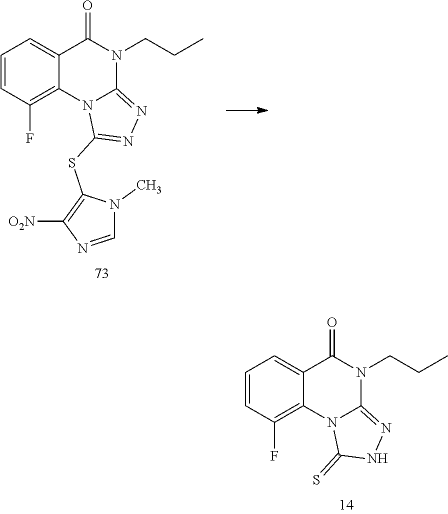 [SMILES: CCCn1c(=O)c2cccc(F)c2n2c(Sc3c([N+](=O)[O-])ncn3C)nnc12 ; CCCn1c(=O)c2cccc(F)c2n2c(=S)[nH]nc12]

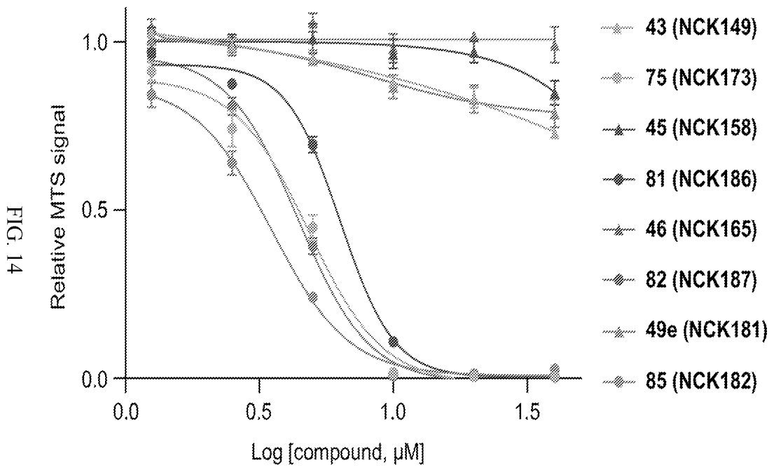

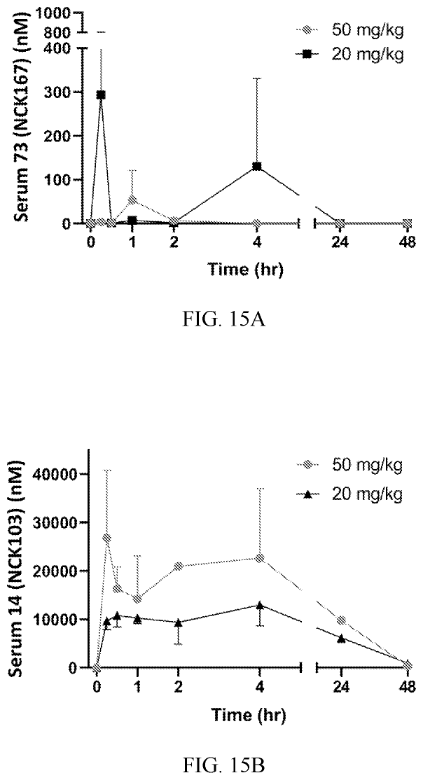

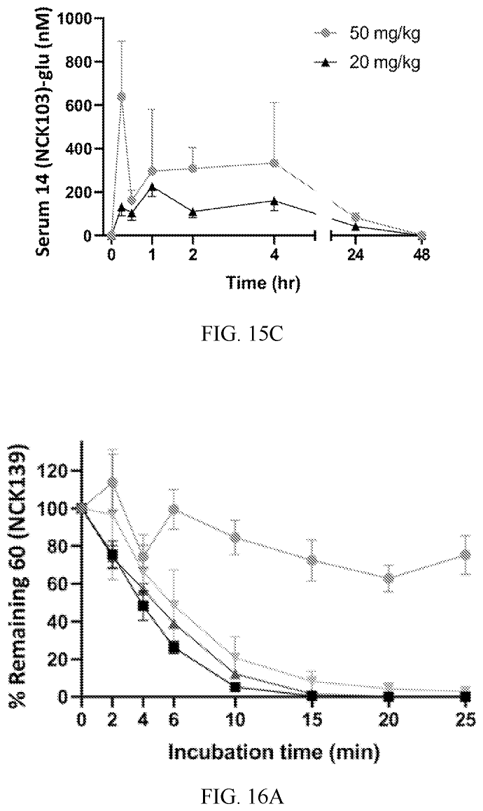

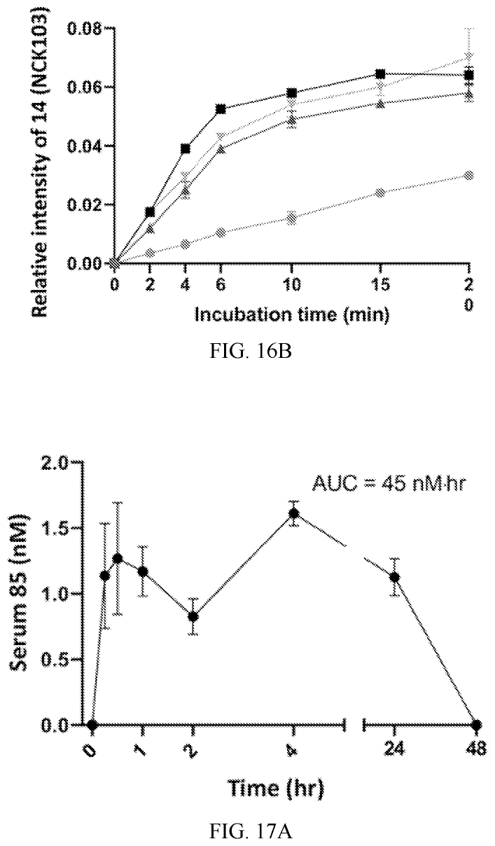

To evaluate metabolic stability information, S-methyl 51 and 5-thio-1-methyl-4-nitroimidazolyl 73 prodrugs were administered orally in male C57BL/6 mice via gavage 50 mg/kg or 20 mg/kg, respectively. The pharmacokinetic (PK) profiles of 51 and its metabolites show that the area under the curve (AUC) and half-life of 51 are 10,810 mg-h/L and 24 min, respectively, while the AUC of 14, demethylated 51, is 413,000 mg-h/L. 51 and 14 were hydroxylated, and 14 was further glucuronidated. 73 was rapidly metabolized to 14 with large differences in serum concentration between individuals (FIGS. 15A-15C). Concentration of 14 derived from 73 showed a rapid increase in blood at 15 min and maintained high concentration up to 4 h. The AUCs of 14 were 523,200 mg-h/L and 317,600 mg-h/L in 50 mg/kg and 20 mg/kg doses, respectively. Compound 14 was further metabolized by glucuronidation. Unlike the S-ethyl-containing prodrug, 60 (NCK139), whose conversion to the active 14 was sensitive to ketoconazole, a CYP3A inhibitor (FIGS. 16A-16B), 73 was refractory to ketoconazole treatment in a mouse liver microsome-based assay. The presence of the 9-F group impeded the hydroxylation, compared to the PK studies in Alverez et al. (J. Med. Chem. 63, 14087-14117 (2020)).

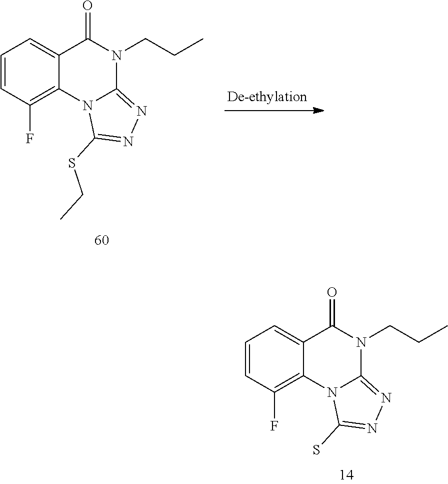 [SMILES: CCCn1c(=O)c2cccc(F)c2n2c(SCC)nnc12 ; CCCn1c(=O)c2cccc(F)c2n2c(S)nnc12]

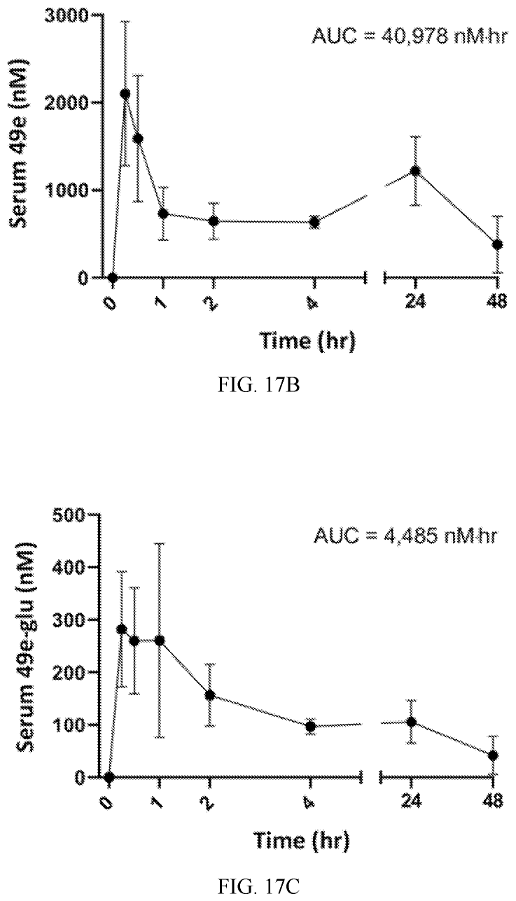

In a second experiment, the metabolic stability of compound 85 (NCK182) was studied, which showed the highest cellular efficacy (GI₅₀ of 3.11 μM) in the L363-based cell proliferation assay. Intraperitoneal injection of the compound in male C57BL/6 mice yielded rapid generation of the active parent drug 49e (FIGS. 17A-17C), suggesting that the prodrug moiety of 85 was quickly cleaved in the system. The active 49e species appeared to be largely stable for up to 2 days.

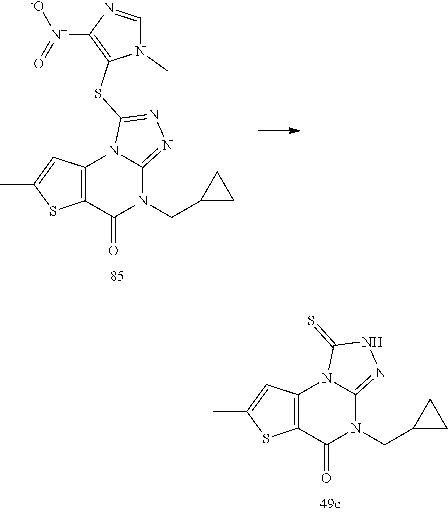

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-15. (canceled)

16. A method for treating cancer in a subject in need thereof comprising administering to the subject a compound of formula (I) or a pharmaceutically acceptable salt thereof, wherein the compound of formula (I) is 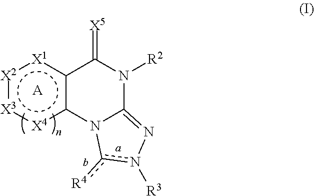
wherein
ring A is phenyl, a 5-membered heteroaryl, or a 6-membered heteroaryl;
X¹, X², X³, and X⁴ are the same or different and each is CR¹, N, S, or O, wherein no more than three of X¹, X², X³, and X⁴ are N, S, or O;
n is 0 or 1; provided that when n is 0, at least one of X¹, X², X³, and X⁴ is N, S, or O;
X⁵ is O or S;
each instance of R¹ is the same or different and each is selected from the group consisting of H, deuterium, C₁₋₆ alkyl, alkenyl, alkynyl, cycloalkyl, hydroxy, alkoxy, cycloalkoxy, halo, haloalkyl, alkylthio, alkylthioalkylenyl, cyano, amino, alkylamino, dialkylamino, amido, aryl, and heterocycloalkyl or
more than one instance of R¹ are linked to form a cycloalkyl or a phenyl, each of which is optionally substituted;
R² is selected from the group consisting of H, alkyl, cycloalkyl, alkenyl, and aryl;
wherein the alkyl, cycloalkyl, alkenyl, and aryl of R² is optionally substituted with one or more substituents selected from the group consisting of deuterium, alkyl, alkoxy, halo, hydroxy, haloalkyl, alkoxy, alkyl, cycloalkyl, heterocycloalkyl, aryl, heteroaryl, amino, alkylamino, dialkylamino, amido, alkylsulfonamido, phosphonato, cyano, thiocyano, carboxylate, a protecting group, an amino acid residue, and a peptide residue;
R³ is selected from the group consisting of H, C₁₋₁₀ alkyl, cycloalkyl, aminoalkyl, alkylaminoalkyl, dialkylaminoalkyl, and arylcarbonylalkyl or R³ is absent;
bond a is a single bond or double bond;
bond b is a single bond or double bond;
provided when bond a is a single bond, then R³ is present, bond b is a double bond, and R⁴ is S, and
when bond a is a double bond, then R³ is absent, bond b is a single bond and R⁴ is H, alkyl, hydroxy, amino, or S—R⁵, wherein R⁵ is selected from the group consisting of C₁₋₁₀ alkyl, cycloalkyl, acetyl, and optionally substituted 4-imidazolyl of the structure 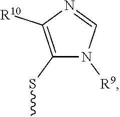
wherein R⁹ is H or alkyl,
and R¹⁰ is H, alkyl, halo, haloalkyl, nitro, or sulfonamido (—SO₂NH₂); and wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl,
provided that
when ring A is phenyl, X¹, X², X³, and X⁴ are each CR¹, X⁵ is O, R² is n-propyl, bond a is a single bond, bond b is a double bond, R³ is hydrogen, and R⁴ is S, then R¹ is not alkylamido (—C(O)NHalkyl);
when ring A is phenyl, X¹, X², X³, and X⁴ are each CH, X⁵ is O, bond a is a double bond, bond b is a single bond, and R³ is absent, then R⁴ is not H or alkyl;
when ring A is phenyl, X¹, X², X³, and X⁴ are each CR¹, X⁵ is O, bond a is a double bond, bond b is a single bond, and R³ is absent, then R⁴ is not alkyl;
when ring A is phenyl, X¹, X², and X⁴ are each CH, X³ is CR¹, n is 1, X⁵ is O, R² is alkyl, bond a is a single bond, bond b is a double bond, R³ is hydrogen, and R⁴ is S, then R¹ at the X³ position is not halo or hydrogen;
when ring A is phenyl, X¹, X³, and X⁴ are each CH, X² is CCH₃, n is 1, X⁵ is O, bond a is a single bond, bond b is a double bond, R³ is hydrogen, and R⁴ is S, then R² is not aryl;
when ring A is phenyl, X¹, X³, and X⁴ are each CH, X² is C(halo), n is 1, X⁵ is O, bond a is a double bond, bond b is a single bond, R² is phenyl, R³ is absent, and R⁴ is SR⁵, then R⁵ is not alkyl;
when ring A is thiophenyl, X¹ is S, X² and X³ are both CR¹, n is 0, X⁵ is O, bond a is a double bond, bond b is a single bond, R³ is absent, R⁴ is SR⁵, and R⁵ is alkyl, then R² is not aryl; and
when ring A is thiophenyl, X¹ is S, X² and X³ are both CH, n is 0, X⁵ is O, bond a is a single bond, bond b is a double bond, R³ is hydrogen, and R⁴ is S, then R² is not n-butyl, benzyl, or —CH₂-Ph-(4-ethyl).

17. The method according to claim 16, wherein bond a is a single bond, R³ is present, bond b is a double bond, and R⁴ is S.

18. The method according to claim 16, wherein bond a is a double bond, R³ is absent, bond b is a single bond, and R⁴ is H, alkyl, hydroxy, amino, or SR⁵, wherein R⁵ is selected from the group consisting of C₁₋₁₀ alkyl, cycloalkyl, acetyl, and optionally substituted 4-imidazolyl of the structure 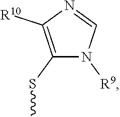
wherein R⁹ is H or alkyl, and R¹⁰ is H, alkyl, halo, haloalkyl, nitro, or sulfonamido.

19. The method according to claim 16, wherein ring A is selected from the group consisting of phenyl, pyridinyl, pyridazinyl, pyrimidyl, pyrazinyl, triazinyl, imidazolyl, 1,2,3-triazolyl, pyrazolyl, furyl, pyrrolyl, thienyl, isothiazolyl, thiazolyl, isoxazolyl, and oxadiazolyl.

20. The method according to claim 19, wherein ring A is selected from the group consisting of 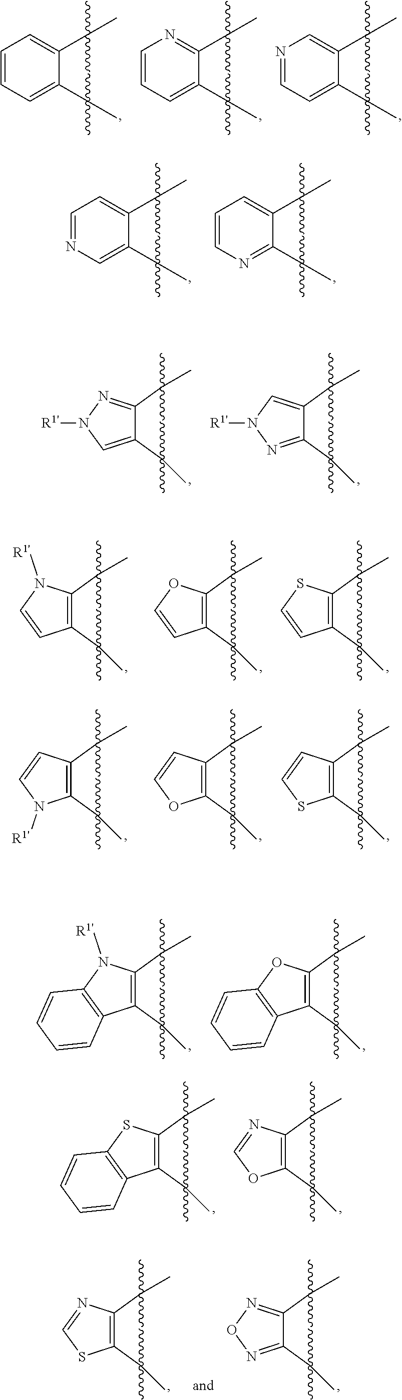
wherein each carbon is substituted with R¹, and R^{1′} is H or alkyl.

21. The method according to claim 16, wherein X⁵ is O.

22. The method according to claim 16, wherein R² is phenyl optionally substituted with alkyl, allyl, or alkyl optionally substituted with hydroxy, alkoxy, haloalkyl, cycloalkyl, heterocycloalkyl, aryl, heteroaryl, alkylsulfonamido, amino, or amido of the formula —NHC(O)(CH₂)ₘR⁶, wherein m is 0-5, and
R⁶ is alkyl, cycloalkyl, heterocycloalkyl, phenyl, and heteroaryl, each of which is optionally substituted with alkyl, alkoxy, hydroxy, halo, haloalkyl, cyano, alkoxycarbonyl (—C(O)O-alkyl), thiocyanato (—NCS), heteroarylalkyl, or a combination thereof.

23. The method according to claim 22, wherein R² is ethyl, n-propyl, —(CH₂)₃OMe, —(CH₂)₃OH, —(CH₂)₂-cyclopropyl, —(CH₂)₂-cyclobutyl, —(CH₂)₂-(4-pyridinyl), —(CH₂)₂—CF₃, —(CH₂)₂—CHF₂, —CH₂—CF₂(Me), —CH₂—CF₂CF₃, —(CH₂)₂-1-morpholino, —(CH₂)₂—NH₂, —(CH₂)₃—NHSO₂CH₃, —(CH₂)₃—NHC(O)cyclopentyl, —(CH₂)₃—NHC(O)CH₂Ph-(2-Cl), —(CH₂)₃—NHC(O)CH₂Ph-(3-OMe), —(CH₂)₃—NHC(O)CH₂Ph-(2-OMe), —(CH₂)₂—NHC(O)-Ph-(4-OH), —(CH₂)₂—NHC(O)-Ph-(3-OMe), —(CH₂)₂—NHC(O)-Ph-(4-CN), —(CH₂)₂—NHC(O)-Ph-(4-I), —(CH₂)₂—NHC(O)-Ph-(C(O)OMe), —(CH₂)₂—NHC(O)-Ph-(NCS), —(CH₂)₂—NHC(O)-Ph-(4-morpholinylmethyl).

24. The method according to claim 16, wherein R³ is H or dialkylaminoalkyl.

25. The method according to claim 16, wherein the cancer comprises cancer cells that overexpress polo-like kinase 1 (Plk 1) relative to normal cells of the same tissue type.

26. The method according to claim 25, wherein the cancer is breast cancer, lung cancer, renal cancer, liver cancer, uterine cancer, prostate cancer, pancreatic cancer, glioma, thyroid carcinoma, head and neck squamous cell carcinoma, melanoma, colorectal cancer, esophageal carcinoma, or ovarian carcinoma.

27. A compound formula (Ib-1) or (Ib-2) or a pharmaceutically acceptable salt thereof,
(i) wherein the compound of formula (Ib-1) has the structure: 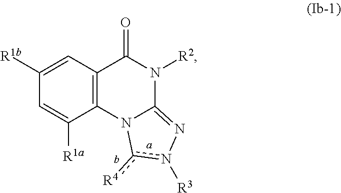
wherein
R^{1a} is H, F, or deuterium;
R^{1b} is H, deuterium, or alkoxy;
R² is selected from the group consisting of alkyl, haloalkyl, cyclopropylalkyl, 2- or 4-pyridinylalkyl, and optionally substituted benzylamidoalkyl, each of which is optionally substituted with deuterium;
R³ is H or absent;
R⁴ is S or SR⁵,
R⁵ is selected from the group consisting of C₁₋₁₀ alkyl, alkylthio (—S-alkyl), and optionally substituted 4-imidazolyl of the structure 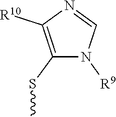
wherein R⁹ is H or alkyl, and R¹⁰ is H, alkyl, halo, haloalkyl, nitro, or sulfonamido; and
wherein the C₁₋₁₀ alkyl of R⁵ is optionally substituted with one or more substituents selected from the group consisting of deuterium, cycloalkyl, hydroxy, cyano, haloalkyl, alkylthio, amino, amido, and phenyl that is optionally substituted with one or more substituents selected from alkyl, halo, and alkenyl,
bond a is a single bond or double bond;
bond b is a single bond or double bond;
provided when bond a is a single bond, then R³ is H, bond b is a double bond, and R⁴ is S, and
when bond a is a double bond, then R³ is absent, bond b is a single bond, and R⁴ is SR⁵,
further provided that
when ring A is phenyl, X¹, X², X³, and X⁴ are each CR¹, X⁵ is O, R² is n-propyl, bond a is a single bond, bond b is a double bond, R³ is hydrogen, and R⁴ is S, then R¹ is not alkylamido;
when ring A is phenyl, X¹, X², and X⁴ are each CH, X³ is CR¹, n is 1, X⁵ is O, R² is alkyl, bond a is a single bond, bond b is a double bond, R³ is hydrogen, and R⁴ is S, then R¹ at the X³ position is not halo or hydrogen;
when ring A is phenyl, X¹, X³, and X⁴ are each CH, X² is CCH₃, n is 1, X⁵ is O, bond a is a single bond, bond b is a double bond, R³ is hydrogen, and R⁴ is S, then R² is not aryl; and
when ring A is phenyl, X¹, X³, and X⁴ are each CH, X² is C(halo), n is 1, X⁵ is O, bond a is a double bond, bond b is a single bond, R² is phenyl, R³ is absent, and R⁴ is SR⁵, then R⁵ is not alkyl, and
(ii) wherein the compound of formula (Ib-2) has the structure: 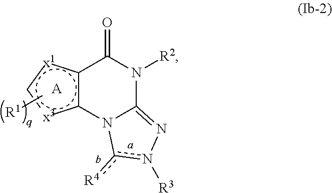
wherein
ring A is a 5-membered heteroaryl;
X¹ and X³ are each CR¹, NR^{1′}, S, or O, provided that at least one of X¹ and X³ is NR^{1′}, S, or O and the other is CR¹;
each instance of R¹ is the same or different and each is selected from the group consisting of H, deuterium, C₁₋₆ alkyl, alkoxy, halo, haloalkyl, alkylthio, alkylthioalkylenyl, cyano, amino, alkylamino, and dialkylamino, or
more than one instance of R¹ are linked to form a cycloalkyl or a phenyl, each of which is optionally substituted;
q is 1 or 2;
R^{1′} is H or alkyl;
R² is selected from the group consisting of H, alkyl, cycloalkyl, alkenyl, and aryl;
wherein the alkyl, cycloalkyl, alkenyl, and aryl of R² is optionally substituted with one or more substituents selected from the group consisting of deuterium, alkyl, alkoxy, halo, hydroxy, haloalkyl, alkoxy, alkyl, cycloalkyl, heterocycloalkyl, aryl, heteroaryl, amino, alkylamino, dialkylamino, amido, alkylsulfonamido, phosphonato, cyano, thiocyano, carboxylate, a protecting group, an amino acid residue, and a peptide residue;
R³ is H or R³ is absent;
bond a is a single bond or double bond;
bond b is a single bond or double bond;
provided when bond a is a single bond, then R³ is present, bond b is a double bond, and R⁴ is S, and
when bond a is a double bond, then R³ is absent, bond b is a single bond, and R⁴ is S—R⁵;
R⁵ is selected from the group consisting of C₁₋₁₀ alkyl, cycloalkyl, acetyl, and optionally substituted 4-imidazolyl of the structure 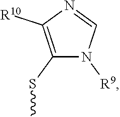
wherein R⁹ is H or alkyl, and R¹⁰ is H, alkyl, halo, haloalkyl, nitro, or sulfonamido (—SO₂NH₂); and
wherein the C₁₋₁₀ alkyl or cycloalkyl of R⁵ is optionally substituted with one or more substituents selected from the group consisting of deuterium, cycloalkyl, hydroxy, cyano, haloalkyl, alkylthio (—S-alkyl), amino, amido, and phenyl that is optionally substituted with one or more substituents selected from alkyl, halo, and alkenyl,
provided that
when bond a is a double bond, bond b is a single bond, R³ is absent, R⁴ is SR⁵, and R⁵ is alkyl, then R² is not aryl; and
when R¹ is H, bond a is a single bond, bond b is a double bond, R³ is hydrogen, and R⁴ is S, then R² is not n-butyl, benzyl, or —CH₂-Ph-(4-ethyl).

28. The compound according to claim 27 that is of formula (Ib-1) and selected from the group consisting of 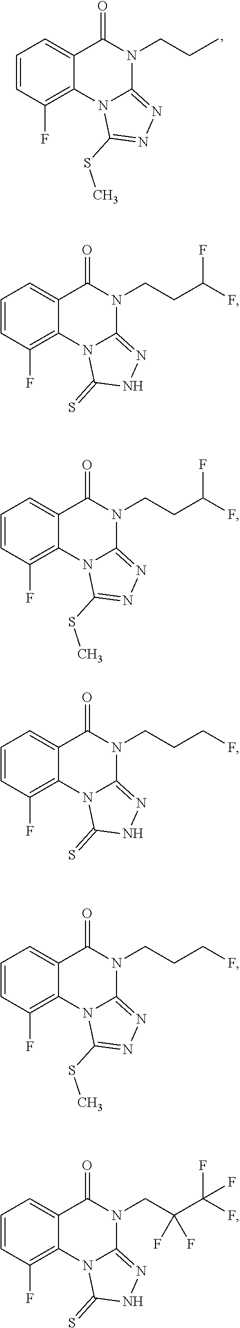 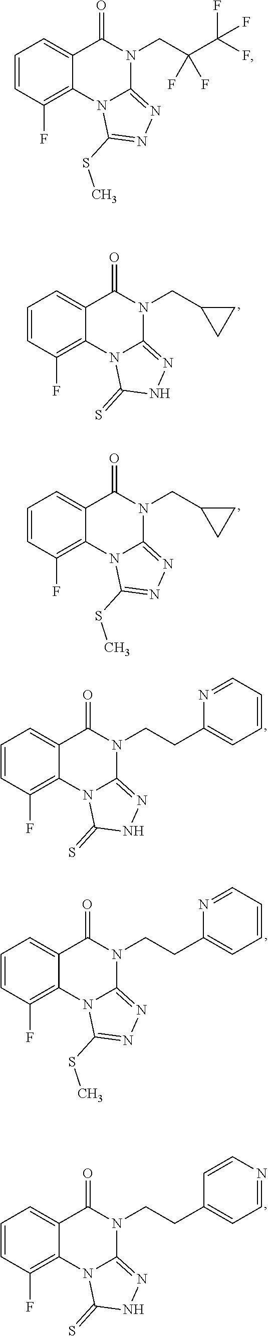 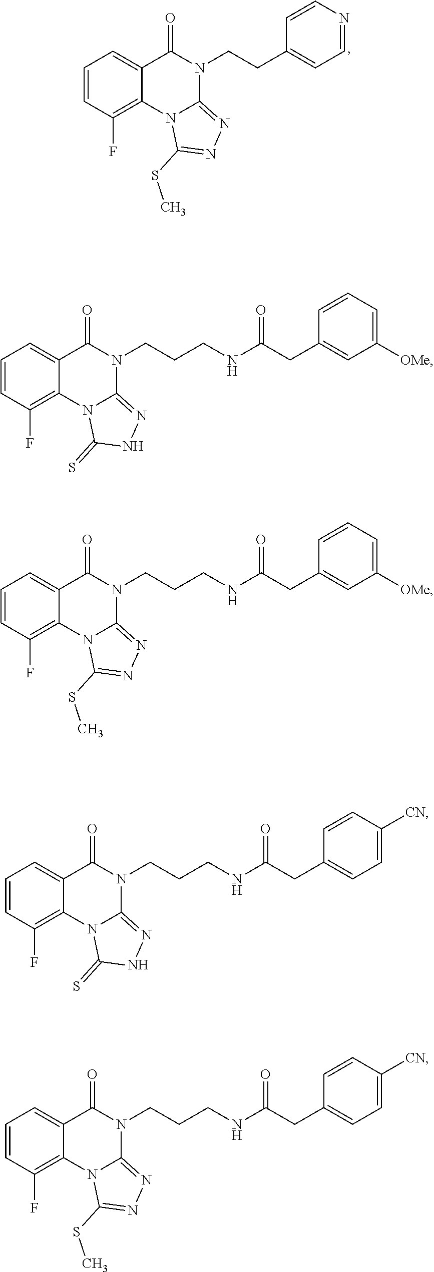 [SMILES: CSc1nnc2n(CCc3ccncc3)c(=O)c3cccc(F)c3n12 ; COc1cccc(CC(=O)NCCCn2c(=O)c3cccc(F)c3n3c(=S)[nH]nc23)c1 ; COc1cccc(CC(=O)NCCCn2c(=O)c3cccc(F)c3n3c(SC)nnc23)c1 ; N#Cc1ccc(CC(=O)NCCCn2c(=O)c3cccc(F)c3n3c(=S)[nH]nc23)cc1 ; CSc1nnc2n(CCCNC(=O)Cc3ccc(C#N)cc3)c(=O)c3cccc(F)c3n12] 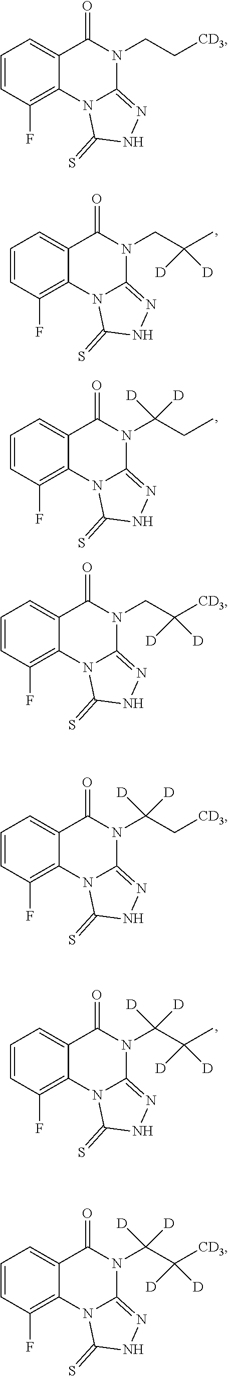 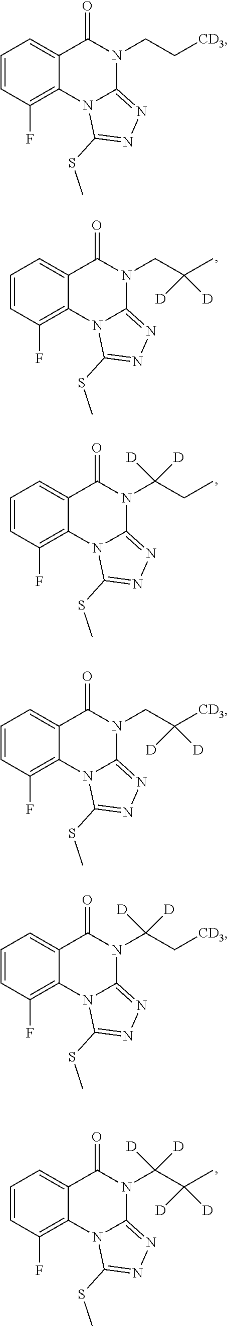 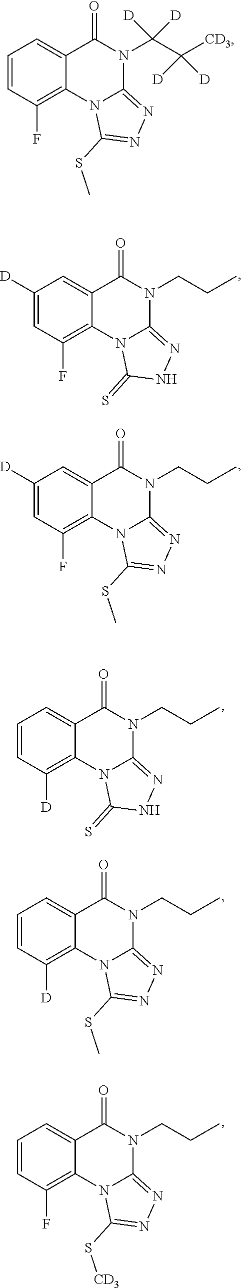 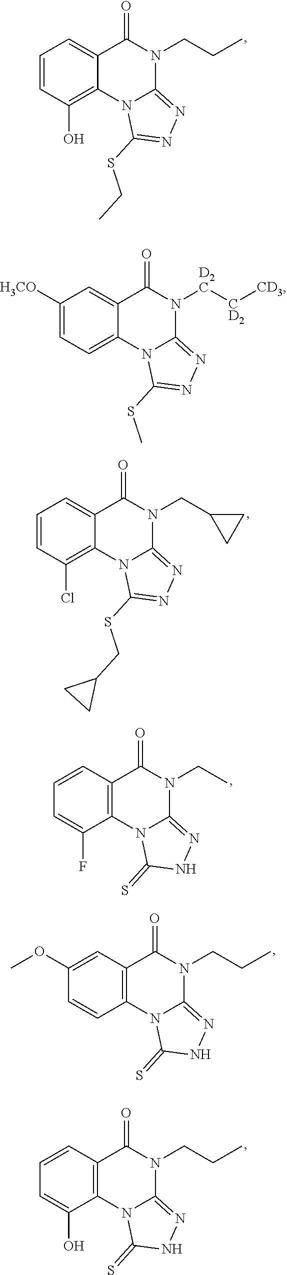 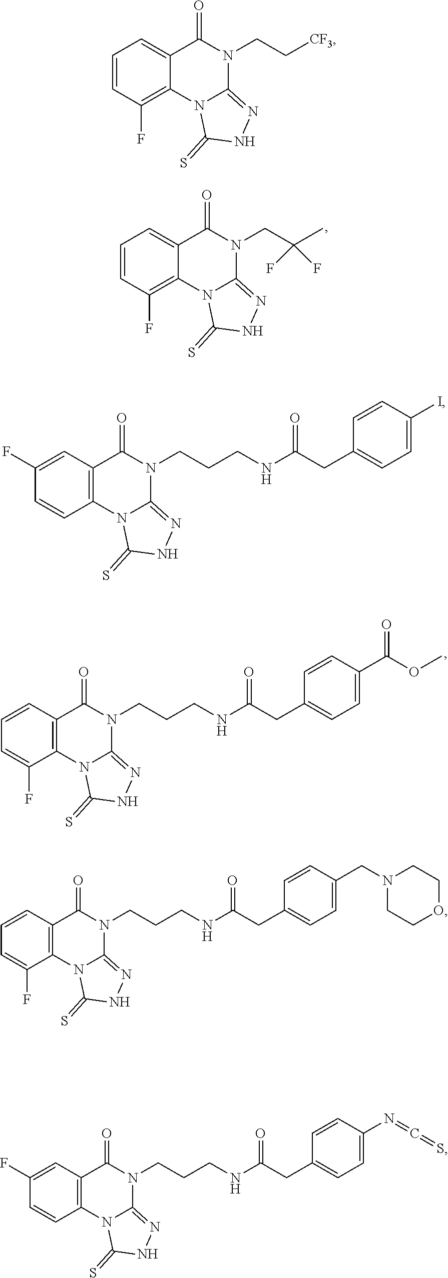 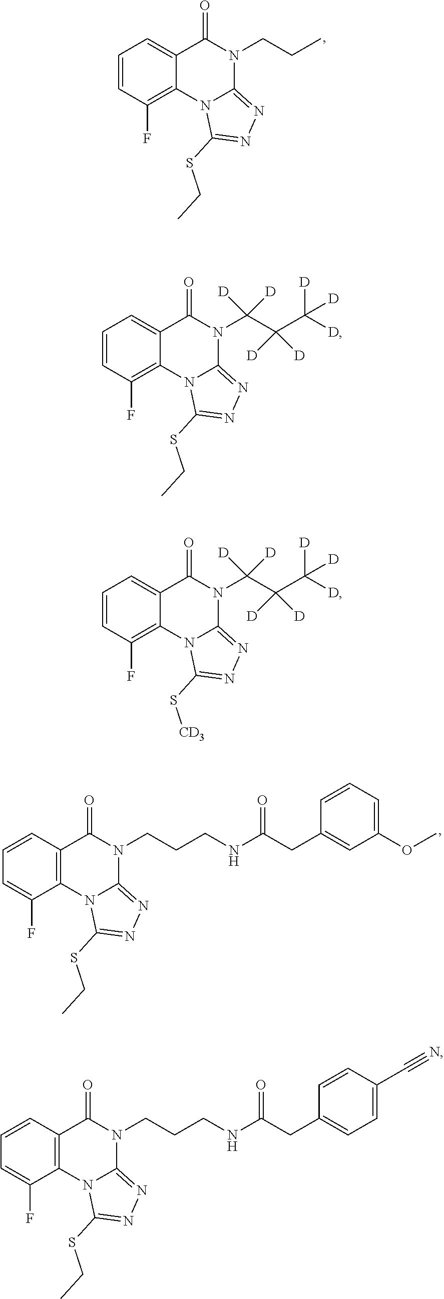 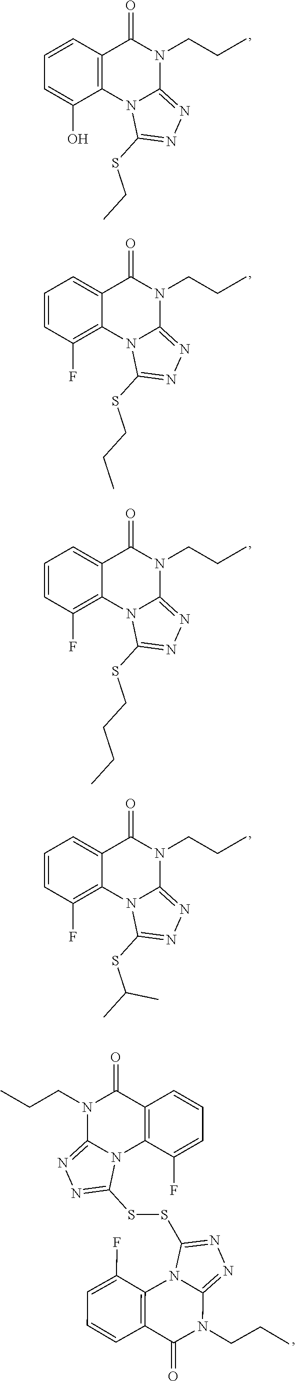 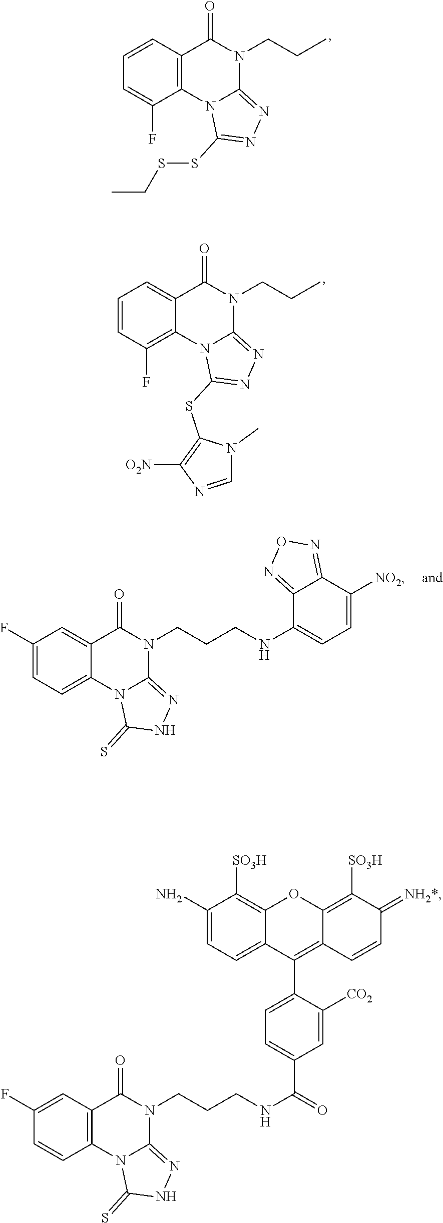
or a pharmaceutically acceptable salt thereof.

29. The compound according to claim 27 that is of formula (Ib-2) and is selected from

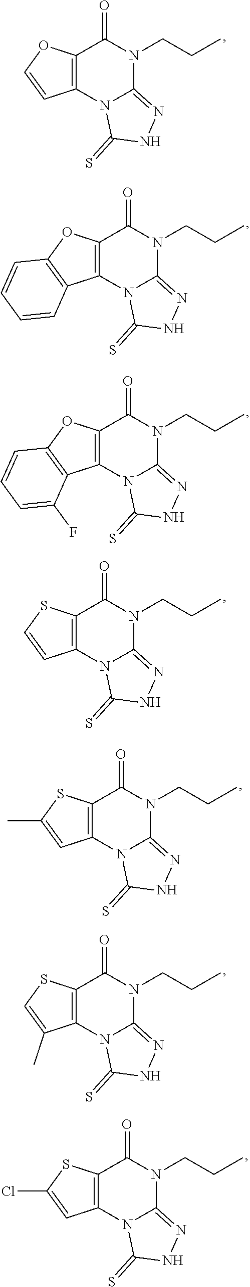

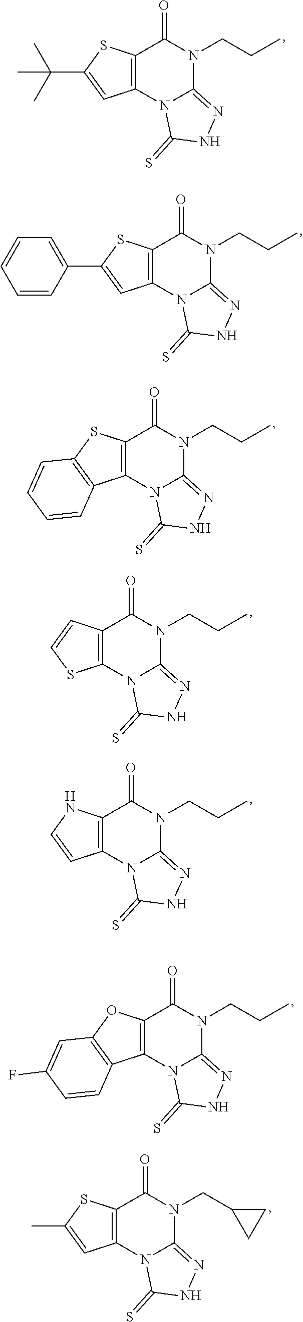

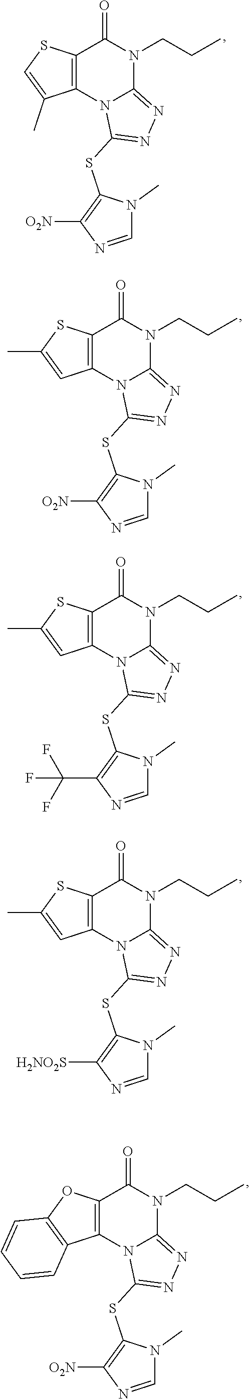

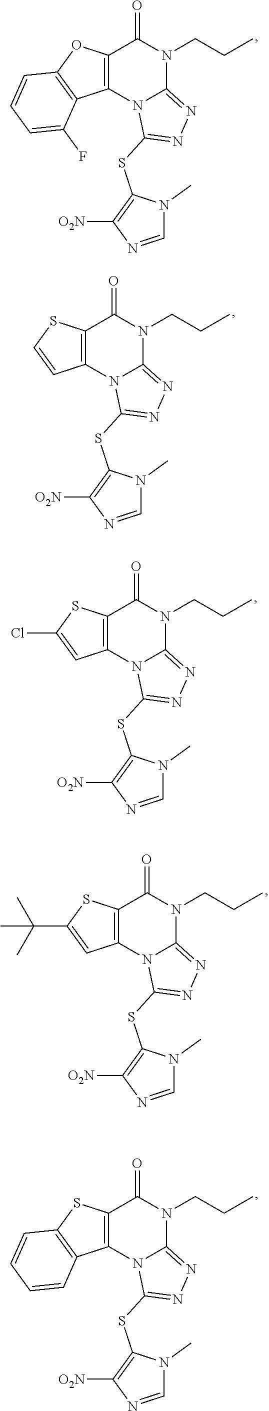

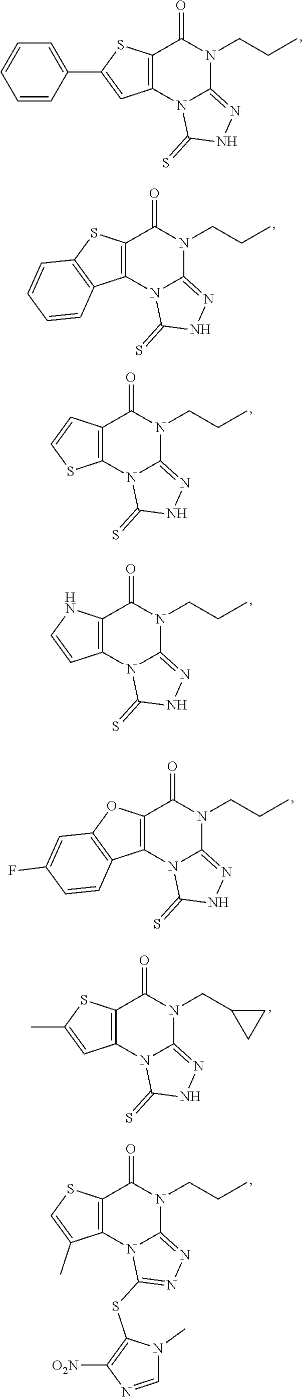

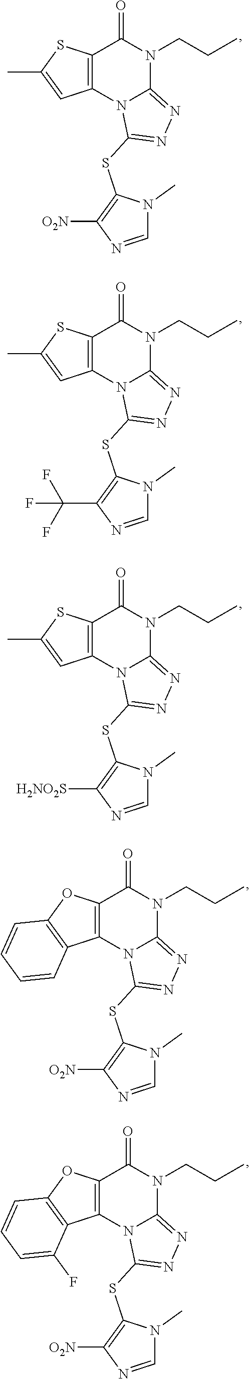

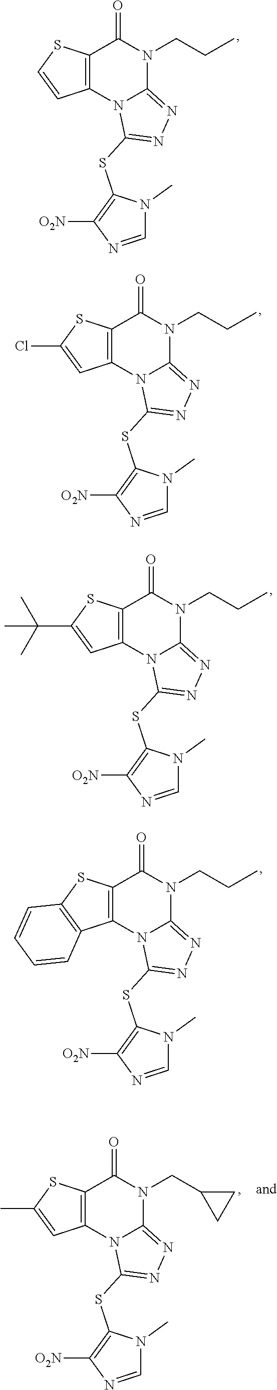

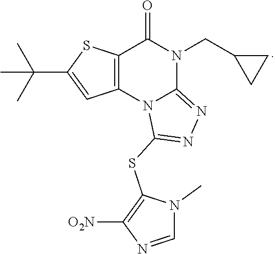 [SMILES: Cn1cnc([N+](=O)[O-])c1Sc1nnc2n(CC3CC3)c(=O)c3sc(C(C)(C)C)cc3n12]

30. A pharmaceutical composition comprising a compound of claim 27, or a pharmaceutically acceptable salt thereof, provided that the compound or salt does not contain deuterium, and a pharmaceutically acceptable carrier.

31. A method of treating cancer in a subject in need thereof comprising administering the subject an effective amount of a compound of formula (Ib-1) or (Ib-2) according to claim 27 or a pharmaceutically acceptable salt thereof, provided that the compound or salt does not contain deuterium.

32. A method of treating cancer in a subject in need thereof comprising administering the subject an effective amount of a pharmaceutical composition according to claim 30.
